(19) United States
(12) Patent Application Publication
(10) Pub. No.: US 2026/0069679 A1
(43) Pub. Date: Mar. 12, 2026

(54) CORONAVIRUS SPIKE PROTEIN-BASED VACCINES

(71) Applicant: PRESIDENT AND FELLOWS OF HARVARD COLLEGE, Cambridge, MA (US)

(72) Inventors: Charis S. PALANDJIAN, Cambridge, MA (US); Kizzmekia S. CORBETT-HELAIRE, Cambridge, MA (US); Christian K. O. DZUVOR, Cambridge, MA (US)

(73) Assignee: PRESIDENT AND FELLOWS OF HARVARD COLLEGE, Cambridge, MA (US)

(21) Appl. No.: 19/335,520

(22) Filed: Sep. 22, 2025

Related U.S. Application Data
(63) Continuation of application No. PCT/US2024/020889, filed on Mar. 21, 2024.
(60) Provisional application No. 63/453,572, filed on Mar. 21, 2023.

(51) Int. Cl. A61K 39/215; A61K 9/51; A61K 39/00; C07K 14/005

(52) U.S. Cl. CPC A61K 39/215; A61K 9/5169; C07K 14/005; A61K 2039/55555; C07K 2319/00; C12N 2770/20022; C12N 2770/20034

(57) ABSTRACT

Described herein are polypeptides and nanoparticles that display polypeptide sequences, e.g., spike protein domains. In some embodiments, the polypeptides and/or nanoparticles can be used to raise or stimulate an immune response, e.g., as a vaccine.

### CROSS-REFERENCE TO RELATED APPLICATIONS

This application is a continuation under 35 U.S.C. § 120 of co-pending International Application No. PCT/US2024/020889 filed Mar. 21, 2024, which designates the U.S. and claims benefit under 35 U.S.C. § 119(e) of U.S. Provisional Application No. 63/453,572 filed Mar. 21, 2023, the contents of which are incorporated herein by reference in their entireties.

### SEQUENCE LISTING

The instant application contains a Sequence Listing which has been submitted electronically in XML file format and is hereby incorporated by reference in its entirety. Said XML copy, created on Jul. 10, 2024, is named 002806-000112WOPT_SL.xml and is 284,119 bytes in size.

### TECHNICAL FIELD

The technology described herein provides a composition and methods of nanoparticles displaying engineered antigens to be used in immunization against different types of coronaviruses.

### BACKGROUND

As illustrated by the coronavirus pandemic, there is an urgent need for novel and improved vaccines for a variety of infectious organisms. This is particularly acute for coronavirus, where there have been multiple instances of spillovers and previously-unknown viruses causing human disease.

Existing technologies are not meeting this need, at least in part because vaccines for one strain often do not provide protection against the next variant, or related viruses. A vaccine technology that will provide strong, and broadly effective immunization is needed.

### SUMMARY

The inventors have discovered new methods for modifying and displaying antigens to induce stronger and more broadly protective immune responses. This technology is demonstrated to provide particularly effective protection for coronaviruses.

In one aspect of any of the embodiments, described herein is a polypeptide comprising: a) a display domain; b) a linker domain; and c) a multimerization domain. In some embodiments of any of the aspects, wherein the display domain comprises at least one domain of a viral spike protein. In some embodiments of any of the aspects, the display domain is at least one of:
i) is a 2P variant;
ii) not comprising a Receptor Binding Domain (RBD);
iii) not comprising a hypervariable immunogenic epitope;
iv) a concatenation of at least a first spike protein sequence and a second spike protein sequence; and
v) a concatenation of at least a first spike protein sequence from a first virus and a second spike protein sequence from a second virus.
   In some embodiments of any of the aspects, the spike protein is a coronavirus spike protein.

In one aspect of any of the embodiments, described herein is a polypeptide comprising: at least one domain of a spike protein; wherein the polypeptide is at least one of:
vi) comprises a stabilized variant of the at least one domain of a spike protein;
vii) not comprising a Receptor Binding Domain (RBD);
viii) not comprising a hypervariable immunogenic epitope;
ix) a concatenation of at least a first spike protein sequence and a second spike protein sequence; and
x) a concatenation of at least a first spike protein sequence from a first virus and a second spike protein sequence from a second virus.

In one aspect of any of the embodiments, described herein is a polypeptide comprising: at least one domain of a spike protein; wherein the at least one domain of a spike protein is at least one of:
i) comprises a stabilized variant of the at least one domain of a spike protein;
ii) not comprising a Receptor Binding Domain (RBD);
iii) not comprising a hypervariable immunogenic epitope;
iv) a concatenation of at least a first spike protein sequence and a second spike protein sequence; and
v) a concatenation of at least a first spike protein sequence from a first virus and a second spike protein sequence from a second virus.

In some embodiments of any of the aspects, the display domain, at least one domain of spike protein, and/or the polypeptide comprises a stabilized variant of the at least one domain of a spike protein and does not comprise a Receptor Binding Domain (RBD). In some embodiments of any of the aspects, the display domain, at least one domain of spike protein, and/or the polypeptide comprises a stabilized variant of the at least one domain of a spike protein and does not comprise a hypervariable immunogenic epitope. In some embodiments of any of the aspects, the display domain, at least one domain of spike protein, and/or the polypeptide does not comprise a Receptor Binding Domain (RBD) and does not comprise a hypervariable immunogenic epitope. In some embodiments of any of the aspects, the display domain, at least one domain of spike protein, and/or the polypeptide comprises a stabilized variant of the at least one domain of a spike protein, does not comprise a Receptor Binding Domain (RBD), and does not comprise a hypervariable immunogenic epitope.

In some embodiments of any of the aspects, the hypervariable immunogenic epitope is a G4 loop. In some embodiments of any of the aspects, the at least one domain of a spike protein comprises a spike protein ectodomain. In some embodiments of any of the aspects, the at least one domain of a spike protein comprises a spike protein stalk domain. In some embodiments of any of the aspects, the at least one domain of a virus spike protein comprises: a virus stalk domain from which the hypervariable immunogenic epitope is absent, and does not comprise the subunit 1 (S1) region of virus spike protein. In some embodiments of any of the aspects, the virus stalk domain comprises SEQ ID NO: 107 or SEQ ID NO: 108.

In some embodiments of any of the aspects, the polypeptide comprises a concatenation of at least a first spike protein sequence and a second spike protein sequence, and the concatenation comprises: a concatenation of at least a first N-Terminal Domain (NTD) sequence and a second NTD sequence. In some embodiments of any of the aspects, the polypeptide comprises a concatenation of at least a first spike protein sequence and a second spike protein sequence, and the concatenation comprises: a concatenation of at least a first RBD sequence and a second RBD sequence. In some embodiments of any of the aspects, the polypeptide comprises a concatenation of at least a first spike protein sequence and a second spike protein sequence, and the concatenation comprises: a concatenation of at least: a first NTD sequence, a first Subdomain 1 (SD1) sequence, and a first Subdomain 2 (SD2) sequence; and a second NTD sequence, a second SD1 sequence, and a second SD2 sequence. In some embodiments of any of the aspects, the polypeptide comprises a concatenation of at least a first spike protein sequence and a second spike protein sequence, and the concatenation comprises: a concatenation of at least: a first RBD sequence, a first SD1 sequence, and a first SD2 sequence; and a second RBD sequence, a second SD1 sequence, and a second SD2 sequence. In some embodiments of any of the aspects, the polypeptide comprises a concatenation of at least a first spike protein sequence and a second spike protein sequence, and the concatenation comprises: a concatenation of at least: a first fusion sequence; and a second fusion sequence.

In some embodiments of any of the aspects, the polypeptide comprises a concatenation of at least a first spike protein sequence from a first virus and a second spike protein sequence from a second virus, and the concatenation comprises: a concatenation of at least a first N-Terminal Domain (NTD) sequence from the first virus and a second NTD sequence from the second virus. In some embodiments of any of the aspects, the polypeptide comprises a concatenation of at least a first spike protein sequence from a first virus and a second spike protein sequence from a second virus, and the concatenation comprises: a concatenation of at least a first RBD sequence from the first virus and a second RBD sequence from the second virus. In some embodiments of any of the aspects, the polypeptide comprises a concatenation of at least a first spike protein sequence from a first virus and a second spike protein sequence from a second virus, and the concatenation comprises: a concatenation of at least: a first NTD sequence from the first virus, a first SD1 sequence from the first virus, and a first SD2 sequence from the first virus; and a second NTD sequence from the second virus, a second SD1 sequence from the second virus, and a second SD2 sequence from the second virus. In some embodiments of any of the aspects, the polypeptide comprises a concatenation of at least a first spike protein sequence from a first virus and a second spike protein sequence from a second virus, and the concatenation comprises: a concatenation of at least: a first RBD sequence from the first virus, a first SD1 sequence from the first virus, and a first SD2 sequence from the first virus; and a second RBD sequence from the second virus, a second SD1 sequence from the second virus, and a second SD2 sequence from the second virus. In some embodiments of any of the aspects, the polypeptide comprises a concatenation of at least a first spike protein sequence from a first virus and a second spike protein sequence from a second virus, and the concatenation comprises: a concatenation of at least: a first fusion sequence from the first virus; and a second fusion sequence from the second virus.

In some embodiments of any of the aspects, the spike protein is a coronavirus spike protein. In some embodiments of any of the aspects, the virus is a coronavirus. In some embodiments of any of the aspects, the first virus is a coronavirus and the second virus is a coronavirus. In some embodiments of any of the aspects, the first virus is the member of a first clade of a genus and the second virus is the member of a second clade of the virus genus. In some embodiments of any of the aspects, the coronavirus spike protein is an alphacoronavirus spike protein. In some embodiments of any of the aspects, the coronavirus spike protein is a betacoronavirus spike protein. In some embodiments of any of the aspects, the coronavirus spike protein is a MERS-CoV spike protein, SARS-CoV-1 spike protein, SARS-CoV-2 spike protein, hCoV-HKU1 spike protein, hCoV-HL63 spike protein, hCoV-229E spike protein, or hCoV-OC43 spike protein.

In some embodiments of any of the aspects, the at least one domain of a virus spike protein comprises a sequence with at least 80% sequence identity to the sequence of one of SEQ ID NOs: 21-27, 78-84, 90-96, 123-134, and 143-150. In some embodiments of any of the aspects, the at least one domain of a virus spike protein comprises the sequence of one of SEQ ID NOs: 21-27, 78-84, 90-96, 123-134, and 143-150.

In some embodiments of any of the aspects, the polypeptide further comprises a multimerization domain. In some embodiments of any of the aspects, the multimerization domain is a trimerization domain. In some embodiments of any of the aspects, the trimerization domain is I53_dn5B. In some embodiments of any of the aspects, the multimerization domain is foldon, Apoferritin, Lumazine Synthase, or I53_dn5B. In some embodiments of any of the aspects, the multimerization domain comprises a sequence with at least 80% sequence identity to the sequence of one of SEQ ID NOs: 17-20, 97, and 122. In some embodiments of any of the aspects, the multimerization domain comprises the sequence of one of SEQ ID NOs: 17-20, 97, and 122.

In some embodiments of any of the aspects, the polypeptide further comprises a linker domain. In some embodiments of any of the aspects, the linker domain is located between the a) the display domain, and c) the multimerization domain. In some embodiments of any of the aspects, the linker domain comprises the sequence of one of SEQ ID NOs: 1-9. In some embodiments of any of the aspects, the linker domain comprises the sequence of one of SEQ ID NOs: 1-7. In some embodiments of any of the aspects, the linker domain comprises the sequence of one of SEQ ID NOs: 2 and 6. In some embodiments of any of the aspects, the linker domain comprises the sequence of SEQ ID NO: 2.

In some embodiments of any of the aspects, the polypeptide comprises a sequence with at least 80% sequence identity to the sequence of one of SEQ ID NOs: 21-77, 85-96, 109-120, and 135-142. In some embodiments of any of the aspects, the polypeptide comprises the sequence of one of SEQ ID NOs: 21-77, 85-96, 109-120, and 135-142.

In one aspect of any of the embodiments, described herein is a polypeptide complex comprising a multimer of: a first polypeptide as described herein; and at least a second polypeptide as described herein. In some embodiments of any of the aspects, the first polypeptide and the second polypeptide are identical. In some embodiments of any of the aspects, the first polypeptide and the second polypeptide are not identical. In some embodiments of any of the aspects, the first polypeptide comprises at least one domain of a virus spike protein of a first virus and the second polypeptide comprises at least one domain of a virus spike protein of a second virus.

In one aspect of any of the embodiments, described herein is a nucleic acid encoding at least one polypeptide as described herein.

In one aspect of any of the embodiments, described herein is a nanoparticle comprising at least one polypeptide or polypeptide complex as described herein. In some embodiments of any of the aspects, the nanoparticle further comprises one or more I53_dn5A or I53-50B.4PT1 polypeptides.

In one aspect of any of the embodiments, described herein is a pharmaceutical composition comprising at least one polypeptide, polypeptide complex, or nanoparticle as described herein. In one aspect of any of the embodiments, described herein is a method of immunizing a subject, the method comprising administering at least one polypeptide, polypeptide complex, nanoparticle, or pharmaceutical composition as described herein to the subject. In one aspect of any of the embodiments, described herein is a method of stimulating an immune response of a subject, the method comprising administering at least one polypeptide, polypeptide complex, nanoparticle, or pharmaceutical composition as described herein to the subject.

### BRIEF DESCRIPTION OF THE DRAWINGS

FIG. 1 depicts a schematic of the experimental workflow in Example 2.

FIGS. 2A-2E depicts a schematic of the experimental workflow in Example 2. (FIG. 2A) Expression optimization. Plasmids encoding for immunogens or antibodies were transiently transfected into Expi293F suspension cells. (FIG. 2B) Protein purification. Secreted proteins in the supernatant of the suspension cells were purified using batch affinity purification. Depending on the immunogen or antibody, strep-tacin, Ni-NTA, GNL Lectin, and Protein A purifications were used. These purified proteins were then run on an SDS-PAGE gel. (FIG. 2C) Biochemical optimization and analysis. Using size exclusion chromatography, the purity, aggregation, and size distribution was assessed (FIG. 2D) Biophysical and structural analysis. Using negative stain- and cryo-electron microscopy, the size distribution, stability, and morphology was assessed. (FIG. 2E) Antigenic characterization using BLI with Octet BLI instrument. Samples are loaded onto a black plate and shown is a recommended layout for determining antigenicity of nanoparticle vaccine candidates. White wells indicate assay buffer. Yellow wells indicate antibody. One antibody well was filled with a negative control antibody that did not bind to the immunogen. Pink wells indicate samples. Samples are prepared with a two-fold serial dilution down the plate. One sample well has assay buffer as a negative control and baseline to subtract from the data. Green wells indicate regeneration buffer. Probes dip into regeneration buffer then assay buffer three times in between runs to regenerate the probes and wash off antibody/samples. Schematic representation of different BLI steps (right). Probes with protein A biosensors were dipped into wells containing the target antibodies (yellow wells), and the antibodies loaded onto the probe. Probes then dipped into wells containing the NPs, and the association of the immunogens to the antibodies was measured. Probes then dipped into assay buffer, and the dissociation from the antibodies was measured. The binding was measured in nm, and an example of binding curve data is represented for each step in the graph below the probes. Created with BioRender.com.

FIGS. 3A-3C depict the rational design of spike component nanoparticles. (FIG. 3A) Schematic representation/diagram of an icosahedral apoferritin model (left) and octahedral lumazine synthase model (right). PDB models (1FHA, 1HQK) were used respectively. The antigen fusion/insertion sites are indicated as circles. Size in nm is indicated by reference bars. (FIG. 3B) Schematic representation of the flexible linker (left) and the rigid helical linker (right), predicted with pep fold 3. (FIG. 3C) Schematic representation/diagram for molecular design of engineered spike component nanoparticle vaccines (left) and a rendered full spike protein (right) using UCSF chimeraX. Shading on the spike protein correspond to domains represented in the constructs. The constructs show the designed spike component fused to a linker and either the Aferr or LuS nanoparticle. For the SS construct, the point mutations in the S2 are denoted by lines. The G4Loop epitope and deletion is indicated by the zoomed box. FIG. 3C discloses SEQ ID NOS 232-234, respectively, in order of appearance.

FIGS. 4A-4I depict the biophysical characterization of nanoparticle vaccine candidates. (FIG. 4A) SDS-PAGE gel of purified antigens. (FIG. 4B) SDS-PAGE gel of apoferritin nanoparticle vaccine candidates. (FIG. 4C) SDS-PAGE Gel of lumazine synthase nanoparticle vaccine candidates. (FIGS. 4A-4C) Molecular weight standards are indicated in kDa. Boxes highlight the bands corresponding to immunogens. (FIG. 4D) Size exclusion chromatography on Superose 6 10/300 column for representative vaccine antigens. (FIG. 4E) Size exclusion chromatography on Superose 6 10/300 column for representative apoferritin vaccine candidates. (FIG. 4F) Size exclusion chromatography on Superose 6 10/300 column for representative lumazine synthase vaccine candidates. (FIG. 4G) Negative stain Electron Microscopy of purified apoferritin nanoparticles. (FIG. 4H) Negative-stain Electron Microscopy of purified lumazine synthase nanoparticles. (FIGS. 4F-4G) The scale bar is 50 nm. In the bottom corner are 2D class averages of images obtained using single-particle EM in cryoSPARC software. (FIG. 4I) Cryo-EM 3D reconstruction model (down) of MERS SS-L6-LuS nanoparticles, refined with icosahedral symmetry. The Lumazine synthase atomic model (pdb:1HQK) was rendered and fitted into the cryo-EM density map (top).

FIGS. 5A-5D depicts the antigenic characterization of nanoparticle vaccine candidates. Biolayer interferometry sensograms of: (FIG. 5A) S2PΔRBD-L6-Aferr (left) and S2PΔRBD-L6-LuS (right) binding to antibodies G2 and G4. (FIG. 5B) SS-L6-Aferr (left) and SS-L6-LuS (right) binding to antibodies IgG22 and IgG72. (FIG. 5C) SSAG4Loop-L6-Aferr (left) and SSAG4Loop-L6-LuS (right) binding to antibodies IgG22 and G4. (FIG. 5D) NTD-L6-Aferr (left) and NTD-L6-LuS (right) binding to antibodies A2 and G2. (FIGS. 5A-5D) Concentration legend shown at bottom; Binding response measured by BLI, with association from seconds 0-180 seconds and dissociation from 180-480 seconds.

FIGS. 6A-6D depicts epitope accessibility and qualitative comparison of the antibody binding to vaccine candidates. Biolayer interferometry sensograms of: (FIG. 6A) NTD antigen (left) and NTD-L2-LuS (right) binding to antibody A2. (FIG. 6B) Bar graph of highest binding (nm) to A2 antibody for 1500 nM and 100 nM of the NTD antigen and 100 nM of the NTD-L2-LuS nanoparticle. (FIG. 6C) SS Antigen (left) and SS-L2-LuS (right) binding to antibody G4. (FIG. 6D) Bar graph of highest binding (nm) to G4 antibody for 1500 nM and 100 nM of the SS antigen and 100 nM of the SS-L2-LuS nanoparticle. (FIG. 6A, 6C) Concentration legend shown at bottom; concentrations 100 nM to 3.125 nM were made by two-fold serial dilutions starting at 100 nM. Binding response measured by BLI, with association from 0-800 seconds and dissociation from 800-1100 seconds (FIG. 6B, 6D).

FIGS. 7A-7D demonstrate that nanoparticle vaccine immunogens with a rigid linker expose the conserved helical stalk epitopes. Biolayer interferometry sensograms of: (FIG. 7A) S2PΔRBD-L2-Aferr (left) and S2PΔRBD-L6-Aferr (right) binding to antibody CC40.8. (FIG. 7B) S2PΔRBD-L2-LuS (left) and S2PΔRBD-L6-LuS (right) binding to antibody CC40.8. (FIG. 7C) S2PΔRBD antigen binding to antibody CC40.8. (FIG. 7D) LuS nanoparticle (no antigen on display) binding to antibody CC40.8. (FIGS. 7A-7D) Concentration legend shown at bottom; concentrations were made by serial dilution starting at 100 nM. Binding response measured by BLI, with association from seconds 0-180 and dissociation from seconds 180-480. Baseline of assay buffer only wells was subtracted from data. Y-axis was aligned to the average of the baseline step. The inter-step correction was aligned to the dissociation step. Data was filtered with Savitzky-Golay filtering to remove high-frequency noise from the data.

FIGS. 8A-8C depict a proposed model of possible nanoparticle vaccine candidates' immune induction inferred from antigenic characterization. (FIG. 8A) Possible B-cell activation via the interaction of antigen (left) or nanoparticle immunogens (right) with BCRs embedded within the B-cell membrane. The arrows indicate activation signals from the respective immunogens. The weak antigen-antibody binding will likely result in weak activation signal/B-cell recognition, response, and antibody production. The strong antibody-nanoparticle immunogen binding due to multivalent antigen interaction/crosslinking with multiple BCRs could induce the production of more antibodies and cross-reactive B-cells. (FIG. 8B) Predicted or anticipated immune outcome induced by antigen stimulation (top) and nanoparticle immunogen stimulation (bottom). (FIG. 8C) A schematic showing that rare antibody (IgG22 or CC40.8) affinity/binding to exposed conserved epitopes on engineered nanoparticle group (L2, right) could enable more BCR access to S2/stalk epitopes and induce more cross-reactive responses compared to respective nanoparticle, NPs group (L6, left) and antigens (SS or S2PΔRBD). Created with BioRender.com.

FIG. 9 depicts future directions of animal immunization and experiments of promising vaccine candidates. BALB/cJ and mutated mouse models will be immunized twice at weeks 0 and 3 with each of the promising nanoparticle immunogens based on antigenicity data. Control mice will be immunized with a bare nanoparticle. Mice will be bled and sampled at week 2 and 5 for serological assessments. Sera will be screened by ELISA for IgG binding to each strain of β-CoV. Neutralizing antibody responses will be evaluated through pseudovirus neutralization assays. Fc mediated effector functions will also be assessed. Upon harvesting and processing the mice splenocytes at week 5, specific cellular responses will also be studied using flow cytometry with a focus on CD8+ and CD4+ T cells in one study. In another study, humanized DPP4-mice (288/330^{+/+}) and K18-human ACE2 transgenic mice will be challenged with a lethal dose of mouse-adapted β-CoV at least four weeks after second immunization (week 9). Following the challenge, mice will be monitored for clinical signs, body weight loss, and survival and euthanized to investigate viral replication, lung discoloration, and viral burden in the upper and lower airways.

FIG. 10 depicts the binding of spike protein to a receptor.

FIG. 11 depicts a schematic of MERS-CoV mutations eluding immunity.

FIGS. 12A-12B depict S2 conservation and variable epitope sequences among lineage C betacoronaviruses. FIG. 12 B discloses SEQ ID NOS 235 and 235-243, respectively, in order of appearance.

FIGS. 13A-13E depict a workflow.

FIG. 14 depicts rational immunogen design. FIG. 14 discloses SEQ ID NOS 232-234, respectively, in order of appearance.

FIGS. 15A-15C depict structural characterization of nanoparticle immunogens.

FIGS. 16A-16C depict antigenic characterization of MERS SS nanoparticle vaccines.

FIG. 17 depicts antigenic characterization of MERS SS nanoparticle vaccines.

FIGS. 18A-18C depict antigenic characterization of MERS SS nanoparticle vaccines.

FIGS. 19A-19E depict antigenic characterization of MERS SS nanoparticle vaccines.

FIGS. 20A-20C characterization of pan-nanoparticle vaccines in mouse models.

FIGS. 21A-21B depict the phylogeny of coronaviruses and the similarity of coronavirus spike proteins.

FIGS. 22A-22E depicts analysis of (FIG. 22A) HCoV-HKU1 next strain analysis, (FIGS. 22B and 22D) dendrograms, and (FIGS. 22C and 22E) sequence identity matrices.

FIG. 23 depicts coronavirus spike protein structure and immunodominant domain (IDD) antigen design.

FIG. 24A depicts schematics of three different nanoparticle platforms. FIG. 24B depicts a schematic of the I53-50 nanoparticle platform. FIG. 24C depicts a schematic of an exemplary spike nanoparticle assembly. FIG. 24D depicts a schematic of an exemplary immunodominant domain nanoparticle assembly.

FIG. 25 depicts simulated 3D models of two exemplary nanoparticles.

FIG. 26A depicts antigen expression and purification results. FIGS. 26B and 26C depict nanoparticle assembly and biochemical characterization.

FIG. 27 depicts structural characterization of nanoparticles.

FIGS. 28A-28B depicts antigenic characterization of nanoparticles.

FIGS. 29A-29B depict antigenic characterization, e.g., the IDD binding response.

FIG. 30 depicts antigen production for S2P.

FIGS. 31A-31B depict nanoparticle structural characterization for IDD (FIG. 31A) and S2P (FIG. 31B).

FIGS. 32A-32B depict antigenic characterization, e.g., the S2P binding response.

FIGS. 33A-33B depict a table of the determination of antibody binding affinities of vaccine immunogens using BLI. a Kₒₙ is the association rate constant. b K_{off} is the dissociation rate constant. c K_{D} is the equilibrium dissociation constant, calculated by dividing K_{off} by Kₒₙ. d NB denotes no detectable binding. e ND denotes that antibody binding to immunogen was not determined. f Values with <sign denote that K_{D} value was beyond the BLI equipment's limit of detection.

### DETAILED DESCRIPTION

As demonstrated herein, the inventors have designed protein display technology that provides improved immunogenicity and/or antigenicity characteristics. One aspect of the improved protein display technology is the use of novel linker domains that provide optimized geometry for protein display and immunological responses to the protein. Other aspects of the improved protein display technology relate to certain modifications of viral spike proteins that provide engineered polypeptides that exhibit improved immunogenicity and/or antigenicity characteristics compared to unmodified spike proteins. For example, the engineered polypeptides described herein induce production of more broadly protective antibodies.

In one aspect of any of the embodiments, described herein is a polypeptide comprising a display domain, a linker domain; and a multimerization domain. As used herein, “display domain” refers to any polypeptide, or domain thereof, that is to be displayed for a protein-protein or protein-cell interaction. In some embodiments of any of the aspects, the display domain can be a known antigen polypeptide, or domain thereof, or antigenic portion thereof. In some embodiments of any of the aspects, the display domain can be a viral antigen polypeptide, or domain thereof, or antigenic portion thereof. In some embodiments of any of the aspects, the display domain can be a bacterial antigen polypeptide, or domain thereof, or antigenic portion thereof. In some embodiments of any of the aspects, the display domain can be a cancer antigen polypeptide, or domain thereof, or antigenic portion thereof. Antigen polypeptides are well known in the art. For example, for further discussion of antigen polypeptides, see Feola et al. Cancers (Basel) 12:1160 (2020), Lu et al. J of Biomed Sci 27:1 (2020), Ong et al. Front Immunol 8:1382 (2017), He et al. Procedia in Vaccinology 6:3-9 (2012), Nuti et al. mBio 2:10.1128 (2011), Murin et al. Nat Microbiol 4:734-747 (2019); each of which is incorporated by reference herein in its entirety.

In some embodiments of any of the aspects, a display domain comprises at least one domain of a viral spike protein. In some embodiments of any of the aspects, a display domain comprises at least one domain of a coronavirus spike protein. In some embodiments of any of the aspects, a display domain comprises least one domain of a viral spike protein which is at least one of
is a 2P variant;
not comprising a Receptor Binding Domain (RBD);
not comprising a hypervariable immunogenic epitope;
a concatenation of at least a first spike protein sequence and a second spike protein sequence; and
a concatenation of at least a first spike protein sequence from a first virus and a second spike protein sequence from a second virus.

In one aspect of any of the embodiments, described herein is a polypeptide comprising at least one domain of a spike protein; wherein the polypeptide is at least one of:
i) is a 2P variant;
ii) not comprising a Receptor Binding Domain (RBD);
iii) not comprising a hypervariable immunogenic epitope;
iv) a concatenation of at least a first spike protein sequence and a second spike protein sequence; and
v) a concatenation of at least a first spike protein sequence from a first virus and a second spike protein sequence from a second virus.

As used herein, “spike protein” refers to a viral protein that projects from the surface of an enveloped virus. Spike proteins often form dimers or trimers and are often rod or club shaped with large ectodomain, a transmembrane domain, and a short domain inside the viral envelope. A spike protein can be an orthomyxovirus spike protein, a paramyxovirus spike protein, a rhabdovirus spike protein, a filovirus spike protein, a coronavirus spike protein, a bunyavirus spike protein, an arenavirus spike protein, or a retrovirus spike protein. In some embodiments of any of the aspects, the viral spike protein is a coronavirus spike protein.

The coronavirus spike protein, as displayed on the viral surface, is a trimeric protein of S1-S2 heterodimers. The subunit 1 (S1) region (corresponding to residues 14-685 of SEQ ID NO: 103) is a hypervariable head domain of the spike protein, and comprises the receptor binding domain (RBD) (corresponding to residues 367-588 of SEQ ID NO: 100 and corresponding to residues 33-528 of SEQ ID NO: 103), the N-terminal domain (NTD) (corresponding to residues 14-306 of SEQ ID NO: 100 and corresponding to residues 13-304 of SEQ ID NO: 103), Subdomain 1 (SD1) (corresponding to residues 528-591 of SEQ ID NO: 103), and Subdomain 2 (SD2) (corresponding to residues 591-685 of SEQ ID NO: 103). The subunit 2 (S2) region (corresponding to residues 686-1273 of SEQ ID NO: 103) is the stem of the spike protein and comprises a fusion domain (corresponding to residues 816-833 of SEQ ID NO: 103), a hypervariable immunogenic epitope or G4 loop (corresponding to residues 1178-1183 of SEQ ID NO: 100 or corresponding to residues 1090-1091 of SEQ ID NO: 103) in a stalk domain (corresponding to residues 762-1291 of SEQ ID NO: 100 or corresponding to residues 686-1211 of SEQ ID NO: 103). The coronavirus ectodomain corresponds to residues 18-1291 of SEQ ID NO: 100 or residues 14-1211 of SEQ ID NO: 103). The foregoing sequences are exemplified with reference to SEQ ID NO: 100 and SEQ ID NO: 103. The corresponding sequences in other coronavirus spike proteins are readily identified by alignment of a spike protein sequence of interest with SEQ ID NO: 100 and/or SEQ ID NO: 103. Further discussion of spike protein structure can be found, e.g., in Hsieh et al. Cell Rep 37(5):109929 (2021), Magazine et al. Viruses 14:640 (2022), Li et al. Annu Rev Virol 3:237-261 (2016), Huang et al. Acta Pharmacol Sin 41:1141-1149 (2020), Schaub et al. Nature Protocols 16:5339-5356 (2021), and Koppisetti et al. J Am Chem Soc 143:13205-13211 (2021); each of which is incorporated by reference herein in its entirety. Further exemplary coronaviruses and their sequences are provided in Table 4.

An exemplary spike protein is the Human betacoronavirus 2c EMC/2012 spike protein (SEQ ID NO: 100), NCBI protein ID AFS88936.1

SEQ ID NO: 100
MIHSVFLLMFLLTPTESYVDVGPDSVKSACIEVDIQQTFFDKTWPRPIDV
SKADGIIYPQGRTYSNITITYQGLFPYQGDHGDMYVYSAGHATGTTPQKL
FVANYSQDVKQFANGFVVRIGAAANSTGTVIISPSTSATIRKIYPAFMLG
SSVGNFSDGKMGRFFNHTLVLLPDGCGTLLRAFYCILEPRSGNHCPAGNS
YTSFATYHTPATDCSDGNYNRNASLNSFKEYFNLRNCTEMYTYNITEDEI
LEWFGITQTAQGVHLFSSRYVDLYGGNMFQFATLPVYDTIKYYSIIPHSI
RSIQSDRKAWAAFYVYKLQPLTELLDESVDGYIRRAIDCGENDLSQLHCS
YESFDVESGVYSVSSFEAKPSGSVVEQAEGVECDESPLLSGTPPQVYNFK
RLVFTNCNYNLTKLLSLFSVNDFTCSQISPAAIASNCYSSLILDYFSYPL
SMKSDLSVSSAGPISQFNYKQSFSNPTCLILATVPHNLTTITKPLKYSYI
NKCSRLLSDDRTEVPQLVNANQYSPCVSIVPSTVWEDGDYYRKQLSPLEG
GGWLVASGSTVAMTEQLQMGFGITVQYGTDTNSVCPKLEFANDTKIASQL
GNCVEYSLYGVSGRGVFQNCTAVGVRQQRFVYDAYQNLVGYYSDDGNYYC
LRACVSVPVSVIYDKETKTHATLFGSVACEHISSTMSQYSRSTRSMLKRR
DSTYGPLQTPVGCVLGLVNSSLFVEDCKLPLGQSLCALPDTPSTLTPRSV
RSVPGEMRLASIAFNHPIQVDQLNSSYFKLSIPTNFSFGVTQEYIQTTIQ
KVTVDCKQYVCNGFQKCEQLLREYGQFCSKINQALHGANLRQDDSVRNLF
ASVKSSQSSPIIPGFGGDENLILLEPVSISTGSRSARSAIEDLLEDKVTI
ADPGYMQGYDDCMQQGPASARDLICAQYVAGYKVLPPLMDVNMEAAYTSS
LLGSIAGVGWTAGLSSFAAIPFAQSIFYRLNGVGITQQVLSENQKLIANK
ENQALGAMQTGFTTTNEAFQKVQDAVNNNAQALSKLASELSNTFGAISAS
IGDIIQRLDVLEQDAQIDRLINGRLTTLNAFVAQQLVRSESAALSAQLAK
DKVNECVKAQSKRSGFCGQGTHIVSFVVNAPNGLYEMHVGYYPSNHIEVV
SAYGLCDAANPTNCIAPVNGYFIKTNNTRIVDEWSYTGSSFYAPEPITSL
NTKYVAPQVTYQNISTNLPPPLLGNSTGIDFQDELDEFFKNVSTSIPNFG
SLTQINTTLLDLTYEMLSLQQVVKALNESYIDLKELGNYTYYNKWPWYIW
LGFIAGLVALALCVFFILCCTGCGTNCMGKLKCNRCCDRYEEYDLEPHKV
HVH

An exemplary spike protein is the SARS-CoV2 spike protein (SEQ ID NO: 103), NCBI protein ID YP_009724390.1.

SEQ ID NO: 103

| | |
|---|---|
| 1 | mfvflvllpl vssqcvnltt rtqlppaytn sftrgvyypd |
| | kvfrssvlhs tqdlflpffs |
| 61 | nvtwfhaihv sgtngtkrfd npvlpfndgv yfasteksni |
| | irgwifgttl dsktqslliv |
| 121 | nnatnvvikv cefqfcndpf lgvyyhknnk swmesefrvy |
| | ssannctfey vsqpflmdle |
| 181 | gkqgnfknlr efvfknidgy fkiyskhtpi nlvrdlpqgf |
| | saleplvdlp iginitrfqt |
| 241 | llalhrsylt pgdsssgwta gaaayyvgyl qprtfllkyn |
| | engtitdavd caldplsetk |
| 301 | ctlksftvek giyqtsnfrv qptesivrfp nitnlcpfge |
| | vfnatrfasv yawnrkrisn |
| 361 | cvadysvlyn sasfstfkcy gvsptklndl cftnvyadsf |
| | virgdevrqi apgqtgkiad |
| 421 | ynyklpddft gcviawnsnn ldskvggnyn ylyrlfrksn |
| | lkpferdist eiyqagstpc |
| 481 | ngvegfncyf plqsygfqpt ngvgyqpyrv vvlsfellha |
| | patvcgpkks tnlvknkcvn |
| 541 | fnfngltgtg vltesnkkfl pfqqfgrdia dttdavrdpq |
| | tleilditpc sfggvsvitp |
| 601 | gtntsnqvav lyqdvnctev pvaihadqlt ptwrvystgs |
| | nvfqtragcl igaehvnnsy |
| 661 | ecdipigagi casyqtqtns prrarsvasq siiaytmslg |
| | aensvaysnn siaiptnfti |
| 721 | svtteilpvs mtktsvdctm yicgdstecs nlllqygsfc |
| | tqlnraltgi aveqdkntqe |
| 781 | vfaqvkqiyk tppikdfggf nfsqilpdps kpskrsfied |
| | llfnkvtlad agfikqygdc |
| 841 | lgdiaardli caqkingltv lpplltdemi aqytsallag |
| | titsgwtfga gaalqipfam |
| 901 | qmayrfngig vtqnvlyenq klianqfnsa igkiqdslss |
| | tasalgklqd vvnqnagaln |
| 961 | tlvkqlssnf gaissvlndi lsrldkveae vqidrlitgr |
| | lqslqtyvtq qliraaeira |
| 1021 | sanlaatkms ecvlgqskrv dfcgkgyhlm sfpqsaphgv |
| | vflhvtyvpa qeknfttapa |
| 1081 | ichdgkahfp regvfvsngt hwfvtqrnfy epqiittdnt |
| | fvsgncdvvi givnntvydp |
| 1141 | lqpeldsfke eldkyfknht spdvdlgdis ginasvvniq |
| | keidrlneva knlneslidl |
| 1201 | qelgkyeqyi kwpwyiwlgf iagliaivmv timlccmtsc |
| | csclkgccsc gscckfdedd |
| 1261 | sepvlkgvkl hyt |

In some embodiments of any of the aspects, the polypeptide comprising at least one spike protein domain comprises at least a spike protein ectodomain.

In some embodiments of any of the aspects, a spike protein ectodomain is a sequence with at least 80% sequence identity to residues 18-1291 of SEQ ID NO: 100. In some embodiments of any of the aspects, a spike protein ectodomain is a sequence with at least 85% sequence identity to residues 18-1291 of SEQ ID NO: 100. In some embodiments of any of the aspects, a spike protein ectodomain is a sequence with at least 90% sequence identity to residues 18-1291 of SEQ ID NO: 100. In some embodiments of any of the aspects, a spike protein ectodomain is a sequence with at least 95% sequence identity to residues 18-1291 of SEQ ID NO: 100. In some embodiments of any of the aspects, a spike protein ectodomain is a sequence with at least 98% sequence identity to residues 18-1291 of SEQ ID NO: 100. In some embodiments of any of the aspects, a spike protein ectodomain is a sequence with at least 99% sequence identity to residues 18-1291 of SEQ ID NO: 100. In some embodiments of any of the aspects, a spike protein ectodomain is the sequence of residues 18-1291 of SEQ ID NO: 100.

In some embodiments of any of the aspects, a spike protein ectodomain is a sequence with at least 80% sequence identity to residues 14-1211 of SEQ ID NO: 103. In some embodiments of any of the aspects, a spike protein ectodomain is a sequence with at least 85% sequence identity to residues 14-1211 of SEQ ID NO: 103. In some embodiments of any of the aspects, a spike protein ectodomain is a sequence with at least 90% sequence identity to residues 14-1211 of SEQ ID NO: 103. In some embodiments of any of the aspects, a spike protein ectodomain is a sequence with at least 95% sequence identity to residues 14-1211 of SEQ ID NO: 103. In some embodiments of any of the aspects, a spike protein ectodomain is a sequence with at least 98% sequence identity to residues 14-1211 of SEQ ID NO: 103. In some embodiments of any of the aspects, a spike protein ectodomain is a sequence with at least 99% sequence identity to residues 14-1211 of SEQ ID NO: 103. In some embodiments of any of the aspects, a spike protein ectodomain is the sequence of residues 14-1211 of SEQ ID NO: 103.

In some embodiments of any of the aspects, the polypeptide comprising at least one spike protein domain comprises at least a spike protein stalk domain.

In some embodiments of any of the aspects, a spike protein stalk domain is a sequence with at least 80% sequence identity to residues 76-1291 of SEQ ID NO: 100. In some embodiments of any of the aspects, a spike protein stalk domain is a sequence with at least 85% sequence identity to residues 76-1291 of SEQ ID NO: 100. In some embodiments of any of the aspects, a spike protein stalk domain is a sequence with at least 90% sequence identity to residues 76-1291 of SEQ ID NO: 100. In some embodiments of any of the aspects, a spike protein stalk domain is a sequence with at least 95% sequence identity to residues 76-1291 of SEQ ID NO: 100. In some embodiments of any of the aspects, a spike protein stalk domain is a sequence with at least 98% sequence identity to residues 76-1291 of SEQ ID NO: 100. In some embodiments of any of the aspects, a spike protein stalk domain is a sequence with at least 99% sequence identity to residues 76-1291 of SEQ ID NO: 100. In some embodiments of any of the aspects, a spike protein stalk domain is the sequence of residues 76-1291 of SEQ ID NO: 100.

In some embodiments of any of the aspects, a spike protein stalk domain is a sequence with at least 80% sequence identity to residues 686-1211 of SEQ ID NO: 103. In some embodiments of any of the aspects, a spike protein stalk domain is a sequence with at least 85% sequence identity to residues 686-1211 of SEQ ID NO: 103. In some embodiments of any of the aspects, a spike protein stalk domain is a sequence with at least 90% sequence identity to residues 686-1211 of SEQ ID NO: 103. In some embodiments of any of the aspects, a spike protein stalk domain is a sequence with at least 95% sequence identity to residues 686-1211 of SEQ ID NO: 103. In some embodiments of any of the aspects, a spike protein stalk domain is a sequence with at least 98% sequence identity to residues 686-1211 of SEQ ID NO: 103. In some embodiments of any of the aspects, a spike protein stalk domain is a sequence with at least 99% sequence identity to residues 686-1211 of SEQ ID NO: 103. In some embodiments of any of the aspects, a spike protein stalk domain is the sequence of residues 686-1211 of SEQ ID NO: 103.

In some embodiments of any of the aspects, the at least one spike protein domain consists of a spike protein stalk domain. In some embodiments of any of the aspects, the at least one spike protein domain comprises a spike protein stalk domain and does not comprise a S1 region. In some embodiments of any of the aspects, the polypeptide comprising at least one spike protein domain comprises a spike protein stalk domain and does not comprise a S1 region. In some embodiments of any of the aspects, the at least one spike protein domain consists of a spike protein stalk domain and does not comprise a S1 region.

In some embodiments of any of the aspects, the polypeptide comprising at least one spike protein domain comprises at least a spike protein S2 region.

In some embodiments of any of the aspects, a spike protein S2 region is a sequence with at least 80% sequence identity to residues 686-1273 of SEQ ID NO: 103. In some embodiments of any of the aspects, a spike protein S2 region is a sequence with at least 85% sequence identity to residues 686-1273 of SEQ ID NO: 103. In some embodiments of any of the aspects, a spike protein S2 region is a sequence with at least 90% sequence identity to residues 686-1273 of SEQ ID NO: 103. In some embodiments of any of the aspects, a spike protein S2 region is a sequence with at least 95% sequence identity to residues 686-1273 of SEQ ID NO: 103. In some embodiments of any of the aspects, a spike protein S2 region is a sequence with at least 98% sequence identity to residues 686-1273 of SEQ ID NO: 103. In some embodiments of any of the aspects, a spike protein S2 region is a sequence with at least 99% sequence identity to residues 686-1273 of SEQ ID NO: 103. In some embodiments of any of the aspects, a spike protein S2 region is the sequence of residues 686-1273 of SEQ ID NO: 103.

In some embodiments of any of the aspects, the polypeptide comprising at least one spike protein domain comprises at least a spike protein RBD.

In some embodiments of any of the aspects, a spike protein RBD is a sequence with at least 80% sequence identity to residues 367-588 of SEQ ID NO: 100. In some embodiments of any of the aspects, a spike protein RBD is a sequence with at least 85% sequence identity to residues 367-588 of SEQ ID NO: 100. In some embodiments of any of the aspects, a spike protein RBD is a sequence with at least 90% sequence identity to residues 367-588 of SEQ ID NO: 100. In some embodiments of any of the aspects, a spike protein RBD is a sequence with at least 95% sequence identity to residues 367-588 of SEQ ID NO: 100. In some embodiments of any of the aspects, a spike protein RBD is a sequence with at least 98% sequence identity to residues 367-588 of SEQ ID NO: 100. In some embodiments of any of the aspects, a spike protein RBD is a sequence with at least 99% sequence identity to residues 367-588 of SEQ ID NO: 100. In some embodiments of any of the aspects, a spike protein RBD is the sequence of residues 367-588 of SEQ ID NO: 100.

In some embodiments of any of the aspects, a spike protein RBD is a sequence with at least 80% sequence identity to residues 33-528 of SEQ ID NO: 103. In some embodiments of any of the aspects, a spike protein RBD is a sequence with at least 85% sequence identity to residues 33-528 of SEQ ID NO: 103. In some embodiments of any of the aspects, a spike protein RBD is a sequence with at least 90% sequence identity to residues 33-528 of SEQ ID NO: 103. In some embodiments of any of the aspects, a spike protein RBD is a sequence with at least 95% sequence identity to residues 33-528 of SEQ ID NO: 103. In some embodiments of any of the aspects, a spike protein RBD is a sequence with at least 98% sequence identity to residues 33-528 of SEQ ID NO: 103. In some embodiments of any of the aspects, a spike protein RBD is a sequence with at least 99% sequence identity to residues 33-528 of SEQ ID NO: 103. In some embodiments of any of the aspects, a spike protein RBD is the sequence of residues 33-528 of SEQ ID NO: 103.

In some embodiments of any of the aspects, the polypeptide comprising at least one spike protein domain comprises at least a spike protein NTD.

In some embodiments of any of the aspects, a spike protein NTD is a sequence with at least 80% sequence identity to residues 18-353 of SEQ ID NO: 100. In some embodiments of any of the aspects, a spike protein NTD is a sequence with at least 85% sequence identity to residues 18-353 of SEQ ID NO: 100. In some embodiments of any of the aspects, a spike protein NTD is a sequence with at least 90% sequence identity to residues 18-353 of SEQ ID NO: 100. In some embodiments of any of the aspects, a spike protein NTD is a sequence with at least 95% sequence identity to residues 18-353 of SEQ ID NO: 100. In some embodiments of any of the aspects, a spike protein NTD is a sequence with at least 98% sequence identity to residues 18-353 of SEQ ID NO: 100. In some embodiments of any of the aspects, a spike protein NTD is a sequence with at least 99% sequence identity to residues 18-353 of SEQ ID NO: 100. In some embodiments of any of the aspects, a spike protein NTD is the sequence of residues 18-353 of SEQ ID NO: 100.

In some embodiments of any of the aspects, a spike protein NTD is a sequence with at least 80% sequence identity to residues 13-304 of SEQ ID NO: 103. In some embodiments of any of the aspects, a spike protein NTD is a sequence with at least 85% sequence identity to residues 13-304 of SEQ ID NO: 103. In some embodiments of any of the aspects, a spike protein NTD is a sequence with at least 90% sequence identity to residues 13-304 of SEQ ID NO: 103. In some embodiments of any of the aspects, a spike protein NTD is a sequence with at least 95% sequence identity to residues 13-304 of SEQ ID NO: 103. In some embodiments of any of the aspects, a spike protein NTD is a sequence with at least 98% sequence identity to residues 13-304 of SEQ ID NO: 103. In some embodiments of any of the aspects, a spike protein NTD is a sequence with at least 99% sequence identity to residues 13-304 of SEQ ID NO: 103. In some embodiments of any of the aspects, a spike protein NTD is the sequence of residues 13-304 of SEQ ID NO: 103.

In some embodiments of any of the aspects, the polypeptide comprising at least one spike protein domain comprises at least a spike protein S1 region.

In some embodiments of any of the aspects, a spike protein S1 region is a sequence with at least 80% sequence identity to residues 14-685 of SEQ ID NO: 103. In some embodiments of any of the aspects, a spike protein S1 region is a sequence with at least 85% sequence identity to residues 14-685 of SEQ ID NO: 103. In some embodiments of any of the aspects, a spike protein S1 region is a sequence with at least 90% sequence identity to residues 14-685 of SEQ ID NO: 103. In some embodiments of any of the aspects, a spike protein S1 region is a sequence with at least 95% sequence identity to residues 14-685 of SEQ ID NO: 103. In some embodiments of any of the aspects, a spike protein S1 region is a sequence with at least 98% sequence identity to residues 14-685 of SEQ ID NO: 103. In some embodiments of any of the aspects, a spike protein S1 region is a sequence with at least 99% sequence identity to residues 14-685 of SEQ ID NO: 103. In some embodiments of any of the aspects, a spike protein S1 region is the sequence of residues 14-685 of SEQ ID NO: 103.

In some embodiments of any of the aspects, the polypeptide comprising at least one spike protein domain comprises at least a coronavirus spike protein ectodomain. In some embodiments of any of the aspects, the polypeptide comprising at least one spike protein domain comprises at least a coronavirus spike protein stalk domain. In some embodiments of any of the aspects, the polypeptide comprising at least one spike protein domain comprises at least a coronavirus spike protein S2 region. In some embodiments of any of the aspects, the polypeptide comprising at least one spike protein domain comprises at least a coronavirus spike protein RBD. In some embodiments of any of the aspects, the polypeptide comprising at least one spike protein domain comprises at least a coronavirus spike protein NTD. In some embodiments of any of the aspects, the polypeptide comprising at least one spike protein domain comprises at least a coronavirus spike protein S1 region.

In some embodiments of any of the aspects, the polypeptide comprising at least one viral spike protein domain comprises a stabilized spike protein ectodomain. In some embodiments of any of the aspects, the polypeptide comprising at least one viral spike protein domain comprises a stabilized stalk domain. In some embodiments of any of the aspects, the polypeptide comprising at least one viral spike protein domain comprises a stabilized S2 region.

In some embodiments of any of the aspects, the spike protein ectodomain is a stabilized spike protein ectodomain. In some embodiments of any of the aspects, spike protein stalk domain is a stabilized stalk domain. In some embodiments of any of the aspects, the spike protein S2 region is a stabilized S2 region.

Some proteins, including spike protein, have a metastable conformation. Certain modifications of such proteins can induce the most stable confirmation of the protein, instead of the metastable conformation. Thus, the term “stabilized” or “pre-fusion stabilized”, in reference to a viral spike protein is a term known in the art, which refers to modified or mutated versions of a spike protein (or portion thereof, e.g., the ectodomain) which is more likely to exist in the most stable confirmation, e.g., as compared to the metastable state. Stabilized spike proteins, as compared to wild-type or metastable proteins, are more homogenous and more readily produced in recombinant or expression systems. One of skill in the art is aware of multiple different mutations or combinations of mutations that provide stabilized spike proteins. For example, the S-2P, HexaPro, rS2d, u152q, S-R/PP/x1, S-R/x2, S-closed+Fd, 2P DS S, N234A, and VFLIP variants of spike protein are known stabilized spike protein variants. Such stabilized spike proteins are described further in, e.g., Schaub et al. Nature Protocols 16:5339-5356 (2021); and Hsieh et al. Cell Rep 37:109929 (2021); each of which is incorporated by reference herein in its entirety. In some embodiments of any of the aspects, the at least one spike protein domain comprises a S-2P, HexaPro, rS2d, u152q, S-R/PP/x1, S-R/x2, S-closed+Fd, 2P DS S, N234A, or VFLIP variant of the at least one spike protein domain.

As used herein, “2P variant” refers to a spike protein, variant, or portion therefore (e.g., a spike protein ectodomain or spike protein stalk domain) in which the residues corresponding to V1060 and L1061 of SEQ ID NO: 100, or the residues corresponding to K986 and V987 of SEQ ID NO: 103 are mutated to prolines. In some embodiments of any of the aspects, a 2P variant comprising at least a portion of the S1 region further comprises a mutation of the furin cleavage site located between the S1 region and the S2 region, e.g., from RRAR (SEQ ID NO: 220) to GSAS (SEQ ID NO: 221). Exemplary 2P variants are provided herein, e.g., SEQ ID NOs: 78-84. One of skill in the art can align (e.g., using widely available tools such as BLAST) one or more of the afore-mentioned sequences to a different ectodomain and/or stalk sequence to determine the location of the corresponding residues in the different ectodomain and/or stalk sequence.

As used herein, “HexaPro” refers to a spike protein, variant, or portion therefore (e.g., a spike protein ectodomain or spike protein stalk domain) in which the residues corresponding to K986, V987, F817P A892, A899, and A942 of SEQ ID NO: 103 are mutated to prolines. In some embodiments of any of the aspects, a HexaPro variant comprising at least a portion of the S1 region further comprises a mutation of the furin cleavage site located between the S1 region and the S2 region, e.g., from RRAR (SEQ ID NO: 220) to GSAS (SEQ ID NO: 221). One of skill in the art can align (e.g., using widely available tools such as BLAST) one or more of the afore-mentioned sequences to a different ectodomain and/or stalk sequence to determine the location of the corresponding residues in the different ectodomain and/or stalk sequence. For further discussion of HexaPro, see, e.g, Hsieh et al. Science 369:1501-1505 (2020).

As used herein, “rS2d” refers to a spike protein, variant, or portion therefore (e.g., a spike protein ectodomain or spike protein stalk domain) in which the residues corresponding to K986, V987, S383, D985 of SEQ ID NO: 103 are mutated to K986P, V987P, S383C, and D985C. In some embodiments of any of the aspects, a rS2d variant comprising at least a portion of the S1 region further comprises a mutation of the furin cleavage site located between the S1 region and the S2 region, e.g., from RRAR (SEQ ID NO: 220) to GSAS (SEQ ID NO: 221). One of skill in the art can align (e.g., using widely available tools such as BLAST) one or more of the afore-mentioned sequences to a different ectodomain and/or stalk sequence to determine the location of the corresponding residues in the different ectodomain and/or stalk sequence.

As used herein, “u1S2q” refers to a spike protein, variant, or portion therefore (e.g., a spike protein ectodomain or spike protein stalk domain) in which the residues corresponding to A570, T572, F855, N856, K986, V987, A570, T572, F855 of SEQ ID NO: 103 are mutated to A570L, T572I, F855Y, N856I, K986P, V987P, A570L, T572I, F855Y, and N856I. In some embodiments of any of the aspects, a u1S2q variant comprising at least a portion of the S1 region further comprises a mutation of the furin cleavage site located between the S1 region and the S2 region, e.g., from RRAR (SEQ ID NO: 220) to GSAS (SEQ ID NO: 221). One of skill in the art can align (e.g., using widely available tools such as BLAST) one or more of the afore-mentioned sequences to a different ectodomain and/or stalk sequence to determine the location of the corresponding residues in the different ectodomain and/or stalk sequence.

As used herein, “S-R/PP/x1” refers to a spike protein, variant, or portion therefore (e.g., a spike protein ectodomain or spike protein stalk domain) in which the residues corresponding to K986, V987, S383, D985 of SEQ ID NO: 103 are mutated to K986P, V987P, S383C, and D985C. In some embodiments of any of the aspects, a S-R/PP/x1 variant comprising at least a portion of the S1 region further comprises a mutation of the furin cleavage site located between the S1 region and the S2 region, e.g., from RRAR to PRRAR. One of skill in the art can align (e.g., using widely available tools such as BLAST) one or more of the afore-mentioned sequences to a different ectodomain and/or stalk sequence to determine the location of the corresponding residues in the different ectodomain and/or stalk sequence.

As used herein, “S-R/x2” refers to a spike protein, variant, or portion therefore (e.g., a spike protein ectodomain or spike protein stalk domain) in which the residues corresponding to K986, G413, and V987 of SEQ ID NO: 103 are mutated to K986P, G413, and V987P. In some embodiments of any of the aspects, a S-R/x2 variant comprising at least a portion of the S1 region further comprises a mutation of the furin cleavage site located between the S1 region and the S2 region, e.g., from RRAR(SEQ ID NO: 220) to PRRAR (SEQ ID NO: 222). One of skill in the art can align (e.g., using widely available tools such as BLAST) one or more of the afore-mentioned sequences to a different ectodomain and/or stalk sequence to determine the location of the corresponding residues in the different ectodomain and/or stalk sequence.

As used herein, “S-closed+Fd” refers to a spike protein, variant, or portion therefore (e.g., a spike protein ectodomain or spike protein stalk domain) in which the residues corresponding to K986, V987, D614, A892, A942, and V987 of SEQ ID NO: 103 are mutated to K986P, V987P, D614N, A892P, A942P, and V987P. In some embodiments of any of the aspects, a S-closed+Fd variant comprising at least a portion of the S1 region further comprises a mutation of the furin cleavage site located between the S1 region and the S2 region, e.g., from RRAR to GSAS. One of skill in the art can align (e.g., using widely available tools such as BLAST) one or more of the afore-mentioned sequences to a different ectodomain and/or stalk sequence to determine the location of the corresponding residues in the different ectodomain and/or stalk sequence.

As used herein, “2P DS S” refers to a spike protein, variant, or portion therefore (e.g., a spike protein ectodomain or spike protein stalk domain) in which the residues corresponding to K986, V987, S383, and D985 of SEQ ID NO: 103 are mutated to K986P, V987P, S383C, and D985C. In some embodiments of any of the aspects, a 2P DS S variant comprising at least a portion of the S1 region further comprises a mutation of the furin cleavage site located between the S1 region and the S2 region, e.g., from RRAR (SEQ ID NO: 220) to SGAG (SEQ ID NO: 223). One of skill in the art can align (e.g., using widely available tools such as BLAST) one or more of the afore-mentioned sequences to a different ectodomain and/or stalk sequence to determine the location of the corresponding residues in the different ectodomain and/or stalk sequence.

As used herein, “N234A” refers to a spike protein, variant, or portion therefore (e.g., a spike protein ectodomain or spike protein stalk domain) in which the residues corresponding to K986, V987, and N234 of SEQ ID NO: 103 are mutated to K986P, V987P, and N234A. One of skill in the art can align (e.g., using widely available tools such as BLAST) one or more of the afore-mentioned sequences to a different ectodomain and/or stalk sequence to determine the location of the corresponding residues in the different ectodomain and/or stalk sequence.

As used herein, “VFLIP” refers to a spike protein, variant, or portion therefore (e.g., a spike protein ectodomain or spike protein stalk domain) in which the residues corresponding to F817, A892, A899, A942, K986, Y707, and T883 of SEQ ID NO: 103 are mutated to F817P, A892P, A899P, A942P, K986P, Y707C, and T883C. In some embodiments of any of the aspects, a VFLIP variant comprising at least a portion of the S1 region further comprises a mutation of the furin cleavage site located between the S1 region and the S2 region, e.g., from RRAR (SEQ ID NO: 220) to GGSGGGS (SEQ ID NO: 224). One of skill in the art can align (e.g., using widely available tools such as BLAST) one or more of the afore-mentioned sequences to a different ectodomain and/or stalk sequence to determine the location of the corresponding residues in the different ectodomain and/or stalk sequence. For further discussion of VFLIP, see, e.g., Olmedillas et al. bioRxiv 05.06.441046 (2021); which is incorporated by reference herein in its entirety.

In some embodiments of any of the aspects, a polypeptide comprising a least one viral spike protein domain, wherein the at least one viral spike protein domain is a stabilized domain comprises a sequence with at least 80% sequence identity to SEQ ID NO: 107. In some embodiments of any of the aspects, a polypeptide comprising a least one viral spike protein domain, wherein the at least one viral spike protein domain is a stabilized domain comprises a sequence with at least 85% sequence identity to SEQ ID NO: 107. In some embodiments of any of the aspects, a polypeptide comprising a least one viral spike protein domain, wherein the at least one viral spike protein domain is a stabilized domain comprises a sequence with at least 90% sequence identity to SEQ ID NO: 107. In some embodiments of any of the aspects, a polypeptide comprising a least one viral spike protein domain, wherein the at least one viral spike protein domain is a stabilized domain comprises a sequence with at least 95% sequence identity to SEQ ID NO: 107. In some embodiments of any of the aspects, a polypeptide comprising a least one viral spike protein domain, wherein the at least one viral spike protein domain is a stabilized domain comprises a sequence with at least 98% sequence identity to SEQ ID NO: 107. In some embodiments of any of the aspects, a polypeptide comprising a least one viral spike protein domain, wherein the at least one viral spike protein domain is a stabilized domain comprises a sequence with at least 98% sequence identity to SEQ ID NO: 107. In some embodiments of any of the aspects, a polypeptide comprising a least one viral spike protein domain, wherein the at least one viral spike protein domain is a stabilized domain comprises the sequence of SEQ ID NO: 107. In some embodiments of any of the aspects, a stabilized viral spike protein domain consists of the sequence of SEQ ID NO: 107.

In some embodiments of any of the aspects, a polypeptide comprising a least one viral spike protein domain, wherein the at least one viral spike protein domain is a stabilized domain comprises a sequence with at least 80% sequence identity to SEQ ID NO: 108. In some embodiments of any of the aspects, a polypeptide comprising a least one viral spike protein domain, wherein the at least one viral spike protein domain is a stabilized domain comprises a sequence with at least 85% sequence identity to SEQ ID NO: 108. In some embodiments of any of the aspects, a polypeptide comprising a least one viral spike protein domain, wherein the at least one viral spike protein domain is a stabilized domain comprises a sequence with at least 90% sequence identity to SEQ ID NO: 108. In some embodiments of any of the aspects, a polypeptide comprising a least one viral spike protein domain, wherein the at least one viral spike protein domain is a stabilized domain comprises a sequence with at least 95% sequence identity to SEQ ID NO: 108. In some embodiments of any of the aspects, a polypeptide comprising a least one viral spike protein domain, wherein the at least one viral spike protein domain is a stabilized domain comprises a sequence with at least 98% sequence identity to SEQ ID NO: 108. In some embodiments of any of the aspects, a polypeptide comprising a least one viral spike protein domain, wherein the at least one viral spike protein domain is a stabilized domain comprises a sequence with at least 98% sequence identity to SEQ ID NO: 108. In some embodiments of any of the aspects, a polypeptide comprising a least one viral spike protein domain, wherein the at least one viral spike protein domain is a stabilized domain comprises the sequence of SEQ ID NO: 108. In some embodiments of any of the aspects, a stabilized viral spike protein domain consists of the sequence of SEQ ID NO: 108.

SEQ ID NO: 107
AFNHPIQVDQLNSSYFKLSIPTNFSFGVTQEYIQTTIQKVCVDCKQYVCN
GFQKCEQLLREYGQFCSKINQALHGANLRQDDSVRNLFASVKSSQSSPII
PGFGGDENLILLEPVSISTGSRSARSAIEDLLEDKVTIADPGYMQGYDDC
MQQGPASARDLICAQYVAGYCVLPPLMDVNMEAAYTSSLLGSIAGSGWTA
GLSSFAAIPFAQMIFYRINGIGITQQVLSENQKLIANKENQALGAMQTGF
TTTNEAFQKVQDAVNNNAQALSKLASELSNTFGAISASIGDIIQRLDPPE
QDAQIDRLINGRLTTLNAFVAQQLVRSEEAAQSAQLAKDKVNECVKAQSK
RSGFCGQGTHIVSFVVNAPYGLYFMHVGYYPSNHIEVVSAYGLCDAANPT
NCIAPVNGYFIKTNNTRIVDEWSYTGSSFYAPEPITSLNTKYVAPQVTYQ
NISTNLPPPLLGNSTGIDFQDELDEFFKNVSTSIPNFGSLTQINTTLLDL
TYEMLSLQQVVKALNESYIDLKELGNYTY
SEQ ID NO: 108
AFNHPIQVDQLNSSYFKLSIPTNFSFGVTQEYIQTTIQKVCVDCKQYVCN
GFQKCEQLLREYGQFCSKINQALHGCNLRQDDSVRNLFASVKSSQSSPII
PGFGGDENLILLEPVSISTGSRSARSAIEDLLEDKCTIADPGYMQGYDDC
MQQGPASARDLICAQYVAGYCVLPPLMDVNMEAAYTSSLLGSIAGSGWTA
GLSSFAAIPFAQMIFYRINGIGITQQVLSENQKLIANKENQALGAMQTGF
TTTNEAFQKCQDAVNNNAQALSKLASELSNTFGAISASIGDIIQRLDPPE
QDAQIDRLINGRLTTLNAFVAQQLVRCEEAAQSAQLAKDKVNECVKAQSK
RSGFCGQGTHIVSFVVNAPYGLYEMHVGYYPSNHIEVVSAYGLCDAANPT
NCIAPVNGYFIKTNNTRIVDEWSYTGSSFYAPEPITSLNTKYVAPQVTYQ
NISTNLPPPLLGNSTGIDFQDELDEFFKNVSTSIPNFGSLTQINTTLLDL
TYEMLSLQQVVKALNESYIDLKELGNYTY

In some embodiments of any of the aspects, the polypeptide comprising at least one spike protein domain does not comprise a spike protein RBD. In some embodiments of any of the aspects, the polypeptide comprising at least one spike protein domain does not comprise a coronavirus spike protein RBD. In some embodiments of any of the aspects, the polypeptide comprising at least one spike protein domain does not comprise a sequence with at least 80% sequence identity to residues 367-588 of SEQ ID NO: 100. In some embodiments of any of the aspects, the polypeptide comprising at least one spike protein domain does not comprise a sequence with at least 85% sequence identity to residues 367-588 of SEQ ID NO: 100. In some embodiments of any of the aspects, the polypeptide comprising at least one spike protein domain does not comprise a sequence with at least 90% sequence identity to residues 367-588 of SEQ ID NO: 100. In some embodiments of any of the aspects, the polypeptide comprising at least one spike protein domain does not comprise a sequence with at least 95% sequence identity to residues 367-588 of SEQ ID NO: 100. In some embodiments of any of the aspects, the polypeptide comprising at least one spike protein domain does not comprise a sequence with at least 98% sequence identity to residues 367-588 of SEQ ID NO: 100. In some embodiments of any of the aspects, the polypeptide comprising at least one spike protein domain does not comprise a sequence with at least 99% sequence identity to residues 367-588 of SEQ ID NO: 100. In some embodiments of any of the aspects, the polypeptide comprising at least one spike protein domain does not comprise the sequence of residues 367-588 of SEQ ID NO: 100.

In some embodiments of any of the aspects, the polypeptide comprising at least one spike protein domain does not comprise a spike protein hypervariable immunogenic epitope. In some embodiments of any of the aspects, the polypeptide comprising at least one spike protein domain does not comprise a coronavirus spike protein hypervariable immunogenic epitope.

As used herein, “hypervariable immunogenic epitope” refers to an epitope, linear or noncontiguous, which provokes an immune response and has a high degree of sequence variability within a population, between strains, between species, or between clades, e.g., as compared to the degree of sequence variability observed for the rest of the protein comprising the hypervariable immunogenic epitope. A hypervariable immunogenic epitope can therefore be identified by alignment and sequence comparison of protein sequences from different samples, which can readily be done by one of skill in the art using freely available resources such as the NCBI sequence database and BLAST tool.

In some embodiments of any of the aspects, the at least one spike protein domain consists of a spike protein stalk domain and does not comprise a hypervariable immunogenic epitope. In some embodiments of any of the aspects, the at least one spike protein domain comprises a spike protein stalk domain, does not comprise a hypervariable immunogenic epitope, and does not comprise a S1 region. In some embodiments of any of the aspects, the polypeptide comprising at least one spike protein domain comprises a spike protein stalk domain, does not comprise a hypervariable immunogenic epitope, and does not comprise a S1 region. In some embodiments of any of the aspects, the at least one spike protein domain consists of a spike protein stalk domain, does not comprise a hypervariable immunogenic epitope, and does not comprise a S1 region.

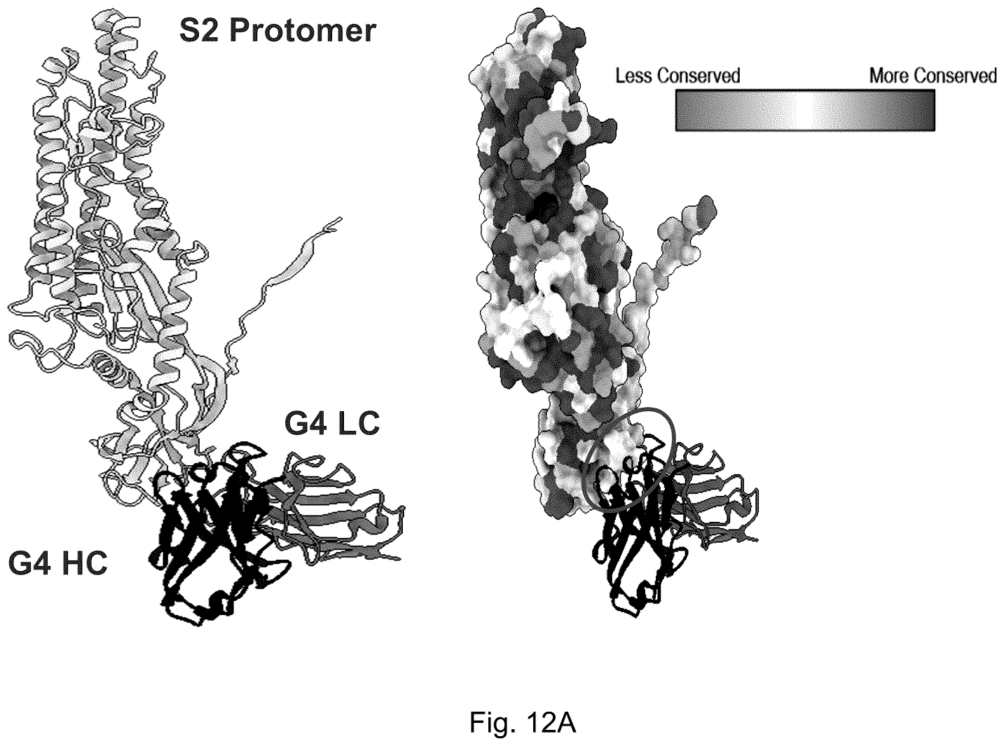

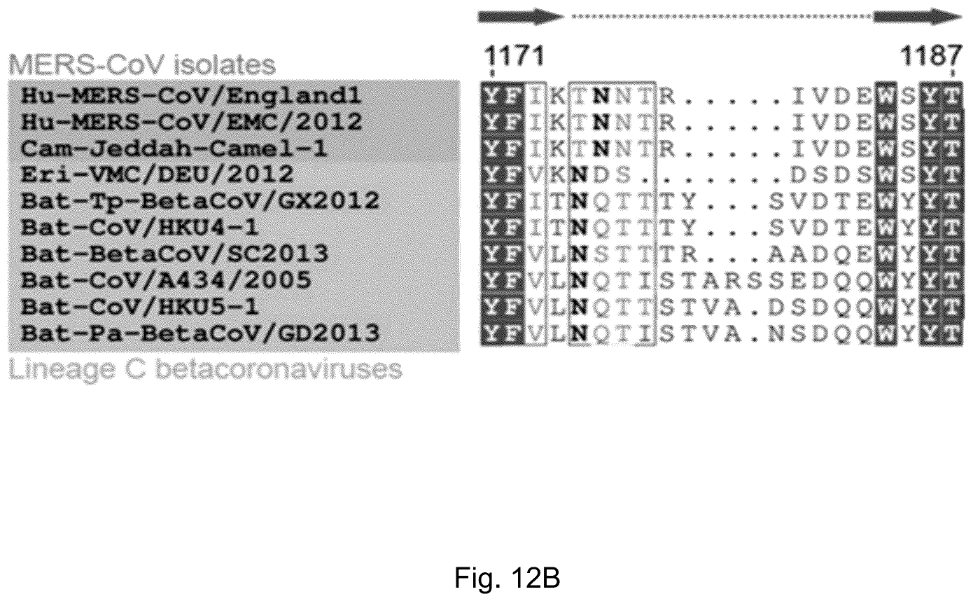

In some embodiments of any of the aspects, the hypervariable immunogenic epitope is the G4 loop of coronavirus spike protein. “G4 loop” refers to a portion of the S2 region of the coronavirus spike protein corresponding to residues 1178-1183 of SEQ ID NO: 100. The hypervariable immunogenic epitope can vary in length and sequence. One of skill in the art can align (e.g., using widely available tools such as BLAST) one or more of the afore-mentioned sequences to a different ectodomain and/or stalk sequence to determine the location of the corresponding residues in the different ectodomain and/or stalk sequence. An exemplary such alignment is shown in FIG. 12B. Alternatively, in some embodiments of any of the aspects, a G4 loop can be the sequence found between a first portion of the stalk sequence and a second portion of the stalk sequence, where the first portion ends with the sequence YFX₁X₂X₃X₄X₅ (where X₁ is I or V; X₂ is K, T, or L; X₃ is T or N; X₄ is N, D, W, Q, or S; and X₅ is N, S, or T) and the second portion begins with the sequence WSYT (SEQ ID NO: 225) (e.g., corresponding to SEQ ID NO: 100). Alternatively, in some embodiments of any of the aspects, a G4 loop can be the sequence found between a first portion of the stalk sequence and a second portion of the stalk sequence, where the first portion ends with the sequence VSNG (SEQ ID NO: 226) and the second portion begins with the sequence WFVT (SEQ ID NO: 227) (e.g, corresponding to SEQ ID NO: 103).

In some embodiments of any of the aspects, the G4 loop is deleted from the polypeptide. In some embodiments of any of the aspects, the polypeptide does not comprise the G4 loop. In some embodiments of any of the aspects, the polypeptide does not comprise the G4 loop and does not comprise a 5′ flanking sequence contiguous with the G4 loop in the wild-type reference sequence. In some embodiments of any of the aspects, the polypeptide does not comprise the G4 loop and does not comprise a 3′ flanking sequence contiguous with the G4 loop in the wild-type reference sequence. In some embodiments of any of the aspects, the polypeptide does not comprise the G4 loop, does not comprise a 5′ flanking sequence contiguous with the G4 loop in the wild-type reference sequence, does not comprise a 3′ flanking sequence contiguous with the G4 loop in the wild-type reference sequence. In some embodiments of any of the aspects, a flanking sequence contiguous with the G4 loop in the wild-type reference sequence is 1 nucleotide in length. In some embodiments of any of the aspects, a flanking sequence contiguous with the G4 loop in the wild-type reference sequence is 2 nucleotides in length. In some embodiments of any of the aspects, a flanking sequence contiguous with the G4 loop in the wild-type reference sequence is 5 nucleotides in length. In some embodiments of any of the aspects, a flanking sequence contiguous with the G4 loop in the wild-type reference sequence is 10 nucleotides in length. In some embodiments of any of the aspects, a flanking sequence contiguous with the G4 loop in the wild-type reference sequence is 15 nucleotides in length. In some embodiments of any of the aspects, a flanking sequence contiguous with the G4 loop in the wild-type reference sequence is 20 nucleotides in length. In some embodiments of any of the aspects, a flanking sequence contiguous with the G4 loop in the wild-type reference sequence is 25 nucleotides in length. In some embodiments of any of the aspects, a flanking sequence contiguous with the G4 loop in the wild-type reference sequence is 30 nucleotides in length. In some embodiments of any of the aspects, a flanking sequence contiguous with the G4 loop in the wild-type reference sequence is 50 nucleotides in length.

In some embodiments of any of the aspects, the polypeptide comprising at least one spike protein domain comprises a sequence with at least 80% sequence identity to SEQ ID NO: 101. In some embodiments of any of the aspects, the polypeptide comprising at least one spike protein domain comprises a sequence with at least 85% sequence identity to SEQ ID NO: 101. In some embodiments of any of the aspects, the polypeptide comprising at least one spike protein domain comprises a sequence with at least 90% sequence identity to SEQ ID NO: 101. In some embodiments of any of the aspects, the polypeptide comprising at least one spike protein domain comprises a sequence with at least 95% sequence identity to SEQ ID NO: 101. In some embodiments of any of the aspects, the polypeptide comprising at least one spike protein domain comprises sequence with at least 98% sequence identity to SEQ ID NO: 101. In some embodiments of any of the aspects, the polypeptide comprising at least one spike protein domain comprises a sequence with at least 99% sequence identity to SEQ ID NO: 101. In some embodiments of any of the aspects, the polypeptide comprising at least one spike protein domain comprises the sequence of SEQ ID NO: 101.

(stalk domain with G4 loop deletion)
SEQ ID NO: 101
IAFNHPIQVDQLNSSYFKLSIPTNFSFGVTQEYIQTTIQKVTVDCKQYVC
NGFQKCEQLLREYGQFCSKINQALHGANLRQDDSVRNLFASVKSSQSSPI
IPGFGGDENLILLEPVSISTGSRSARSAIEDLLEDKVTIADPGYMQGYDD
CMQQGPASARDLICAQYVAGYKVLPPLMDVNMEAAYTSSLLGSIAGVGWT
AGLSSFAAIPFAQSIFYRLNGVGITQQVLSENQKLIANKENQALGAMQTG
FTTTNEAFQKVQDAVNNNAQALSKLASELSNTFGAISASIGDIIQRLDVL
EQDAQIDRLINGRLTTLNAFVAQQLVRSESAALSAQLAKDKVNECVKAQS
KRSGFCGQGTHIVSFVVNAPNGLYFMHVGYYPSNHIEVVSAYGLCDAANP
TNCIAPVNGYFIKTNNWSYTGSSFYAPEPITSLNTKYVAPQVTYQNISTN
LPPPLLGNSTGIDFQDELDEFFKNVSTSIPNFGSLTQINTTLLDLTYEML
SLQQVVKALNESYIDLKELGNYTY
SEQ ID NO: 106
svasq siiaytmslg aensvaysnn siaiptnfti
svtteilpvs mtktsvdctm yicgdstecs nlllqygsfc
tqlnraltgi aveqdkntqe vfaqvkqiyk tppikdfggf
nfsqilpdps kpskrsfied llfnkvtlad agfikqygdc
lgdiaardli caqkfngltv lpplltdemi aqytsallag
titsgwtfga gaalqipfam qmayrfngig vtqnvlyenq
klianqfnsa igkiqdslss tasalgklqd vvnqnaqaln
tlvkqlssnf gaissvlndi lsrldkveae vqidrlitgr
lqslqtyvtq qliraaeira sanlaatkms ecvlgqskrv
dfcgkgyhlm sfpqsaphgv vflhvtyvpa qeknfttapa
ichdgkahfp regvfvsng wfvtqrnfy epqiittdnt
fvsgncdvvi givnntvydp lqpeldsfke eldkyfknht
spdvdlgdis ginasvvniq keidrlneva knlneslidl
qelgkyeqyi k

In some embodiments of any of the aspects, the polypeptide comprising at least one spike protein domain comprises a fusion domain. In some embodiments of any of the aspects, a fusion domain comprises a sequence with at least 80% sequence identity to residues 816-833 of SEQ ID NO: 103. In some embodiments of any of the aspects, a fusion domain comprises a sequence with at least 85% sequence identity to residues 816-833 of SEQ ID NO: 103. In some embodiments of any of the aspects, a fusion domain comprises a sequence with at least 90% sequence identity to residues 816-833 of SEQ ID NO: 103. In some embodiments of any of the aspects, a fusion domain comprises a sequence with at least 95% sequence identity to residues 816-833 of SEQ ID NO: 103. In some embodiments of any of the aspects, a fusion domain comprises sequence with at least 98% sequence identity to residues 816-833 of SEQ ID NO: 103. In some embodiments of any of the aspects, a fusion domain comprises a sequence with at least 99% sequence identity to residues 816-833 of SEQ ID NO: 103. In some embodiments of any of the aspects, a fusion domain comprises the sequence of residues 816-833 of SEQ ID NO: 103.

In some embodiments of any of the aspects, a fusion domain comprises a sequence with at least 80% sequence identity to SEQ ID NO: 104. In some embodiments of any of the aspects, a fusion domain comprises a sequence with at least 85% sequence identity to SEQ ID NO: 104. In some embodiments of any of the aspects, a fusion domain comprises a sequence with at least 90% sequence identity to SEQ ID NO: 104. In some embodiments of any of the aspects, a fusion domain comprises a sequence with at least 95% sequence identity to SEQ ID NO: 104. In some embodiments of any of the aspects, a fusion domain comprises sequence with at least 98% sequence identity to SEQ ID NO: 104. In some embodiments of any of the aspects, a fusion domain comprises a sequence with at least 99% sequence identity to SEQ ID NO: 104. In some embodiments of any of the aspects, a fusion domain comprises the sequence of SEQ ID NO: 104. In some embodiments of any of the aspects, a fusion domain consist of the sequence of SEQ ID NO: 104.

In some embodiments of any of the aspects, a fusion domain comprises a sequence with at least 80% sequence identity to SEQ ID NO: 105. In some embodiments of any of the aspects, a fusion domain comprises a sequence with at least 85% sequence identity to SEQ ID NO: 105. In some embodiments of any of the aspects, a fusion domain comprises a sequence with at least 90% sequence identity to SEQ ID NO: 105. In some embodiments of any of the aspects, a fusion domain comprises a sequence with at least 95% sequence identity to SEQ ID NO: 105. In some embodiments of any of the aspects, a fusion domain comprises sequence with at least 98% sequence identity to SEQ ID NO: 105. In some embodiments of any of the aspects, a fusion domain comprises a sequence with at least 99% sequence identity to SEQ ID NO: 105. In some embodiments of any of the aspects, a fusion domain comprises the sequence of SEQ ID NO: 105. In some embodiments of any of the aspects, a fusion domain consist of the sequence of SEQ ID NO: 105.

| SEQ ID NO: 104 | |
|---|---|
| | SFIEDLLFNKVTLADAGFIKQYGDCLGDIAARDLICAQKENG |
| SEQ ID NO: 105 | |
| | SFIEDLLFNKVTLADAGFIKQYGDCLGDVAARDLICAQKFNG |

In some embodiments of any of the aspects, the polypeptide comprising at least one spike protein domain comprises a concatenation of at least a first spike protein sequence and a second spike protein sequence. As used herein, “concatenation” refers to an engineered sequence which comprises a series of connected sequences, where the concatenation does not occur in nature. The series can comprise at least 2 sequences. In some embodiments of any of the aspects, a concatenation can be repeated, e.g., 2 concatenations, 3 concatenations, 4 concatenation, or 5 or more concatenations. The two or more sequences of a concatenation can be contiguous or connected by a linker sequence. A concatenation can be created by deleted intervening sequences that occur in a natural protein, or by combining a first sequence from one natural source and a second sequence from a second natural course.

In some embodiments of any of the aspects, the polypeptide comprises a concatenation of at least a first spike protein sequence and a second spike protein sequence, and the concatenation comprises: a concatenation of at least a first N-Terminal Domain (NTD) sequence and a second NTD sequence. In some embodiments of any of the aspects, the polypeptide comprises a concatenation of at least a first spike protein sequence and a second spike protein sequence, and the concatenation comprises a concatenation of at least a first RBD sequence and a second RBD sequence. In some embodiments of any of the aspects, the polypeptide comprises a concatenation of at least a first spike protein sequence and a second spike protein sequence, and the concatenation comprises a concatenation of at least: a) a first NTD sequence, a first Subdomain 1 (SD1) sequence, and a first Subdomain 2 (SD2) sequence; and b) a second NTD sequence, a second SD1 sequence, and a second SD2 sequence. In some embodiments of any of the aspects, the polypeptide comprises a concatenation of at least a first spike protein sequence and a second spike protein sequence, and the concatenation comprises a concatenation of at least: a) a first NTD sequence, a first Subdomain 1 (SD1) sequence, and a first Subdomain 2 (SD2) sequence wherein there is no RBD sequence; and b) a second NTD sequence, a second SD1 sequence, and a second SD2 sequence wherein there is no RBD sequence. In some embodiments of any of the aspects, the polypeptide comprises a concatenation of at least a first spike protein sequence and a second spike protein sequence, and the concatenation comprises a concatenation of at least: a) a first RBD sequence, a first SD1 sequence, and a first SD2 sequence; and b) a second RBD sequence, a second SD1 sequence, and a second SD2 sequence. In some embodiments of any of the aspects, the polypeptide comprises a concatenation of at least a first spike protein sequence and a second spike protein sequence, and the concatenation comprises a concatenation of at least: a) a first RBD sequence, a first SD1 sequence, and a first SD2 sequence wherein there is no NTD sequence; and b) a second RBD sequence, a second SD1 sequence, and a second SD2 sequence wherein there is no NTD sequence. In some embodiments of any of the aspects, the polypeptide comprises a concatenation of at least a first spike protein sequence and a second spike protein sequence, and the concatenation comprises a concatenation of at least: a) a first RBD sequence, a first SD1 sequence, and a first SD2 sequence wherein there is no NTD sequence intervening between the first RBD sequence and the first SD1 sequence; and b) a second RBD sequence, a second SD1 sequence, and a second SD2 sequence wherein there is no NTD sequence intervening between the second RBD sequence and the second SD1 sequence. In some embodiments of any of the aspects, the polypeptide comprises a concatenation of at least a first spike protein sequence and a second spike protein sequence, and the concatenation comprises a concatenation of at least: a first fusion sequence; and a second fusion sequence. In any of the foregoing, the first and sequence sequences are not identical. In any of the foregoing, the first and sequence sequences can be the sequences of a first virus and a second virus. In any of the foregoing, the first and sequence sequences are not identical. In any of the foregoing, the first and sequence sequences can be the sequences of a first virus strain and a second virus strain. In any of the foregoing, the first and sequence sequences are not identical. In any of the foregoing, the first and sequence sequences can be the sequences of a first virus species and a second virus species.

In some embodiments of any of the aspects, the polypeptide comprising at least one viral spike protein domain comprises a concatenation of at least a first spike protein sequence from a first virus and a second spike protein sequence from a second virus. In some embodiments of any of the aspects, the polypeptide comprising at least one viral spike protein domain comprises a concatenation of at least a first spike protein sequence from a first virus and a second spike protein sequence from a second virus and the concatenation comprises: a concatenation of at least a first N-Terminal Domain (NTD) sequence from the first virus and a second NTD sequence from the second virus. In some embodiments of any of the aspects, the polypeptide comprising at least one viral spike protein domain comprises a concatenation of at least a first spike protein sequence from a first virus and a second spike protein sequence from a second virus and the concatenation comprises: a concatenation of at least a first RBD sequence from the first virus and a second RBD sequence from the second virus. In some embodiments of any of the aspects, the polypeptide comprising at least one viral spike protein domain comprises a concatenation of at least a first spike protein sequence from a first virus and a second spike protein sequence from a second virus and the concatenation comprises: a concatenation of at least: a) a first NTD sequence from the first virus, a first SD1 sequence from the first virus, and a first SD2 sequence from the first virus; and b) a second NTD sequence from the second virus, a second SD1 sequence from the second virus, and a second SD2 sequence from the second virus. In some embodiments of any of the aspects, the polypeptide comprising at least one viral spike protein domain comprises a concatenation of at least a first spike protein sequence from a first virus and a second spike protein sequence from a second virus and the concatenation comprises: a concatenation of at least: a) a first RBD sequence from the first virus, a first SD1 sequence from the first virus, and a first SD2 sequence from the first virus; and b) a second RBD sequence from the second virus, a second SD1 sequence from the second virus, and a second SD2 sequence from the second virus. In some embodiments of any of the aspects, the polypeptide comprising at least one viral spike protein domain comprises a concatenation of at least a first spike protein sequence from a first virus and a second spike protein sequence from a second virus and the concatenation comprises: a concatenation of at least: a) a first fusion sequence from the first virus; and b) a second fusion sequence from the second virus.

In some embodiments of any of the aspects, the at least one domain of a virus spike protein comprises a sequence with at least 80% sequence identity to the sequence of one of SEQ ID NOs: 21-27, 78-84, 90-96, 123-134, and 143-150. In some embodiments of any of the aspects, the at least one domain of a virus spike protein comprises a sequence with at least 85% sequence identity to the sequence of one of SEQ ID NOs: 21-27, 78-84, 90-96, 123-134, and 143-150. In some embodiments of any of the aspects, the at least one domain of a virus spike protein comprises a sequence with at least 90% sequence identity to the sequence of one of SEQ ID NOs: 21-27, 78-84, 90-96, 123-134, and 143-150. In some embodiments of any of the aspects, the at least one domain of a virus spike protein comprises a sequence with at least 95% sequence identity to the sequence of one of SEQ ID NOs: 21-27, 78-84, 90-96, 123-134, and 143-150. In some embodiments of any of the aspects, the at least one domain of a virus spike protein comprises a sequence with at least 98% sequence identity to the sequence of one of SEQ ID NOs: 21-27, 78-84, 90-96, 123-134, and 143-150. In some embodiments of any of the aspects, the at least one domain of a virus spike protein comprises a sequence with at least 99% sequence identity to the sequence of one of SEQ ID NOs: 21-27, 78-84, 90-96, 123-134, and 143-150. In some embodiments of any of the aspects, the at least one domain of a virus spike protein comprises the sequence of one of SEQ ID NOs: 21-27, 78-84, 90-96, 123-134, and 143-150.

In some embodiments of any of the aspects, the at least one domain of a virus spike protein comprises a sequence with at least 80% sequence identity to the sequence of SEQ ID NO: 21. In some embodiments of any of the aspects, the at least one domain of a virus spike protein comprises a sequence with at least 85% sequence identity to the sequence of SEQ ID NO: 21. In some embodiments of any of the aspects, the at least one domain of a virus spike protein comprises a sequence with at least 90% sequence identity to the sequence of SEQ ID NO: 21. In some embodiments of any of the aspects, the at least one domain of a virus spike protein comprises a sequence with at least 95% sequence identity to the sequence of SEQ ID NO: 21. In some embodiments of any of the aspects, the at least one domain of a virus spike protein comprises a sequence with at least 98% sequence identity to the sequence of SEQ ID NO: 21. In some embodiments of any of the aspects, the at least one domain of a virus spike protein comprises a sequence with at least 99% sequence identity to the sequence of SEQ ID NO: 21. In some embodiments of any of the aspects, the at least one domain of a virus spike protein comprises the sequence of SEQ ID NO: 21.

In some embodiments of any of the aspects, the at least one domain of a virus spike protein comprises a sequence with at least 80% sequence identity to the sequence of SEQ ID NO: 22. In some embodiments of any of the aspects, the at least one domain of a virus spike protein comprises a sequence with at least 85% sequence identity to the sequence of SEQ ID NO: 22. In some embodiments of any of the aspects, the at least one domain of a virus spike protein comprises a sequence with at least 90% sequence identity to the sequence of SEQ ID NO: 22. In some embodiments of any of the aspects, the at least one domain of a virus spike protein comprises a sequence with at least 95% sequence identity to the sequence of SEQ ID NO: 22. In some embodiments of any of the aspects, the at least one domain of a virus spike protein comprises a sequence with at least 98% sequence identity to the sequence of SEQ ID NO: 22. In some embodiments of any of the aspects, the at least one domain of a virus spike protein comprises a sequence with at least 99% sequence identity to the sequence of SEQ ID NO: 22. In some embodiments of any of the aspects, the at least one domain of a virus spike protein comprises the sequence of SEQ ID NO: 22.

In some embodiments of any of the aspects, the at least one domain of a virus spike protein comprises a sequence with at least 80% sequence identity to the sequence of SEQ ID NO: 23. In some embodiments of any of the aspects, the at least one domain of a virus spike protein comprises a sequence with at least 85% sequence identity to the sequence of SEQ ID NO: 23. In some embodiments of any of the aspects, the at least one domain of a virus spike protein comprises a sequence with at least 90% sequence identity to the sequence of SEQ ID NO: 23. In some embodiments of any of the aspects, the at least one domain of a virus spike protein comprises a sequence with at least 95% sequence identity to the sequence of SEQ ID NO: 23. In some embodiments of any of the aspects, the at least one domain of a virus spike protein comprises a sequence with at least 98% sequence identity to the sequence of SEQ ID NO: 23. In some embodiments of any of the aspects, the at least one domain of a virus spike protein comprises a sequence with at least 99% sequence identity to the sequence of SEQ ID NO: 23. In some embodiments of any of the aspects, the at least one domain of a virus spike protein comprises the sequence of SEQ ID NO: 23.

In some embodiments of any of the aspects, the at least one domain of a virus spike protein comprises a sequence with at least 80% sequence identity to the sequence of SEQ ID NO: 24. In some embodiments of any of the aspects, the at least one domain of a virus spike protein comprises a sequence with at least 85% sequence identity to the sequence of SEQ ID NO: 24. In some embodiments of any of the aspects, the at least one domain of a virus spike protein comprises a sequence with at least 90% sequence identity to the sequence of SEQ ID NO: 24. In some embodiments of any of the aspects, the at least one domain of a virus spike protein comprises a sequence with at least 95% sequence identity to the sequence of SEQ ID NO: 24. In some embodiments of any of the aspects, the at least one domain of a virus spike protein comprises a sequence with at least 98% sequence identity to the sequence of SEQ ID NO: 24. In some embodiments of any of the aspects, the at least one domain of a virus spike protein comprises a sequence with at least 99% sequence identity to the sequence of SEQ ID NO: 24. In some embodiments of any of the aspects, the at least one domain of a virus spike protein comprises the sequence of SEQ ID NO: 24.

In some embodiments of any of the aspects, the at least one domain of a virus spike protein comprises a sequence with at least 80% sequence identity to the sequence of SEQ ID NO: 25. In some embodiments of any of the aspects, the at least one domain of a virus spike protein comprises a sequence with at least 85% sequence identity to the sequence of SEQ ID NO: 25. In some embodiments of any of the aspects, the at least one domain of a virus spike protein comprises a sequence with at least 90% sequence identity to the sequence of SEQ ID NO: 25. In some embodiments of any of the aspects, the at least one domain of a virus spike protein comprises a sequence with at least 95% sequence identity to the sequence of SEQ ID NO: 25. In some embodiments of any of the aspects, the at least one domain of a virus spike protein comprises a sequence with at least 98% sequence identity to the sequence of SEQ ID NO: 25. In some embodiments of any of the aspects, the at least one domain of a virus spike protein comprises a sequence with at least 99% sequence identity to the sequence of SEQ ID NO: 25. In some embodiments of any of the aspects, the at least one domain of a virus spike protein comprises the sequence of SEQ ID NO: 25.

In some embodiments of any of the aspects, the at least one domain of a virus spike protein comprises a sequence with at least 80% sequence identity to the sequence of SEQ ID NO: 26. In some embodiments of any of the aspects, the at least one domain of a virus spike protein comprises a sequence with at least 85% sequence identity to the sequence of SEQ ID NO: 26. In some embodiments of any of the aspects, the at least one domain of a virus spike protein comprises a sequence with at least 90% sequence identity to the sequence of SEQ ID NO: 26. In some embodiments of any of the aspects, the at least one domain of a virus spike protein comprises a sequence with at least 95% sequence identity to the sequence of SEQ ID NO: 26. In some embodiments of any of the aspects, the at least one domain of a virus spike protein comprises a sequence with at least 98% sequence identity to the sequence of SEQ ID NO: 26. In some embodiments of any of the aspects, the at least one domain of a virus spike protein comprises a sequence with at least 99% sequence identity to the sequence of SEQ ID NO: 26. In some embodiments of any of the aspects, the at least one domain of a virus spike protein comprises the sequence of SEQ ID NO: 26.

In some embodiments of any of the aspects, the at least one domain of a virus spike protein comprises a sequence with at least 80% sequence identity to the sequence of SEQ ID NO: 27. In some embodiments of any of the aspects, the at least one domain of a virus spike protein comprises a sequence with at least 85% sequence identity to the sequence of SEQ ID NO: 27. In some embodiments of any of the aspects, the at least one domain of a virus spike protein comprises a sequence with at least 90% sequence identity to the sequence of SEQ ID NO: 27. In some embodiments of any of the aspects, the at least one domain of a virus spike protein comprises a sequence with at least 95% sequence identity to the sequence of SEQ ID NO: 27. In some embodiments of any of the aspects, the at least one domain of a virus spike protein comprises a sequence with at least 98% sequence identity to the sequence of SEQ ID NO: 27. In some embodiments of any of the aspects, the at least one domain of a virus spike protein comprises a sequence with at least 99% sequence identity to the sequence of SEQ ID NO: 27. In some embodiments of any of the aspects, the at least one domain of a virus spike protein comprises the sequence of SEQ ID NO: 27.

In some embodiments of any of the aspects, the at least one domain of a virus spike protein comprises a sequence with at least 80% sequence identity to the sequence of SEQ ID NO: 78. In some embodiments of any of the aspects, the at least one domain of a virus spike protein comprises a sequence with at least 85% sequence identity to the sequence of SEQ ID NO: 78. In some embodiments of any of the aspects, the at least one domain of a virus spike protein comprises a sequence with at least 90% sequence identity to the sequence of SEQ ID NO: 78. In some embodiments of any of the aspects, the at least one domain of a virus spike protein comprises a sequence with at least 95% sequence identity to the sequence of SEQ ID NO: 78. In some embodiments of any of the aspects, the at least one domain of a virus spike protein comprises a sequence with at least 98% sequence identity to the sequence of SEQ ID NO: 78. In some embodiments of any of the aspects, the at least one domain of a virus spike protein comprises a sequence with at least 99% sequence identity to the sequence of SEQ ID NO: 78. In some embodiments of any of the aspects, the at least one domain of a virus spike protein comprises the sequence of SEQ ID NO: 78.

In some embodiments of any of the aspects, the at least one domain of a virus spike protein comprises a sequence with at least 80% sequence identity to the sequence of SEQ ID NO: 79. In some embodiments of any of the aspects, the at least one domain of a virus spike protein comprises a sequence with at least 85% sequence identity to the sequence of SEQ ID NO: 79. In some embodiments of any of the aspects, the at least one domain of a virus spike protein comprises a sequence with at least 90% sequence identity to the sequence of SEQ ID NO: 79. In some embodiments of any of the aspects, the at least one domain of a virus spike protein comprises a sequence with at least 95% sequence identity to the sequence of SEQ ID NO: 79. In some embodiments of any of the aspects, the at least one domain of a virus spike protein comprises a sequence with at least 98% sequence identity to the sequence of SEQ ID NO: 79. In some embodiments of any of the aspects, the at least one domain of a virus spike protein comprises a sequence with at least 99% sequence identity to the sequence of SEQ ID NO: 79. In some embodiments of any of the aspects, the at least one domain of a virus spike protein comprises the sequence of SEQ ID NO: 79.

In some embodiments of any of the aspects, the at least one domain of a virus spike protein comprises a sequence with at least 80% sequence identity to the sequence of SEQ ID NO: 80. In some embodiments of any of the aspects, the at least one domain of a virus spike protein comprises a sequence with at least 85% sequence identity to the sequence of SEQ ID NO: 80. In some embodiments of any of the aspects, the at least one domain of a virus spike protein comprises a sequence with at least 90% sequence identity to the sequence of SEQ ID NO: 80. In some embodiments of any of the aspects, the at least one domain of a virus spike protein comprises a sequence with at least 95% sequence identity to the sequence of SEQ ID NO: 80. In some embodiments of any of the aspects, the at least one domain of a virus spike protein comprises a sequence with at least 98% sequence identity to the sequence of SEQ ID NO: 80. In some embodiments of any of the aspects, the at least one domain of a virus spike protein comprises a sequence with at least 99% sequence identity to the sequence of SEQ ID NO: 80. In some embodiments of any of the aspects, the at least one domain of a virus spike protein comprises the sequence of SEQ ID NO: 80.

In some embodiments of any of the aspects, the at least one domain of a virus spike protein comprises a sequence with at least 80% sequence identity to the sequence of SEQ ID NO: 81. In some embodiments of any of the aspects, the at least one domain of a virus spike protein comprises a sequence with at least 85% sequence identity to the sequence of SEQ ID NO: 81. In some embodiments of any of the aspects, the at least one domain of a virus spike protein comprises a sequence with at least 90% sequence identity to the sequence of SEQ ID NO: 81. In some embodiments of any of the aspects, the at least one domain of a virus spike protein comprises a sequence with at least 95% sequence identity to the sequence of SEQ ID NO: 81. In some embodiments of any of the aspects, the at least one domain of a virus spike protein comprises a sequence with at least 98% sequence identity to the sequence of SEQ ID NO: 81. In some embodiments of any of the aspects, the at least one domain of a virus spike protein comprises a sequence with at least 99% sequence identity to the sequence of SEQ ID NO: 81. In some embodiments of any of the aspects, the at least one domain of a virus spike protein comprises the sequence of SEQ ID NO: 81.

In some embodiments of any of the aspects, the at least one domain of a virus spike protein comprises a sequence with at least 80% sequence identity to the sequence of SEQ ID NO: 82. In some embodiments of any of the aspects, the at least one domain of a virus spike protein comprises a sequence with at least 85% sequence identity to the sequence of SEQ ID NO: 82. In some embodiments of any of the aspects, the at least one domain of a virus spike protein comprises a sequence with at least 90% sequence identity to the sequence of SEQ ID NO: 82. In some embodiments of any of the aspects, the at least one domain of a virus spike protein comprises a sequence with at least 95% sequence identity to the sequence of SEQ ID NO: 82. In some embodiments of any of the aspects, the at least one domain of a virus spike protein comprises a sequence with at least 98% sequence identity to the sequence of SEQ ID NO: 82. In some embodiments of any of the aspects, the at least one domain of a virus spike protein comprises a sequence with at least 99% sequence identity to the sequence of SEQ ID NO: 82. In some embodiments of any of the aspects, the at least one domain of a virus spike protein comprises the sequence of SEQ ID NO: 82.

In some embodiments of any of the aspects, the at least one domain of a virus spike protein comprises a sequence with at least 80% sequence identity to the sequence of SEQ ID NO: 83. In some embodiments of any of the aspects, the at least one domain of a virus spike protein comprises a sequence with at least 85% sequence identity to the sequence of SEQ ID NO: 83. In some embodiments of any of the aspects, the at least one domain of a virus spike protein comprises a sequence with at least 90% sequence identity to the sequence of SEQ ID NO: 83. In some embodiments of any of the aspects, the at least one domain of a virus spike protein comprises a sequence with at least 95% sequence identity to the sequence of SEQ ID NO: 83. In some embodiments of any of the aspects, the at least one domain of a virus spike protein comprises a sequence with at least 98% sequence identity to the sequence of SEQ ID NO: 83. In some embodiments of any of the aspects, the at least one domain of a virus spike protein comprises a sequence with at least 99% sequence identity to the sequence of SEQ ID NO: 83. In some embodiments of any of the aspects, the at least one domain of a virus spike protein comprises the sequence of SEQ ID NO: 83.

In some embodiments of any of the aspects, the at least one domain of a virus spike protein comprises a sequence with at least 80% sequence identity to the sequence of SEQ ID NO: 84. In some embodiments of any of the aspects, the at least one domain of a virus spike protein comprises a sequence with at least 85% sequence identity to the sequence of SEQ ID NO: 84. In some embodiments of any of the aspects, the at least one domain of a virus spike protein comprises a sequence with at least 90% sequence identity to the sequence of SEQ ID NO: 84. In some embodiments of any of the aspects, the at least one domain of a virus spike protein comprises a sequence with at least 95% sequence identity to the sequence of SEQ ID NO: 84. In some embodiments of any of the aspects, the at least one domain of a virus spike protein comprises a sequence with at least 98% sequence identity to the sequence of SEQ ID NO: 84. In some embodiments of any of the aspects, the at least one domain of a virus spike protein comprises a sequence with at least 99% sequence identity to the sequence of SEQ ID NO: 84. In some embodiments of any of the aspects, the at least one domain of a virus spike protein comprises the sequence of SEQ ID NO: 84.

In some embodiments of any of the aspects, the at least one domain of a virus spike protein comprises a sequence with at least 80% sequence identity to the sequence of SEQ ID NO: 90. In some embodiments of any of the aspects, the at least one domain of a virus spike protein comprises a sequence with at least 85% sequence identity to the sequence of SEQ ID NO: 90. In some embodiments of any of the aspects, the at least one domain of a virus spike protein comprises a sequence with at least 90% sequence identity to the sequence of SEQ ID NO: 90. In some embodiments of any of the aspects, the at least one domain of a virus spike protein comprises a sequence with at least 95% sequence identity to the sequence of SEQ ID NO: 90. In some embodiments of any of the aspects, the at least one domain of a virus spike protein comprises a sequence with at least 98% sequence identity to the sequence of SEQ ID NO: 90. In some embodiments of any of the aspects, the at least one domain of a virus spike protein comprises a sequence with at least 99% sequence identity to the sequence of SEQ ID NO: 90. In some embodiments of any of the aspects, the at least one domain of a virus spike protein comprises the sequence of SEQ ID NO: 90.

In some embodiments of any of the aspects, the at least one domain of a virus spike protein comprises a sequence with at least 80% sequence identity to the sequence of SEQ ID NO: 91. In some embodiments of any of the aspects, the at least one domain of a virus spike protein comprises a sequence with at least 85% sequence identity to the sequence of SEQ ID NO: 91. In some embodiments of any of the aspects, the at least one domain of a virus spike protein comprises a sequence with at least 90% sequence identity to the sequence of SEQ ID NO: 91. In some embodiments of any of the aspects, the at least one domain of a virus spike protein comprises a sequence with at least 95% sequence identity to the sequence of SEQ ID NO: 91. In some embodiments of any of the aspects, the at least one domain of a virus spike protein comprises a sequence with at least 98% sequence identity to the sequence of SEQ ID NO: 91. In some embodiments of any of the aspects, the at least one domain of a virus spike protein comprises a sequence with at least 99% sequence identity to the sequence of SEQ ID NO: 91. In some embodiments of any of the aspects, the at least one domain of a virus spike protein comprises the sequence of SEQ ID NO: 91.

In some embodiments of any of the aspects, the at least one domain of a virus spike protein comprises a sequence with at least 80% sequence identity to the sequence of SEQ ID NO: 92. In some embodiments of any of the aspects, the at least one domain of a virus spike protein comprises a sequence with at least 85% sequence identity to the sequence of SEQ ID NO: 92. In some embodiments of any of the aspects, the at least one domain of a virus spike protein comprises a sequence with at least 90% sequence identity to the sequence of SEQ ID NO: 92. In some embodiments of any of the aspects, the at least one domain of a virus spike protein comprises a sequence with at least 95% sequence identity to the sequence of SEQ ID NO: 92. In some embodiments of any of the aspects, the at least one domain of a virus spike protein comprises a sequence with at least 98% sequence identity to the sequence of SEQ ID NO: 92. In some embodiments of any of the aspects, the at least one domain of a virus spike protein comprises a sequence with at least 99% sequence identity to the sequence of SEQ ID NO: 92. In some embodiments of any of the aspects, the at least one domain of a virus spike protein comprises the sequence of SEQ ID NO: 92.

In some embodiments of any of the aspects, the at least one domain of a virus spike protein comprises a sequence with at least 80% sequence identity to the sequence of SEQ ID NO: 93. In some embodiments of any of the aspects, the at least one domain of a virus spike protein comprises a sequence with at least 85% sequence identity to the sequence of SEQ ID NO: 93. In some embodiments of any of the aspects, the at least one domain of a virus spike protein comprises a sequence with at least 90% sequence identity to the sequence of SEQ ID NO: 93. In some embodiments of any of the aspects, the at least one domain of a virus spike protein comprises a sequence with at least 95% sequence identity to the sequence of SEQ ID NO: 93. In some embodiments of any of the aspects, the at least one domain of a virus spike protein comprises a sequence with at least 98% sequence identity to the sequence of SEQ ID NO: 93. In some embodiments of any of the aspects, the at least one domain of a virus spike protein comprises a sequence with at least 99% sequence identity to the sequence of SEQ ID NO: 93. In some embodiments of any of the aspects, the at least one domain of a virus spike protein comprises the sequence of SEQ ID NO: 93.

In some embodiments of any of the aspects, the at least one domain of a virus spike protein comprises a sequence with at least 80% sequence identity to the sequence of SEQ ID NO: 94. In some embodiments of any of the aspects, the at least one domain of a virus spike protein comprises a sequence with at least 85% sequence identity to the sequence of SEQ ID NO: 94. In some embodiments of any of the aspects, the at least one domain of a virus spike protein comprises a sequence with at least 90% sequence identity to the sequence of SEQ ID NO: 94. In some embodiments of any of the aspects, the at least one domain of a virus spike protein comprises a sequence with at least 95% sequence identity to the sequence of SEQ ID NO: 94. In some embodiments of any of the aspects, the at least one domain of a virus spike protein comprises a sequence with at least 98% sequence identity to the sequence of SEQ ID NO: 94. In some embodiments of any of the aspects, the at least one domain of a virus spike protein comprises a sequence with at least 99% sequence identity to the sequence of SEQ ID NO: 94. In some embodiments of any of the aspects, the at least one domain of a virus spike protein comprises the sequence of SEQ ID NO: 94.

In some embodiments of any of the aspects, the at least one domain of a virus spike protein comprises a sequence with at least 80% sequence identity to the sequence of SEQ ID NO: 95. In some embodiments of any of the aspects, the at least one domain of a virus spike protein comprises a sequence with at least 85% sequence identity to the sequence of SEQ ID NO: 95. In some embodiments of any of the aspects, the at least one domain of a virus spike protein comprises a sequence with at least 90% sequence identity to the sequence of SEQ ID NO: 95. In some embodiments of any of the aspects, the at least one domain of a virus spike protein comprises a sequence with at least 95% sequence identity to the sequence of SEQ ID NO: 95. In some embodiments of any of the aspects, the at least one domain of a virus spike protein comprises a sequence with at least 98% sequence identity to the sequence of SEQ ID NO: 95. In some embodiments of any of the aspects, the at least one domain of a virus spike protein comprises a sequence with at least 99% sequence identity to the sequence of SEQ ID NO: 95. In some embodiments of any of the aspects, the at least one domain of a virus spike protein comprises the sequence of SEQ ID NO: 95.

In some embodiments of any of the aspects, the at least one domain of a virus spike protein comprises a sequence with at least 80% sequence identity to the sequence of SEQ ID NO: 96. In some embodiments of any of the aspects, the at least one domain of a virus spike protein comprises a sequence with at least 85% sequence identity to the sequence of SEQ ID NO: 96. In some embodiments of any of the aspects, the at least one domain of a virus spike protein comprises a sequence with at least 90% sequence identity to the sequence of SEQ ID NO: 96. In some embodiments of any of the aspects, the at least one domain of a virus spike protein comprises a sequence with at least 95% sequence identity to the sequence of SEQ ID NO: 96. In some embodiments of any of the aspects, the at least one domain of a virus spike protein comprises a sequence with at least 98% sequence identity to the sequence of SEQ ID NO: 96. In some embodiments of any of the aspects, the at least one domain of a virus spike protein comprises a sequence with at least 99% sequence identity to the sequence of SEQ ID NO: 96. In some embodiments of any of the aspects, the at least one domain of a virus spike protein comprises the sequence of SEQ ID NO: 96.

In some embodiments of any of the aspects, the at least one domain of a virus spike protein comprises a sequence with at least 80% sequence identity to the sequence of SEQ ID NO: 123. In some embodiments of any of the aspects, the at least one domain of a virus spike protein comprises a sequence with at least 85% sequence identity to the sequence of SEQ ID NO: 123. In some embodiments of any of the aspects, the at least one domain of a virus spike protein comprises a sequence with at least 90% sequence identity to the sequence of SEQ ID NO: 123. In some embodiments of any of the aspects, the at least one domain of a virus spike protein comprises a sequence with at least 95% sequence identity to the sequence of SEQ ID NO: 123. In some embodiments of any of the aspects, the at least one domain of a virus spike protein comprises a sequence with at least 98% sequence identity to the sequence of SEQ ID NO: 123. In some embodiments of any of the aspects, the at least one domain of a virus spike protein comprises a sequence with at least 99% sequence identity to the sequence of SEQ ID NO: 123. In some embodiments of any of the aspects, the at least one domain of a virus spike protein comprises the sequence of SEQ ID NO: 123.

In some embodiments of any of the aspects, the at least one domain of a virus spike protein comprises a sequence with at least 80% sequence identity to the sequence of SEQ ID NO: 124. In some embodiments of any of the aspects, the at least one domain of a virus spike protein comprises a sequence with at least 85% sequence identity to the sequence of SEQ ID NO: 124. In some embodiments of any of the aspects, the at least one domain of a virus spike protein comprises a sequence with at least 90% sequence identity to the sequence of SEQ ID NO: 124. In some embodiments of any of the aspects, the at least one domain of a virus spike protein comprises a sequence with at least 95% sequence identity to the sequence of SEQ ID NO: 124. In some embodiments of any of the aspects, the at least one domain of a virus spike protein comprises a sequence with at least 98% sequence identity to the sequence of SEQ ID NO: 124. In some embodiments of any of the aspects, the at least one domain of a virus spike protein comprises a sequence with at least 99% sequence identity to the sequence of SEQ ID NO: 124. In some embodiments of any of the aspects, the at least one domain of a virus spike protein comprises the sequence of SEQ ID NO: 124.

In some embodiments of any of the aspects, the at least one domain of a virus spike protein comprises a sequence with at least 80% sequence identity to the sequence of SEQ ID NO: 125. In some embodiments of any of the aspects, the at least one domain of a virus spike protein comprises a sequence with at least 85% sequence identity to the sequence of SEQ ID NO: 125. In some embodiments of any of the aspects, the at least one domain of a virus spike protein comprises a sequence with at least 90% sequence identity to the sequence of SEQ ID NO: 125. In some embodiments of any of the aspects, the at least one domain of a virus spike protein comprises a sequence with at least 95% sequence identity to the sequence of SEQ ID NO: 125. In some embodiments of any of the aspects, the at least one domain of a virus spike protein comprises a sequence with at least 98% sequence identity to the sequence of SEQ ID NO: 125. In some embodiments of any of the aspects, the at least one domain of a virus spike protein comprises a sequence with at least 99% sequence identity to the sequence of SEQ ID NO: 125. In some embodiments of any of the aspects, the at least one domain of a virus spike protein comprises the sequence of SEQ ID NO: 125.

In some embodiments of any of the aspects, the at least one domain of a virus spike protein comprises a sequence with at least 80% sequence identity to the sequence of SEQ ID NO: 126. In some embodiments of any of the aspects, the at least one domain of a virus spike protein comprises a sequence with at least 85% sequence identity to the sequence of SEQ ID NO: 126. In some embodiments of any of the aspects, the at least one domain of a virus spike protein comprises a sequence with at least 90% sequence identity to the sequence of SEQ ID NO: 126. In some embodiments of any of the aspects, the at least one domain of a virus spike protein comprises a sequence with at least 95% sequence identity to the sequence of SEQ ID NO: 126. In some embodiments of any of the aspects, the at least one domain of a virus spike protein comprises a sequence with at least 98% sequence identity to the sequence of SEQ ID NO: 126. In some embodiments of any of the aspects, the at least one domain of a virus spike protein comprises a sequence with at least 99% sequence identity to the sequence of SEQ ID NO: 126. In some embodiments of any of the aspects, the at least one domain of a virus spike protein comprises the sequence of SEQ ID NO: 126.

In some embodiments of any of the aspects, the at least one domain of a virus spike protein comprises a sequence with at least 80% sequence identity to the sequence of SEQ ID NO: 127. In some embodiments of any of the aspects, the at least one domain of a virus spike protein comprises a sequence with at least 85% sequence identity to the sequence of SEQ ID NO: 127. In some embodiments of any of the aspects, the at least one domain of a virus spike protein comprises a sequence with at least 90% sequence identity to the sequence of SEQ ID NO: 127. In some embodiments of any of the aspects, the at least one domain of a virus spike protein comprises a sequence with at least 95% sequence identity to the sequence of SEQ ID NO: 127. In some embodiments of any of the aspects, the at least one domain of a virus spike protein comprises a sequence with at least 98% sequence identity to the sequence of SEQ ID NO: 127. In some embodiments of any of the aspects, the at least one domain of a virus spike protein comprises a sequence with at least 99% sequence identity to the sequence of SEQ ID NO: 127. In some embodiments of any of the aspects, the at least one domain of a virus spike protein comprises the sequence of SEQ ID NO: 127.

In some embodiments of any of the aspects, the at least one domain of a virus spike protein comprises a sequence with at least 80% sequence identity to the sequence of SEQ ID NO: 128. In some embodiments of any of the aspects, the at least one domain of a virus spike protein comprises a sequence with at least 85% sequence identity to the sequence of SEQ ID NO: 128. In some embodiments of any of the aspects, the at least one domain of a virus spike protein comprises a sequence with at least 90% sequence identity to the sequence of SEQ ID NO: 128. In some embodiments of any of the aspects, the at least one domain of a virus spike protein comprises a sequence with at least 95% sequence identity to the sequence of SEQ ID NO: 128. In some embodiments of any of the aspects, the at least one domain of a virus spike protein comprises a sequence with at least 98% sequence identity to the sequence of SEQ ID NO: 128. In some embodiments of any of the aspects, the at least one domain of a virus spike protein comprises a sequence with at least 99% sequence identity to the sequence of SEQ ID NO: 128. In some embodiments of any of the aspects, the at least one domain of a virus spike protein comprises the sequence of SEQ ID NO: 128.

In some embodiments of any of the aspects, the at least one domain of a virus spike protein comprises a sequence with at least 80% sequence identity to the sequence of SEQ ID NO: 129. In some embodiments of any of the aspects, the at least one domain of a virus spike protein comprises a sequence with at least 85% sequence identity to the sequence of SEQ ID NO: 129. In some embodiments of any of the aspects, the at least one domain of a virus spike protein comprises a sequence with at least 90% sequence identity to the sequence of SEQ ID NO: 129. In some embodiments of any of the aspects, the at least one domain of a virus spike protein comprises a sequence with at least 95% sequence identity to the sequence of SEQ ID NO: 129. In some embodiments of any of the aspects, the at least one domain of a virus spike protein comprises a sequence with at least 98% sequence identity to the sequence of SEQ ID NO: 129. In some embodiments of any of the aspects, the at least one domain of a virus spike protein comprises a sequence with at least 99% sequence identity to the sequence of SEQ ID NO: 129. In some embodiments of any of the aspects, the at least one domain of a virus spike protein comprises the sequence of SEQ ID NO: 129.

In some embodiments of any of the aspects, the at least one domain of a virus spike protein comprises a sequence with at least 80% sequence identity to the sequence of SEQ ID NO: 130. In some embodiments of any of the aspects, the at least one domain of a virus spike protein comprises a sequence with at least 85% sequence identity to the sequence of SEQ ID NO: 130. In some embodiments of any of the aspects, the at least one domain of a virus spike protein comprises a sequence with at least 90% sequence identity to the sequence of SEQ ID NO: 130. In some embodiments of any of the aspects, the at least one domain of a virus spike protein comprises a sequence with at least 95% sequence identity to the sequence of SEQ ID NO: 130. In some embodiments of any of the aspects, the at least one domain of a virus spike protein comprises a sequence with at least 98% sequence identity to the sequence of SEQ ID NO: 130. In some embodiments of any of the aspects, the at least one domain of a virus spike protein comprises a sequence with at least 99% sequence identity to the sequence of SEQ ID NO: 130. In some embodiments of any of the aspects, the at least one domain of a virus spike protein comprises the sequence of SEQ ID NO: 130.

In some embodiments of any of the aspects, the at least one domain of a virus spike protein comprises a sequence with at least 80% sequence identity to the sequence of SEQ ID NO: 131. In some embodiments of any of the aspects, the at least one domain of a virus spike protein comprises a sequence with at least 85% sequence identity to the sequence of SEQ ID NO: 131. In some embodiments of any of the aspects, the at least one domain of a virus spike protein comprises a sequence with at least 90% sequence identity to the sequence of SEQ ID NO: 131. In some embodiments of any of the aspects, the at least one domain of a virus spike protein comprises a sequence with at least 95% sequence identity to the sequence of SEQ ID NO: 131. In some embodiments of any of the aspects, the at least one domain of a virus spike protein comprises a sequence with at least 98% sequence identity to the sequence of SEQ ID NO: 131. In some embodiments of any of the aspects, the at least one domain of a virus spike protein comprises a sequence with at least 99% sequence identity to the sequence of SEQ ID NO: 131. In some embodiments of any of the aspects, the at least one domain of a virus spike protein comprises the sequence of SEQ ID NO: 131.

In some embodiments of any of the aspects, the at least one domain of a virus spike protein comprises a sequence with at least 80% sequence identity to the sequence of SEQ ID NO: 132. In some embodiments of any of the aspects, the at least one domain of a virus spike protein comprises a sequence with at least 85% sequence identity to the sequence of SEQ ID NO: 132. In some embodiments of any of the aspects, the at least one domain of a virus spike protein comprises a sequence with at least 90% sequence identity to the sequence of SEQ ID NO: 132. In some embodiments of any of the aspects, the at least one domain of a virus spike protein comprises a sequence with at least 95% sequence identity to the sequence of SEQ ID NO: 132. In some embodiments of any of the aspects, the at least one domain of a virus spike protein comprises a sequence with at least 98% sequence identity to the sequence of SEQ ID NO: 132. In some embodiments of any of the aspects, the at least one domain of a virus spike protein comprises a sequence with at least 99% sequence identity to the sequence of SEQ ID NO: 132. In some embodiments of any of the aspects, the at least one domain of a virus spike protein comprises the sequence of SEQ ID NO: 132.

In some embodiments of any of the aspects, the at least one domain of a virus spike protein comprises a sequence with at least 80% sequence identity to the sequence of SEQ ID NO: 133. In some embodiments of any of the aspects, the at least one domain of a virus spike protein comprises a sequence with at least 85% sequence identity to the sequence of SEQ ID NO: 133. In some embodiments of any of the aspects, the at least one domain of a virus spike protein comprises a sequence with at least 90% sequence identity to the sequence of SEQ ID NO: 133. In some embodiments of any of the aspects, the at least one domain of a virus spike protein comprises a sequence with at least 95% sequence identity to the sequence of SEQ ID NO: 133. In some embodiments of any of the aspects, the at least one domain of a virus spike protein comprises a sequence with at least 98% sequence identity to the sequence of SEQ ID NO: 133. In some embodiments of any of the aspects, the at least one domain of a virus spike protein comprises a sequence with at least 99% sequence identity to the sequence of SEQ ID NO: 133. In some embodiments of any of the aspects, the at least one domain of a virus spike protein comprises the sequence of SEQ ID NO: 133.

In some embodiments of any of the aspects, the at least one domain of a virus spike protein comprises a sequence with at least 80% sequence identity to the sequence of SEQ ID NO: 134. In some embodiments of any of the aspects, the at least one domain of a virus spike protein comprises a sequence with at least 85% sequence identity to the sequence of SEQ ID NO: 134. In some embodiments of any of the aspects, the at least one domain of a virus spike protein comprises a sequence with at least 90% sequence identity to the sequence of SEQ ID NO: 134. In some embodiments of any of the aspects, the at least one domain of a virus spike protein comprises a sequence with at least 95% sequence identity to the sequence of SEQ ID NO: 134. In some embodiments of any of the aspects, the at least one domain of a virus spike protein comprises a sequence with at least 98% sequence identity to the sequence of SEQ ID NO: 134. In some embodiments of any of the aspects, the at least one domain of a virus spike protein comprises a sequence with at least 99% sequence identity to the sequence of SEQ ID NO: 134. In some embodiments of any of the aspects, the at least one domain of a virus spike protein comprises the sequence of SEQ ID NO: 134.

In some embodiments of any of the aspects, the at least one domain of a virus spike protein comprises a sequence with at least 80% sequence identity to the sequence of SEQ ID NO: 143. In some embodiments of any of the aspects, the at least one domain of a virus spike protein comprises a sequence with at least 85% sequence identity to the sequence of SEQ ID NO: 143. In some embodiments of any of the aspects, the at least one domain of a virus spike protein comprises a sequence with at least 90% sequence identity to the sequence of SEQ ID NO: 143. In some embodiments of any of the aspects, the at least one domain of a virus spike protein comprises a sequence with at least 95% sequence identity to the sequence of SEQ ID NO: 143. In some embodiments of any of the aspects, the at least one domain of a virus spike protein comprises a sequence with at least 98% sequence identity to the sequence of SEQ ID NO: 143. In some embodiments of any of the aspects, the at least one domain of a virus spike protein comprises a sequence with at least 99% sequence identity to the sequence of SEQ ID NO: 143. In some embodiments of any of the aspects, the at least one domain of a virus spike protein comprises the sequence of SEQ ID NO: 143.

In some embodiments of any of the aspects, the at least one domain of a virus spike protein comprises a sequence with at least 80% sequence identity to the sequence of SEQ ID NO: 144. In some embodiments of any of the aspects, the at least one domain of a virus spike protein comprises a sequence with at least 85% sequence identity to the sequence of SEQ ID NO: 144. In some embodiments of any of the aspects, the at least one domain of a virus spike protein comprises a sequence with at least 90% sequence identity to the sequence of SEQ ID NO: 144. In some embodiments of any of the aspects, the at least one domain of a virus spike protein comprises a sequence with at least 95% sequence identity to the sequence of SEQ ID NO: 144. In some embodiments of any of the aspects, the at least one domain of a virus spike protein comprises a sequence with at least 98% sequence identity to the sequence of SEQ ID NO: 144. In some embodiments of any of the aspects, the at least one domain of a virus spike protein comprises a sequence with at least 99% sequence identity to the sequence of SEQ ID NO: 144. In some embodiments of any of the aspects, the at least one domain of a virus spike protein comprises the sequence of SEQ ID NO: 144.

In some embodiments of any of the aspects, the at least one domain of a virus spike protein comprises a sequence with at least 80% sequence identity to the sequence of SEQ ID NO: 145. In some embodiments of any of the aspects, the at least one domain of a virus spike protein comprises a sequence with at least 85% sequence identity to the sequence of SEQ ID NO: 145. In some embodiments of any of the aspects, the at least one domain of a virus spike protein comprises a sequence with at least 90% sequence identity to the sequence of SEQ ID NO: 145. In some embodiments of any of the aspects, the at least one domain of a virus spike protein comprises a sequence with at least 95% sequence identity to the sequence of SEQ ID NO: 145. In some embodiments of any of the aspects, the at least one domain of a virus spike protein comprises a sequence with at least 98% sequence identity to the sequence of SEQ ID NO: 145. In some embodiments of any of the aspects, the at least one domain of a virus spike protein comprises a sequence with at least 99% sequence identity to the sequence of SEQ ID NO: 145. In some embodiments of any of the aspects, the at least one domain of a virus spike protein comprises the sequence of SEQ ID NO: 145.

In some embodiments of any of the aspects, the at least one domain of a virus spike protein comprises a sequence with at least 80% sequence identity to the sequence of SEQ ID NO: 146. In some embodiments of any of the aspects, the at least one domain of a virus spike protein comprises a sequence with at least 85% sequence identity to the sequence of SEQ ID NO: 146. In some embodiments of any of the aspects, the at least one domain of a virus spike protein comprises a sequence with at least 90% sequence identity to the sequence of SEQ ID NO: 146. In some embodiments of any of the aspects, the at least one domain of a virus spike protein comprises a sequence with at least 95% sequence identity to the sequence of SEQ ID NO: 146. In some embodiments of any of the aspects, the at least one domain of a virus spike protein comprises a sequence with at least 98% sequence identity to the sequence of SEQ ID NO: 146. In some embodiments of any of the aspects, the at least one domain of a virus spike protein comprises a sequence with at least 99% sequence identity to the sequence of SEQ ID NO: 146. In some embodiments of any of the aspects, the at least one domain of a virus spike protein comprises the sequence of SEQ ID NO: 146.

In some embodiments of any of the aspects, the at least one domain of a virus spike protein comprises a sequence with at least 80% sequence identity to the sequence of SEQ ID NO: 147. In some embodiments of any of the aspects, the at least one domain of a virus spike protein comprises a sequence with at least 85% sequence identity to the sequence of SEQ ID NO: 147. In some embodiments of any of the aspects, the at least one domain of a virus spike protein comprises a sequence with at least 90% sequence identity to the sequence of SEQ ID NO: 147. In some embodiments of any of the aspects, the at least one domain of a virus spike protein comprises a sequence with at least 95% sequence identity to the sequence of SEQ ID NO: 147. In some embodiments of any of the aspects, the at least one domain of a virus spike protein comprises a sequence with at least 98% sequence identity to the sequence of SEQ ID NO: 147. In some embodiments of any of the aspects, the at least one domain of a virus spike protein comprises a sequence with at least 99% sequence identity to the sequence of SEQ ID NO: 147. In some embodiments of any of the aspects, the at least one domain of a virus spike protein comprises the sequence of SEQ ID NO: 147.

In some embodiments of any of the aspects, the at least one domain of a virus spike protein comprises a sequence with at least 80% sequence identity to the sequence of SEQ ID NO: 148. In some embodiments of any of the aspects, the at least one domain of a virus spike protein comprises a sequence with at least 85% sequence identity to the sequence of SEQ ID NO: 148. In some embodiments of any of the aspects, the at least one domain of a virus spike protein comprises a sequence with at least 90% sequence identity to the sequence of SEQ ID NO: 148. In some embodiments of any of the aspects, the at least one domain of a virus spike protein comprises a sequence with at least 95% sequence identity to the sequence of SEQ ID NO: 148. In some embodiments of any of the aspects, the at least one domain of a virus spike protein comprises a sequence with at least 98% sequence identity to the sequence of SEQ ID NO: 148. In some embodiments of any of the aspects, the at least one domain of a virus spike protein comprises a sequence with at least 99% sequence identity to the sequence of SEQ ID NO: 148. In some embodiments of any of the aspects, the at least one domain of a virus spike protein comprises the sequence of SEQ ID NO: 148.

In some embodiments of any of the aspects, the at least one domain of a virus spike protein comprises a sequence with at least 80% sequence identity to the sequence of SEQ ID NO: 149. In some embodiments of any of the aspects, the at least one domain of a virus spike protein comprises a sequence with at least 85% sequence identity to the sequence of SEQ ID NO: 149. In some embodiments of any of the aspects, the at least one domain of a virus spike protein comprises a sequence with at least 90% sequence identity to the sequence of SEQ ID NO: 149. In some embodiments of any of the aspects, the at least one domain of a virus spike protein comprises a sequence with at least 95% sequence identity to the sequence of SEQ ID NO: 149. In some embodiments of any of the aspects, the at least one domain of a virus spike protein comprises a sequence with at least 98% sequence identity to the sequence of SEQ ID NO: 149. In some embodiments of any of the aspects, the at least one domain of a virus spike protein comprises a sequence with at least 99% sequence identity to the sequence of SEQ ID NO: 149. In some embodiments of any of the aspects, the at least one domain of a virus spike protein comprises the sequence of SEQ ID NO: 149.

In some embodiments of any of the aspects, the at least one domain of a virus spike protein comprises a sequence with at least 80% sequence identity to the sequence of SEQ ID NO: 150. In some embodiments of any of the aspects, the at least one domain of a virus spike protein comprises a sequence with at least 85% sequence identity to the sequence of SEQ ID NO: 150. In some embodiments of any of the aspects, the at least one domain of a virus spike protein comprises a sequence with at least 90% sequence identity to the sequence of SEQ ID NO: 150. In some embodiments of any of the aspects, the at least one domain of a virus spike protein comprises a sequence with at least 95% sequence identity to the sequence of SEQ ID NO: 150. In some embodiments of any of the aspects, the at least one domain of a virus spike protein comprises a sequence with at least 98% sequence identity to the sequence of SEQ ID NO: 150. In some embodiments of any of the aspects, the at least one domain of a virus spike protein comprises a sequence with at least 99% sequence identity to the sequence of SEQ ID NO: 150. In some embodiments of any of the aspects, the at least one domain of a virus spike protein comprises the sequence of SEQ ID NO: 150.

In some embodiments of any of the aspects, the viral spike protein is a coronavirus spike protein. In some embodiments of any of the aspects, the viral spike protein is an alphacoronavirus spike protein. In some embodiments of any of the aspects, the viral spike protein is a betacoronavirus spike protein. In some embodiments of any of the aspects, the viral spike protein is a MERS-CoV spike protein, SARS-CoV-1 spike protein, SARS-CoV-2 spike protein, hCoV-HKU1 spike protein, hCoV-HL63 spike protein, hCoV-229E spike protein, or hCoV-OC43 spike protein.

In some embodiments of any of the aspects, the virus (e.g., the first or second virus, independently) is a coronavirus. In some embodiments of any of the aspects, the virus (e.g., the first or second virus, independently) is an alphacoronavirus. In some embodiments of any of the aspects, the virus (e.g., the first or second virus, independently) is a betacoronavirus. In some embodiments of any of the aspects, the virus (e.g., the first or second virus, independently) is MERS-CoV, SARS-CoV-1, SARS-CoV-2, hCoV-HKU1, hCoV-HL63, hCoV-229E, or hCoV-OC43. In some embodiments of any of the aspects, the virus (e.g., the first or second virus, independently) is SARS-CoV-2 Omicron XBB.1.5 or SARS-CoV-2 Delta.

In some embodiments of any of the aspects, the first virus is a coronavirus and the second virus is a coronavirus. In some embodiments of any of the aspects, the first virus is a member of a first clade of a viral genus and the second virus is a member of a second clade of the viral genus, e.g., the first virus and second virus are members of the same viral genus but are members of different clades within that genus.

As demonstrated herein, the polypeptides described herein are particularly efficacious when assembled into nanoparticles. Nanoparticles formed by polypeptide multimerization domains are of significant utility and are shown herein to provide excellent immunogenicity for the polypeptides described herein. Accordingly, in some embodiments of any of the aspects, a polypeptide described herein can comprise a multimerization domain. As used herein, a “multimerization domain” is a polypeptide domain that will form a multimer, either by direct binding to other multimerization domains, or by binding to a nanoparticle. In some embodiments of any of the aspects, the multimerization domain forms dimers. In some embodiments of any of the aspects, the multimerization domain forms trimers.

Exemplary multimerization domains are known in the art and include foldon, Apoferritin, Lumazine Synthase, and I53_dn5B. Further discussion of suitable multimerization domains can be found in the art at, e.g., Ueda, G. et al. Elife 9, e57659 (2020) and Azharuddin et al. Trends in Biotechnology 40:1195-1212 (2022); each of which is incorporated by reference herein in its entirety.

In some embodiments of any of the aspects, the multimerization domain comprises I53_dn5B. In some embodiments of any of the aspects, the multimerization domain consists essentially of I53_dn5B. In some embodiments of any of the aspects, the multimerization domain consists of I53_dn5B.

As used herein, “foldon” refers to a trimerization domain having the sequence:

(SEQ ID NO: 97)
GYIPEAPRDGQAYVRKDGEWVLLSTFL.

In some embodiments of any of the aspects the multimerization domain comprises a sequence with at least 80% sequence identity to the sequence of one of SEQ ID NOs: 17-20, 97, and 122. In some embodiments of any of the aspects the multimerization domain comprises a sequence with at least 85% sequence identity to the sequence of one of SEQ ID NOs: 17-20, 97, and 122. In some embodiments of any of the aspects the multimerization domain comprises a sequence with at least 90% sequence identity to the sequence of one of SEQ ID NOs: 17-20, 97, and 122. In some embodiments of any of the aspects the multimerization domain comprises a sequence with at least 95% sequence identity to the sequence of one of SEQ ID NOs: 17-20, 97, and 122. In some embodiments of any of the aspects the multimerization domain comprises a sequence with at least 98% sequence identity to the sequence of one of SEQ ID NOs: 17-20, 97, and 122. In some embodiments of any of the aspects the multimerization domain comprises a sequence with at least 99% sequence identity to the sequence of one of SEQ ID NOs: 17-20, 97, and 122. In some embodiments of any of the aspects the multimerization domain comprises the sequence of one of SEQ ID NOs: 17-20, 97, and 122.

In some embodiments of any of the aspects the multimerization domain comprises a sequence with at least 80% sequence identity to the sequence of SEQ ID NO: 17. In some embodiments of any of the aspects the multimerization domain comprises a sequence with at least 85% sequence identity to the sequence of SEQ ID NO: 17. In some embodiments of any of the aspects the multimerization domain comprises a sequence with at least 90% sequence identity to the sequence of SEQ ID NO: 17. In some embodiments of any of the aspects the multimerization domain comprises a sequence with at least 95% sequence identity to the sequence of SEQ ID NO: 17. In some embodiments of any of the aspects the multimerization domain comprises a sequence with at least 98% sequence identity to the sequence SEQ ID NO: 17. In some embodiments of any of the aspects the multimerization domain comprises a sequence with at least 99% sequence identity to the sequence of SEQ ID NO: 17. In some embodiments of any of the aspects the multimerization domain comprises the sequence of SEQ ID NO: 17.

In some embodiments of any of the aspects the multimerization domain comprises a sequence with at least 80% sequence identity to the sequence of SEQ ID NO: 18. In some embodiments of any of the aspects the multimerization domain comprises a sequence with at least 85% sequence identity to the sequence of SEQ ID NO: 18. In some embodiments of any of the aspects the multimerization domain comprises a sequence with at least 90% sequence identity to the sequence of SEQ ID NO: 18. In some embodiments of any of the aspects the multimerization domain comprises a sequence with at least 95% sequence identity to the sequence of SEQ ID NO: 18. In some embodiments of any of the aspects the multimerization domain comprises a sequence with at least 98% sequence identity to the sequence SEQ ID NO: 18. In some embodiments of any of the aspects the multimerization domain comprises a sequence with at least 99% sequence identity to the sequence of SEQ ID NO: 18. In some embodiments of any of the aspects the multimerization domain comprises the sequence of SEQ ID NO: 18.

In some embodiments of any of the aspects the multimerization domain comprises a sequence with at least 80% sequence identity to the sequence of SEQ ID NO: 19. In some embodiments of any of the aspects the multimerization domain comprises a sequence with at least 85% sequence identity to the sequence of SEQ ID NO: 19. In some embodiments of any of the aspects the multimerization domain comprises a sequence with at least 90% sequence identity to the sequence of SEQ ID NO: 19. In some embodiments of any of the aspects the multimerization domain comprises a sequence with at least 95% sequence identity to the sequence of SEQ ID NO: 19. In some embodiments of any of the aspects the multimerization domain comprises a sequence with at least 98% sequence identity to the sequence SEQ ID NO: 19. In some embodiments of any of the aspects the multimerization domain comprises a sequence with at least 99% sequence identity to the sequence of SEQ ID NO: 19. In some embodiments of any of the aspects the multimerization domain comprises the sequence of SEQ ID NO: 19.

In some embodiments of any of the aspects the multimerization domain comprises a sequence with at least 80% sequence identity to the sequence of SEQ ID NO: 20. In some embodiments of any of the aspects the multimerization domain comprises a sequence with at least 85% sequence identity to the sequence of SEQ ID NO: 20. In some embodiments of any of the aspects the multimerization domain comprises a sequence with at least 90% sequence identity to the sequence of SEQ ID NO: 20. In some embodiments of any of the aspects the multimerization domain comprises a sequence with at least 95% sequence identity to the sequence of SEQ ID NO: 20. In some embodiments of any of the aspects the multimerization domain comprises a sequence with at least 98% sequence identity to the sequence SEQ ID NO: 20. In some embodiments of any of the aspects the multimerization domain comprises a sequence with at least 99% sequence identity to the sequence of SEQ ID NO: 20. In some embodiments of any of the aspects the multimerization domain comprises the sequence of SEQ ID NO: 20.

In some embodiments of any of the aspects the multimerization domain comprises a sequence with at least 80% sequence identity to the sequence of SEQ ID NO: 97. In some embodiments of any of the aspects the multimerization domain comprises a sequence with at least 85% sequence identity to the sequence of SEQ ID NO: 97. In some embodiments of any of the aspects the multimerization domain comprises a sequence with at least 90% sequence identity to the sequence of SEQ ID NO: 97. In some embodiments of any of the aspects the multimerization domain comprises a sequence with at least 95% sequence identity to the sequence of SEQ ID NO: 97. In some embodiments of any of the aspects the multimerization domain comprises a sequence with at least 98% sequence identity to the sequence SEQ ID NO: 97. In some embodiments of any of the aspects the multimerization domain comprises a sequence with at least 99% sequence identity to the sequence of SEQ ID NO: 97. In some embodiments of any of the aspects the multimerization domain comprises the sequence of SEQ ID NO: 97.

In some embodiments of any of the aspects the multimerization domain comprises a sequence with at least 80% sequence identity to the sequence of SEQ ID NO: 122. In some embodiments of any of the aspects the multimerization domain comprises a sequence with at least 85% sequence identity to the sequence of SEQ ID NO: 122. In some embodiments of any of the aspects the multimerization domain comprises a sequence with at least 90% sequence identity to the sequence of SEQ ID NO: 122. In some embodiments of any of the aspects the multimerization domain comprises a sequence with at least 95% sequence identity to the sequence of SEQ ID NO: 122. In some embodiments of any of the aspects the multimerization domain comprises a sequence with at least 98% sequence identity to the sequence SEQ ID NO: 122. In some embodiments of any of the aspects the multimerization domain comprises a sequence with at least 99% sequence identity to the sequence of SEQ ID NO: 122. In some embodiments of any of the aspects the multimerization domain comprises the sequence of SEQ ID NO: 122.

In some embodiments of any of the aspects, a polypeptide described herein does not comprise a multimerization domain. Polypeptides described herein can be loaded onto, covalently linked to, encapsulated into, or formed into a nanoparticle by any method known in the art. See, e.g., Azharuddin et al. Trends in Biotechnology 40:1195-1212 (2022); which is incorporated by reference herein. For example, metal, carbon nanotube, liposome, micelles, dendrimer, polymers, and biomacromolecules are known to form suitable nanoparticles.

In some embodiments of any of the aspects, a polypeptide described herein further comprises a linker domain. In some embodiments of any of the aspects, the linker domain is located between the display domain and the multimerization domain. In some embodiments of any of the aspects, the linker domain is located between the at least one viral spike protein domain and the multimerization domain. In some embodiments of any of the aspects, the polypeptide comprises, from N-terminus to C-terminus: the display domain, the linker domain, and the multimerization domain. In some embodiments of any of the aspects, the polypeptide comprises, from N-terminus to C-terminus: the at least one viral spike protein domain, the linker domain, and the multimerization domain.

As used herein, “linker domain” refers to an oligo- or polypeptide region from about 2 to 100 amino acids in length, which links together any of the domains or sequences of the polypeptides as described herein. In some embodiments of any of the aspects, linkers can include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable.

Exemplary linker sequences are provided herein, e.g., SEQ ID Nos: 1-9. In some embodiments of any of the aspects, the linker domain comprises the sequence of one of SEQ ID NOs: 1-9. In some embodiments of any of the aspects, the linker domain comprises the sequence of one of SEQ ID NOs: 1-7. In some embodiments of any of the aspects, the linker domain comprises the sequence of one of SEQ ID NOs: 2 and 6. In some embodiments of any of the aspects, the linker domain comprises the sequence of SEQ ID NO: 2. In some embodiments of any of the aspects, the linker domain comprises the sequence of SEQ ID NO: 6.

In some embodiments of any of the aspects the polypeptide comprises a sequence with at least 80% sequence identity to the sequence of one of SEQ ID NOs: 21-77, 85-96, 109-120, and 135-142. In some embodiments of any of the aspects the multimerization domain comprises a sequence with at least 85% sequence identity to the sequence of one of SEQ ID NOs: 21-77, 85-96, 109-120, and 135-142. In some embodiments of any of the aspects the multimerization domain comprises a sequence with at least 90% sequence identity to the sequence of one of SEQ ID NOs: 21-77, 85-96, 109-120, and 135-142. In some embodiments of any of the aspects the multimerization domain comprises a sequence with at least 95% sequence identity to the sequence of one of SEQ ID NOs: 21-77, 85-96, 109-120, and 135-142. In some embodiments of any of the aspects the multimerization domain comprises a sequence with at least 98% sequence identity to the sequence of one of SEQ ID NOs: 21-77, 85-96, 109-120, and 135-142. In some embodiments of any of the aspects the multimerization domain comprises a sequence with at least 99% sequence identity to the sequence of one of SEQ ID NOs: 21-77, 85-96, 109-120, and 135-142. In some embodiments of any of the aspects the multimerization domain comprises the sequence of one of SEQ ID NOs: 21-77, 85-96, 109-120, and 135-142.

The polypeptides described herein, e.g., when comprising a multimerization domain, can form polypeptide complexes. A polypeptide complex is a molecule in which at least two peptide chains are bound or associated with each other by at least one bond or molecular interaction that is not a peptide bond. Accordingly, in one aspect of any of the embodiments, described herein is a polypeptide complex comprising a multimer of: a first polypeptide described herein; and at least a second polypeptide described herein. In some embodiments of any of the aspects, the first polypeptide and the at least second polypeptide are identical. In some embodiments of any of the aspects, the first polypeptide and the at least second polypeptide are not identical.

In some embodiments of any of the aspects, the polypeptide complex comprises a first polypeptide described herein, a second polypeptide described herein, and a third polypeptide described herein. In some embodiments of any of the aspects, the first polypeptide, the second polypeptide, and the third polypeptide are identical. In some embodiments of any of the aspects, two of the first polypeptide, the second polypeptide, and the third polypeptide are identical. In some embodiments of any of the aspects, none of the first polypeptide, the second polypeptide, and the third polypeptide are identical to each other.

In some embodiments of any of the aspects, the first polypeptide of a polypeptide complex comprises at least one domain of a virus spike protein of a first virus and the at least a second polypeptide comprises at least one domain of a virus spike protein of a second virus.

The polypeptides and polypeptides complexes described herein can be provided in the form of a nanoparticle. As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. The term “nanoparticles” also encompasses peptide complexes having the size of a nanoparticle.

Accordingly, in one aspect of any of the embodiments, described herein is a nanoparticle comprising at least one polypeptide or polypeptide complex as described herein. In some embodiments of any of the aspects, a nanoparticle can comprise a substrate, e.g. a peptide or non-peptide substance that the multimerization domain(s) bind to as part of the nanoparticle formation. For example, the I53_dn5B multimerization domain binds to I53_dn5A polypeptides to form multimers (see, e.g., Ueda, G. et al. Elife 9, e57659 (2020), which is incorporated by reference herein in its entirety). In some embodiments of any of the aspects, the nanoparticle comprises one or more I53_dn5A or I53-50B.4PT1 polypeptides.

In some embodiments of any of the aspects, a polypeptide described herein can further comprise a purification tag, e.g., an affinity tag. As used herein, “affinity tag” refers to a moiety, molecule, polypeptide sequence, or nucleic acid sequence that binds with or is bound by an affinity purification reagent. In some embodiments of any of the aspects, an affinity tag is an epitope. An affinity tag can provide a convenient means for isolating or purifying a polypeptide. When present, the affinity tag can be located anywhere in the polypeptide. For example, the affinity tag can be at the N-terminal, C-terminal or at an internal position of a polypeptide. In some embodiments of any of the aspects, an affinity tag is at a position N-terminal of the at least one viral spike protein domain and/or multimerization domain. In some embodiments of any of the aspects, an affinity tag is at a position C-terminal of the at least one viral spike protein domain and/or multimerization domain.

A number of affinity tags are known in the art. These are usually divided into 3 classes according to their size: small tags have a maximum of 12 amino acids, medium-sized ones have a maximum of 60 and large ones have more than 60. The small tags include the Arg-tag, the His-tag, the avidin biotin, or streptavidin (Strep)-tag, the Flag-tag, the T7-tag, the V5-peptide-tag and the c-Myc-tag, the medium-sized ones include the S-tag, the HAT-tag, the calmodulin-binding peptide, the chitin-binding domain (CBD) and some cellulose-binding domains. The latter can contain up to 189 amino acids and are then regarded, like the glutathione-S-transferase (GST)- and maltose binding protein (MBP)-tag, as large affinity tags.

Some exemplary affinity tag sequences include, but are not limited to, 6-HIS tag (HHHHHH (SEQ ID NO: 200), c-Myc epitope (EQKLISEEDL, SEQ ID NO: 201), Flag-tag (DYKDDDDK, SEQ ID NO: 202), AU1 HA tag (YPYDVPDYA, SEQ ID NO: 203), (DTYRYI, SEQ ID NO: 204), E tag (GAPVPYPDPLEPR, SEQ ID NO: 205); calmodulin tag (or CBP tag, KRRWKKNFIAVSAANRFKKISSSGAL, SEQ ID NO: 206), NE tag (TKENPRSNQEESYDDNESC, SEQ ID NO: 207); S tag (KETAAAKFERQHMDS, SEQ ID NO: 208); Streptavidin-Binding Peptide (SBP) tag (MDEKTTGWRGGHVVEGLAGELEQLRARLEHHPQGQREP, SEQ ID NO: 209); Strep tag (AWRHPQFGG, SEQ ID NO: 210) with high affinity for streptavidin; Spot tag (PDRVRAVSHWSS, SEQ ID NO: 211); pilin-C tag (TDKDMTITFTNKKDA, SEQ ID NO: 212); tetracysteine (TC) tag (CCPGCC, SEQ ID NO: 213); Ty tag (EVHTNQDPLD, SEQ ID NO: 214); V5 tag (GKPIPNPLLGLDST, SEQ ID NO: 215); and vesicular stomatitis virus (VSV) tag (YTDIEMNRLGK, SEQ ID NO: 216).

In some embodiments of any of the embodiments, the affinity tag is a His tag. In some embodiments, the tag comprises a His6-tag (HHHHHH, SEQ ID NO: 200), a His8-tag (HHHHHHHH, SEQ ID NO: 217), a His10-tag (HHHHHHHHHH, SEQ ID NO: 218), or a His14-tag (HHHHHHHHHHHHHH, SEQ ID NO: 219). In some embodiments of any of the aspects, the affinity tag is a His-Sumo tag.

In some embodiments of any of the aspects, a purification tag can comprise, consist of, or consist essentially of the sequence WSHPQFEK (SEQ ID NO: 98).

In some embodiments of any of the aspects, a polypeptide described herein can further comprise a leader sequence and/or signal peptide. Signal peptides are terminally (usually N-terminally) located peptide sequences that provide for passage of the protein into or through a membrane. Different signal peptides can be of use in different applications. For example, as regards a cellular system for the production of polypeptide as described herein, a secretory signal peptide can permit increased yields and ease of purification. As a further example, as regards cells which produce polypeptides as described herein and which are administered for therapeutic purposes to a subject, e.g., a peptide signaling for secretion from the first cell can increase the amount of peptide reaching the target environment. Signal peptides are known in the art, see, e.g., Owji et al. Eur J Cell Biol 94:422-441 (2018); which is incorporated by reference herein in its entirety.

In some embodiments of any of the aspects, a leader sequence or signal peptide can comprise, consist of, or consist essentially of the N-terminal human CD5 leader sequence (MPMGSLQPLATLYLLGMLVASVLA; SEQ ID NO: 99).

In some embodiments of any of the aspects, described herein is a nucleic acid sequence encoding a polypeptide described herein. In some embodiments of any of the aspects, described herein is a nucleic acid molecule comprising a sequence encoding promoter operably linked to a sequence encoding a polypeptide described herein. In some embodiments of any of the aspects, a nucleic acid sequence described herein can be in a vector, or a nucleic acid molecule described herein can be a vector. In some embodiments of any of the aspects, a nucleic acid sequence described herein can be in an expression vector, or a nucleic acid molecule described herein can be an expression vector. In some embodiments of any of the aspects, the nucleic acid sequence is optimized for mammalian expression. In some embodiments of any of the aspects, the nucleic acid sequence is optimized for expression in HEK293 or Expi293F cells. In some embodiments of any of the aspects, the vector is pCDNA 3.1 In some embodiments of any of the aspects, the vector is pCDNA 3.1(+).

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

In one aspect of any of the embodiments, described herein is a pharmaceutical composition comprising a polypeptide, polypeptide complex, or nanoparticle as described herein. In some embodiments of any of the aspects, the technology described herein relates to a pharmaceutical composition comprising a polypeptide, polypeptide complex, or nanoparticle as described herein and optionally a pharmaceutically acceptable carrier.

In some embodiments of any of the aspects, the active ingredients of the pharmaceutical composition comprises a polypeptide, polypeptide complex, or nanoparticle as described herein. In some embodiments of any of the aspects, the active ingredients of the pharmaceutical composition consist essentially of a polypeptide, polypeptide complex, or nanoparticle as described herein. In some embodiments of any of the aspects, the active ingredients of the pharmaceutical composition consist of a polypeptide, polypeptide complex, or nanoparticle as described herein.

In some embodiments of any of the aspects, a pharmaceutical composition described herein further comprises an adjuvant. As used herein in the context of immunization, immune response and vaccination, the term “adjuvant” refers to any substance than when used in combination with a specific antigen that produces a more robust immune response than the antigen alone. When incorporated into a vaccine formulation, an adjuvant acts generally to accelerate, prolong, or enhance the quality of specific immune responses to the vaccine antigen(s).

Adjuvants typically promote the accumulation and/or activation of accessory cells or factors to enhance antigen-specific immune responses and thereby enhance the efficacy of vaccines, i.e., antigen-containing or encoding compositions used to induce protective immunity against the antigen.

Adjuvants, in general, include adjuvants that create a depot effect, immune-stimulating adjuvants, and adjuvants that create a depot effect and stimulate the immune system. An adjuvant that creates a depot effect is an adjuvant that causes the antigen to be slowly released in the body, thus prolonging the exposure of immune cells to the antigen. This class of adjuvants includes but is not limited to alum (e.g., aluminum hydroxide, aluminum phosphate); emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720; AirLiquide, Paris, France); MF-59 (a squalene-in-water emulsion stabilized with Span 85 and Tween 80; Chiron Corporation, Emeryville, Calif.); and PROVAX™ (an oil-in-water emulsion containing a stabilizing detergent and a micelle-forming agent; IDEC Pharmaceuticals Corporation, San Diego, Calif).

An immune-stimulating adjuvant is an adjuvant that causes activation of a cell of the immune system. It may, for instance, cause an immune cell to produce and secrete cytokines and interferons. This class of adjuvants includes but is not limited to saponins purified from the bark of the Q. saponaria tree, such as QS21 (a glycolipid that elutes in the 21st peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). This class of adjuvants also includes CpG DNA.

Adjuvants that create a depot effect and stimulate the immune system are those compounds which have both of the above-identified functions. This class of adjuvants includes but is not limited to ISCOMS (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxypropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

In some embodiments of any of the aspects, the active ingredients of the pharmaceutical composition comprises a polypeptide, polypeptide complex, or nanoparticle as described herein and at least one adjuvant. In some embodiments of any of the aspects, the active ingredients of the pharmaceutical composition consist essentially of a polypeptide, polypeptide complex, or nanoparticle as described herein and at least one adjuvant. In some embodiments of any of the aspects, the active ingredients of the pharmaceutical composition consist of a polypeptide, polypeptide complex, or nanoparticle as described herein and at least one adjuvant.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent as described herein.

In some embodiments of any of the aspects, the pharmaceutical composition comprising an adjuvant comprising a polypeptide, polypeptide complex, or nanoparticle as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a polypeptide, polypeptide complex, or nanoparticle as described herein are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Conventional dosage forms generally provide rapid or immediate active agent release from the formulation. Depending on the pharmacology and pharmacokinetics of the active agent, use of conventional dosage forms can lead to wide fluctuations in the concentrations of the active agent in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control an active agent's onset of action, duration of action, plasma levels within the therapeutic window, and peak blood levels. In particular, controlled- or extended-release dosage forms or formulations can be used to ensure that the maximum effectiveness of an active agent is achieved while minimizing potential adverse effects and safety concerns, which can occur both from under-dosing (i.e., going below the minimum therapeutic levels) as well as exceeding the toxicity level for the active agent. In some embodiments of any of the aspects, the polypeptide, polypeptide complex, or nanoparticle as described herein can be administered in a sustained release formulation.

Controlled-release pharmaceutical products have a common goal of improving therapy over that achieved by their non-controlled release counterparts. Ideally, the use of an optimally designed controlled-release preparation in medical treatment is characterized by a minimum of active agent substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of an active ingredient that promptly produces the desired therapeutic effect, and gradually and continually release other amounts of the active ingredient to maintain this level of therapeutic or prophylactic effect over an extended period of time. In order to maintain this constant level of active agent in the body, the active agent must be released from the dosage form at a rate that will replace the amount of active agent being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the pharmaceutical composition described herein is a vaccine. The term “vaccine” used herein is defined as a composition used to elicit an immune response against an antigen (e.g., the display domain and/or at least one viral spike protein domain described herein) within the composition in order to protect or treat an organism against disease. The terms “vaccine composition” and “vaccine” are used interchangeably. The term “vaccinate” refers to the act of administering a vaccine to a subject.

In one aspect of any of the embodiments, described herein is a method of stimulating an immune response of a subject, the method comprising administering a polypeptide, polypeptide complex, nanoparticle, or pharmaceutical composition as described herein to the subject. In one aspect of any of the embodiments, described herein is a method of immunizing a subject, the method comprising administering a polypeptide, polypeptide complex, nanoparticle, or pharmaceutical composition described herein to the subject.

In one aspect of any of the embodiments, described herein is a polypeptide, polypeptide complex, nanoparticle, or pharmaceutical composition as described herein for use in a method of stimulating an immune response of a subject. Optionally, in some embodiments, the method of the foregoing aspect is not a method for treatment of the human or animal body by surgery or therapy practised on the human or animal body

In one aspect of any of the embodiments, described herein is a polypeptide, polypeptide complex, nanoparticle, or pharmaceutical composition as described herein for use in a method of immunizing a subject. Optionally, in some embodiments, the method of the foregoing aspect is not a method for treatment of the human or animal body by surgery or therapy practised on the human or animal body

In some embodiments of any of the aspects wherein the antigen comprises a coronavirus spike protein domain, the subject may have or be at risk of having a coronavirus infection, which may be Middle East Respiratory Syndrome, SARS-CoV-1, SARS-CoV-2, or SARS-CoV-2 any variants of concern. In some such embodiments, the disease in the subject may be a coronavirus infection. In some embodiments of any of the aspects wherein the antigen comprises a coronavirus spike protein domain, the method or use may be for inducing an immune response against coronavirus, optionally to prevent coronavirus infection. In some embodiments of any of the aspects wherein the antigen comprises a coronavirus spike protein domain, the method or use may be for prevention or treatment of a coronavirus infection. In some embodiments of any of the aspects wherein the antigen comprises a coronavirus spike protein domain, the method or use may be for immunizing a subject against coronavirus, optionally to prevent coronavirus infection.

The term “immunize” as used herein is defined as elicit an immune response, e.g., either a cellular (T-cell) or humoral (B-cell or antibody) response, or both, as measured by standard assays known to one skilled in the art.

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus (e.g., to an antigen and/or adjuvant). In some embodiments of the aspects described herein, the response is specific for a particular antigen (an “antigen-specific response”) and refers to a response by a CD4 T cell, CD8 T cell, or B cell via their antigen-specific receptor. In some embodiments of the aspects described herein, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. Stimulation of an immune response refers to an induction or increase of the immune response.

An immune response to an antigen and/or adjuvant can be the development in a subject of a humoral and/or a cell-mediated immune response to molecules present in the antigen or vaccine composition of interest. For purposes of the present invention, a “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

In some embodiments of any of the aspects, the immune response comprises an increase in Ig levels in the subject. Humans have four Ig subclasses; IgG1, IgG2, IgG3, and IgG4. IgG2 and IgG3 are most indicative of inflammatory and desired vaccine-induced responses, respectively. In some embodiments of any of the aspects, the Ig is IgG2, IgG3, or IgG2a. In some embodiments of any of the aspects, the Ig is IgG2. In some embodiments of any of the aspects, the Ig is IgG3. In some embodiments of any of the aspects, the Ig is IgG2a. In some embodiments of any of the aspects, the Ig is an Ig that specifically binds the display domain and/or at least one viral spike protein domain. In some embodiments of any of the aspects, the Ig is an IgG2a that specifically binds the binds the display domain and/or at least one viral spike protein domain.

In some embodiments of any of the aspects, the immune response comprises a CD4+ T cell response in the subject. In some embodiments of any of the aspects, an immune response can be cytokine production by CD4+ T cells. In some embodiments of any of the aspects, cytokine production by a CD4+ T cell can comprise production of one or more of IL-2 (proliferation); IL-2, IFN-γ, TNF, TNF-β (Th1); IL-4, IL-5, IL-9 and IL-13 (Th2); IL-1-β, IL-17A, IL-17E, IL-17F, IL-21, IL-22, IL-23 (Th17); IL-6, IL21, (Tfh); TGF-β, IL-10, IL-35 (multiple and Tregs). In some embodiments of any of the aspects, an immune response can be an increase in the level of CD4+ T cells, e.g., antigen-specific CD4+ cells.

In some embodiments of any of the aspects, the immune response comprises a CD8+ T cell response in the subject. In some embodiments of any of the aspects, an immune response can be cytokine production by CD8+ T cells. In some embodiments of any of the aspects, cytokine production by a CD8+ T cell can comprise production of one or more of IL-2, IFN-γ, TNF, and IL-10.

In some embodiments of any of the aspects, an immune response can be the release of perform and/or granzymes by CD8+ T cells. In some embodiments of any of the aspects, an immune response can be an increase in the level of CD8+ T cells.

In some embodiments of any of the aspects, the immune response comprises a Th1 cell response in the subject. In some embodiments of any of the aspects, an immune response can be cytokine production by Th1 cells. In some embodiments of any of the aspects, an immune response can be an increase in the level of Th1 cells, e.g., antigen-specific Th1 cells.

In some embodiments of any of the aspects, the immune response comprises a NK cell response in the subject. In some embodiments of any of the aspects, an NK cell response comprises the production of one or more of IFN-γ and TNF. In some embodiments of any of the aspects, an immune response can be the release of perform and/or granzymes by NK cells. In some embodiments of any of the aspects, an immune response can be an increase in the level of NK cells.

In some embodiments of any of the aspects, the immune response stimulates or is an increase of the production of an interferon gamma (IFNγ) response from T cells in the subject, e.g., an increase in IFNγ levels.

In some embodiments of any of the aspects, the immune response initiates or comprises an increase in phagocytosis via the Fc region of each IgG subclass via improved affinity for phagocyte membrane Fc-gamma-receptors (FcγR).

In some embodiments of any of the aspects, the immune response comprises immunization of the subject against the antigen (e.g., binds the display domain and/or at least one viral spike protein domain) or an organism comprising the antigen or a portion of the antigen.

In some embodiments of any of the aspects, the methods described herein can further comprise administering a second agent and/or treatment to the subject, e.g. as part of a combinatorial therapy.

The term “treatment” (including variations thereof, e.g., “treat” or “treated”) as used herein means any one or more of the following: (i) the prevention of infection or re-infection, as in a traditional vaccine, (ii) the reduction in the severity of, or, in the elimination of symptoms, and (iii) the substantial or complete elimination of the pathogen or disorder in question. Hence, treatment may be effected prophylactically (prior to infection) or therapeutically (following infection). In some embodiments of any of the aspects, the treatment is prophylactic. In some embodiments of any of the aspects, compositions and methods are provided that treat, including prophylactically and/or therapeutically immunize, a host animal against an infection (e.g., a bacterium or virus). The methods disclosed herein are useful for conferring prophylactic and/or therapeutic immunity to a subject. The methods disclosed herein can also be practiced on subjects for biomedical research applications.

The compositions and methods described herein can be administered to a subject in need of vaccination, immunization, and/or stimulation of an immune response. In some embodiments of any of the aspects, the methods described herein comprise administering an effective amount of compositions described herein, e.g. to a subject in order to stimulate an immune response or provide protection against the relevant pathogen the antigen was derived from. Providing protection against the relevant pathogen is stimulating the immune system such that later exposure to the antigen (e.g., on or in a live pathogen) triggers a more effective immune response than if the subject was naïve to the antigen. Protection can include faster clearance of the pathogen, reduced severity and/or time of symptoms, and/or lack of development of disease or symptoms. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or topical, administration. Administration can be local or systemic. In some embodiments of any of the aspects, the administration can be intramuscular or subcutaneous.

The term “effective amount” as used herein refers to the amount of a polypeptide, polypeptide complex, or nanoparticle described herein needed to stimulate the immune system or to provide a protective effect against subsequent infections, and relates to a sufficient amount of pharmacological composition to provide the desired effect. The term “therapeutically effective amount” therefore refers to an amount of the polypeptide, polypeptide complex, or nanoparticle described herein (and optionally, an adjuvant) that is sufficient to provide a particular immune stimulatory effect when administered to a typical subject. An effective amount as used herein, in various contexts, would also include an amount sufficient to delay the development of a symptom of the disease, alter the course of a symptom of the disease (for example but not limited to, slowing the progression of a symptom of the disease), or prevent a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a composition which achieves a half-maximal inhibition of symptoms or induction of desired responses) as determined in cell culture, or in an appropriate animal model. Levels in plasma can be measured, for example, by high performance liquid chromatography. The effects of any particular dosage can be monitored by a suitable bioassay, e.g., assay for antibody titers, among others. The dosage can be determined by a physician and adjusted, as necessary, to suit observed effects of the treatment.

In some embodiments of any of the aspects, an effective dose of a composition comprising a polypeptide, polypeptide complex, or nanoparticle as described herein can be administered to a patient once. In some embodiments of any of the aspects, an effective dose of the polypeptide, polypeptide complex, or nanoparticle as described herein can be administered to a patient repeatedly. For systemic administration, subjects can be administered a therapeutic amount of the composition, such as, e.g., 0.01 mg/kg, 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the a polypeptide, polypeptide complex, or nanoparticle as described herein. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses over a period of weeks or months.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for immune response among others. The dosage can be determined by a physician and adjusted, as necessary, to suit observed effects of the treatment.

The dosage ranges for the administration of a polypeptide, polypeptide complex, or nanoparticle as described herein depend upon, for example, the form of the polypeptide, polypeptide complex, or nanoparticle, its potency, and the extent to which symptoms, markers, or indicators of a response described herein are desired to be induced, for example the percentage induction desired for an immune response. The dosage should not be so large as to cause adverse side effects, such as inflammatory responses. Generally, the dosage will vary with the age, condition, and sex of the patient and can be determined by one of skill in the art. The dosage can also be adjusted by the individual physician in the event of any complication.

The efficacy of a polypeptide, polypeptide complex, or nanoparticle as described herein in, e.g. to induce a response as described herein (e.g. an immune response or immunization) can be determined by the skilled clinician. However, a treatment is considered “effective treatment,” as the term is used herein, if one or more of the signs or symptoms of a condition described herein are altered in a beneficial manner, other clinically accepted signs or symptoms are improved, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Immune responses can be detected by a variety of methods known to those skilled in the art, including but not limited to, antibody production, cytotoxicity assay, proliferation assay and cytokine release assays. For example, samples of blood can be drawn from the immunized mammal and analyzed for the presence of antibodies against the antigen administered in the respective vaccine and the titer of these antibodies can be determined by methods known in the art.

Efficacy of an agent can be determined by assessing physical indicators of a desired response, (e.g., immune response, cytokine production, antibody titers, etc.). It is well within the ability of one skilled in the art to monitor efficacy of administration and/or treatment by measuring any one of such parameters, or any combination of parameters. Efficacy can be assessed in animal models of a condition described herein, for example immunization of mice or monkeys. When using an experimental animal model, efficacy of treatment is evidenced when a statistically significant change in a marker is observed.

In vitro and animal model assays are provided herein which allow the assessment of a given dose of an adjuvant and/or antigen. By way of non-limiting example, the effects of a dose of adjuvant can be assessed by measuring the antibody titers.

In one aspect, described herein is method of producing an antibody, the method comprising the steps of: a) injecting a mammal with a polypeptide, polypeptide complex, nanoparticle, or pharmaceutical composition described herein, and b) isolating an antibody specific for the polypeptide, polypeptide complex, nanoparticle, or pharmaceutical composition described herein or producing a monoclonal antibody specific for the polypeptide, polypeptide complex, nanoparticle, or pharmaceutical composition described herein from at least one cell of the mammal. In some embodiments of any of the aspects, the mammal is a mouse. In some embodiments of any of the aspects, the mammal is a rabbit. In some embodiments of any of the aspects, the mammal comprises a humanized immune system. Methods of isolating antibodies and/or antibody-producing cells are known in the art, and can include, by way of non-limiting example, producing a monoclonal antibody via, e.g., the production of hybridomas or phage display. See, e.g., Little et al. Immunology Today 2000 21:364-370; Pasqualini et al. PNAS 2004 101:257-259; Reichert et al. Nature Reviews Drug Discovery 2007 6:349-356; and Wang et al. Antibody Technology Journal 2011 1:1-4; each of which is incorporated by reference herein in its entirety.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of infection or immunization. A subject can be male or female.

A subject can be one who has been previously diagnosed with or identified as suffering from or having a condition in need of treatment (e.g. risk of infection) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established. Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments, a polypeptide as described herein can be chemically synthesized and mutations can be incorporated as part of the chemical synthesis process.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide of nucleic acid as described herein can be engineered. In some embodiments of any of the aspects, a polypeptide or nucleic acid as described herein is not a naturally-occurring polypeptide or nucleic acid. In some embodiments of any of the aspects, a polypeptide, nucleic acid, or cell as described herein can be modified. As used herein, “modified” refers to the aspect of having been manipulated by the hand of man. For example, a polypeptide is considered to be “modified” when at least one aspect of the polypeptide, e.g., its sequence, has been manipulated by the hand of man to differ from the aspect as it exists in nature.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

In some embodiments of any of the aspects, a polypeptide or nucleic acid as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. an infection. The term “treating” includes reducing or alleviating at least one adverse effect or symptom of a condition, disease or disorder associated with an infection. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to infection. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies.

As used herein, the term “antigen” refers to a substance or substances alone or in combination that when introduced into a subject, e.g., a mammalian subject, induces production of antibodies that bind to at least a fraction of the antigen molecules. An antigen will comprise an epitope.

As used herein, an “epitope” can be formed on a polypeptide both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics.

“Avidity” is the measure of the strength of binding between an antibody and the pertinent antigen. Avidity is related to both the affinity between an antigenic determinant and its antigen binding site on the antigen-binding molecule, and the number of pertinent binding sites present on the antigen-binding molecule. Typically, antibodies will bind to their cognate or specific antigen with a dissociation constant (KD of 10⁻⁵ to 10⁻¹² moles/liter or less, such as 10⁻⁷ to 10⁻¹² moles/liter or less, or 10⁻⁸ to 10⁻¹² moles/liter (i.e., with an association constant (KA) of 10⁵ to 10¹² liter/moles or more, such as 10⁷ to 10¹² liter/moles or 10⁸ to 10¹² liter/moles). Any KD value greater than 10⁻⁴ mol/liter (or any KA value lower than 10⁴ M⁻¹) is generally considered to indicate non-specific binding. The KD for biological interactions which are considered meaningful (e.g., specific) are typically in the range of 10⁻¹⁰ M (0.1 nM) to 10⁻⁵ M (10000 nM). The stronger an interaction, the lower is its KD. For example, a binding site on an antibody will bind to the desired antigen with an affinity less than 500 nM, such as less than 200 nM, or less than 10 nM, such as less than 500 pM. Specific binding of an antibody to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

Accordingly, as used herein, “selectively binds” or “specifically binds” refers to the ability of a first entity such as a peptide (e.g., an antibody or portion thereof) to bind to a second entity, such as an antigen present in a polypeptide described herein, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the first entity and the concentration of the first entity. The person of ordinary skill in the art can determine appropriate conditions under which an antibody described herein selectively binds an antigen using any suitable methods, such as titration of an antibody in a suitable binding assay. A first entity specifically bound to a second entity (e.g., an antigen) is not displaced by a non-similar competitor. In certain embodiments, an antibody is said to specifically bind an antigen when it preferentially recognizes the antigen in a complex mixture of proteins and/or macromolecules.

As used herein, the term “administering,” refers to the placement of a compound or composition as disclosed herein into a subject by a method or route which results in at least partial delivery of the agent at a desired site. Pharmaceutical compositions comprising the polypeptides, polypeptide complexes, and nanoparticles disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Bruce Alberts et al., Molecular Biology of the Cell, published by W.W. Norton & Company, 2022 (ISBN 0393884821, 978-0393884821); John M. Lackie eat al. (eds.), The Dictionary of Cell and Molecular Biology, 5^{th} Edition, published by Academic Press, 2013 (ISBN 0123849314, 978-0123849311); Nalini Chandar et al., Lippincott Illustrated Reviews: Cell and Molecular Biology, 3^{rd} Edition, published by LWW, 2023 (ISBN 1975180895, 978-1975180898); Teresa Atwood et al., Oxford Dictionary of Biochemistry and Molecular Biology, 2^{nd} Edition, published by Oxford University Press, 2006; Robert S. Porter et al. (eds.), The Encyclopedia of Molecular Cell Biology and Molecular Medicine, published by Blackwell Science Ltd., 1999-2012 (ISBN 9783527600908); Johnathan Law et al., (eds.), A Dictionary of Chemistry, 8^{th} Edition, published by Oxford University Press, 2020 (ISBN 9780198841227, 9780191876783); Robert C. King et al. (eds.), A Dictionary of Genetics, 8^{th} Edition, published by Oxford University Press, 2013 (ISBN 9780199766444, 9780199376865); Richard Cammack et al. (eds.), Oxford Dictionary of Biochemistry and Molecular Biology, 2^{nd} Edition, published by Oxford University Press, 2006 (ISBN 9780198529170, 9780191727641); John Lackie et al. (eds.), A Dictionary of Biomedicine, 2^{nd} Edition, published by Oxford University Press, 2019 (ISBN 9780191829116); Lodish et al., Molecular Cell Biology, 8^{th} Edition, published by W.H. Freeman, 2016 (ISBN 1464183392, 978-1464183393); Abul K. Abbas et al., Cellular and Molecular Immunology, 10^{th} Edition, published by Elsevier, 2021 (ISBN 0323757480, 978-0323757485); Kenneth M. Murphy et al., Janeway's Immunobiology, 10^{th} Edition, published by W. W. Norton & Company, 2022 (ISBN 0393884899, 978-0393884890); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 0444569464); Laboratory Methods in Enzymology: DNA, Jon Lorsch (ed.) Elsevier, 2013 (ISBN 0124199542); Frederick M. Ausubel (ed.), Current Protocols in Molecular Biology (CPMB), John Wiley and Sons, 1987-2010 (ISBN 047150338X, 9780471503385); Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A polypeptide comprising:
   a) a display domain;
   b) a linker domain; and
   c) a multimerization domain.
2. The polypeptide of any one of the preceding paragraphs, wherein the display domain comprises at least one domain of a viral spike protein.
3. The polypeptide of paragraph 2, wherein the display domain is at least one of:
   i) is a 2P variant;
   ii) not comprising a Receptor Binding Domain (RBD);
   iii) not comprising a hypervariable immunogenic epitope;
   iv) a concatenation of at least a first spike protein sequence and a second spike protein sequence; and
   v) a concatenation of at least a first spike protein sequence from a first virus and a second spike protein sequence from a second virus.
4. The polypeptide of any one of paragraphs 2-3, wherein the spike protein is a coronavirus spike protein.
5. A polypeptide comprising:
   at least one domain of a spike protein;
   wherein the polypeptide is at least one of:
      i) comprises a stabilized variant of the at least one domain of a spike protein;
      ii) not comprising a Receptor Binding Domain (RBD);
      iii) not comprising a hypervariable immunogenic epitope;
      iv) a concatenation of at least a first spike protein sequence and a second spike protein sequence; and
      v) a concatenation of at least a first spike protein sequence from a first virus and a second spike protein sequence from a second virus.
6. The polypeptide of any one of the preceding paragraphs, wherein the hypervariable immunogenic epitope is a G4 loop.
7. The polypeptide of any one of the preceding paragraphs, wherein the at least one domain of a spike protein comprises a spike protein ectodomain.
8. The polypeptide of any one of the preceding paragraphs, wherein the at least one domain of a spike protein comprises a spike protein stalk domain.
9. The polypeptide of any one of the preceding paragraphs, wherein the at least one domain of a virus spike protein comprises:
   a virus stalk domain from which the hypervariable immunogenic epitope is absent, and
   does not comprise the subunit 1 (S1) region of virus spike protein.
10. The polypeptide of any one of the preceding paragraphs, where the virus stalk domain comprises SEQ ID NO: 107 or SEQ ID NO: 108.
11. The polypeptide of any one of the preceding paragraphs, wherein the polypeptide comprises a concatenation of at least a first spike protein sequence and a second spike protein sequence, and the concatenation comprises:
   a concatenation of at least a first N-Terminal Domain (NTD) sequence and a second NTD sequence.
12. The polypeptide of any one of the preceding paragraphs, wherein the polypeptide comprises a concatenation of at least a first spike protein sequence and a second spike protein sequence, and the concatenation comprises:
   a concatenation of at least a first RBD sequence and a second RBD sequence.
13. The polypeptide of any one of the preceding paragraphs, wherein the polypeptide comprises a concatenation of at least a first spike protein sequence and a second spike protein sequence, and the concatenation comprises:
   a concatenation of at least:
   a) a first NTD sequence, a first Subdomain 1 (SD1) sequence, and a first Subdomain 2 (SD2) sequence; and
   b) a second NTD sequence, a second SD1 sequence, and a second SD2 sequence.
14. The polypeptide of any one of the preceding paragraphs, wherein the polypeptide comprises a concatenation of at least a first spike protein sequence and a second spike protein sequence, and the concatenation comprises:
   a concatenation of at least:
   a) a first RBD sequence, a first SD1 sequence, and a first SD2 sequence; and
   b) a second RBD sequence, a second SD1 sequence, and a second SD2 sequence.
15. The polypeptide of any one of the preceding paragraphs, wherein the polypeptide comprises a concatenation of at least a first spike protein sequence and a second spike protein sequence, and the concatenation comprises:
   a concatenation of at least:
   a) a first fusion sequence; and
   b) a second fusion sequence.
16. The polypeptide of any one of the preceding paragraphs, wherein the polypeptide comprises a concatenation of at least a first spike protein sequence from a first virus and a second spike protein sequence from a second virus, and the concatenation comprises:
   a concatenation of at least a first N-Terminal Domain (NTD) sequence from the first virus and a second NTD sequence from the second virus.
17. The polypeptide of any one of the preceding paragraphs, wherein the polypeptide comprises a concatenation of at least a first spike protein sequence from a first virus and a second spike protein sequence from a second virus, and the concatenation comprises:
   a concatenation of at least a first RBD sequence from the first virus and a second RBD sequence from the second virus.
18. The polypeptide of any one of the preceding paragraphs, wherein the polypeptide comprises a concatenation of at least a first spike protein sequence from a first virus and a second spike protein sequence from a second virus, and the concatenation comprises:
   a concatenation of at least:
   a) a first NTD sequence from the first virus, a first SD1 sequence from the first virus, and a first SD2 sequence from the first virus; and
   b) a second NTD sequence from the second virus, a second SD1 sequence from the second virus, and a second SD2 sequence from the second virus.
19. The polypeptide of any one of the preceding paragraphs, wherein the polypeptide comprises a concatenation of at least a first spike protein sequence from a first virus and a second spike protein sequence from a second virus, and the concatenation comprises:
   a concatenation of at least:
   a) a first RBD sequence from the first virus, a first SD1 sequence from the first virus, and a first SD2 sequence from the first virus; and
   b) a second RBD sequence from the second virus, a second SD1 sequence from the second virus, and a second SD2 sequence from the second virus.
20. The polypeptide of any one of the preceding paragraphs, wherein the polypeptide comprises a concatenation of at least a first spike protein sequence from a first virus and a second spike protein sequence from a second virus, and the concatenation comprises:
   a concatenation of at least:
   a) a first fusion sequence from the first virus; and
   b) a second fusion sequence from the second virus.
21. The polypeptide of any one of the preceding paragraphs, wherein the spike protein is a coronavirus spike protein.
22. The polypeptide of any one of the preceding paragraphs, wherein the virus is a coronavirus.
23. The polypeptide of any one of the preceding paragraphs, wherein the first virus is a coronavirus and the second virus is a coronavirus.
24. The polypeptide of any one of the preceding paragraphs, wherein the first virus is the member of a first clade of a genus and the second virus is the member of a second clade of the virus genus.
25. The polypeptide of any one of the preceding paragraphs wherein the coronavirus spike protein is an alphacoronavirus spike protein.
26. The polypeptide of any one of the preceding paragraphs wherein the coronavirus spike protein is an betacoronavirus spike protein.
27. The polypeptide of any one of the preceding paragraphs wherein the coronavirus spike protein is a MERS-CoV spike protein, SARS-CoV-1 spike protein, SARS-CoV-2 spike protein, hCoV-HKU1 spike protein, hCoV-HL63 spike protein, hCoV-229E spike protein, or hCoV-OC43 spike protein.
28. The polypeptide of any one of the preceding paragraphs, wherein the at least one domain of a virus spike protein comprises a sequence with at least 80% sequence identity to the sequence of one of SEQ ID NOs: 21-27, 78-84, 90-96, 123-134, and 143-150.
29. The polypeptide of any one of the preceding paragraphs, wherein the at least one domain of a virus spike protein comprises the sequence of one of SEQ ID NOs: 21-27, 78-84, 90-96, 123-134, and 143-150.
30. The polypeptide of any one of the preceding paragraphs, further comprising a multimerization domain.
31. The polypeptide of any one of the preceding paragraphs, wherein the multimerization domain is a trimerization domain.
32. The polypeptide of any one of the preceding paragraphs, wherein the trimerization domain is I53_dn5B.
33. The polypeptide of any one of the preceding paragraphs, wherein the multimerization domain is foldon, Apoferritin, Lumazine Synthase, or I53_dn5B.
34. The polypeptide of any one of the preceding paragraphs, wherein the multimerization domain comprises a sequence with at least 80% sequence identity to the sequence of one of SEQ ID NOs: 17-20, 97, and 122.
35. The polypeptide of any one of the preceding paragraphs, wherein the multimerization domain comprises the sequence of one of SEQ ID NOs: 17-20, 97, and 122.
36. The polypeptide of any one of the preceding paragraphs, further comprising a linker domain.
37. The polypeptide of any one of the preceding paragraphs, wherein the linker domain is located between the a) the display domain, and c) the multimerization domain.
38. The polypeptide of any one of the preceding paragraphs, wherein the linker domain comprises the sequence of one of SEQ ID NOs: 1-9.
39. The polypeptide of any one of the preceding paragraphs, wherein the linker domain comprises the sequence of one of SEQ ID NOs: 1-7.
40. The polypeptide of any one of the preceding paragraphs, wherein the linker domain comprises the sequence of one of SEQ ID NOs: 2 and 6.
41. The polypeptide of any one of the preceding paragraphs, wherein the linker domain comprises the sequence of SEQ ID NO: 2.
42. The polypeptide of any one of the preceding paragraphs, wherein the polypeptide comprises a sequence with at least 80% sequence identity to the sequence of one of SEQ ID NOs: 21-77, 85-96, 109-120, and 135-142.
43. The polypeptide of any one of the preceding paragraphs, wherein the polypeptide comprises the sequence of one of SEQ ID NOs: 21-77, 85-96, 109-120, and 135-142.
44. A polypeptide complex comprising a multimer of:
   a) a first polypeptide of any one of the preceding paragraphs; and
   b) at least a second polypeptide of any one of the preceding paragraphs.
45. The polypeptide complex of paragraph 44, wherein the first polypeptide and the second polypeptide are identical.
46. The polypeptide complex of any one of paragraphs 44-45, wherein the first polypeptide and the second polypeptide are not identical.
47. The polypeptide complex of any one of paragraphs 44-46, wherein the first polypeptide comprises at least one domain of a virus spike protein of a first virus and the second polypeptide comprises at least one domain of a virus spike protein of a second virus.
48. A nucleic acid encoding the polypeptide of any one of the preceding paragraphs.
49. A nanoparticle comprising at least one polypeptide or polypeptide complex of any one of the preceding paragraphs.
50. The nanoparticle of paragraph 49, comprising one or more I53_dn5A or I53-50B.4PT1 polypeptides.
51. A pharmaceutical composition comprising the polypeptide, polypeptide complex, or nanoparticle of any one of the preceding paragraphs.
52. A method of immunizing a subject, the method comprising administering a polypeptide, polypeptide complex, nanoparticle, or pharmaceutical composition of any one of the preceding paragraphs to the subject.
53. A method of stimulating an immune response of a subject, the method comprising administering a polypeptide, polypeptide complex, nanoparticle, or pharmaceutical composition of any one of the preceding paragraphs to the subject.

The technology described herein is further illustrated by the following examples which in no way should be construed as being further limiting.

### EXAMPLES

### Example 1

Spike sequences
MERS SS.V2 ΔG4 loopmut1
(SEQ ID NO: 90)
QAFNHPIQVDQLNSSYFKLSIPTNFSFGVTQEYIQTTIQKVCVDCKQYVCNGFQKCEQLLREY
GQFCSKINQALHGCNLRQDDSVRNLFASVKSSQSSPIIPGFGGDFNLTLLEPVSISTGSRSARSA
IEDLLFDKCTIADPGYMQGYDDCMQQGPASARDLICAQYVAGYCVLPPLMDVNMEAAYTSS
LLGSIAGSGWTAGLSSFAAIPFAQMIFYRLNGIGITQQVLSENQKLIANKFNQALGAMQTGFT
TTNEAFHKCQDAVNNNAQALSKLASELSNTFGAISASIGDIIQRLDPPEQDAQIDRLINGRLTT
LNAFVAQQLVRCEEAAQSAQLAKDKVNECVKAQSKRSGFCGQGTHIVSFVVNAPYGLYFM
HVGYYPSNHIEVVSAYGLCDAANPTNCIAPVNGTFIKGGSGGNSTGGSGGWNYTGSSFYAPE
PITSLNTKYVAPQVTYQNISTNLPPPLLGNSTGIDFQDELDEFFKNVSTSIPNFGSLTQINTTLLD
LTYEMLSLQQVVKALNESYIDLKELGNYTYGSGYIPEAPRDGQAYVRKDGEWVLLSTFLG
MERS SS.V2 ΔG4 loopmut2
(SEQ ID NO: 91)
QAFNHPIQVDQLNSSYFKLSIPTNFSFGVTQEYIQTTIQKVCVDCKQYVCNGFQKCEQLLREY
GQFCSKINQALHGCNLRQDDSVRNLFASVKSSQSSPIIPGFGGDFNLTLLEPVSISTGSRSARSA
IEDLLFDKCTIADPGYMQGYDDCMQQGPASARDLICAQYVAGYCVLPPLMDVNMEAAYTSS
LLGSIAGSGWTAGLSSFAAIPFAQMIFYRLNGIGITQQVLSENQKLIANKFNQALGAMQTGFT
TTNEAFHKCQDAVNNNAQALSKLASELSNTFGAISASIGDIIQRLDPPEQDAQIDRLINGRLTT
LNAFVAQQLVRCEEAAQSAQLAKDKVNECVKAQSKRSGFCGQGTHIVSFVVNAPYGLYFM
HVGYYPSNHIEVVSGGSGGSGGSGGSPQVTYQNISTNLPPPLLGNSTGIDFQDELDEFFKNVST
SIPNFGSLTQINTTLLDLTYEMLSLQQVVKALNESYIDLKELGNYTYGSGYIPEAPRDGQAYV
RKDGEWVLLSTFLG
MERS SS.V2 ΔG4 loopmut3
(SEQ ID NO: 92)
QAFNHPIQVDQLNSSYFKLSIPTNFSFGVTQEYIQTTIQKVCVDCKQYVCNGFQKCEQLLREY
GQFCSKINQALHGCNLRQDDSVRNLFASVKSSQSSPIIPGFGGDFNLTLLEPVSISTGSRSARSA
IEDLLFDKCTIADPGYMQGYDDCMQQGPASARDLICAQYVAGYCVLPPLMDVNMEAAYTSS
LLGSIAGSGWTAGLSSFAAIPFAQMIFYRLNGIGITQQVLSENQKLIANKFNQALGAMQTGFT
TTNEAFHKCQDAVNNNAQALSKLASELSNTFGAISASIGDIIQRLDPPEQDAQIDRLINGRLTT
LNAFVAQQLVRCEEAAQSAQLAKDKVNECVKAQSKRSGFCGQGTHIVSFVVNAPYGLYFM
HVGYYPSNHIEVVSAYGLCDAANPTNCIAPVNGTGGGGSGGGGSNATGSSFYAPEPITSLNT
KYVAPQVTYQNISTNLPPPLLGNSTGIDFQDELDEFFKNVSTSIPNFGSLTQINTTLLDLTYEML
SLQQVVKALNESYIDLKELGNYTYGSGYIPEAPRDGQAYVRKDGEWVLLSTFLG
MERS SS.V2 ΔG4 loopmut4
(SEQ ID NO: 93)
QAFNHPIQVDQLNSSYFKLSIPTNFSFGVTQEYIQTTIQKVCVDCKQYVCNGFQKCEQLLREY
GQFCSKINQALHGCNLRQDDSVRNLFASVKSSQSSPIIPGFGGDFNLTLLEPVSISTGSRSARSA
IEDLLFDKCTIADPGYMQGYDDCMQQGPASARDLICAQYVAGYCVLPPLMDVNMEAAYTSS
LLGSIAGSGWTAGLSSFAAIPFAQMIFYRLNGIGITQQVLSENQKLIANKFNQALGAMQTGFT
TTNEAFHKCQDAVNNNAQALSKLASELSNTFGAISASIGDIIQRLDPPEQDAQIDRLINGRLTT
LNAFVAQQLVRCEEAAQSAQLAKDKVNECVKAQSKRSGFCGQGTHIVSFVVNAPYGLYFM
HVGYYPSNHIEVVSAYGLCDAANPTNCIAPVNGTGGGGSGGGGSNATGSSFYAPENITSLNT
KYVAPQVTYQNISTNLPPPLLGNSTGIDFQDELDEFFKNVSTSIPNFGSLTQINTTLLDLTYEML
SLQQVVKALNESYIDLKELGNYTYGSGYIPEAPRDGQAYVRKDGEWVLLSTFLGRS
SARS-2 S2P dRBD-F3CH2S native
(SEQ ID NO: 94)
SSQCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVFKNIDG
YFKIYSKHTPINLVRDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIYQTSNFRVQPTESI
VRFPNITNLCPFG
GGSGGKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRA
GCLIGAEHVNNSYECDIPIGAGICASYQTQTNSPGSASSVASQSIIAYTMSLGAENSVAYSNNSI
AIPTNFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANLAATK
MSECVLGQSKRVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQGSGYIPE
APRDGQAYVRKDGEWVLLSTFLGRSLEVLFQGPGHHHHHHHHSAWSHPQFEKGGGSGGGG
SGGSAWSHPQFEK
PL-SARS-2 S2P dRBD-F3CH2S
(SEQ ID NO: 95)
SSQCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVFKNIDG
YFKIYSKHTPINLVRDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIYQTSNFRVQPTESI
VRFPNITNLCPFG
GGSGGKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRA
GCLIGAEHVNNSYECDIPIGAGICASYQTQTNSPGSASSVASQSIIAYTMSLGAENSVAYSNNSI
AIPTNFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANLAATK
MSECVLGQSKRVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQGSGYIPE
APRDGQAYVRKDGEWVLLSTFLGRSLEVLFQGPGHHHHHHHHSAWSHPQFEKGGGSGGGG
SGGSAWSHPQFEK
PL-SARS S2P dRBD
(SEQ ID NO: 96)
SSQCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCND
PFLGVYYHKNNKSWMESEFRVYSSANNCTFEYVSQPFLMDLEGKQGNFKNLREFVFKNIDG
YFKIYSKHTPINLVRDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIYQTSNFRVQPTESI
VRFPNITNLCPFG
GGSGGKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRA
GCLIGAEHVNNSYECDIPIGAGICASYQTQTNSPGSASSVASQSIIAYTMSLGAENSVAYSNNSI
AIPTNFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANLAATK
MSECVLGQSKRVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQGSGYIPE
APRDGQAYVRKDGEWVLLSTFLG

A) Engineered Spikes. For SEQ ID Nos: 21-27, the leader sequence is the bolded text at the beginning of the sequence, followed by the engineered antigen in unbolded text, the linker in bold text (if present), and finally the purification sequence in unbolded text (if present).

MERS SS.V2 ΔG4 loopmut1-His-Strep
(SEQ ID NO: 21)
MRPTWAWWLFLVLLLALWAPARGQAFNHPIQVDQLNSSYFKLSIPTNFSFGVTQEYIQTTI
QKVCVDCKQYVCNGFQKCEQLLREYGQFCSKINQALHGCNLRQDDSVRNLFASVKSSQSSPI
IPGFGGDFNLTLLEPVSISTGSRSARSAIEDLLFDKCTIADPGYMQGYDDCMQQGPASARDLIC
AQYVAGYCVLPPLMDVNMEAAYTSSLLGSIAGSGWTAGLSSFAAIPFAQMIFYRLNGIGITQ
QVLSENQKLIANKFNQALGAMQTGFTTTNEAFHKCQDAVNNNAQALSKLASELSNTFGAIS
ASIGDIIQRLDPPEQDAQIDRLINGRLTTLNAFVAQQLVRCEEAAQSAQLAKDKVNECVKAQS
KRSGFCGQGTHIVSFVVNAPYGLYFMHVGYYPSNHIEVVSAYGLCDAANPTNCIAPVNGTFI
KGGSGGNSTGGSGGWNYTGSSFYAPEPITSLNTKYVAPQVTYQNISTNLPPPLLGNSTGIDFQ
DELDEFFKNVSTSIPNFGSLTQINTTLLDLTYEMLSLQQVVKALNESYIDLKELGNYTYGSGYI
PEAPRDGQAYVRKDGEWVLLSTFLG
MERS SS.V2 ΔG4 loopmut2-His-Strep
(SEQ ID NO: 22)
MRPTWAWWLFLVLLLALWAPARGQAFNHPIQVDQLNSSYFKLSIPTNFSFGVTQEYIQTTI
QKVCVDCKQYVCNGFQKCEQLLREYGQFCSKINQALHGCNLRQDDSVRNLFASVKSSQSSPI
IPGFGGDFNLTLLEPVSISTGSRSARSAIEDLLFDKCTIADPGYMQGYDDCMQQGPASARDLIC
AQYVAGYCVLPPLMDVNMEAAYTSSLLGSIAGSGWTAGLSSFAAIPFAQMIFYRLNGIGITQ
QVLSENQKLIANKFNQALGAMQTGFTTTNEAFHKCQDAVNNNAQALSKLASELSNTFGAIS
ASIGDIIQRLDPPEQDAQIDRLINGRLTTLNAFVAQQLVRCEEAAQSAQLAKDKVNECVKAQS
KRSGFCGQGTHIVSFVVNAPYGLYFMHVGYYPSNHIEVVSGGSGGSGGSGGSPQVTYQNIST
NLPPPLLGNSTGIDFQDELDEFFKNVSTSIPNFGSLTQINTTLLDLTYEMLSLQQVVKALNESYI
DLKELGNYTYGSGYIPEAPRDGQAYVRKDGEWVLLSTFLG
MERS SS.V2 ΔG4 loopmut3-His-Strep
(SEQ ID NO: 23)
MRPTWAWWLFLVLLLALWAPARGQAFNHPIQVDQLNSSYFKLSIPTNFSFGVTQEYIQTTI
QKVCVDCKQYVCNGFQKCEQLLREYGQFCSKINQALHGCNLRQDDSVRNLFASVKSSQSSPI
IPGFGGDFNLTLLEPVSISTGSRSARSAIEDLLFDKCTIADPGYMQGYDDCMQQGPASARDLIC
AQYVAGYCVLPPLMDVNMEAAYTSSLLGSIAGSGWTAGLSSFAAIPFAQMIFYRLNGIGITQ
QVLSENQKLIANKFNQALGAMQTGFTTTNEAFHKCQDAVNNNAQALSKLASELSNTFGAIS
ASIGDIIQRLDPPEQDAQIDRLINGRLTTLNAFVAQQLVRCEEAAQSAQLAKDKVNECVKAQS
KRSGFCGQGTHIVSFVVNAPYGLYFMHVGYYPSNHIEVVSAYGLCDAANPTNCIAPVNGTG
GGGSGGGGSNATGSSFYAPEPITSLNTKYVAPQVTYQNISTNLPPPLLGNSTGIDFQDELDEFF
KNVSTSIPNFGSLTQINTTLLDLTYEMLSLQQVVKALNESYIDLKELGNYTYGSGYIPEAPRDG
QAYVRKDGEWVLLSTFLGGGGGS AEAAAKEAAAKEAAAKA
GGGGSMQIYEGKLTAEGLRFGIVASRFNHALVDRLVEGAIDCIVRHGGREEDITLVRVPGSW
EIPVAAGELARKEDIDAVIAIGVLIRGATPHFDYIASEVSKGLANLSLELRKPITFGVITADTLE
QAIERAGTKHGNKGWEAALSAIEMANLFKSLR
MERS SS.V2 ΔG4 loopmut4-His-Strep
(SEQ ID NO: 24)
MRPTWAWWLFLVLLLALWAPARGQAFNHPIQVDQLNSSYFKLSIPTNFSFGVTQEYIQTTI
QKVCVDCKQYVCNGFQKCEQLLREYGQFCSKINQALHGCNLRQDDSVRNLFASVKSSQSSPI
IPGFGGDFNLTLLEPVSISTGSRSARSAIEDLLFDKCTIADPGYMQGYDDCMQQGPASARDLIC
AQYVAGYCVLPPLMDVNMEAAYTSSLLGSIAGSGWTAGLSSFAAIPFAQMIFYRLNGIGITQ
QVLSENQKLIANKFNQALGAMQTGFTTTNEAFHKCQDAVNNNAQALSKLASELSNTFGAIS
ASIGDIIQRLDPPEQDAQIDRLINGRLTTLNAFVAQQLVRCEEAAQSAQLAKDKVNECVKAQS
KRSGFCGQGTHIVSFVVNAPYGLYFMHVGYYPSNHIEVVSAYGLCDAANPTNCIAPVNGTG
GGGSGGGGSNATGSSFYAPENITSLNTKYVAPQVTYQNISTNLPPPLLGNSTGIDFQDELDEFF
KNVSTSIPNFGSLTQINTTLLDLTYEMLSLQQVVKALNESYIDLKELGNYTYGSGYIPEAPRDG
QAYVRKDGEWVLLSTFLGRS
LEVLFQGPGHHHHHHHHSAWSHPQFEKGGGSGGGGSGGSAWSHPQFEK
SARS-2 S2P dRBD-F3CH2S native
(SEQ ID NO: 25)
MFVFLVLLPLVSSQCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNATNV
VIKVCEFQFCNDPFLGVYYHKNNKSWMESEFRVYSSANNCTFEYVSQPFLMDLEGKQGNFK
NLREFVFKNIDGYFKIYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCTLKSFTVEKGIYQ
TSNFRVQPTESIVRFPNITNLCPFG
GGSGGKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRA
GCLIGAEHVNNSYECDIPIGAGICASYQTQTNSPGSASSVASQSIIAYTMSLGAENSVAYSNNSI
AIPTNFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANLAATK
MSECVLGQSKRVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQGSGYIPE
APRDGQAYVRKDGEWVLLSTFLGRSLEVLFQGPGHHHHHHHHSAWSHPQFEKGGGSGGGG
SGGSAWSHPQFEK
PL-SARS-2 S2P dRBD-F3CH2S
(SEQ ID NO: 26)
MDSKGSSQKGSRLLLLLVVSNLLLPQGVLASSQCVNLTTRTQLPPAYTNSFTRGVYYPDK
VFRSSVLHSTQDLFLPFFSNVTWFHAIHVSGTNGTKRFDNPVLPFNDGVYFASTEKSNIIRGWI
FGTTLDSKTQSLLIVNNATNVVIKVCEFQFCNDPFLGVYYHKNNKSWMESEFRVYSSANNCT
FEYVSQPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDAVDCAL
DPLSETKCTLKSFTVEKGIYQTSNFRVQPTESIVRFPNITNLCPFG
GGSGGKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRA
GCLIGAEHVNNSYECDIPIGAGICASYQTQTNSPGSASSVASQSIIAYTMSLGAENSVAYSNNSI
AIPTNFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANLAATK
MSECVLGQSKRVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQGSGYIPE
APRDGQAYVRKDGEWVLLSTFLGRSLEVLFQGPGHHHHHHHHSAWSHPQFEKGGGSGGGG
SGGSAWSHPQFEK
PL-SARS S2P dRBD-L2-Aferr_no puritag
(SEQ ID NO: 27)
MDSKGSSQKGSRLLLLLVVSNLLLPQGVLASSQCVNLTTRTQLPPAYTNSFTRGVYYPDK
VFRSSVLHSTQDLFLPFFSNVTWFHAIHVSGTNGTKRFDNPVLPFNDGVYFASTEKSNIIRGWI
FGTTLDSKTQSLLIVNNATNVVIKVCEFQFCNDPFLGVYYHKNNKSWMESEFRVYSSANNCT
FEYVSQPFLMDLEGKQGNFKNLREFVFKNIDGYFKIYSKHTPINLVRDLPQGFSALEPLVDLPI
GINITRFQTLLALHRSYLTPGDSSSGWTAGAAAYYVGYLQPRTFLLKYNENGTITDAVDCAL
DPLSETKCTLKSFTVEKGIYQTSNFRVQPTESIVRFPNITNLCPFG
GGSGGKSTNLVKNKCVNFNFNGLTGTGVLTESNKKFLPFQQFGRDIADTTDAVRDPQTLEIL
DITPCSFGGVSVITPGTNTSNQVAVLYQDVNCTEVPVAIHADQLTPTWRVYSTGSNVFQTRA
GCLIGAEHVNNSYECDIPIGAGICASYQTQTNSPGSASSVASQSIIAYTMSLGAENSVAYSNNSI
AIPTNFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVVNQNAQALNTLVK
QLSSNFGAISSVLNDILSRLDPPEAEVQIDRLITGRLQSLQTYVTQQLIRAAEIRASANLAATK
MSECVLGQSKRVDFCGKGYHLMSFPQSAPHGVVFLHVTYVPAQEKNFTTAPAICHDGKAHF
PREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQGSGYIPE
APRDGQAYVRKDGEWVLLSTFLGGGGGSAEAAAKEAAAKEAAAKA
GGGGSMSSQIRQNYSTDVEAAVNSLVNLYLQASYTYLSLGFYFDRDDVALEGVSHFFRELA
EEKREGYERLLKMQNQRGGRALFQDIKKPAEDEWGKTPDAMKAAMALEKKLNQALLDLH
ALGSARTDPHLCDFLETHFLDEEVKLIKKMGDHLTNLHRLGGPEAGLGEYLFERLTLKHD

Nanoparticle Constructs. For SEQ ID Nos: 28-43, the leader sequence is the unbolded text at the beginning of the sequence, followed by the Streptag-Octohistidine-HRV3C in bold, the engineered antigen in unbolded text, the linker in bold text, and finally the nanoparticle sequence in unbolded text. For SEQ ID Nos: 44-53 and 55-77, the leader sequence is the unbolded text at the beginning of the sequence, followed by the engineered antigen in bolded text, the linker in unbolded text, and finally the nanoparticle sequence in bold text. For SEQ ID NO: 54, the leader sequence is the unbolded text at the beginning of the sequence, followed by the engineered antigen in bolded text, the linker in unbolded text, followed by the nanoparticle sequence in bold text, and then finally the streptag-ocathistine-HRV3C in unbolded text.

LSO3C-MERS NTD-L1-AFerr
(SEQ ID NO: 28)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGGSGGGGSGGGGSGGGGSGGGGSMSSQIRQNYSTDVEAA
VNSLVNLYLQASYTYLSLGFYFDRDDVALEGVSHFFRELAEEKREGYERLLKMQNQRGGRA
LFQDIKKPAEDEWGKTPDAMKAAMALEKKLNQALLDLHALGSARTDPHLCDFLETHFLDEE
VKLIKKMGDHLTNLHRLGGPEAGLGEYLFERLTLKHD
LSO3C-MERS NTD-L2-AFerr
(SEQ ID NO: 29)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGGS AEAAAKEAAAKEAAAKA
GGGGSMSSQIRQNYSTDVEAAVNSLVNLYLQASYTYLSLGFYFDRDDVALEGVSHFFRELA
EEKREGYERLLKMQNQRGGRALFQDIKKPAEDEWGKTPDAMKAAMALEKKLNQALLDLH
ALGSARTDPHLCDFLETHFLDEEVKLIKKMGDHLTNLHRLGGPEAGLGEYLFERLTLKHD
LSO3C-MERS NTD-L3-AFerr
(SEQ ID NO: 30)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGGSAEAAAKEAAAKEAAAKEAAAKEAAAKAGGGGSMSS
QIRQNYSTDVEAAVNSLVNLYLQASYTYLSLGFYFDRDDVALEGVSHFFRELAEEKREGYER
LLKMQNQRGGRALFQDIKKPAEDEWGKTPDAMKAAMALEKKLNQALLDLHALGSARTDP
HLCDFLETHFLDEEVKLIKKMGDHLTNLHRLGGPEAGLGEYLFERLTLKHD
LSO3C-MERS NTD-L4-AFerr
(SEQ ID NO: 31)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGSSSSS GSSSSS
GSSSSSMSSQIRQNYSTDVEAAVNSLVNLYLQASYTYLSLGFYFDRDDVALEGVSHFFRELA
EEKREGYERLLKMQNQRGGRALFQDIKKPAEDEWGKTPDAMKAAMALEKKLNQALLDLH
ALGSARTDPHLCDFLETHFLDEEVKLIKKMGDHLTNLHRLGGPEAGLGEYLFERLTLKHD
LSO3C-MERS NTD-L5-AFerr
(SEQ ID NO: 32)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGSA EAAAK EAAAK EAAAKA GGGGSA EAAAK EAAAK
EAAAKAGSMSSQIRQNYSTDVEAAVNSLVNLYLQASYTYLSLGFYFDRDDVALEGVSHFFR
ELAEEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERLTLKH
D
LSO3C-MERS NTD-L6-AFerr
(SEQ ID NO: 33)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGSGGGGSGGGGSGLSKMSSQIRQNYSTDVEAAVNSLVNLY
LQASYTYLSLGFYFDRDDVALEGVSHFFRELAEEKREGYERLLKMQNQRGGRALFQDIKKP
AEDEWGKTPDAMKAAMALEKKLNQALLDLHALGSARTDPHLCDFLETHFLDEEVKLIKKM
GDHLTNLHRLGGPEAGLGEYLFERLTLKHD
LSO3C-MERS NTD-L7-AFerr
(SEQ ID NO: 34)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGSSSSS AEAAAKEAAAKEAAAKA
GSSSSSMSSQIRQNYSTDVEAAVNSLVNLYLQASYTYLSLGFYFDRDDVALEGVSHFFRELA
EEKREGYERLLKMQNQRGGRALFQDIKKPAEDEWGKTPDAMKAAMALEKKLNQALLDLH
ALGSARTDPHLCDFLETHFLDEEVKLIKKMGDHLTNLHRLGGPEAGLGEYLFERLTLKHD
LSO3C-MERS NTD-L1-H.pylori Ferr
(SEQ ID NO: 35)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGGSGGGGSGGGGSGGGGSGGGGSMLSKDIIKLLNEQVNKE
MNSSNLYMSMSSWCYTHSLDGAGLFLFDHAAEEYEHAKKLIVFLNENNVPVQLTSISAPEHK
FEGLTQIFQKAYEHEQHISESINNIVDHAIKSKDHATFNFLQWYVAEQHEEEVLFKDILDKIELI
GNENHGLYLADQYVKGIAKSRKS
LSO3C-MERS NTD-L1-LuS
(SEQ ID NO: 36)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGGSGGGGSGGGGSGGGGSGGGGSMQIYEGKLTAEGLRFGI
VASRFNHALVDRLVEGAIDCIVRHGGREEDITLVRVPGSWEIPVAAGELARKEDIDAVIAIGV
LIRGATPHFDYIASEVSKGLANLSLELRKPITFGVITADTLEQAIERAGTKHGNKGWEAALSAI
EMANLFKSLR
LSO3C-MERS NTD-L2-H.pylori Ferr
(SEQ ID NO: 37)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGGS AEAAAKEAAAKEAAAKA
GGGGSMLSKDIIKLLNEQVNKEMNSSNLYMSMSSWCYTHSLDGAGLFLFDHAAEEYEHAK
KLIVFLNENNVPVQLTSISAPEHKFEGLTQIFQKAYEHEQHISESINNIVDHAIKSKDHATFNFL
QWYVAEQHEEEVLFKDILDKIELIGNENHGLYLADQYVKGIAKSRKS
LSO3C-MERS NTD-L7-H.pylori Ferr
(SEQ ID NO: 38)
MDAMKRGLCCVLLLCGAVFVSPSQEIHARFRRGAR
WSHPQFEKHHHHHHHHLEVLFQGPYVDVGPDSVKSACIEVDIQQTFFDKTWPRPIDVSKA
DGIIYPQGRTYSNITITYQGLFPYQGDHGDMYVYSAGHATGTTPQKLFVANYSQDVKQFANG
FVVRIGAAANSTGTVIISPSTSATIRKIYPAFMLGSSVGNFSDGKMGRFFNHTLVLLPDGCGTL
LRAFYCILEPRSGNHCPAGNSYTSFATYHTPATDCSDGNYNRNASLNSFKEYFNLRNCTFMY
TYNITEDEILEWFGITQTAQGVHLFSSRYVDLYGGNMFQFATLPVYDTIKYYSIIPHSIRSIQSD
RKAWAAFYVYKLQPLTFLLDFSVDGYIRRAIDCGFNDLSQLHCSYGSSSSS
AEAAAKEAAAKEAAAKA
GSSSSSMLSKDIIKLLNEQVNKEMNSSNLYMSMSSWCYTHSLDGAGLFLFDHAAEEYEHAK
KLIVFLNENNVPVQLTSISAPEHKFEGLTQIFQKAYEHEQHISESINNIVDHAIKSKDHATFNFL
QWYVAEQHEEEVLFKDILDKIELIGNENHGLYLADQYVKGIAKSRKS
LSO3C-MERS NTD-L6-H.pylori Ferr
(SEQ ID NO: 39)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGSGGGGSGGGGSGLSKMLSKDIIKLLNEQVNKEMNSSNLY
MSMSSWCYTHSLDGAGLFLFDHAAEEYEHAKKLIVFLNENNVPVQLTSISAPEHKFEGLTQIF
QKAYEHEQHISESINNIVDHAIKSKDHATFNFLQWYVAEQHEEEVLFKDILDKIELIGNENHG
LYLADQYVKGIAKSRKS
LSO3C-MERS NTD-L2-LuS
(SEQ ID NO: 40)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGGSAEAAAKEAAAKEAAAKAGGGGSMQIYEGKLTAEGLR
FGIVASRFNHALVDRLVEGAIDCIVRHGGREEDITLVRVPGSWEIPVAAGELARKEDIDAVIAI
GVLIRGATPHFDYIASEVSKGLANLSLELRKPITFGVITADTLEQAIERAGTKHGNKGWEAAL
SAIEMANLFKSLR
LSO3C-MERS NTD-L7-LuS
(SEQ ID NO: 41)
MDAMKRGLCCVLLLCGAVFVSPSQEIHARFRRGARWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGSSSSS AEAAAKEAAAKEAAAKA
GSSSSSMQIYEGKLTAEGLRFGIVASRFNHALVDRLVEGAIDCIVRHGGREEDITLVRVPGSW
EIPVAAGELARKEDIDAVIAIGVLIRGATPHFDYIASEVSKGLANLSLELRKPITFGVITADTLE
QAIERAGTKHGNKGWEAALSAIEMANLFKSLR
LSO3C MERS NTD-L2-LuS Modified A
(SEQ ID NO: 42)
MPMGSLQPLATLYLLGMLVASVLAWSHPQFEKHHHHHHHHLEVLFQGP
YVDVGPDSVKSACIEVDIQQTFFDKTWPRPIDVSKADGIIYPQGRTYSNITITYQGLFPYQGDH
GDMYVYSAGHATGTTPQKLFVANYSQDVKQFANGFVVRIGAAANSTGTVIISPSTSATIRKIY
PAFMLGSSVGNFSDGKMGRFFNHTLVLLPDGCGTLLRAFYCILEPRSGNHCPAGNSYTSFAT
YHTPATDCSDGNYNRNASLNSFKEYFNLRNCTFMYTYNITEDEILEWFGITQTAQGVHLFSSR
YVDLYGGNMFQFATLPVYDTIKYYSIIPHSIRSIQSDRKAWAAFYVYKLQPLTFLLDFSVDGYI
RRAIDCGFNDLSQLHCSYGGGGS AEAAAKEAAAKEAAAKA
GGGGSMQIYEGKLTAEGLRFGIVASRFNHALVDRLVEGAIDAIVRHGGREEDITLVRVPGSW
EIPVAAGELARKEDIDAVIAIGVLIRGATPHFDYIASEVSKGLAQLSLELRKPITFGVITADTLE
QAIERAGTKHGNKGWEAALSAIEMANLFKSLR
LSO3C MERS NTD-L6-LuS Modified A
(SEQ ID NO: 43)
MPMGSLQPLATLYLLGMLVASVLAWSHPQFEKHHHHHHHHLEVLFQGPYVDVGPDSVK
SACIEVDIQQTFFDKTWPRPIDVSKADGIIYPQGRTYSNITITYQGLFPYQGDHGDMYVYSAG
HATGTTPQKLFVANYSQDVKQFANGFVVRIGAAANSTGTVIISPSTSATIRKIYPAFMLGSSVG
NFSDGKMGRFFNHTLVLLPDGCGTLLRAFYCILEPRSGNHCPAGNSYTSFATYHTPATDCSD
GNYNRNASLNSFKEYFNLRNCTFMYTYNITEDEILEWFGITQTAQGVHLFSSRYVDLYGGNM
FQFATLPVYDTIKYYSIIPHSIRSIQSDRKAWAAFYVYKLQPLTFLLDFSVDGYIRRAIDCGEND
LSQLHCSYGGGSGGGGSGGGGSGLSKMQIYEGKLTAEGLRFGIVASRFNHALVDRLVEGAI
DAIVRHGGREEDITLVRVPGSWEIPVAAGELARKEDIDAVIAIGVLIRGATPHFDYIASEVSKG
LAQLSLELRKPITFGVITADTLEQAIERAGTKHGNKGWEAALSAIEMANLFKSLR
LSO3C-MERS SS V2-L2-LuS
(SEQ ID NO: 44)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGYIPEAPRDGQAYVRKDGEWVLLSTFLGGGGGS
AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
LSO3C-MERS SS V2-L4-LuS
(SEQ ID NO: 45)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGYIPEAPRDGQAYVRKDGEWVLLSTFLGGSSSSS GSSSSS
GSSSSSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
LSO3C-MERS SS V2-L6-LuS
(SEQ ID NO: 46)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGYIPEAPRDGQAYVRKDGEWVLLSTFLGGGGSGGGGSGGGGS
GLSKMQIYEGKLTAEGLRFGIVASRFNHALVDRLVEGAIDCIVRHGGREEDITLVRVPGS
WEIPVAAGELARKEDIDAVIAIGVLIRGATPHFDYIASEVSKGLANLSLELRKPITFGVIT
ADTLEQAIERAGTKHGNKGWEAALSAIEMANLFKSLR
LSO3C-MERS SS V2-L7-LuS
(SEQ ID NO: 47)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGYIPEAPRDGQAYVRKDGEWVLLSTFLGGSSSSS
AEAAAKEAAAKEAAAKA
GSSSSSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
LSO3C-MERS SSV2 dFd-L2-LuS_nopuritag
(SEQ ID NO: 48)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGGGGSAEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
MERS SSV2 dFd-L2-AFerr_nopuritag
(SEQ ID NO: 49)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGGGGS AEAAAKEAAAKEAAAKA
GGGGSMSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFE
RLTLKHD
MERS SS V2-L2-AFerr_nopuritag
(SEQ ID NO: 50)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGYIPEAPRDGQAYVRKDGEWVLLSTFLGGGGGS
AEAAAKEAAAKEAAAKA GGGGS
MSSQIRQNYSTDVEAAVNSLVNLYLQASYTYLSLGFYFDRDDVALEGVSHFFRELAEEK
REGYERLLKMQNQRGGRALFQDIKKPAEDEWGKTPDAMKAAMALEKKLNQALLDLH
ALGSARTDPHLCDFLETHFLDEEVKLIKKMGDHLTNLHRLGGPEAGLGEYLFERLTLK
HD
MERS SS V2-L6-AFerr_nopuritag
(SEQ ID NO: 51)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGYIPEAPRDGQAYVRKDGEWVLLSTFLGGGGSGGGGSGGGGS
GLSKMSSQIRQNYSTDVEAAVNSLVNLYLQASYTYLSLGFYFDRDDVALEGVSHFFRELA
EEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERL
TLKHD
MERS SS V2-L2-H.pylori Ferr nopuritag
(SEQ ID NO: 52)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGYIPEAPRDGQAYVRKDGEWVLLSTFLGGGGGS
AEAAAKEAAAKEAAAKA
GGGGSMLSKDIIKLLNEQVNKEMNSSNLYMSMSSWCYTHSLDGAGLFLFDHAAEEYEH
AKKLIVFLNENNVPVQLTSISAPEHKFEGLTQIFQKAYEHEQHISESINNIVDHAIKSKDH
ATFNFLQWYVAEQHEEEVLFKDILDKIELIGNENHGLYLADQYVKGIAKSRKS
MERS SS V2-L6-H.pylori Ferr_nopuritag
(SEQ ID NO: 53)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKTNNTRIVDEWSYTGSSFYAPEPITSLNTKYVAPQVTYQNIS
TNLPPPLLGNSTGIDFQDELDEFFKNVSTSIPNFGSLTQINTTLLDLTYEMLSLQQVVKAL
NESYIDLKELGNYTYGSGYIPEAPRDGQAYVRKDGEWVLLSTFLGGGGSGGGGSGGGGS
GLSKMLSKDIIKLLNEQVNKEMNSSNLYMSMSSWCYTHSLDGAGLFLFDHAAEEYEHA
KKLIVFLNENNVPVQLTSISAPEHKFEGLTQIFQKAYEHEQHISESINNIVDHAIKSKDHA
TFNFLQWYVAEQHEEEVLFKDILDKIELIGNENHGLYLADQYVKGIAKSRKS
LSO3C-MERS SS V2 AG4 loop-L2-LuS
(SEQ ID NO: 54)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGGS
AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
LEVLFQGPGHHHHHHHHSAWSHPQFEK
LSO3C-MERS SS.V2 AG4 loop-L2-LuS
(SEQ ID NO: 55)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGGS
AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
LSO3C-MERS SS V2 ΔG4 loop dFd-L2-LuS_nopuritag
(SEQ ID NO: 56)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGGGGS AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
MERS SSV2 ΔG4 loop dFd-L2-AFerr nopuritag
(SEQ ID NO: 57)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGGGGS AEAAAKEAAAKEAAAKA
GGGGSSSQIRQNYSTDVEAAVNSLVNLYLQASYTYLSLGFYFDRDDVALEGVSHFFRELA
EEKREGYERLLKMQNQRGGRALFQDIKKPAEDEWGKTPDAMKAAMALEKKLNQALL
DLHALGSARTDPHLCDFLETHFLDEEVKLIKKMGDHLTNLHRLGGPEAGLGEYLFERL
TLKHD
MERS SSV2 ΔG4 loop-L2-AFerr nopuritag
(SEQ ID NO: 58)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGGS
AEAAAKEAAAKEAAAKA
GGGGSMSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFE
RLTLKHD
MERS SSV2 ΔG4 loop-L6-AFerr nopuritag
(SEQ ID NO: 59)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGSGGGGSGGGGSGLS
KMSSQIRQNYSTDVEAAVNSLVNLYLQASYTYLSLGFYFDRDDVALEGVSHFFRELAEE
KREGYERLLKMQNQRGGRALFQDIKKPAEDEWGKTPDAMKAAMALEKKLNQALLDL
HALGSARTDPHLCDFLETHFLDEEVKLIKKMGDHLTNLHRLGGPEAGLGEYLFERLTL
KHD
MERS SSV2 ΔG4 loop-L2-Hpylori Ferr_nopuritag
(SEQ ID NO: 60)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGGS
AEAAAKEAAAKEAAAKA
GGGGSMLSKDIIKLLNEQVNKEMNSSNLYMSMSSWCYTHSLDGAGLFLFDHAAEEYEH
AKKLIVFLNENNVPVQLTSISAPEHKFEGLTQIFQKAYEHEQHISESINNIVDHAIKSKDH
ATFNFLQWYVAEQHEEEVLFKDILDKIELIGNENHGLYLADQYVKGIAKSRKS
MERS SSV2 ΔG4 loop-L6-Hpylori Ferr_nopuritag
(SEQ ID NO: 61)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGSGGGGSGGGGSGLS
K
MLSKDIIKLLNEQVNKEMNSSNLYMSMSSWCYTHSLDGAGLFLFDHAAEEYEHAKKLI
VFLNENNVPVQLTSISAPEHKFEGLTQIFQKAYEHEQHISESINNIVDHAIKSKDHATFNF
LQWYVAEQHEEEVLFKDILDKIELIGNENHGLYLADQYVKGIAKSRKS
LSO3C-MERS SS V2 ΔG4 loop-L6-LuS nopuritag
(SEQ ID NO: 62)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGSGGGGSGGGGSGLS
KMQIYEGKLTAEGLRFGIVASRFNHALVDRLVEGAIDCIVRHGGREEDITLVRVPGSWE
IPVAAGELARKEDIDAVIAIGVLIRGATPHFDYIASEVSKGLANLSLELRKPITFGVITADT
LEQAIERAGTKHGNKGWEAALSAIEMANLFKSLR
LSO3C-MERS SS V2 ΔG4 loop-L4-LuS no puritag
(SEQ ID NO: 63)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGYFIKGGSGGWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGSSSSSGSSSSSGSSSSSM
QIYEGKLTAEGLRFGIVASRFNHALVDRLVEGAIDCIVRHGGREEDITLVRVPGSWEIPV
AAGELARKEDIDAVIAIGVLIRGATPHFDYIASEVSKGLANLSLELRKPITFGVITADTLE
QAIERAGTKHGNKGWEAALSAIEMANLFKSLR
MERS SS.V2 ΔG4 loopmut1-L2-LuS No puritag
(SEQ ID NO: 64)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGTFIKGGSGGNSTGGSGGWNYTGSSFYAPEPITSLNTKYVAPQVT
YQNISTNLPPPLLGNSTGIDFQDELDEFFKNVSTSIPNFGSLTQINTTLLDLTYEMLSLQQ
VVKALNESYIDLKELGNYTYGSGYIPEAPRDGQAYVRKDGEWVLLSTFLGGGGGS
AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
MERS SS.V2 ΔG4 loopmut2-L2-LuS No puritag
(SEQ ID NO: 65)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLEDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVS
GGSGGSGGSGGSPQVTYQNISTNLPPPLLGNSTGIDFQDELDEFFKNVSTSIPNFGSLTQI
NTTLLDLTYEMLSLQQVVKALNESYIDLKELGNYTYGSGYIPEAPRDGQAYVRKDGEW
VLLSTFLGGGGGS AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
MERS SS.V2 ΔG4 loopmut3-L2-LuS No puritag
(SEQ ID NO: 66)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKENQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGTGGGGSGGGGSNATGSSFYAPEPITSLNTKYVAPQVTYQNISTN
LPPPLLGNSTGIDFQDELDEFFKNVSTSIPNFGSLTQINTTLLDLTYEMLSLQQVVKALNE
SYIDLKELGNYTYGSGYIPEAPRDGQAYVRKDGEWVLLSTFLGGGGGS
AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
MERS SS.V2 ΔG4 loopmut4-L2-LuS No puritag
(SEQ ID NO: 67)
MRPTWAWWLFLVLLLALWAPARGQAFNHPIQVDQLNSSYFKLSIPTNFSFGVTQEYIQTT
IQKVCVDCKQYVCNGFQKCEQLLREYGQFCSKINQALHGCNLRQDDSVRNLFASVKSS
QSSPIIPGFGGDFNLTLLEPVSISTGSRSARSAIEDLLFDKCTIADPGYMQGYDDCMQQGP
ASARDLICAQYVAGYCVLPPLMDVNMEAAYTSSLLGSIAGSGWTAGLSSFAAIPFAQMIF
YRLNGIGITQQVLSENQKLIANKFNQALGAMQTGFTTTNEAFHKCQDAVNNNAQALSK
LASELSNTFGAISASIGDIIQRLDPPEQDAQIDRLINGRLTTLNAFVAQQLVRCEEAAQSA
QLAKDKVNECVKAQSKRSGFCGQGTHIVSFVVNAPYGLYFMHVGYYPSNHIEVVSAYG
LCDAANPTNCIAPVNGTGGGGSGGGGSNATGSSFYAPENITSLNTKYVAPQVTYQNISTN
LPPPLLGNSTGIDFQDELDEFFKNVSTSIPNFGSLTQINTTLLDLTYEMLSLQQVVKALNE
SYIDLKELGNYTYGSGYIPEAPRDGQAYVRKDGEWVLLSTFLGGGGGS
AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
SARS_2 S2_6P HexaPro-L2-LuS_nopuritag
(SEQ ID NO: 68)
MRGLGTCLATLAGLLLTAAGSVASQSIIAYTMSLGAENSVAYSNNSIAIPTNFTISVTTEILP
VSMTKTSVDCTMYICGDSTECSNLLLQYGSFCTQLNRALTGIAVEQDKNTQEVFAQVK
QIYKTPPIKDFGGFNFSQILPDPSKPSKRSPIEDLLFNKVTLADAGFIKQYGDCLGDIAAR
DLICAQKFNGLTVLPPLLTDEMIAQYTSALLAGTITSGWTFGAGPALQIPFPMQMAYRF
NGIGVTQNVLYENQKLIANQFNSAIGKIQDSLSSTPSALGKLQDVVNQNAQALNTLVKQ
LSSNFGAISSVLNDILSRLDPPEAEVQIDRLITGRLQSLQTYVTQQLIRAAEIRASANLAAT
KMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQGSGYIPEAPRDGQAYVRKDGEWVLLSTFLGRSGGGGS AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
SARS-CoV-2 S2_Vflip disulfide-L2-LuS_nopuritag
(SEQ ID NO: 69)
MRGLGTCLATLAGLLLTAAGSVASQSIIAYTMSLGAENSVACSNNSIAIPTNFTISVTTEILP
VSMTKTSVDCTMYICGDSTECSNLLLQYGSFCTQLNRALTGIAVEQDKNTQEVFAQVK
QIYKTPPIKDFGGFNFSQILPDPSKPSKRSPIEDLLFNKVTLADAGFIKQYGDCLGDIAAR
DLICAQKFNGLTVLPPLLTDEMIAQYTSALLAGTICSGWTFGAGPALQIPFPMQMAYRF
NGIGVTQNVLYENQKLIANQFNSAIGKIQDSLSSTPSALGKLQDVVNQNAQALNTLVKQ
LSSNCGAISSVLNDILSRLDKPEAEVQIDRLITCRLQSLQTYVTQQLIRAAEIRASANLAAT
KMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQGSGYIPEAPRDGQAYVRKDGEWVLLSTFLGRSGGGGS AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
MERS S2P-dRBD-L2-LuS (native)
(SEQ ID NO: 70)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRI
GAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESGV
YSVSSFEAKPSGSVVEQAEGGSGGKLEFANDTKIASQLGNCVEYSLYGVSGRGVFQNCT
AVGVRQQRFVYDAYQNLVGYYSDDGNYYCLRACVSVPVSVIYDKETKTHATLFGSVAC
EHISSTMSQYSRSTRSMLKRRDSTYGPLQTPVGCVLGLVNSSLFVEDCKLPLGQSLCALP
DTPSTLTPASVGSVPGEMRLASIAFNHPIQVDQLNSSYFKLSIPTNFSFGVTQEYIQTTIQK
VTVDCKQYVCNGFQKCEQLLREYGQFCSKINQALHGANLRQDDSVRNLFASVKSSQSS
PIIPGFGGDFNLTLLEPVSISTGSRSARSAIEDLLFDKVTIADPGYMQGYDDCMQQGPASA
RDLICAQYVAGYKVLPPLMDVNMEAAYTSSLLGSIAGVGWTAGLSSFAAIPFAQSIFYRL
NGVGITQQVLSENQKLIANKFNQALGAMQTGFTTTNEAFHKVQDAVNNNAQALSKLAS
ELSNTFGAISASIGDIIQRLDPPEQDAQIDRLINGRLTTLNAFVAQQLVRSESAALSAQLA
KDKVNECVKAQSKRSGFCGQGTHIVSFVVNAPNGLYFMHVGYYPSNHIEVVSAYGLCD
AANPTNCIAPVNGYFIKTNNTRIVDEWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGGS
AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
MERS S2P-dRBD-L6-LuS (native) no puritag
(SEQ ID NO: 71)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRI
GAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESGV
YSVSSFEAKPSGSVVEQAEGGSGGKLEFANDTKIASQLGNCVEYSLYGVSGRGVFQNCT
AVGVRQQRFVYDAYQNLVGYYSDDGNYYCLRACVSVPVSVIYDKETKTHATLFGSVAC
EHISSTMSQYSRSTRSMLKRRDSTYGPLQTPVGCVLGLVNSSLFVEDCKLPLGQSLCALP
DTPSTLTPASVGSVPGEMRLASIAFNHPIQVDQLNSSYFKLSIPTNFSFGVTQEYIQTTIQK
VTVDCKQYVCNGFQKCEQLLREYGQFCSKINQALHGANLRQDDSVRNLFASVKSSQSS
PIIPGFGGDFNLTLLEPVSISTGSRSARSAIEDLLFDKVTIADPGYMQGYDDCMQQGPASA
RDLICAQYVAGYKVLPPLMDVNMEAAYTSSLLGSIAGVGWTAGLSSFAAIPFAQSIFYRL
NGVGITQQVLSENQKLIANKFNQALGAMQTGFTTTNEAFHKVQDAVNNNAQALSKLAS
ELSNTFGAISASIGDIIQRLDPPEQDAQIDRLINGRLTTLNAFVAQQLVRSESAALSAQLA
KDKVNECVKAQSKRSGFCGQGTHIVSFVVNAPNGLYFMHVGYYPSNHIEVVSAYGLCD
AANPTNCIAPVNGYFIKTNNTRIVDEWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGSGGGGSGGGGSGLS
KMQIYEGKLTAEGLRFGIVASRFNHALVDRLVEGAIDCIVRHGGREEDITLVRVPGSWE
IPVAAGELARKEDIDAVIAIGVLIRGATPHFDYIASEVSKGLANLSLELRKPITFGVITADT
LEQAIERAGTKHGNKGWEAALSAIEMANLFKSLR
MERS S2P-dRBD-dFd-L2-LuS (native) nopuritag
(SEQ ID NO: 72)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRI
GAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESGV
YSVSSFEAKPSGSVVEQAEGGSGGKLEFANDTKIASQLGNCVEYSLYGVSGRGVFQNCT
AVGVRQQRFVYDAYQNLVGYYSDDGNYYCLRACVSVPVSVIYDKETKTHATLFGSVAC
EHISSTMSQYSRSTRSMLKRRDSTYGPLQTPVGCVLGLVNSSLFVEDCKLPLGQSLCALP
DTPSTLTPASVGSVPGEMRLASIAFNHPIQVDQLNSSYFKLSIPTNFSFGVTQEYIQTTIQK
VTVDCKQYVCNGFQKCEQLLREYGQFCSKINQALHGANLRQDDSVRNLFASVKSSQSS
PIIPGFGGDFNLTLLEPVSISTGSRSARSAIEDLLFDKVTIADPGYMQGYDDCMQQGPASA
RDLICAQYVAGYKVLPPLMDVNMEAAYTSSLLGSIAGVGWTAGLSSFAAIPFAQSIFYRL
NGVGITQQVLSENQKLIANKENQALGAMQTGFTTTNEAFHKVQDAVNNNAQALSKLAS
ELSNTFGAISASIGDIIQRLDPPEQDAQIDRLINGRLTTLNAFVAQQLVRSESAALSAQLA
KDKVNECVKAQSKRSGFCGQGTHIVSFVVNAPNGLYFMHVGYYPSNHIEVVSAYGLCD
AANPTNCIAPVNGYFIKTNNTRIVDEWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGGGGS AEAAAKEAAAKEAAAKA
GGGGSMQIYEGKLTAEGLRFGIVASRFNHALVDRLVEGAIDCIVRHGGREEDITLVRVP
GSWEIPVAAGELARKEDIDAVIAIGVLIRGATPHFDYIASEVSKGLANLSLELRKPITFGVI
TADTLEQAIERAGTKHGNKGWEAALSAIEMANLFKSLR
MERS S2P-dRBD-L2-AFerr nopuritag
(SEQ ID NO: 73)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRI
GAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESGV
YSVSSFEAKPSGSVVEQAEGGSGGKLEFANDTKIASQLGNCVEYSLYGVSGRGVFQNCT
AVGVRQQRFVYDAYQNLVGYYSDDGNYYCLRACVSVPVSVIYDKETKTHATLFGSVAC
EHISSTMSQYSRSTRSMLKRRDSTYGPLQTPVGCVLGLVNSSLFVEDCKLPLGQSLCALP
DTPSTLTPASVGSVPGEMRLASIAFNHPIQVDQLNSSYFKLSIPTNFSFGVTQEYIQTTIQK
VTVDCKQYVCNGFQKCEQLLREYGQFCSKINQALHGANLRQDDSVRNLFASVKSSQSS
PIIPGFGGDFNLTLLEPVSISTGSRSARSAIEDLLFDKVTIADPGYMQGYDDCMQQGPASA
RDLICAQYVAGYKVLPPLMDVNMEAAYTSSLLGSIAGVGWTAGLSSFAAIPFAQSIFYRL
NGVGITQQVLSENQKLIANKFNQALGAMQTGFTTTNEAFHKVQDAVNNNAQALSKLAS
ELSNTFGAISASIGDIIQRLDPPEQDAQIDRLINGRLTTLNAFVAQQLVRSESAALSAQLA
KDKVNECVKAQSKRSGFCGQGTHIVSFVVNAPNGLYFMHVGYYPSNHIEVVSAYGLCD
AANPTNCIAPVNGYFIKTNNTRIVDEWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGGS
AEAAAKEAAAKEAAAKA
GGGGSMSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFE
RLTLKHD
LSO3C-MERS S2P-dRBD-L6-AFerr nopuritag
(SEQ ID NO: 74)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRI
GAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESGV
YSVSSFEAKPSGSVVEQAEGGSGGKLEFANDTKIASQLGNCVEYSLYGVSGRGVFQNCT
AVGVRQQRFVYDAYQNLVGYYSDDGNYYCLRACVSVPVSVIYDKETKTHATLFGSVAC
EHISSTMSQYSRSTRSMLKRRDSTYGPLQTPVGCVLGLVNSSLFVEDCKLPLGQSLCALP
DTPSTLTPASVGSVPGEMRLASIAFNHPIQVDQLNSSYFKLSIPTNFSFGVTQEYIQTTIQK
VTVDCKQYVCNGFQKCEQLLREYGQFCSKINQALHGANLRQDDSVRNLFASVKSSQSS
PIIPGFGGDFNLTLLEPVSISTGSRSARSAIEDLLFDKVTIADPGYMQGYDDCMQQGPASA
RDLICAQYVAGYKVLPPLMDVNMEAAYTSSLLGSIAGVGWTAGLSSFAAIPFAQSIFYRL
NGVGITQQVLSENQKLIANKENQALGAMQTGFTTTNEAFHKVQDAVNNNAQALSKLAS
ELSNTFGAISASIGDIIQRLDPPEQDAQIDRLINGRLTTLNAFVAQQLVRSESAALSAQLA
KDKVNECVKAQSKRSGFCGQGTHIVSFVVNAPNGLYFMHVGYYPSNHIEVVSAYGLCD
AANPTNCIAPVNGYFIKTNNTRIVDEWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGSGGGGSGGGGSGLS
KMSSQIRQNYSTDVEAAVNSLVNLYLQASYTYLSLGFYFDRDDVALEGVSHFFRELAEE
KREGYERLLKMQNQRGGRALFQDIKKPAEDEWGKTPDAMKAAMALEKKLNQALLDL
HALGSARTDPHLCDFLETHFLDEEVKLIKKMGDHLTNLHRLGGPEAGLGEYLFERLTL
KHD
MERS S2P-dRBD-dFd-L2-Aferr (native)
(SEQ ID NO: 75)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRI
GAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESGV
YSVSSFEAKPSGSVVEQAEGGSGGKLEFANDTKIASQLGNCVEYSLYGVSGRGVFQNCT
AVGVRQQRFVYDAYQNLVGYYSDDGNYYCLRACVSVPVSVIYDKETKTHATLFGSVAC
EHISSTMSQYSRSTRSMLKRRDSTYGPLQTPVGCVLGLVNSSLFVEDCKLPLGQSLCALP
DTPSTLTPASVGSVPGEMRLASIAFNHPIQVDQLNSSYFKLSIPTNFSFGVTQEYIQTTIQK
VTVDCKQYVCNGFQKCEQLLREYGQFCSKINQALHGANLRQDDSVRNLFASVKSSQSS
PIIPGFGGDFNLTLLEPVSISTGSRSARSAIEDLLFDKVTIADPGYMQGYDDCMQQGPASA
RDLICAQYVAGYKVLPPLMDVNMEAAYTSSLLGSIAGVGWTAGLSSFAAIPFAQSIFYRL
NGVGITQQVLSENQKLIANKFNQALGAMQTGFTTTNEAFHKVQDAVNNNAQALSKLAS
ELSNTFGAISASIGDIIQRLDPPEQDAQIDRLINGRLTTLNAFVAQQLVRSESAALSAQLA
KDKVNECVKAQSKRSGFCGQGTHIVSFVVNAPNGLYFMHVGYYPSNHIEVVSAYGLCD
AANPTNCIAPVNGYFIKTNNTRIVDEWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGGGGS AEAAAKEAAAKEAAAKA
GGGGSMSSQIRQNYSTDVEAAVNSLVNLYLQASYTYLSLGFYFDRDDVALEGVSHFFRE
LAEEKREGYERLLKMQNQRGGRALFQDIKKPAEDEWGKTPDAMKAAMALEKKLNQA
LLDLHALGSARTDPHLCDFLETHFLDEEVKLIKKMGDHLTNLHRLGGPEAGLGEYLFE
RLTLKHD
MERS S2P-dRBD-L2-Hpylori Ferr_nopuritag
(SEQ ID NO: 76)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRI
GAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESGV
YSVSSFEAKPSGSVVEQAEGGSGGKLEFANDTKIASQLGNCVEYSLYGVSGRGVFQNCT
AVGVRQQRFVYDAYQNLVGYYSDDGNYYCLRACVSVPVSVIYDKETKTHATLFGSVAC
EHISSTMSQYSRSTRSMLKRRDSTYGPLQTPVGCVLGLVNSSLFVEDCKLPLGQSLCALP
DTPSTLTPASVGSVPGEMRLASIAFNHPIQVDQLNSSYFKLSIPTNFSFGVTQEYIQTTIQK
VTVDCKQYVCNGFQKCEQLLREYGQFCSKINQALHGANLRQDDSVRNLFASVKSSQSS
PIIPGFGGDFNLTLLEPVSISTGSRSARSAIEDLLFDKVTIADPGYMQGYDDCMQQGPASA
RDLICAQYVAGYKVLPPLMDVNMEAAYTSSLLGSIAGVGWTAGLSSFAAIPFAQSIFYRL
NGVGITQQVLSENQKLIANKFNQALGAMQTGFTTTNEAFHKVQDAVNNNAQALSKLAS
ELSNTFGAISASIGDIIQRLDPPEQDAQIDRLINGRLTTLNAFVAQQLVRSESAALSAQLA
KDKVNECVKAQSKRSGFCGQGTHIVSFVVNAPNGLYFMHVGYYPSNHIEVVSAYGLCD
AANPTNCIAPVNGYFIKTNNTRIVDEWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGGS
AEAAAKEAAAKEAAAKA
GGGGSMLSKDIIKLLNEQVNKEMNSSNLYMSMSSWCYTHSLDGAGLFLFDHAAEEYEH
AKKLIVFLNENNVPVQLTSISAPEHKFEGLTQIFQKAYEHEQHISESINNIVDHAIKSKDH
ATFNFLQWYVAEQHEEEVLFKDILDKIELIGNENHGLYLADQYVKGIAKSRKS
MERS S2P-dRBD-L6-Hpylori Ferr_nopuritag
(SEQ ID NO: 77)
MIHSVFLLMFLLTPTESYVDVGPDSVKSACIEVDIQQTFFDKTWPRPIDVSKADGIIYPQGR
TYSNITITYQGLFPYQGDHGDMYVYSAGHATGTTPQKLFVANYSQDVKQFANGFVVRI
GAAANSTGTVIISPSTSATIRKIYPAFMLGSSVGNFSDGKMGRFFNHTLVLLPDGCGTLL
RAFYCILEPRSGNHCPAGNSYTSFATYHTPATDCSDGNYNRNASLNSFKEYFNLRNCTF
MYTYNITEDEILEWFGITQTAQGVHLFSSRYVDLYGGNMFQFATLPVYDTIKYYSIIPHSI
RSIQSDRKAWAAFYVYKLQPLTFLLDFSVDGYIRRAIDCGFNDLSQLHCSYESFDVESGV
YSVSSFEAKPSGSVVEQAEGGSGGKLEFANDTKIASQLGNCVEYSLYGVSGRGVFQNCT
AVGVRQQRFVYDAYQNLVGYYSDDGNYYCLRACVSVPVSVIYDKETKTHATLFGSVAC
EHISSTMSQYSRSTRSMLKRRDSTYGPLQTPVGCVLGLVNSSLFVEDCKLPLGQSLCALP
DTPSTLTPASVGSVPGEMRLASIAFNHPIQVDQLNSSYFKLSIPTNFSFGVTQEYIQTTIQK
VTVDCKQYVCNGFQKCEQLLREYGQFCSKINQALHGANLRQDDSVRNLFASVKSSQSS
PIIPGFGGDFNLTLLEPVSISTGSRSARSAIEDLLFDKVTIADPGYMQGYDDCMQQGPASA
RDLICAQYVAGYKVLPPLMDVNMEAAYTSSLLGSIAGVGWTAGLSSFAAIPFAQSIFYRL
NGVGITQQVLSENQKLIANKFNQALGAMQTGFTTTNEAFHKVQDAVNNNAQALSKLAS
ELSNTFGAISASIGDIIQRLDPPEQDAQIDRLINGRLTTLNAFVAQQLVRSESAALSAQLA
KDKVNECVKAQSKRSGFCGQGTHIVSFVVNAPNGLYFMHVGYYPSNHIEVVSAYGLCD
AANPTNCIAPVNGYFIKTNNTRIVDEWSYTGSSFYAPEPITSLNTKYVAPQVTYQNISTNL
PPPLLGNSTGIDFQDELDEFFKNVSTSIPNFGSLTQINTTLLDLTYEMLSLQQVVKALNES
YIDLKELGNYTYGSGYIPEAPRDGQAYVRKDGEWVLLSTFLGGGGSGGGGSGGGGSGLS
KMLSKDIIKLLNEQVNKEMNSSNLYMSMSSWCYTHSLDGAGLFLFDHAAEEYEHAKKL
IVFLNENNVPVQLTSISAPEHKFEGLTQIFQKAYEHEQHISESINNIVDHAIKSKDHATFNF
LQWYVAEQHEEEVLFKDILDKIELIGNENHGLYLADQYVKGIAKSRKS

### Linker Summary

Complete Sequence of Designed Linkers

| | | | | SEQ ID |
|---|---|---|---|---|
| Linker | Type | Short form | Complete sequence | NO |
| L1 | Flexible | (GGGGS)ₓ₅ | GGGGS GGGGS GGGGS GGGGS GGGGS | 1 |
| | Long | | | |
| L2 | Helical | GGGGS | GGGGS AEAAAKEAAAKEAAAKA GGGGS | 2 |
| | | A(EAAAK)₃A- | | |
| | | (GGGGS) | | |
| L3 | Helical | A(EAAAK)₅A- | GGGGSAEAAAKEAAAKEAAAKEAAAKEA | 3 |
| | | (GGGGS) | AAKA GGGGS | |
| L4 | Semi | (GSSSSS)₃ | GSSSSS GSSSSS GSSSSS | 4 |
| | flexible | | | |
| L5 | Hybrid | GSA(EAAAK)₃ | GSA EAAAK EAAAK EAAAKA GGGGSA | 5 |
| | | A-(GGGGS)- | EAAAK EAAAK EAAAKAGS | |
| | | A(EAAAK)₃AGS | | |
| L6 | Flexible | GGGSGGGGSG | GGGSGGGGSGGGGSG | 6 |
| | Short | GGGSGLSK | | |
| L7 | Hybrid | GSSSSS | GSSSSS AEAAAKEAAAKEAAAKA GSSSSS | 7 |
| | | A(EAAAK)₃A- | | |
| | | (GSSSSS) | | |

### Signal Peptide/Leader Sequence Used.

| | Signal peptide /Leader | | |
|---|---|---|---|
| | Sequence Type | Amino acid Sequence | SEQ ID NO |
| A | Tissue plasminogen | MDAMKRGLCCVLLLCGAVFVSPSQEIHARFRRGAR | 10 |
| | activator (TPA) signal | | |
| | sequence | | |
| B | Modified bovine | MDSKGSSQKGSRLLLLLVVSNLLLPQGVLA | 11 |
| | prolactin | | |
| C | Ching Cell Report | MRPTWAWWLFLVLLLALWAPARG | 12 |
| | (SS V2 paper) | | |
| D | MERS S native signal | MIHSVFLLMFLLTPTES | 13 |
| | peptide | | |
| E | N- | MPMGSLQPLATLYLLGMLVASVLA | 14 |
| | terminal human CD5 | | |
| | leader sequence | | |
| F | mu-phosphatase signal | MRGLGTCLATLAGLLLTAAG | 15 |
| | peptide | | |
| G | SARS-CoV-2 S native | MFVFLVLLPLVSSQC | 16 |
| | signal peptide | | |

A was used for both full MERS spike and NTD NPs sequence containing purification tags;
C was used for SSV2 NPs Secretion; B was previously used for secreting SSV2 NPs containing purification tags but had difficult secreting.
D was used for secretion of full MERS spike (S2P dRBD) NPs without purification tags
F was used for secretion of SARS-CoV-2 S2 hexapro and S2 Vflip with further disulfide stabilizing mutations which contained no signal peptide.
F was used for secretion of MERS RBD NPs containing purification tags
G was used for secretion of SARS-CoV-2 spike ( SARS S2P dRBD) NPs without purification tags

### Nanoparticle Sequences

| | | SEQ ID |
|---|---|---|
| Selected NPS | Sequences | NO |
| Human ApoFerritin | MSSQIRQNYSTDVEAAVNSLVNLYLQASYTYLSLGFYFDRDDVA | 17 |
| LC (1-175) | LEGVSHFFRELAEEKREGYERLLKMQNQRGGRALFQDIKKPAED | |
| 24 mer | EWGKTPDAMKAAMALEKKLNQALLDLHALGSARTDPHLCDFLE | |
| | THFLDEEVKLIKKMGDHLTNLHRLGGPEAGLGEYLFERLTLKHD | |
| H. pylori ferritin | MLSKDIIKLLNEQVNKEMNSSNLYMSMSSWCYTHSLDGAGLFLF | 18 |
| NPs (residues 1- | DHAAEEYEHAKKLIVFLNENNVPVQLTSISAPEHKFEGLTQIFQKA | |
| 168) - 24 mer | YEHEQHISESINNIVDHAIKSKDHATFNFLQWYVAEQHEEEVLFK | |
| | DILDKIELIGNENHGLYLADQYVKGIAKSRKS | |
| Aquifex aeolicus | MQIYEGKLTAEGLRFGIVASRFNHALVDRLVEGAIDCIVRHGGRE | 19 |
| Lumazine synthase | EDITLVRVPGSWEIPVAAGELARKEDIDAVIAIGVLIRGATPHFDYI | |
| (LuS) | ASEVSKGLANLSLELRKPITFGVITADTLEQAIERAGTKHGNKGW | |
| 60 mer | EAALSAIEMANLFKSLR | |
| Aquifex aeolicus | MQIYEGKLTAEGLRFGIVASRFNHALVDRLVEGAIDCIVRHGGRE | 20 |
| Lumazine synthase | EDITLVRVPGSWEIPVAAGELARKEDIDAVIAIGVLIRGATPHFDYI | |
| (LuS) - Modified | ASEVSKGLANLSLELRKPITFGVITADTLEQAIERAGTKHGNKGW | |
| 60 mer | EAALSAIEMANLFKSLR | |

### Example 2: Antigenic Characterization of Rationally Designed Pan-Beta Coronavirus Nanoparticle Vaccines

The recurrent spillovers of pathogenic betacoronaviruses (β-CoVs) and the emergence of SARS-CoV-2 variants in humans have caused serious morbidity and mortality globally. While an arsenal of vaccines has been developed or licensed, the antigenic discordance between the current vaccines and circulating variants has diminished their efficacies and utility. This is largely due to many escape mutations within the immunodominant receptor-binding domain (RBD) and exposed variable epitopes of the spike protein within these β-CoVs. In contrast, the non-RBD domains (N-terminal domain (NTD) and S2) are relatively conserved, and immunogens featuring these domains may provide broader protection against β-CoVs and reduce the need for annual vaccine reformulation and immunization. Broadly, using biolayer interferometry (BLI), this disclosure presents novel nanoparticle vaccine candidates that could potentially protect against diverse β-CoVs.

Described herein is the rational design of non-RBD antigens and their precision structural presentation with linkers (flexible and rigid) and nanoparticles (apoferritin and lumazine synthase) to favor induction of cross-reactive response using the MERS spike protein as a prototype. These immunogens were produced in mammalian cells and characterized via various biochemical and biophysical techniques.

The present work robustly explores the antigenicity of the designed immunogens using BLI. It was found that nanoparticle immunogens enhanced the binding of the engineered antigens even at a low dose, suggesting mechanisms of better B-cell activation and production of more B-cells and protective antibodies. Furthermore, spike nanoparticle immunogens designed with a rigid linker exposed certain conserved S2 stalk epitopes targeted by potent neutralizing antibodies, suggesting rare but broad B-cell receptor targeting, priming unmutated stalk B-cell precursors of broadly neutralizing antibody (bnAb) lineages and immune induction of cross-reactive responses. This disclosure highlights the advantages of precisely displaying β-CoV spike antigen components using nanoparticles and establishes an understanding of their antigenic profiles. Future directions of this research include further engineering of some nanoparticle vaccine candidates and in vivo immunogenicity and protective studies of lead or promising candidates in animal models.

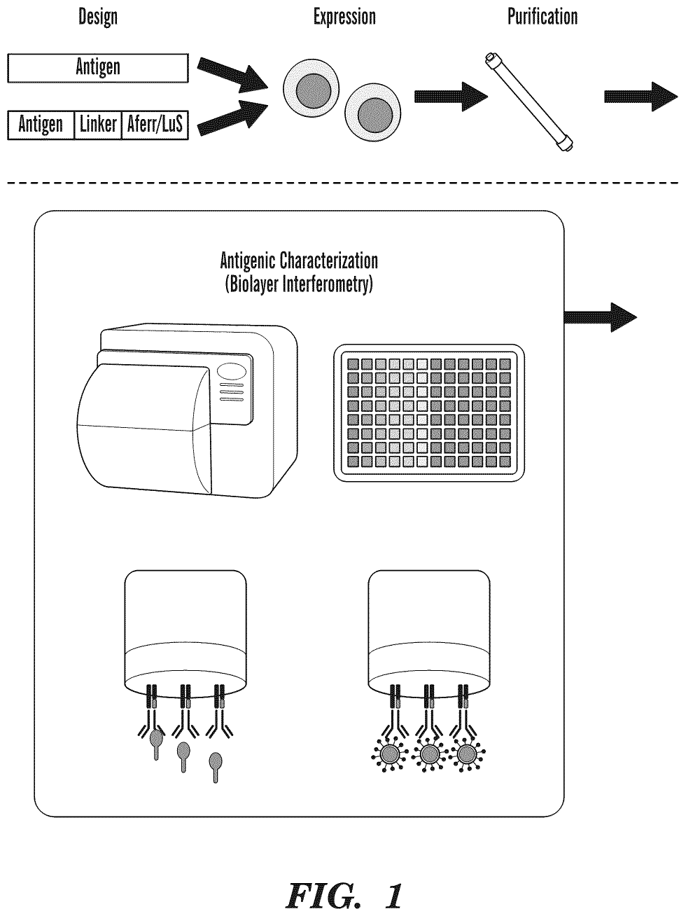

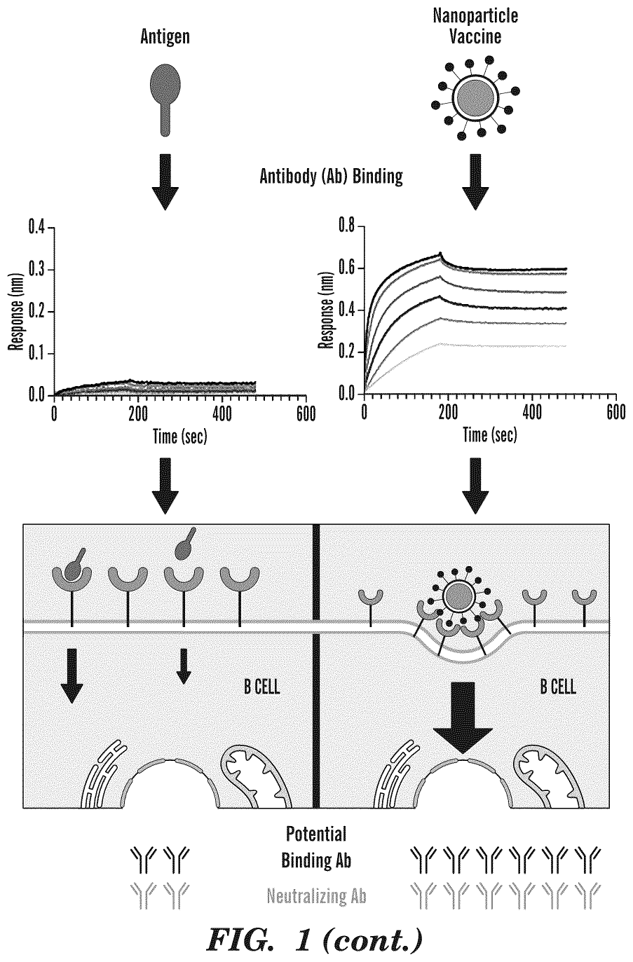

By 1) rationally designing and producing novel pan-betaCoronavirus vaccine immunogens, 2) using biolayer interferometry to characterize their antigenicity, and 3) informing and predicting their possible immune induction (FIG. 1), the inventors were able to identify nanoparticle vaccine candidates that could potentially protect against diverse beta Coronaviruses, which pose a dire public health threat.

### LIST OF ABBREVIATIONS

Ab Antibody
ACE2 Angiotensin-converting enzyme 2
Aferr Apoferritin
BLI Biolayer interferometry
bnAb Broadly neutralizing antibody
CoV Coronavirus
MERS Middle East respiratory syndrome
SARS Severe Acute Respiratory Syndrome
DPP4 Dipeptidyl peptidase-4
L2 Helical and rigid linker
L6 Flexible linker
LuS Lumazine synthase
NTD N-terminal domain
RBD Receptor binding domain
S2P Pre-fusion stabilized two-proline spike
SS Stabilized stem

### Introduction

Since emerging in 2019, severe acute respiratory syndrome coronavirus 2 (SARS-CoV-2) has resulted in over 6 million deaths (Dong et al., 2020), emphasizing how emerging and re-emerging coronaviruses (CoVs) pose continuous threats to human health. While humans move towards SARS-CoV-2 endemicity, unfortunately 26 viral families have been described with potential to transmit to humans, threatening to cause future pandemics (Graham and Sullivan, 2018). Furthermore, the threat posed by CoVs remain largely unmitigated. The inevitable declassification of SARS-CoV-2 to another endemic human CoV raises concern that humans will be poorly prepared for pandemics that eventually morph into endemics, as this has been the outcome for most respiratory virus pandemics (Cohen et al., 2022b). Prior to the COVID-19 pandemic, Middle East respiratory syndrome coronavirus (MERS-CoV) emerged in 2012 and led to the MERS epidemic, which still poses a pandemic threat. Since its first detection in Saudi Arabia, over 2,600 cases have been detected in over 27 countries (ECDC, 2022). Infection with MERS-CoV can range from mild symptoms to death, and has had a case-fatality rate of 32.7% (Zhang et al., 2021). Presently, strategies to drive broad immunity against MERS-CoV are lacking: there is no approved vaccine for MERS-CoV. We must be prepared to respond to the next coronavirus threat.

Moreover, the concurrent prevalence of MERS-CoV and SARS-CoV-2 presents a concern for the emergence of future novel betaCoronavirus (3-CoV) clades by means of genetic recombination or coinfection due to ACE2 receptor-sharing between SARS-CoV-2 and MERS-CoV relatives (Xiong et al., 2022). These MERS-CoVs use angiotensin-converting enzyme 2 (ACE2) orthologues as entry receptors instead their conventional dipeptidyl peptidase-4 (DPP4). The possibility for a MERS-CoV-2 or SARS-CoV-3-like CoV, combining the high mortality of MERS-CoV and high transmissibility of SARS-CoV-2, underscores the urgent need to develop pan-p-CoV vaccines (Su et al., 2022). Indeed, several cases of coinfection between MERS-CoV and SARS-CoV-2 were recently reported in Saudi Arabia (Elhazmi et al., 2021), perhaps suggesting recombination between these two β-CoVs. Thus, there is a demand to develop next-generation precision approaches to provide long-term and broad immunity against MERS-CoV and other β-CoVs (Wang et al., 2023). The inventors aim to combine novel immunogen designs and precision nanoparticle presentation to induce broad protection in response to this need. Described herein is the evaluation of the antigenicity of various engineered precision nanoparticle vaccine candidates.

### Coronaviruses

CoVs comprise a family of large, single-stranded, enveloped, positive-sense RNA viruses with strains known to infect mammals and avian species. Their phylogeny is divided into four genera α, β, γ, and δ with β-CoV having four sub lineages A, B, C. and D (Woo et al., 2009). There are seven CoVs known to infect humans (hCoVs), and four of these strains circulate endemically (Corman et al., 2018). hCoV-229E and hCoV-NL63 are from the genus α (α-hCoVs), and hCoV-OC43 and hCoV-HKU1 are from the genus β (β-hCoVs) lineage A (Cui et al., 2019). These hCoVs circulate globally and account for 10-30% of upper respiratory tract infections in adults. These diseases are generally mild, but can become more severe in infants, elderly, and immunocompromised patients (Paules et al., 2020). In contrast, MERS-CoV and SARS-CoV are highly pathogenic and have emerged in the past 15 years from animal reservoirs, causing outbreaks with high fatalities in human populations (Peiris et al., 2003; Zaki et al., 2012). Both viruses are β-CoVs, MERS-CoV from lineage C, and SARS-CoV from lineage B (Cui et al., 2019). MERS-CoV still circulates across the Arabian Peninsula, most commonly transmitted from infected animals to humans or from humans to humans (Mackay and Arden, 2015).

The adaptation of hCoVs to different host species has resulted in the emergence of the novel SARS-CoV-2, the causative agent of the Coronavirus disease 2019 (COVID-19) pandemic responsible for over 600 million infections and 6 million deaths worldwide (Dong et al., 2020). As evidenced by the advancing COVID-19 pandemic, SARS-CoV-2 will likely continue to evolve to eventually circulate as the third endemic β-hCoV (Kissler et al., 2020). Furthermore, the emergence of SARS-CoV-2 variants of concern (CDC, 2022), particularly delta and omicron variants, highlights the threat of viral escape from vaccine-induced antibodies from currently available COVID-19 vaccines (Morens et al., 2021).

The concurrent prevalence of endemic hCoVs, MERS-CoV, and SARS-CoV-2 raises concerns about a potential spillover of new clades into humans with possibly higher transmissibility and mortality rates. There are currently no approved human vaccines against MERS-CoV or the endemic hCoVs. Therefore, there is a demonstrated need to prepare against these threats to avoid future public health, social, and economic ravages of coronavirus pandemics that morph into epidemics or endemics.

### Nanoparticle Platform, Linker Selection, and Engineering

Displaying multivalent antigens on nanoparticles is a recognized strategy to increase and enhance immunogenicity. However, the interplay between the suitable nanoparticles and engineered antigens to allow exposure of conserved or cross-reactive epitopes has not been extensively explored.

To address the goal of design optimization, the inventors utilize rationally-designed multivalent antigens tethered to engineered linkers and self-assembling nanoparticles, hence dubbed “precision nanoparticle vaccines.” Nanoparticles are nanoscale-sized (10-150 nm diameter), tunable, highly stable, and symmetric particles (Lee and Wang, 2006; Vu et al., 2021). Nanoparticles can play diverse physiological roles, but they are of particular interest for vaccine development because they may be used as a self-assembling platform to display a particular spike antigen in a well-arranged and stable matrix, thus mimicking the repetitive nature of a virus' surface architecture (Castón and Carrascosa, 2013; López-Sagaseta et al., 2016).

The mechanisms behind the benefits of nanoparticle vaccines stem from their optimal size and their interaction with the immune system being comparable to that of viruses (Bachmann and Jennings, 2010; Chattopadhyay et al., 2017). The adaptability of nanoparticle vaccines also makes them highly promising as a next-generation vaccine platform, allowing for approaches to drive more robust neutralizing antibody responses, broader antibody-based immunity against viral evolution and variation (Vu et al., 2021), protection from premature degradation of the antigen, and improved intracellular uptake (Poon and Patel, 2020). Nanoparticle vaccines are also easy to produce in Escherichia coli (E. coli), making them suitable for large-scale production. Although there are no FDA-approved commercial nanoparticle vaccines, many researchers are developing vaccines for infectious diseases like human immunodeficiency virus (HIV) or influenza with this platform and are reporting promising preclinical and clinical results (López-Sagaseta et al., 2016). The present work with displaying CoV antigens on nanoparticles will contribute to the ongoing research on this emerging platform and the discussion surrounding the strategic display of CoV antigens.

The individual components of the precision nanoparticle vaccine are the self-assembling nanoparticle itself, the linker, and the engineered antigen that the nanoparticle displays. There are many self-assembling protein nanoparticles, but it was chosen to utilize the ferritin-based and lumazine synthase-based nanoparticles for instant vaccine candidates because of several potential benefits.

Ferritin self-assembles into nanoparticles that exhibit strong thermal and chemical stability and are therefore suitable to carry and display immunogens. These nanoparticles possess octahedral symmetry and can display 24 monomers or 8 units with three-fold axis symmetry, making them favorable to present trimeric antigens (López-Sagaseta et al., 2016). One research group generated ferritin nanoparticles displaying a multivalent array of influenza virus hemagglutinin (HA) in its native trimeric conformation, and they achieved more potent immune responses than the current commercial vaccines (Kanekiyo et al., 2013). Based on this promising data and the trimeric nature of the CoV spike protein, Ferritin nanoparticles were selected for the instant design. Furthermore, human apoferritin (Aferr) as opposed to insect or bacterial ferritin is utilized to avoid anti-scaffold antibody responses and guide the immune response to the displayed engineered spike domains (Houser et al., 2022).

Lumazine Synthase (LuS) is an enzyme involved in riboflavin synthesis and is the other particulate base with which we are engineering the instant nanoparticle vaccine candidates. LuS forms an icosahedral particle that can display 60 monomers or 20 trimers (López-Sagaseta et al., 2016). One research group used the LuS nanoparticle to increase the potency and breadth of the immune response coverage against HIV by activating both germline and mature B-cells. In addition, their engineered optimized sub-component of the wild-type gp120 antigen from the envelope glycoprotein was displayed on the surface of LuS nanoparticles (Jardine et al., 2013).

The next aspect of design optimization focused on linker selection in order to spatially expose the conserved epitopes on the antigen and induce broadly neutralizing antibodies. There are conventional flexible linkers, which have been used previously in nanoparticle vaccine designs. However, despite being suitable to allow proper and independent folding of the components, these linkers are unstructured. In contrast, rigid or helical linkers are more structured, which may permit spike antigen projection at optimal distances, conformations, and orientations. These display advantages conferred by structured linkers could lead to enhanced B-cell engagement or activation, bivalent or multivalent cross linking, and exposure of neutralization epitopes in the conserved S2 domain (Nguyen and Tolia, 2021). The length of the linkers were taken into consideration, as one study which designed a nanoparticle vaccine for SARS-CoV-2 found that longer linkers enabled better access to the RBD epitopes targeted by antibodies ACE2, CR3022, and S309 (Walls et al., 2020a). Both a rigid linker and a flexible linker were tested in the instant nanoparticle designs in order to evaluate their relative advantages.

Using Aferr- and LuS-based nanoparticles along with these linkers, MERS-CoV antigens were displayed and the antigenicity of the various nanoparticle vaccine candidates assessed.

### Rational and Novel Immunogen Design Strategy

CoVs interact and infect cells via spike proteins on the viral surface. The spike protein has two subunits: The S1 subunit is essential for host cell receptor engagement and contains the receptor binding domain (RBD) and the N-terminal domain (NTD). The more conserved S2 subunit determines membrane fusion and viral entry (Walls et al., 2020b). The current approved SARS-CoV-2 vaccines induce potent neutralizing antibody responses by primarily targeting the immunodominant RBD epitopes of the S1 subunit (Duan et al., 2020). However, the lack of sequence conservation across different CoV S1 subunits implies that vaccines based on the S1 would fail to offer broad protection against CoVs other than SARS-CoV-2 (Walls et al., 2020b). Furthermore, as SARS-CoV-2 evolves, many mutations have accumulated in the RBD, NTD, and S2, potentially rendering current vaccines ineffective (Harvey et al., 2021). Recently, the newly emerged omicron variants containing more than 15 escape mutations within the RBD evaded RBD-targeted neutralizing antibodies and resist neutralization by the parental mRNA vaccine or bivalent booster (Cao et al., 2022; Greaney et al., 2021; Kurhade et al., 2023). On the contrary, anti-NTD responses against omicron- and variant-reactive memory B-cells were induced or even boosted following bi-valent vaccination or natural infection with variants (Wang et al., 2022). Moreover, incorporating NTD into the SARS-CoV-2 RBD protein vaccine has been demonstrated to stimulate and enhance immunity against variants of concern compared to the RBD dimer (Montgomerie et al., 2023). While omicron variants possess a few mutations in the S2 region, some S2 antibodies have been demonstrated to neutralize these variants (Hu et al., 2022).

The inventors therefore aim to engineer and utilize non-RBD epitopes to elicit a broad response and avoid escape mutations. The S2 subunit is highly conserved across multiple CoV variants and different β-CoVs (Huang et al., 2020; Wang et al., 2021a), thus making it very appealing for a pan-CoV vaccine antigen candidate. To mention a few studies, Dacon and colleagues found that rare, convergent antibodies that target the stem helix can neutralize diverse β-CoVs (Dacon et al., 2022), and Zhou and colleagues recently isolated a large panel of broadly neutralizing anti-S2 antibodies that protect against all three human β-CoVs that cause severe disease: SARS-CoV, SARS-CoV-2, and MERS-CoV (Zhou et al., 2022a). Several other studies have also found S2-targeting broadly neutralizing antibodies (bnAb) and conserved sites across β-CoVs (Hurlburt et al., 2022; Pinto et al., 2021; Sauer et al., 2021; Song et al., 2021; Zhou et al., 2022b). However, these kinds of antibodies are rare in humans after infection and may require rational targeted vaccination strategies to elicit them (Dacon et al., 2022; Zhou et al., 2022a). Previously, anti-S2 antibodies were isolated after random immunization with diverse spikes from different CoVs (Hurlburt et al., 2022; Ng et al., 2022; Sauer et al., 2021; Wang et al., 2021a). Moreover, whilst S2 targeted vaccination has recently been demonstrated in the case of SARS-CoV-2, these approaches also involved random S2 vaccination and elicited weaker virus neutralization (Ma et al., 2022; Ng et al., 2022). Therefore, a practical, effective vaccine strategy targeting S2 has not yet been identified.

One strategy to overcome these hurdles involves priming the immune system with engineered and stabilized S2-derived immunogens in order to elicit responses from these antibodies (similar to those rarely observed due to natural infection or current vaccines), which are broadly neutralizing against multiple β-CoVs. For example, by removing the S1 subunit from MERS-CoV, Hsieh and colleagues developed stabilized S stem (SS) antigens that successfully elicited cross-reactive β-CoV antibody responses and protected against lethal MERS-CoV challenge in mice. The group's best performing SS construct was version 2 of their SS construct, containing an additional stabilizing disulfide bond that induced stronger neutralizing response than the prior version (Hsieh et al., 2021), corroborating the importance of antigen stabilization to eliciting more potent neutralizing antibodies.

Thus, it is hypothesized that displaying these rationally designed and stable antigens on certain nanoparticles will enhance the breadth and strength of the cross-reactive response across multiple CoV strains. The nanoparticle will permit access to the conserved epitopes for targeted B-cell activation, stimulation, and elicitation of rare but potent neutralizing antibodies. In addition, described herein is an engineered MERS-SSΔG4loop antigen as it has been found that G4, the first MERS S2 directed neutralizing antibody, recognizes a variable loop in the MERS-CoV spike (Pallesen et al., 2017). By deleting this loop, the goal is to elicit more cross-reactive or broadly neutralizing antibody responses against multiple CoVs.

In summary, described herein is the rational display of engineered antigens from three categories: MERS-NTD, stabilized stem (SS), and MERS-S2PΔRBD, an engineered prefusion stabilized S antigen without RBD. These antigens are displayed by tethering by two different engineered linkers to Aferr and LuS nanoparticles (Table 1), and then the antigenicity of the resulting nanoparticle vaccine candidates is characterized. Nanoparticles with MERS-RBD were also designed to be used as comparators in future work. Similar MERS RBD nanoparticles have been recently explored (Lin et al., 2019; Okba et al., 2020; Rodon et al., 2022). However, the recent emergence of MERS-CoV variants (Xiong et al., 2022) raises concern over their efficacy as future vaccine candidates, further corroborating the importance of the MERS-S2PΔRBD design.

TABLE 1
Components used in this thesis for designed nanoparticle vaccine candidates.

| Individual Components | Type | Reference |
|---|---|---|
| Self-Assembling | Apoferritin (Aferr) | (Houser et al., 2022) |
| Nanoparticle | Lumazine Synthase (LuS) | (Jardine et al., 2013) |
| Linkers | L2: Helical/Rigid | Corbett Lab |
| | L6: Flexible | Corbett Lab |
| Engineered Antigen | MERS-RBD | (Pallesen et al., 2017; |
| | | Wang et al., 2015) |
| | MERS-NTD | (Wang et al., 2019) |
| | MERS-SS | (Hsieh et al., 2021) |
| | MERS-SSΔG4loop | Corbett Lab |
| | MERS-S2PΔRBD | Corbett Lab |

### Experimental Hypothesis and Design Approach

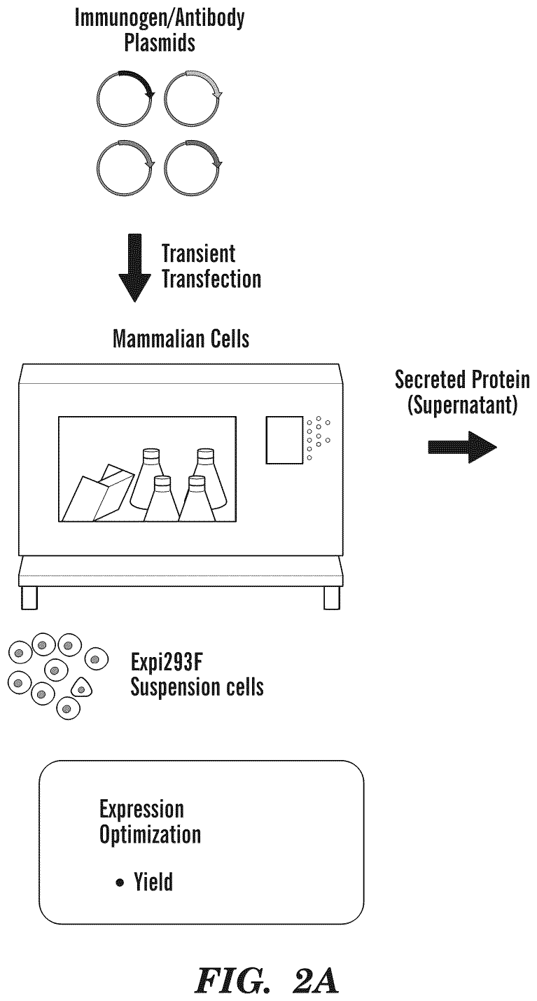

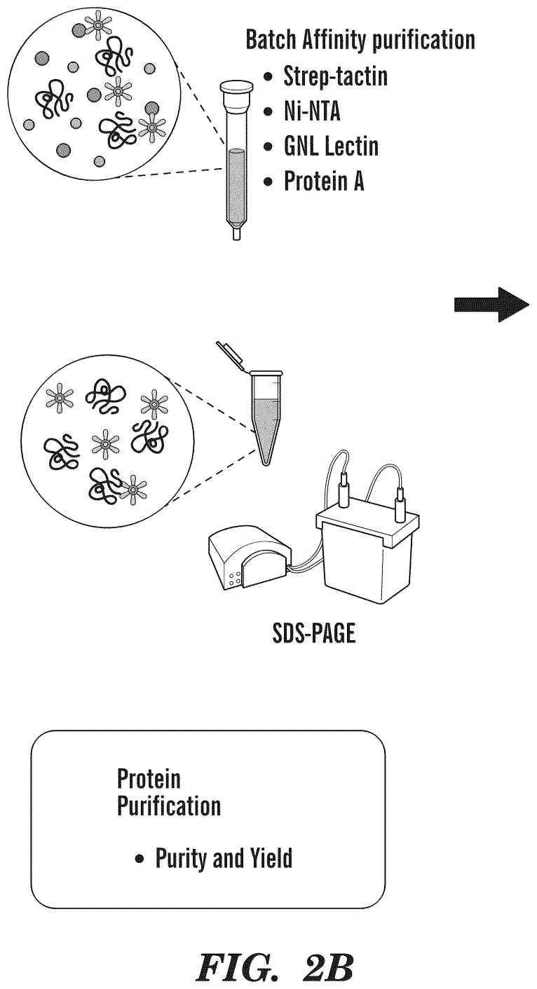

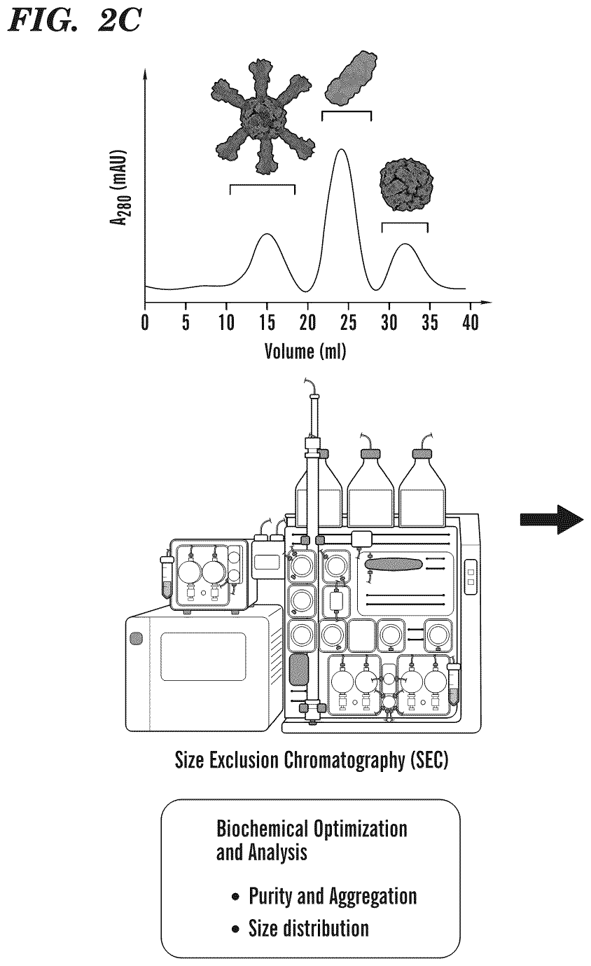

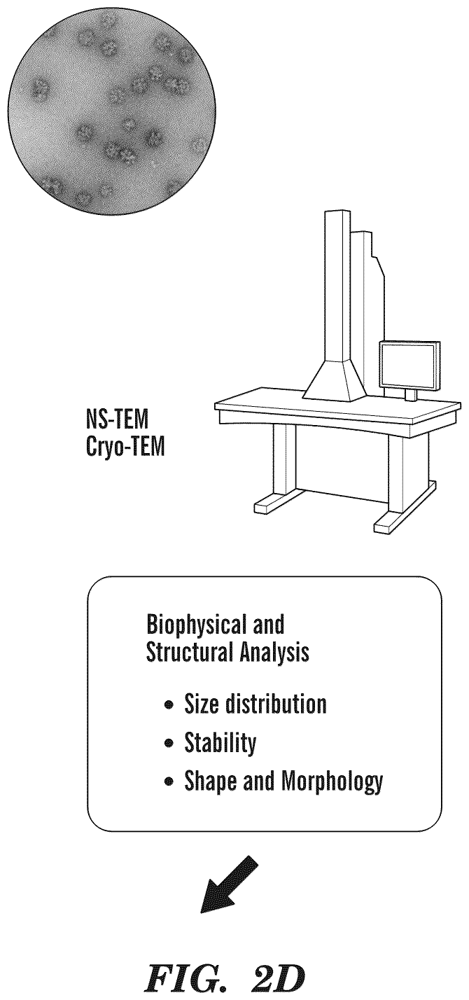

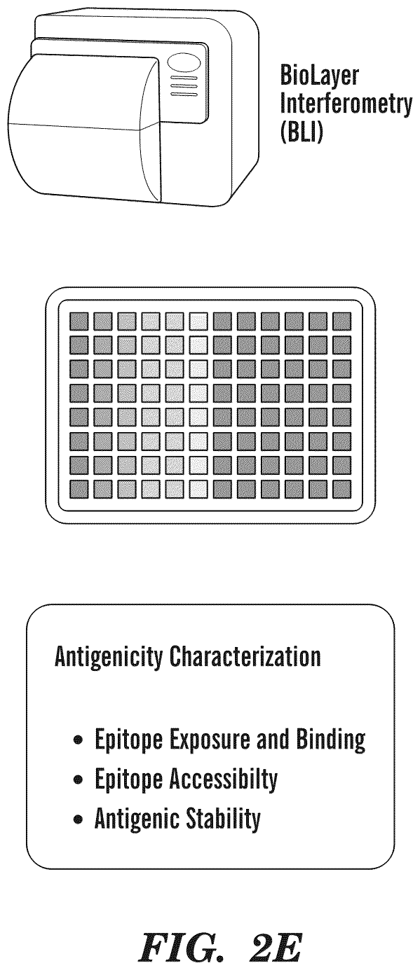

It is hypothesized herein that nanoparticles displaying these non-RBD MERS-CoV antigens will stimulate broader immunity to more conserved epitopes across β-CoVs compared to the spike trimer domain antigens alone. It is also hypothesized that combining specific nanoparticles and selected linkers will alter or shape the antigenicity of the engineered spike domains displayed on the nanoparticles. To explore these hypotheses and characterize the antigenicity of these nanoparticle vaccines, an integrated workflow was designed and harnessed (FIG. 2). Briefly, designed nanoparticle immunogens and antibody plasmids were transfected into mammalian cells. Next, respective batch affinity purifications were performed to isolate the immunogens and antibodies, confirming their presence in Sodium dodecyl sulfate-polyacrylamide gel electrophoresis (SDS-PAGE). The nanoparticle vaccines candidates were further purified using size exclusion chromatography (SEC) to optimize and analyze their purity and aggregation. Next, utilizing transmission electron microscopy (TEM), the biophysical and structural characteristics such as size, morphology, and aggregation of the nanoparticle were analyzed. Finally, using biolayer interferometry (BLI) and monoclonal antibodies (Table 2), the antigenicity of the nanoparticle vaccine candidates was characterized by investigating antibody binding, epitope exposure, and accessibility.

TABLE 2
Antibodies used for antigenicity characterization.

| Epitope | Antibody | Reference |
|---|---|---|
| NTD | G2 and A2 | (Wang et al., 2015) |
| RBD | F11 and D12 | (Wang et al., 2015) |
| S2 Loop | G4 | (Pallesen et al., 2017) |
| S2 Stem Helical Stalk | IgG22 | (Hsieh et al., 2021) |
| | CC40.8 | (Zhou et al., 2022b) |
| S2 Proximity to HR2 | IgG72 | (Hsieh et al., 2021) |

Biolayer interferometry (BLI) is a powerful technique that directly measures biomolecular interactions without labelling any of the reagents (Noy-Porat et al., 2021). By utilizing BLI, binding affinity will be measured through association and dissociation rate constants of the precision nanoparticles to the various monoclonal antibodies. By comparing these values to the antigen's binding affinity, it can be determined if the nanoparticle display is advantageous in stably exposing conserved epitopes, and enhancing B-cell activation, stimulation, and possible cross-reactive responses. In accordance, nanoparticles displaying the conserved region of many CoV RBDs were shown to bind strongly to B-cell receptors, dividing and producing more antibodies and cross-reactive B-cells (Callaway, 2023; Cohen et al., 2021; Cohen et al., 2022a; Lee et al., 2023). In addition, BLI experiments will provide knowledge about which nanoparticle, linker, and antigen combination will yield the most cross-reactive antibody responses and/or broadest protection.

The current lack of vaccines for MERS-CoV leaves the population vulnerable to this virus and its evolution as well as the broader family of β-CoVs. SARS-CoV-2, in particular, poses a major threat as variants escape current highly specific vaccines, and the virus evolves to endemicity in humans while establishing reservoirs in other species. By assessing the antigenicity of various engineered precision nanoparticle vaccines, the inventors aim to evaluate which vaccine candidates will induce broad protection against β-CoVs. Additionally, the inventors aim to establish a fundamental understanding of precision nanoparticle vaccines, their antigenicity signature, as well as the immunological response they elicit, providing knowledge which potentially may be applied to other viral families.

### Materials and Methods

### Materials

The details of materials used in herein are provided in the key resources table.

### Immunogen Design, Expression, and Purification

Cell Culture and Maintenance. Suspension Expi293F cells were cultured and maintained in Expi Expression medium (Thermo Fisher Scientific) to a density of 3-5×10⁶ cells per mL in a 37° C. shaker (INFORS HT Multitron) supplied with 70% humidity, 8% CO₂, and rotating continuously at 120 rpm for small cultures and 90 rpm for large cultures. The cells were diluted and subcultured every 3-4 days to 2-3×10⁵ viable cells per mL in a fresh and prewarmed (37° C.) Expi Expression medium using sterile Polycarbonate Erlenmeyer Flasks (Corning).

Gene Synthesis and DNA Plasmid Construction. All nanoparticle constructs or genes used in this thesis were human codon-optimized, synthesized, and ligated into Ndel and EcoRI restriction sites of pcDNA 3.1(+) mammalian expression vector by GenScript (Piscataway, NJ, USA) for protein production in Expi293F™ cells. The MERS-CoV S-domain nanoparticle (Apoferrtin, pdb:1FHA and Lumazine Synthase,pdb:1HQK) constructs were obtained from the MERS-CoV EMC/2012 spike protein (GenBank accession number JX869059, residues 18-1294), to include the following domains: NTD subunit (residues 18-353), RBD subunit (residues 367-588), SS subunits (residues 762-1291 with stabilizing mutations (Hsieh et al., 2021) and S ectodomain ΔRBD (18-1291). An S2P construct (MERS Spike with two proline mutations) was used as a template to generate the set of nanoparticle designs. The RBD and NTD NP constructs were modified to incorporate a N-terminal Kozak sequence, an artificial signal peptide (N-terminal human CD5 leader sequence, MPMGSLQPLATLYLLGMLVASVLA (SEQ ID NO: 99)), an octa-histidine (HHHHHHHH (SEQ ID NO: 217)) tag and strep-tag II (WSHPQFEK (SEQ ID NO: 98)) for purification. A human rhinovirus 3C protease recognition site (GSRSLEVLFQGP (SEQ ID NO: 228)) was inserted to enable tag cleavage after purification. The MERS SS and S2PΔRBD NP constructs included their native N-terminal leader sequences and a c-terminal T4 foldon trimerization domain (GYIPEAPRDGQAYVRKDGEWVLLSTFL (SEQ ID NO: 97)) prior to the nanoparticle fusion using linkers (Flexible and Rigid/Helical). Previously designed plasmids obtained from Vaccine Research Center, VRC (details available in the resource table) were used to express spike antigens (MERS S2PΔRBD, MERS SS, MERS NTD, MERS RBD) and bare nanoparticles (Ferritin and Lumazine Synthase). The plasmids expressing heavy and light chains of the monoclonal antibodies (A2, G2, F11, D12, G4, IgG22, IgG72, CC40.8) were obtained from the VRC with sources detailed in the resource table.

DNA Plasmid Preparation. The recombinant plasmid DNA of all protein constructs as described above were transformed into E. coli Stbl3 or DH5a competent cells, cultured in Luria Broth (LB) medium and extracted on large-scale using either endo free Maxiprep or Megaprep kits (QIAGEN Plasmid Plus) according to standard protocol. Briefly, 100 mL of E. coli containing the constructs were pelleted by centrifugation at 6000 g at 4° C. for 15 min, and 8 mL of resuspension buffer was added to each clone. Next, 8 mL of lysis buffer was added with subsequent incubation. After vigorous mixing, 8 mL of neutralization buffer was added. Following this short incubation, the lysed cells were separated using a plunger and filter to obtain the cleared supernatant for simple bind-wash-elute step. The cleared supernatant was then combined with 5 mL of binding buffer, loaded into QIAGEN plasmid plus columns, connected to a vacuum manifold, and washed. The cleared supernatant and wash buffers were drawn, and the column membranes were dried, followed by elution of highly concentrated DNA by centrifugation at 10,000 g for 1 min.

Recombinant Immunogen Expression. Spike and nanoparticle immunogens were produced by transient transfection of respective plasmids using PEI-MAX (Polyscience) or Expifectamine293 transfection kit (Thermo Fisher Scientific). All immunogens were expressed in Expi293F cells according to protocol as described in Weidenbacher, Waltari et al., 2022. Briefly, Expi293F cells were grown in media containing 66% Freestyle/33% Expi media (Thermo Fisher Scientific) using Corning Polycarbonate Erlenmeyer Flasks shaked at 37° C. in 8% CO₂. The day prior to transfection, cells diluted to a density of 3×10⁶ cells per mL in fresh prewarmed media and grown overnight. On the day of transfection, cells were diluted to a density of 3-3.5×10⁶ cells per mL. For PEI-MAX transfection, cells were transfected with 1:3 (v/v) consisting of 1 mg plasmid: 1 mg/mL PEI-MAX in Opti-MEM, Reduced Serum Medium. Then, an enhancer (Hyclone SFM4HEK293 liquid medium) is added 16-18 h post-transfection. For Expifectamine transfections, transfection mixtures were made using kit components. For example, for a 450 mL-transfection, 1.3 mL Expifectamine 293 transfection reagent diluted in 24 mL Opti-MEM medium was combined and mixed with 450 μg of plasmid DNA diluted in 24 mL Opti-MEM medium filtered via 0.2 μm filter. After 20 min incubation, the DNA-transfection reagent cocktail was added to the cells and cultured. The cells were harvested after 4-5 days by centrifugation at 7500 g for 30 min, and the supernatant containing the secreted proteins was filtered using 0.45 μm pore-size vacuum-driven filter. The respective secreted proteins were purified accordingly as described below.

Purification of Tagged Proteins: Ni-NTA Affinity Chromatography. NTD Nanoparticles (LuS constructs) and RBD antigen constructs were purified using Ni-NTA agarose beads (Qiagen) through their octa-histidine tags (SEQ ID NO: 217). Prior to loading into the column, 250 mL of filtered supernatants containing secreted proteins, diluted with 250 mL of binding buffer (20 mM Tris, 250 mM NaCl, 20 mM Imidazole) were mixed with 10 mL of Ni-NTA Agarose beads (Qiagen) and kept on a shaker in 4° C. overnight to allow the protein to bind to the beads. After loading, flow through is collected and unbound proteins or impurities were washed with 5 column volumes (CVs) of PBS supplemented with 20 mM imidazole. The bound protein nanoparticles or antigens were eluted with 5 CVs of elution buffer (20 mM Tris, 250 mM NaCl, 250 mM Imidazole). The purified proteins were concentrated and buffer-exchanged in Tris-buffered saline/0.02% NaN₃, TBS (RBD) or TBS/0.02% NaN₃ supplemented with 5% glycerol/1 mM EDTA (to mitigate aggregation problems) using centrifugal concentrators with appropriate cut-off for further purification.

Purification of Tagged Proteins: Strep-tactin Affinity Chromatography. NTD Nanoparticles (Aferr constructs) and remaining spike antigens were purified through their strep-tag II or Twin Strep-tag resin StrepTactin Superflow high capacity 50% suspension according to the manufacturer's gravity flow protocol. Briefly, 10 mL of StrepTactin resin (50% suspension) was packed into a disposable column and equilibrated with 3 column volumes (CVs) of wash buffer (100 mM Tris/HCl, 150 mM NaCl, 1 mM EDTA). The filtered supernatant was loaded onto the column, and the flow through was collected. The column was then washed with 2 CVs of wash buffer to remove any unbound protein or impurities. The bound proteins were eluted with 5 CVs of elution buffer (100 mM Tris/HCl, 150 mM NaCl, 1 mM EDTA, 2.5 mM D-desthiobiotin). The purified proteins were concentrated and buffer-exchanged in Tris-buffered saline, TBS/0.02% NaN₃ using centrifugal concentrators with appropriate cut-off for further purification.

Purification of Untagged Proteins: GNA Lectin Affinity and Ion-Exchange Chromatography. The untagged nanoparticle constructs (SS and S2PΔRBD) were purified through Galanthus nivalis lectin (GNL) affinity chromatography. Briefly, cleared 500 mL supernatant was combined with 10 mL Galanthus nivalis lectin agarose beads and incubated overnight with shaking at 4° C. The protein-Agarose suspension was then loaded onto a disposable column and washed with 10 Column volumes (CVs) of PBS buffer to remove unbound proteins and impurities. Bound proteins were eluted with 1 M methyl-α-D mannopyranoside in PBS buffer (pH 7.4). Next, the eluates were diluted thrice with buffer (50 mM Tris-HCl, ˜50 mM NaCl, pH 7.0), loaded on an anion exchange column (HiPrep Q HP 16/10) and washed with buffer (50 mM Tris-HCl, ˜50 mM NaCl, pH 7.0) on an AKTA system (Cytiva). The bound nanoparticles were eluted with a linear NaCl gradient using elution buffer (50 mM Tris-HCl, 1M NaCl, pH 7.0). The eluted nanoparticle fractions were concentrated and buffer-exchanged in Tris-buffered saline, TBS/0.02% NaN₃, using centrifugal concentrators with appropriate cut-off for further purification.

Recombinant Antibodies Expression and Purification. Monoclonal antibodies were produced in Expi293F cells by transient co-transfection of plasmids encoding heavy chain (HC) and light chain (LC) in a 1:1 ratio using the Expifectamine293 transfection kit (ThermoFisher Scientific) in accordance with the manufacturer's protocol. After 5 days, the cell supernatants were harvested, filtered (with 0.2 μm filter) and run over Pierce Protein A agarose-packed disposable columns. The columns were extensively washed with PBS and antibodies were eluted with 0.1 M glycine pH 2.9 (elution buffer) into a 1/10^{th} volume of neutralization buffer (1 M Tris-HCl pH 8.0). The purified antibodies were buffer exchanged in PBS using Amicon Ultra-15 Centrifugal Filter Unit (30 KDa cutoff).

Size Exclusion Chromatography (SEC). The above affinity purified immunogens were further purified by size exclusion chromatography using Superose 6 Increase 10/300 GL (Cytiva) on an AKTA pure system (Cytiva). Briefly, concentrated protein samples at volumes ranging from 0.5-1 mL were injected using a 1-mL loop onto Superose 6 10/300 GL column pre-equilibrated with TBS buffer (50 mM Tris, 150 mM NaCl. 0.02% pH 7.5) at a flow rate of 0.5 mL/min. The chromatograms were obtained at 4° C. with a 0.5 mL/min flow rate using de-gassed TBS buffer as the mobile phase, while observing absorbance at 280 nm. The immunogens eluted in sharp and uniform peaks approximately 3-4 mL wide. After each run, 1.5 mL fractions flanking the peak extreme were collected for following biophysical characterization. The column was calibrated with the molecular weight calibration standards to enable the determination of the actual sizes of the immunogens.

### Protein Characterization and Analytical Assays

Determination of Protein Concentration. The protein concentrations were determined by absorbance measurements (A₂₈₀) with NanoDrop Lite Spectrophotometer (Thermofisher) using the molecular weight and extinction coefficient as calculated by the online ExPASy ProtParam™ tool (expasy.org/protparam).

Sodium Dodecyl Sulfate Polyacrylamide Gel Electrophoresis (SDS PAGE) Analysis. The purity and molecular weights under denaturing conditions of the immunogens were analyzed by SDS-PAGE and Coomassie blue-staining. Briefly, 40 μL of each immunogen was mixed with 10 μL of reducing Nu-PAGE lithium dodecyl sulfate (LDS) and heated at 95° C. for 10 mins in a LSE™ Digital Dry Bath Heater (Corning). 20 μL of the immunogen-dye mixtures and 7 μL of PageRuler Plus Prestained Protein Ladder were loaded into the wells of 4-12% Bis-Tris NuPAGE precast gel for SDS-PAGE separation. Gels were run in MOPS buffer (GenScript) for 1 h at 130 V until samples ran to the bottom of the gel. After the run, the gels were stained for 10 mins, de-stained in deionized water overnight and imaged by UV light Gel Doc imager.

Octet biolayer Interferometry (BLI) Binding/Antigenicity Assays. The antigenicity, epitope accessibility, and binding kinetics of immunogens were studied and measured using a label-free bio-layer interferometry (BLI) detection system (Octet RH96 System, Sartorius-ForteBio) following manufacturer's protocol. All assays were performed in a kinetic buffer (1×HBS-EP+ Buffer, cytiva) at 30° C. in a solid black 384-well microplate (Greiner Bio-one) with agitation set at 1,000 rpm. Prior to the assays, immunogens at 100 nM (unless stated) were serially diluted at 2-fold dilution series of 4-6 in the kinetics buffer. All reagents including diluted immunogens, buffer, and antibodies/analytes (G2, A2, IgG22, IgG72, CC40.8, G4) were applied to the plates at 100 μL per well. All biosensors were hydrated in kinetics buffer for 10 mins prior to use. Assays were performed on the Octet RH96 system briefly as follows: hydrated Protein A biosensors (Sartorius/FortéBio) were dipped into monoclonal antibodies (IgG) diluted to 20 μg/mL in kinetics buffer in the loading step for 180 s, followed by 60 s baseline step in kinetics buffer to wash off unbound antibodies. After that, the IgG-loaded biosensor tips were dipped into the immunogens for 180 s in an association step, and subsequently dipped back into kinetics buffer for additional 300 s in a dissociation step. To serve as negative controls, biosensors were prepared under various conditions: either they were loaded with MERS RBD antibodies (F11, D12) and dipped into immunogens, or they were loaded with other antibodies (G2, A2, IgG22, IgG72, CC40.8) and dipped into kinetics buffer. MERS RBD was used to validate the functionalities of the F11 or D12 antibodies. Biosensors were regenerated with 0.2 M glycine pH 2.7, washed with kinetic buffer after each kinetics assay and reused for maximum of 10 runs according to manufacturer's instructions. Octet data were acquired in the Octet BLI Discovery (version 13) acquisition software and data obtained were processed as described below.

BLI Data Processing. Data analysis and curve fitting were carried out using Octet analysis Studio Software (Sartorius, Version 13.0). First, the raw data was processed by subtracting the reference well with the negative control from each sample well by average. Y-axis was aligned to the average of the baseline step. The inter-step correction was aligned to the dissociation step. Data was filtered with Savitzky-Golay filtering to remove high-frequency noise. Kinetic analysis for association and dissociation steps was performed and fitted with a binding model ratio of 1:1 for calculation of kinetic parameters (K_{D}, Kₐ, K_{d}). The mean K_{D} values were validated by averaging all binding curves that matched the theoretical fit with a Full R² values>0.95 and Chi² values<3.

### Structural Characterization

Negative-stained Transmission Electron microscopy (NS-TEM). Purified bare nanoparticles and nanoparticle immunogens were diluted to 70 μg/mL in Tris-buffered saline (TBS) for TEM analysis as described. Briefly, 4 μL of protein samples were applied to freshly glow-discharged carbon-coated 400-mesh copper grids (Electron Microscopy Sciences, EMS) and incubated for 1 min. The grids were blotted, submerged in 20 μL of distilled water, then excess water was blotted with Whatman no. 1 filter paper. Next, the grids were stained by dipping into 20 μL of 0.75% (w/v) uranyl formate stain, immediately blotted to remove excess, then stained again for 30 s. After that, the grids were allowed to air-dry for 1 min prior to storage or imaging. The grids were screened, and micrographs were recorded using CM10 Transmission electron microscope (Phillips CM10) equipped with a tungsten filament and Gatan UltraScan 894 (2k×2k) CCD camera operating at 100 kV. Grids were imaged at 52,000× magnification. All image processing for the 2D averaged images was done using cryoSPARC V 4.1 following cryosparc tutorial guide (guide.cryosparc.com/processing-data/tutorials-and-case-studies/negative-stain-data).

Cryo-electron Microscopy (Cryo-EM), Processing, and 3D Reconstruction. Purified MERS SS LuS nanoparticles at 1-3 mg/mL were applied to freshly glow-discharged copper grid (1.2 μm/1.3 μm, 400 Mesh Copper, Quantifoil). The grids were blotted for 4 s at 4° C. and 95% humidity, plunged frozen using a Vitrobot Mark IV (ThermoScientific), rapidly cryocooled in liquid ethane and stored in liquid nitrogen until screening or imaging. The grids were imaged at 1.1 Å per pixel using Talos Arctica cryo-electron microscope (Thermo Fisher Scientific) operated at 200 kV at Harvard Cryo-center for Structural Biology. Micrographs (defocus of −0.6, −2 μm) were recorded on post-GIF Gatan K3 camera using SerialEM version 4.1. A total dose of (50.12 e/Å²) and 49 frames were obtained (1.02 e/A2 per frame) with an exposure time of 4.89 s. Collected data set of 5000 stacked movies was processed using single-particle Cryo-EM in cryoSPARC v4.1 package as follows: all movies were imported into cryoSPARC v 4.1 (Punjani et al., 2017) and motion corrected using patch motion correction module and contrast transfer function (CTF) was evaluated in patch CTF estimation. Next, the particles (˜800) were manually picked from the 150 micrographs for 2D classification. Five 2D classes were obtained and used as a template to automatically pick a total of 12,100 particles. After another iterative 2D classification, 6500 particles were selected for Ab-initio 3D reconstruction (using C1 symmetry) and homogenous refinement, enforced with or without octahedral symmetry. Both symmetries yield the same cryo-EM map with well-resolved LuS core and stable MERS SS protruding from the core. The cryo-EM map was visualized and docked with LuS atomic model (pdb: 1HQK) using UCSF chimeraX.

Quantification and statistical analysis. All experiments (protein expression, affinity purifications, SEC, BLI, NS-TEM, Cryo-TEM) were performed numerous times and repeated in independent biological replicates. The replication attempts were all successful. The statistical analysis was performed using GraphPad Prism 9.0 software package (GraphPad Software). All BLI data analyzed using the Octet studio data analysis software (Sartorius-ForteBio, version 13) were exported and visualized using GraphPad Prism version 9.2. The sensograms were plotted using GraphPad Prism version 9 software.

Image analyses. The electron micrographs analysis and addition of scale bars was performed using Fiji (Image J) software package.

TABLE 3
Key Resources Table.

| | | |
|---|---|---|
| REAGENTS OR RESOURCES | SOURCE | IDENTIFIER |

Antibodies

| | | |
|---|---|---|
| G2 | (Wang et al., 2015) | N/A |
| G4 | (Wang et al., 2015) | N/A |
| D12 | (Wang et al., 2015) | N/A |
| F11 | (Wang et al., 2015) | N/A |
| IgG22 | (Hsieh et al., 2021) | N/A |
| IgG72 | (Hsieh et al., 2021) | N/A |
| CC40.8 | (Zhou et al., 2022a) | N/A |

Experimental model: cell line

| | | |
|---|---|---|
| Expi293F Suspension Cells | ThermoFisher Scientific | Cat#A14527 |

Bacterial and Virus strains

| | | |
|---|---|---|
| High Efficiency DH5α competent cells | New England Biolab (NEB) | Cat# C2987H |
| Top10 competent cells | ThermoFisher Scientific | Cat# C404010 |

Chemicals, Peptides, and Recombinant Proteins

| | | |
|---|---|---|
| 40 kDa linear polyethyleneimine | Polysciences | Cat# 24765-1 |
| ExpiFectamine 293 transfection reagent | ThermoFisher Scientific | Cat# A14525 |
| Imidazole | ThermoFisher Scientific | Cat# AC122020050 |
| FreeStyle 293 Expression Medium | GIBCO | Cat# 12338002 |
| Expi293 Expression Medium | GIBCO | Cat# A1435101 |
| OPTI-MEM, Reduced Serum Medium | ThermoFisher: Scientific | Cat# 11058021 |
| PBS | ThermoFisher: Scientific | Cat# 10010023 |
| Glycerol | ThermoFisher Scientific | Cat# BP229-1 |
| Methyl-α -D mannopyranoside | Sigma Aldrich | Cat# 617-04-9 |
| Tris | Sigma-Aldrich | Cat# GE17-1321-01 |
| QIAGEN Plasmid Maxi Kit | QIAGEN | Cat# 12162 |
| 4-12% Bis-Tris NuPAGE precast gel | ThermoFisher Scientific | Cat# NP0326BOX |
| PageRuler Plus Prestained Protein | ThermoFisher Scientific | Cat# 26619 |
| Ladder, 10 to 250 kDa | | |
| NuPAGE LDS Sample Buffer (4X) | ThermoFisher Scientific | Cat# NP0007 |
| Coomassie Brilliant Blue R-250 | Biorad | Cat# 1610436 |
| Staining Solution | | |
| Sodium azide, 99+% | ThermoFisher Scientific | Cat# 447810250 |
| Ampicillin sodium salt | Sigma-Aldrich | Cat# A9518 |
| Kanamycin Sulfate | Sigma-Aldrich | Cat# K1876 |
| Sodium Chloride | Sigma-Aldrich | Cat# S9888-1KG |
| Hyclone SFM4HEK293 | Cytiva | Cat# SH30521.02 |
| 10× Buffer E: Strep-Tactin Elution | IBA Lifesciences | Cat# 2-1000 |
| buffer with D-desthiobiotin | | |
| 10× Buffer R; Strep-Tactin | IBA Lifesciences | Cat# 2-1002 |
| regeneration buffer with HABA | | |
| 10× Buffer W: Strep-Tactin Wash | IBA Lifesciences | Cat# 2-1003 |
| buffer | | |
| Uranyl formate | SPI Chem | Cat# 02545-AA |
| NuPAGE ™ MOPS SDS Running | ThermoFisher Scientific | Cat # NP0001 |
| Buffer (20X) | | |
| Gel Filtration Calibration Kits (LMW, | Cytiva | Cat# 28403841; |
| HMW) | | Cat# 28403842 |
| HBS-EP + Buffer 10× | Cytiva | Cat # BR100669 |

Recombinant DNA

| | | |
|---|---|---|
| All Genscript Nanoparticle Constructs - | This thesis | N/A |
| pCDNA 3.1(+) | | |
| pαH-MERS S-2P ΔRBD F3CH2S | NIH-VRC | JSM 823 |
| pαH MERS SS_mut5_DS His (SS.V2) | NIH-VRC | JSM 744 |
| pαH MERS SS_DS ΔG4_loop (SS.V3) | NIH-VRC | N/A |
| pαH MERS NTD-3CH2S | NIH-VRC | JSM 881 |
| pαH MERS RBD-3C-HIS | NIH-VRC | N/A |
| Empty Ferritin NP | NIH-VRC | VRC 3419 |
| Empty LuS NP | NIH-VRC | VRC 4068 |

Software and Algorithms

| | | |
|---|---|---|
| GraphPad Prism (Version 9.2.0) | GraphPad | graphpad.com |
| Octet Data Analysis software (V11.1) | Sartorius (FortéBio) | sartorius.com |
| SerialEM | (Mastronarde, 2005) | bio3d.colorado.edu/SerialEM |
| CryoSPARC version 4 | (Punjani et al., 2017) | v4.1.2 |
| UCSF Chimera X Version 1.5 | (Goddard et al., 2018) | rbvi.ucsf.edu/chimerax/ |
| ImageJ | NIH | imagej.nih.gov/ij/ |
| Pep fold 3 | (Lamiable et al., 2016) | bioserv.rpbs.univ-paris- |
| | | diderot.fr/services/PEP- |
| | | FOLD3/ |

Other

| | | |
|---|---|---|
| Strep-Tactin Superflow resin | IBA Lifesciences | Cat# 2-1206-010 |
| Pierce Protein A agarose | ThermoFisher Scientific | Cat# 20334 |
| Ni NTA resin | QIAGEN | Cat# 88221 |
| Superose 6 Increase 10/300 GL | Cytiva | Cat# 29091596 |
| HiPrep Q HP 16/10 | Cytiva | Cat# 29018182 |
| HiTrap Q HP, 5 × 5 ml | Cytiva | Cat# 17115401 |
| Galanthus Nivalis Lectin (GNL), | Vector Labs | Cat# AL-1243-5 |
| Agarose bound | | |
| Carbon Film 200 Mesh, Copper | Electron Microscopy | Cat# CF200-CU |
| | Sciences | |
| Quantifoil −1.2 μm/1.3 μm, 400 Mesh | Electron Microscopy | Cat# 658-400-CU-100 |
| Copper | Sciences | |
| Filter paper | Cytiva | Cat# 1004047 |
| BD Luer-Lok 1-mL Syringe | BD | Cat# BD309628 |
| Anti-Human IgG Fc Capture (AHC) | Sartorius (FortéBio) | Cat# 18-5063 |
| Biosensors | | |
| Octet Anti-Penta-HIS (HIS1K) | Sartorius (FortéBio) | Cat# 18-5120 |
| Biosensors | | |
| Octet ProA Biosensors | Sartorius (FortéBio) | Cat# 18-5010 |
| Pall Acrodisc Sterile Syringe Filters | PALL Laboratory | Cat# 4614 |
| Empty Disposable Gravity Flow | Malvergent Bioscience | Cat# 11-0260-025 |
| Columns, Fritted, 30 mL, with 20 μm | | |
| frits | | |
| Silicone Tubing | Biorad | Cat# 7318212 |
| Amicon Ultra-15 Centrifugal Filter | Millipore | Cat# UFC910008 |
| Unit (10-100 KDa cutoff) | | |
| Corning Polycarbonate Erlenmeyer | Corning | Cat# 431147 |
| Flasks | | |
| Greiner Bio-One 96-well black flat- | VWR | Cat# 82050-784 |
| bottom PP, 200 μL | | |
| Greiner Bio-One 384-well black flat- | VWR | Cat# 82051-318 |
| bottom PP, 80-120 μL | | |

### Results

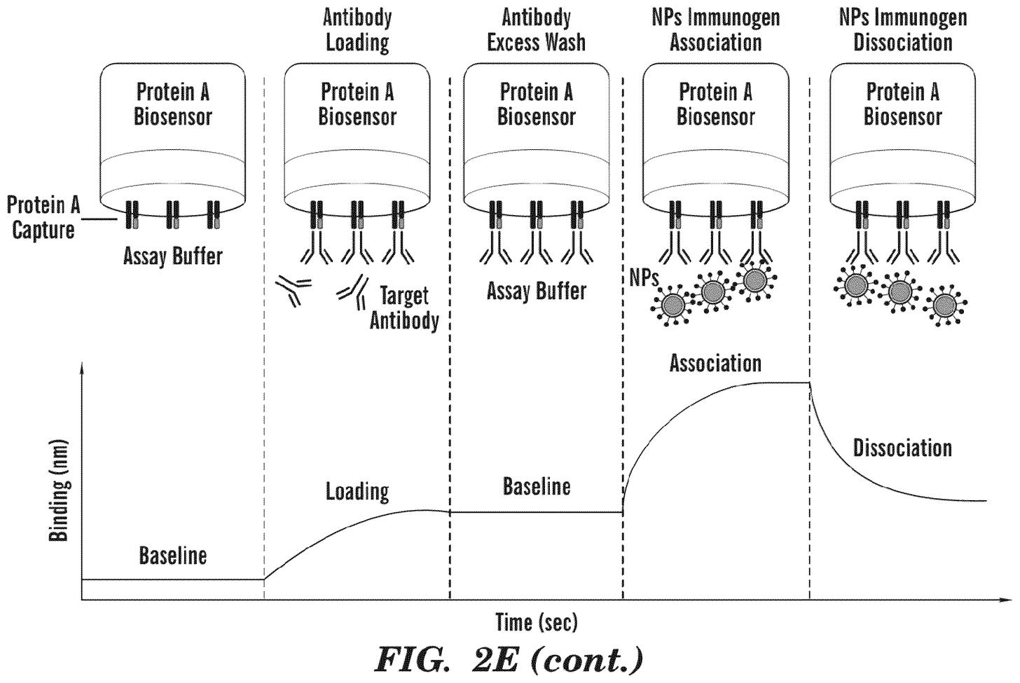

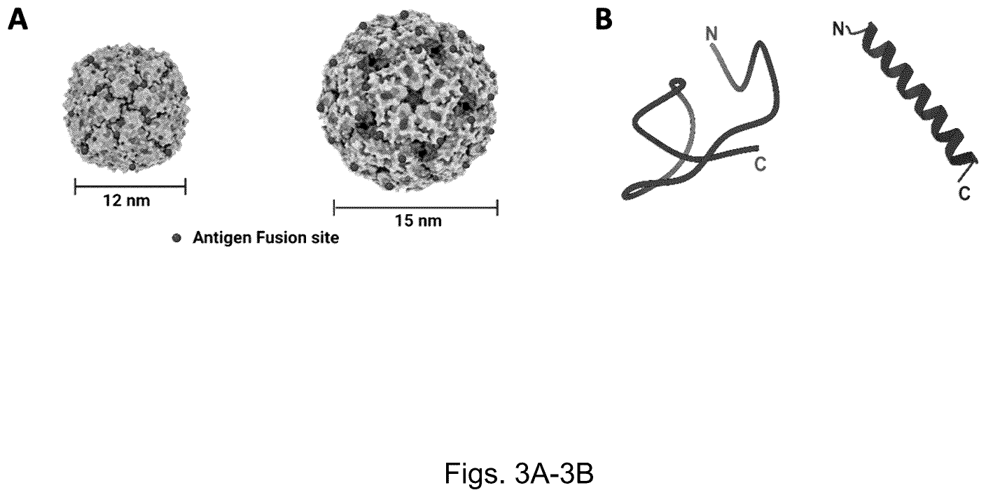

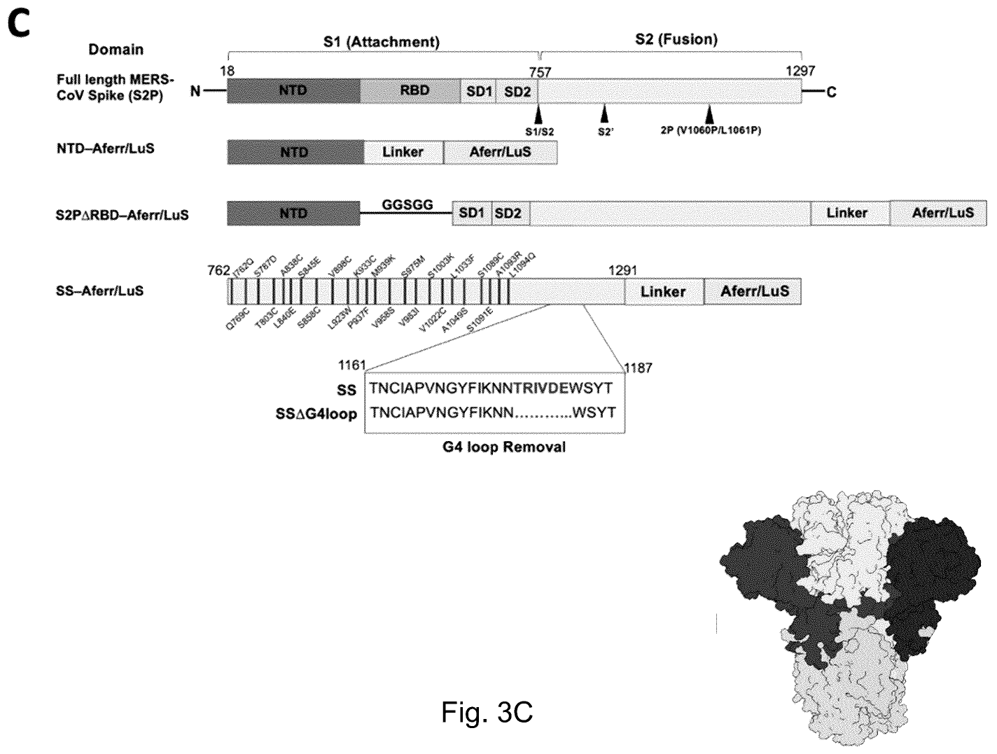

Design, Expression, and Characterization of non-RBD Nanoparticle Immunogens. Due to their relative conservation (Barnes et al., 2020; Hsieh et al., 2021; Pallesen et al., 2017; Wang et al., 2022; Xiong et al., 2022), it was hypothesized herein that non-RBD vaccine immunogens would confer broader protection against β-CoVs. Therefore, using the initial MERS-CoV EMC/2012 spike protein (GenBank accession number JX869059, residues 18-1294) containing two proline mutations (Pallesen et al., 2017), the inventors engineered four classes of antigens lacking the RBD domain, namely NTD (residues 18-353), S2P ectodomain ΔRBD (residues 18-1291), SS (residues 762-1291 with stabilizing mutations (Hsieh et al., 2021)), and SSAG4Loop (SS lacking the G4 antibody variable epitope (Pallesen et al., 2017)), and presented them using nanoparticles: Aferr and LuS (FIG. 3A). Aferr and LuS nanoparticles can display 20 and 60 monomers on their surface respectively. Therefore, these two nanoparticles could exhibit differential antigenic profiles.

The nanoparticles were genetically fused to the C-terminal region of the engineered antigens using two of our in-house designed linkers (rigid, L2 and flexible, L6) depicted in FIG. 3B. The molecular designs of the classes of representative spike-domain nanoparticle constructs as immunogens for expression are shown in FIG. 3C.

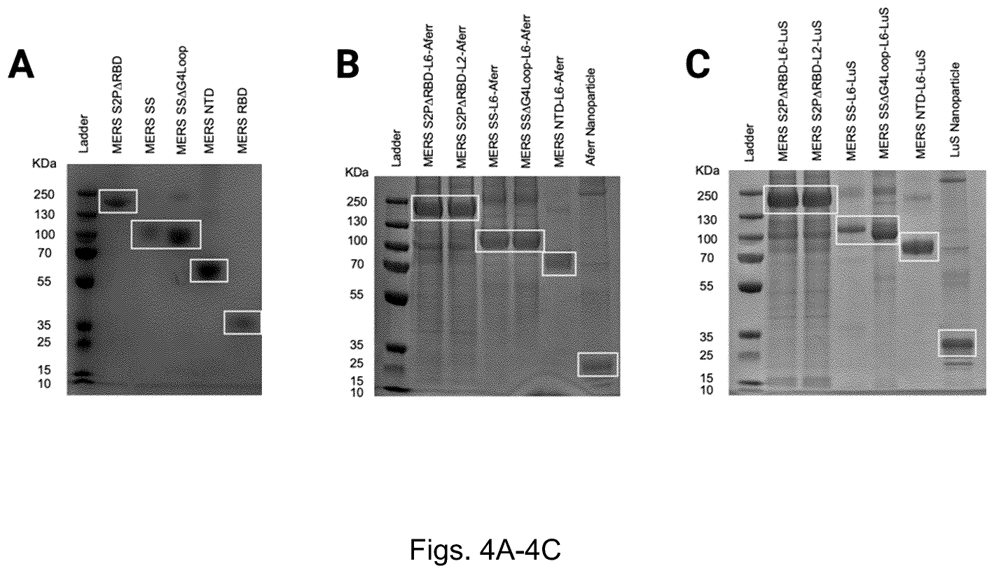

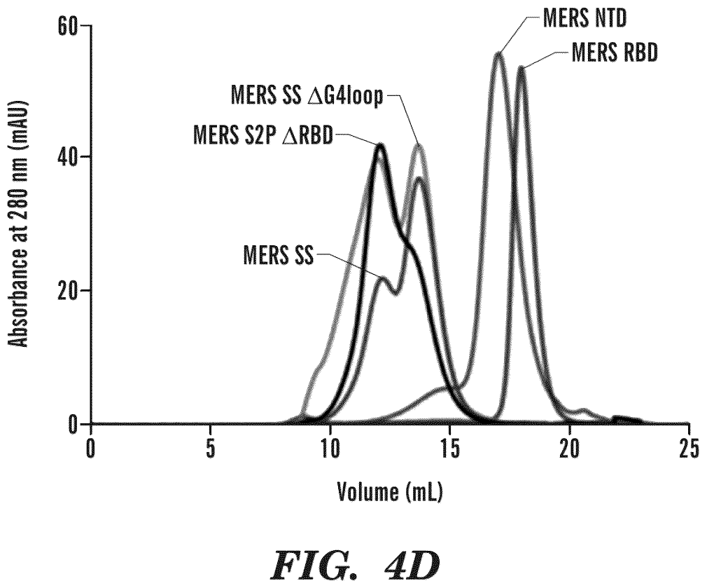

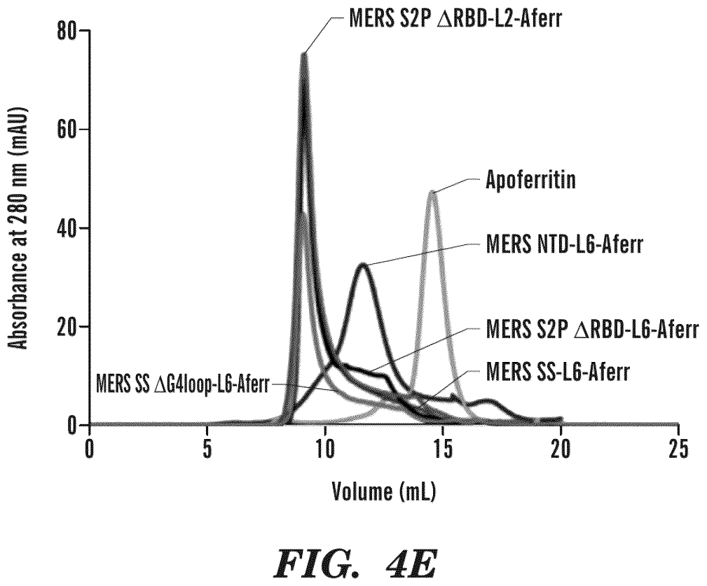

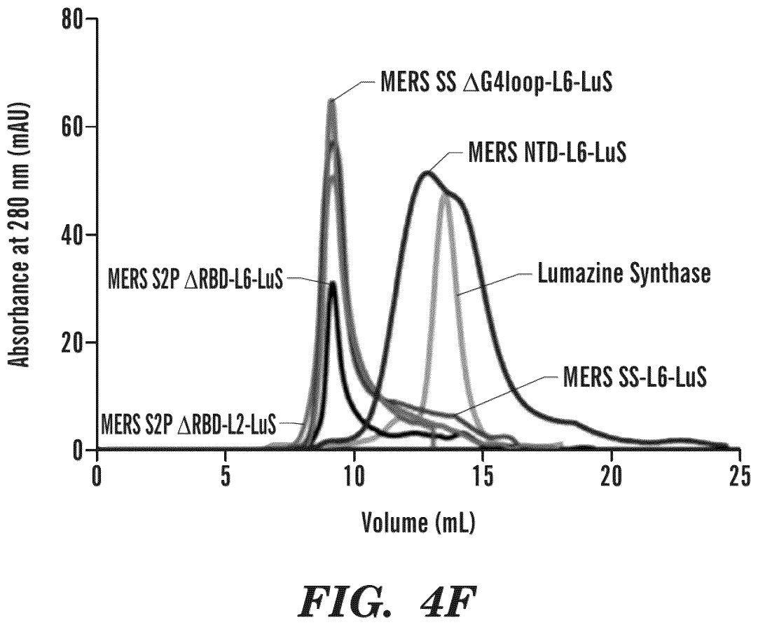

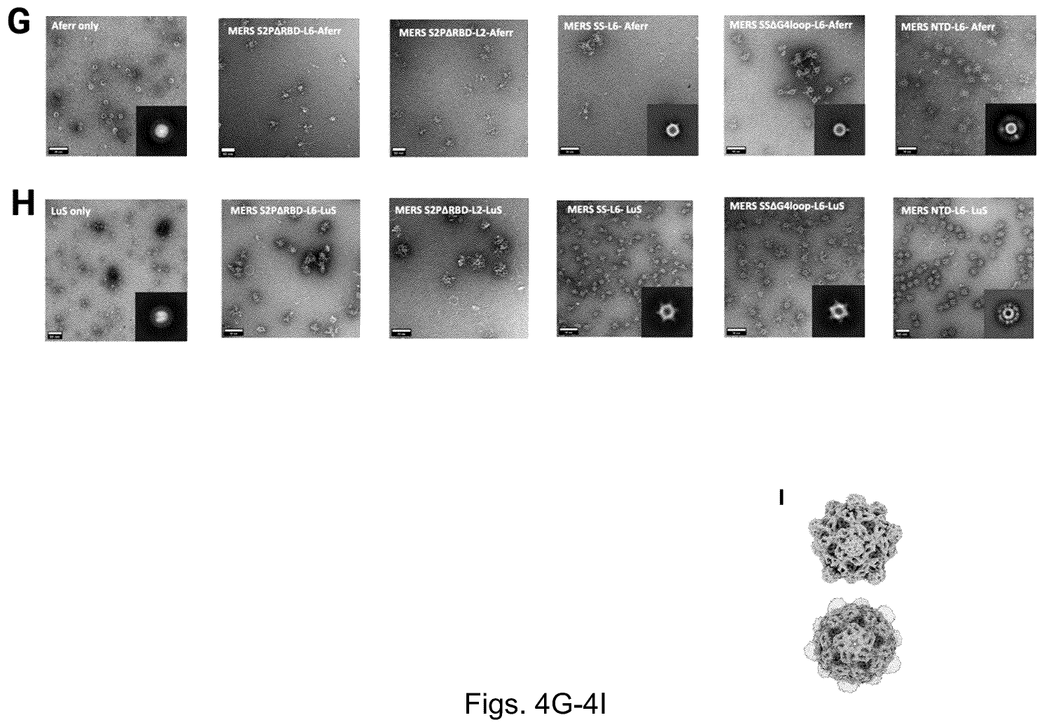

Following the designs, the spike vaccine immunogens were expressed in Expi293F cells for 4-6 days at 34° C. and 37° C. and purified with respective batch affinity chromatography as detailed in the methods. The constructs showed decent expression levels at high purity as assessed by SDS-PAGE (FIGS. 4A-4C). Each lane had a notable band corresponding to the predicted or expected molecular weight of the immunogen. The purified immunogens were further subjected to size exclusion chromatography (SEC). Each immunogen's chromatogram revealed distinct peaks that corresponded to the actual molecular weight or size of the immunogens using various proteins of known molecular weights as standards (FIGS. 4D-4F). Next, negative-stain electron microscopy (NS-TEM) was carried out to assess the nanoparticle formation and morphology of the nanoparticle immunogens. NS-TEM confirms the homogeneity and monodispersity of Aferr (FIG. 4G) and LuS (FIG. 4H) nanoparticles. Notably, the globular shape of protruding spike domain antigens displayed on the nanoparticle are visible in both raw micrographs and 2D class averages compared to bare Aferr or LuS nanoparticles alone. In further structural analysis of the MERS SS-L6-LuS nanoparticle immunogen, Cryo-EM confirmed that the nanoparticle structure consisted of a well resolved LuS nanoparticle core with SS antigens protruding from it (FIG. 4I, top), regardless of the symmetry enforced during processing. Upon docking LuS NPs atomic model (pdb:1HQK) into the 3D reconstructed cryo-EM map at a resolution of ˜4 Å (FIG. 4I, bottom), the MERS SS-L6-LuS showed the stabilized prefusion SS protruding from the LuS molecule, further reflecting the similarly ordered number of evenly distributed spike density on the virus surface. Together, these data establish that non-RBD spike antigens can be engineered, displayed on nanoparticles, and self-assembled into homogenous and monodisperse immunogens with high efficiency.

### Antigenic Characterization of the Nanoparticle Vaccine Candidates

Next, it was sought to assess the antigenicity of these immunogens using BLI via an experimental approach which roughly mimics B-cell receptor (BCR)-immunogen interaction that is essential for B-cell activation. The experimental process of BLI is described in FIG. 2E. Briefly, antibodies were loaded onto a protein A probe and subsequently dipped in wells containing immunogens in the association step, then transferred into wells containing kinetics buffer in the dissociation step. For each run, there was a negative control of an antibody that does not bind to the immunogen of interest. Antibodies used for antigenicity characterization are listed in Table 2. Antibody F11 targets the RBD epitope of the spike protein. Therefore, it was used as a negative control for all the engineered non-RBD immunogens. Antibodies, G2 and A2 target the spike NTD and were used to characterize NTD and S2PΔRBD-based immunogens. G4 targets a variable epitope in the relatively conserved S2 domain. IgG22 and CC40.8 target S2 stem helical stalk, and IgG72 targets S2 proximal to HR2. These antibodies were used to characterize the S2PΔRBD, SS, and SSΔG4Loop-based immunogens.

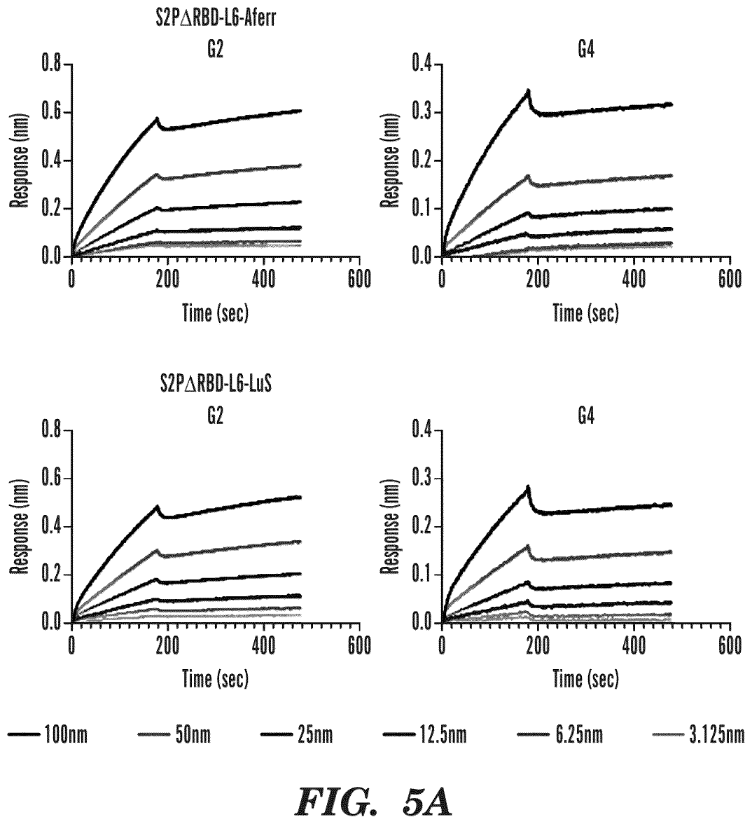

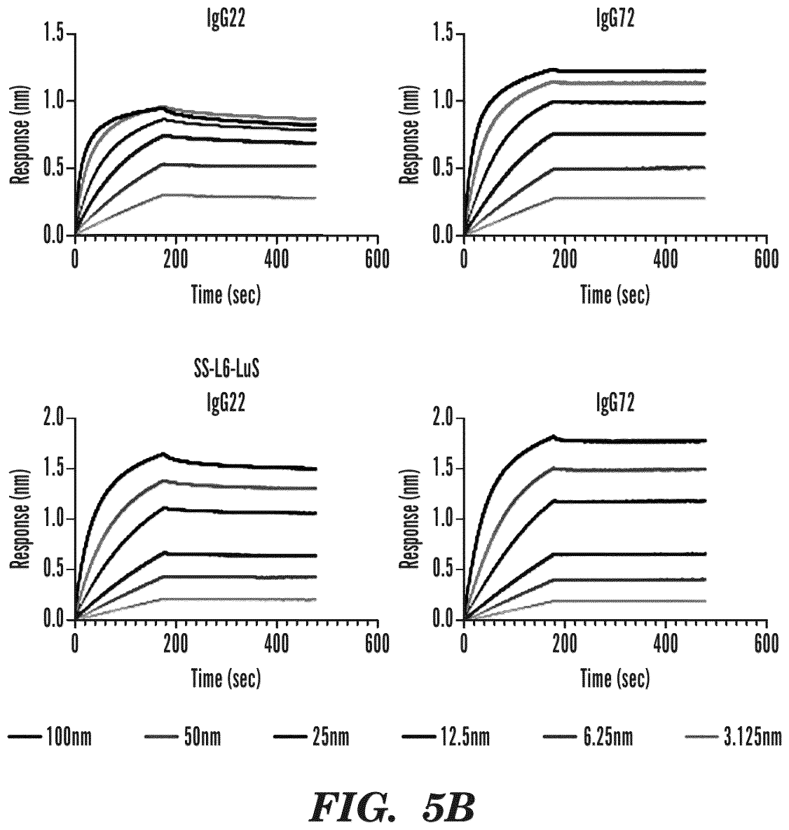

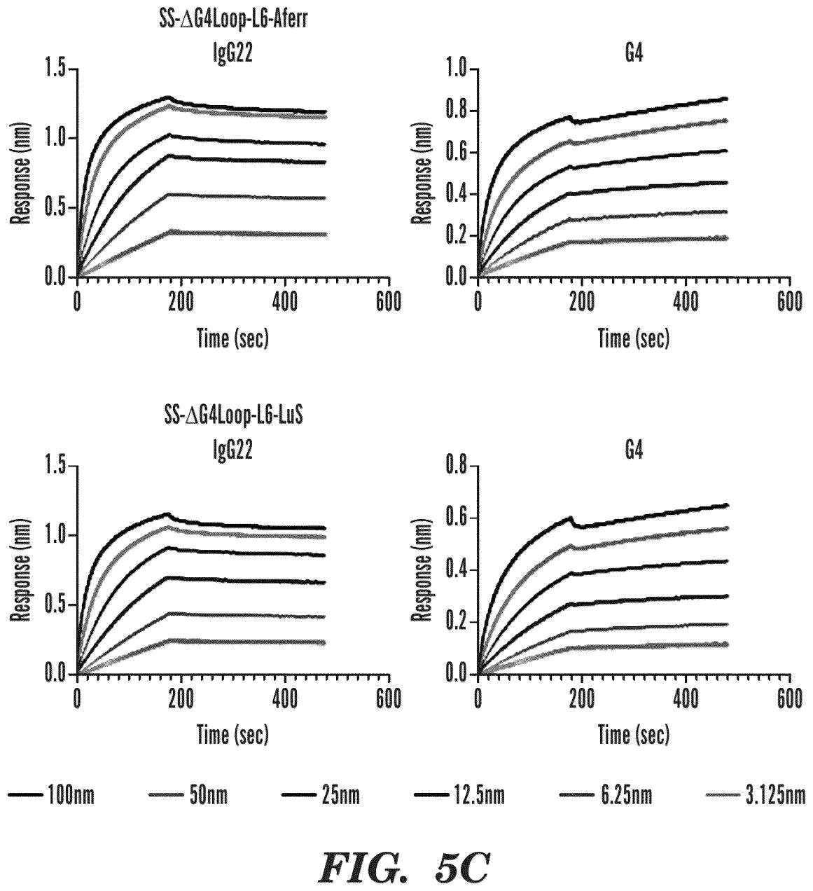

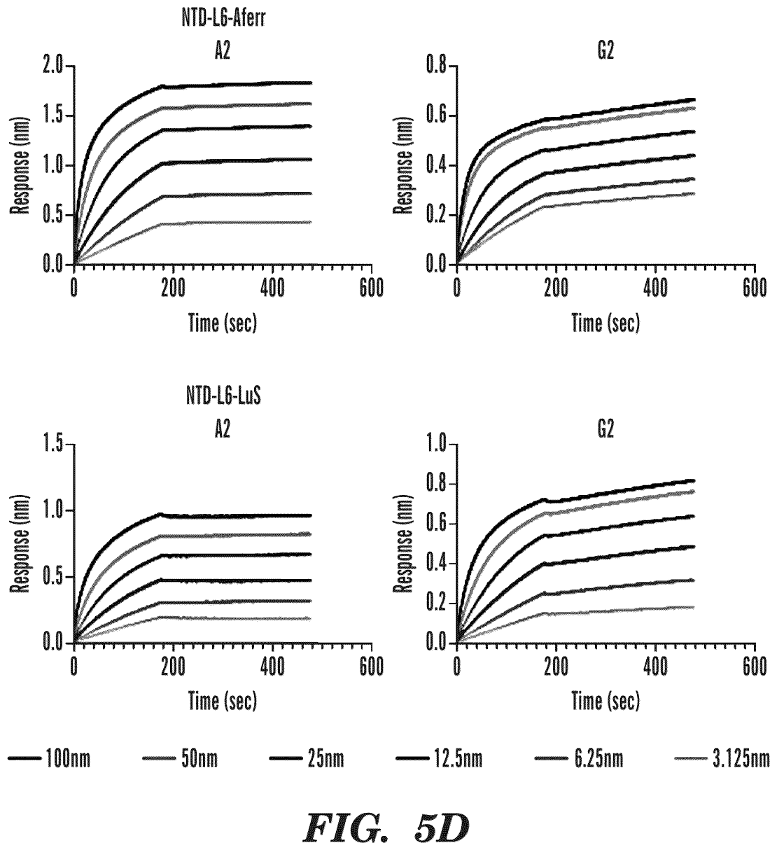

Binding curves or sensograms generated from BLI experiments are shown in FIGS. 5A-5D. These curves allow for qualitative comparisons of antibody binding across the different immunogens. Analysis of these curves yielded the association constant (Kₒₙ), dissociation constant (K_{off}), equilibrium dissociation constant (K_{D}) and R² value of each fitted sensogram (FIGS. 45A-45B). The K_{D} values were obtained by dividing K_{off} by Kₒₙ, therefore, the lower the K_{D}, the stronger the antibody binding. Interestingly, the different nanoparticle immunogens showed enhanced binding to the tested antibodies compared to the engineered antigens as evident by the K_{D}s (FIGS. 45A-45B), suggesting better B-cell activation and production of more B-cells and protective antibodies. For instance, the K_{D}s of G2 binding to NTD nanoparticle immunogens and NTD antigen were ˜1 pM and 42 nM respectively. Also, SS nanoparticle immunogens bound to IgG22 and CC40.8 with K_{D} values˜0.1-0.8 nM and ˜3-7 nM respectively, but the corresponding antigens had no binding. Surprisingly, the G4 antibody still bound to the SSAG4Loop nanoparticles (FIG. 5E) but not the antigen (FIGS. 45A-45B), despite the deleted variable epitope. Taken together, these data further confirm that nanoparticle display of the engineered spike antigens in their native conformation expose epitopes of potent broadly neutralizing antibodies, a feat unattainable in some engineered spike immunogens.

### Precision Nanoparticle Display and Epitope Accessibility

The multivalent nature of nanoparticle vaccines has been demonstrated to skew the specificity of vaccine-elicited neutralizing antibodies (Powell et al., 2022). Moreover, the magnitude and quality of humoral responses induced by the nanoparticle immunogens may be attenuated due to the inaccessibility of key epitopes because of their dense multivalent antigen array (Brouwer et al., 2019; Hutchinson et al., 2022). Nonetheless, immunogens that bind strongly to B-cells will divide, flourish, and produce more antibodies and B-cells (Callaway, 2023; Nguyen and Tolia, 2021).

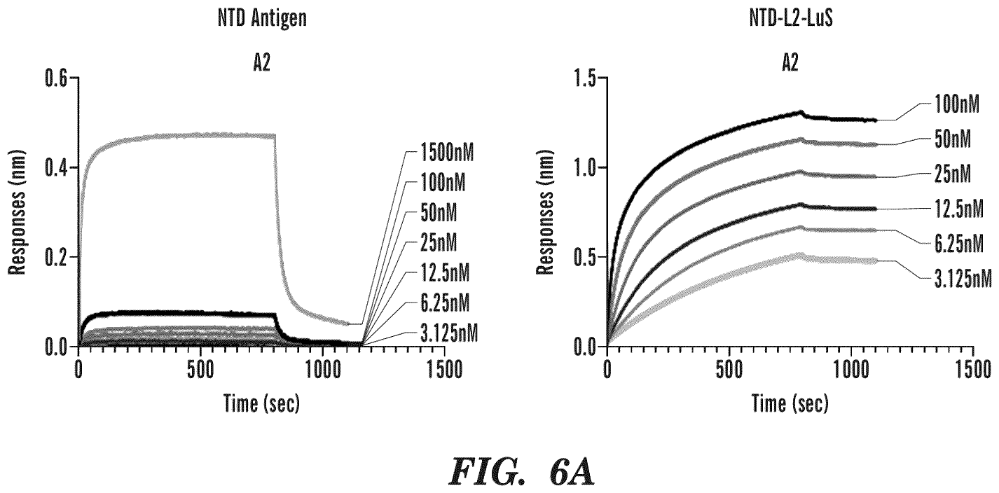

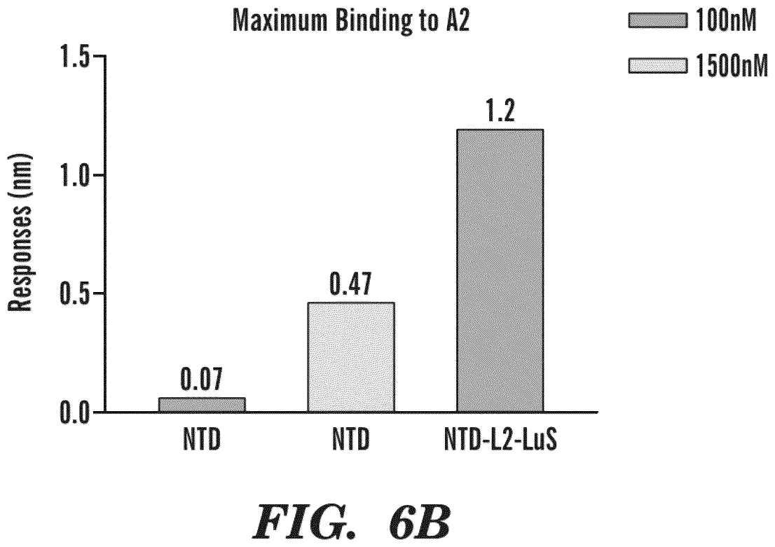

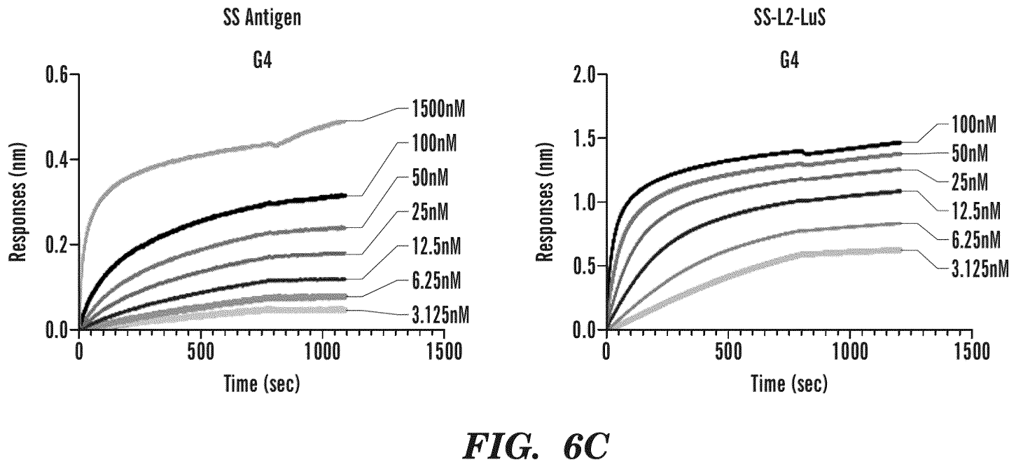

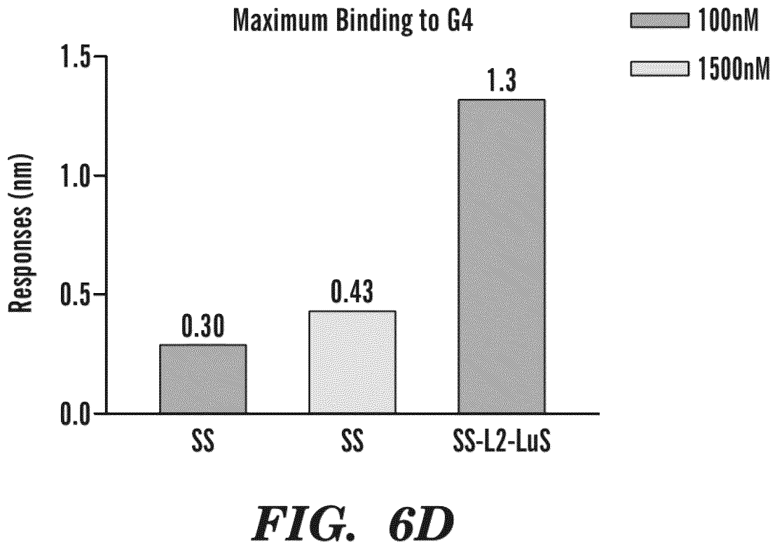

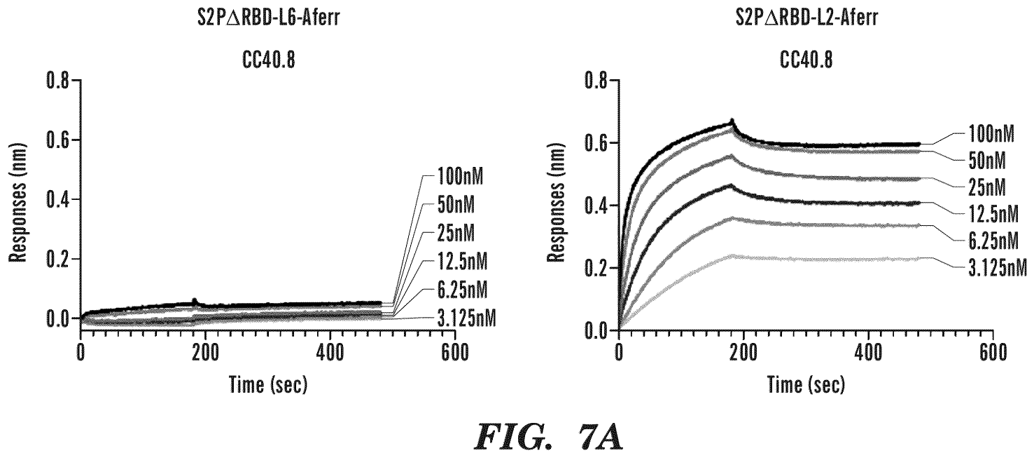

To investigate these effects, BLI experiments were conducted with a longer association time of 800 seconds (instead of the 180 seconds as used previously) to assess antibody binding to specific immunogens. This longer association time allows the immunogen and antibody to reach a maximum level of binding. A higher concentration of 1500 nM of the antigen was also assessed for binding, along with the typical 2-fold dilution series from 100 nM. This BLI experiment was done to compare A2 antibody binding to the NTD and NTD-L2-LuS (FIG. 6A), as well as G4 antibody binding to SS and SS-L2-LuS (FIG. 6C). The differences in the antibody binding response were analyzed and plotted (FIGS. 6B, 6D). In the former, 100 nM of the nanoparticle had a maximum binding of 1.2 nm, compared to 1500 nM of the antigen with maximum binding of 0.47 nm (FIG. 6B). In the latter, 100 nM of the nanoparticle had a maximum binding of 1.3 nm, compared to 1500 nM of the antigen alone with a maximum binding of 0.43 nm (FIG. 6D). In summary, nanoparticle immunogens bound to respective antibodies with higher binding signals by several order of magnitude (2-3-fold increase) compared to antigens at similar or higher (15×) concentration. This notable increase in binding response highlights the advantages of using precision nanoparticle platforms to expose key specific epitopes that may lead to stronger B-cell activation.

In addition, a key limitation of current vaccines is their inability to induce certain rare convergent S2-targeting cross-reactive antibodies (Dacon et al., 2022; Zhou et al., 2022b). A major reason is that these S2 stem bnAb sites on the native-like spikes found in the current vaccines are occluded in the context of different β-CoVs, leading to restricted access of the B-cell receptors to this conserved domain or epitopes. Therefore, immunogens that take advantage of germline targeting may favor the priming of these S2 stem bnAb B-cell responses. Notwithstanding, it might be challenging to access the S2 stem bnAb sites on the spike immunogens for effective engagement with the desired B-cell responses. Nevertheless, it was hypothesized by the inventors that rationally displaying spike antigens in a conformation that mimics the spike-decorated virion surface might accommodate such features and may favorably prime S2 stem bnAb B-cell precursors.

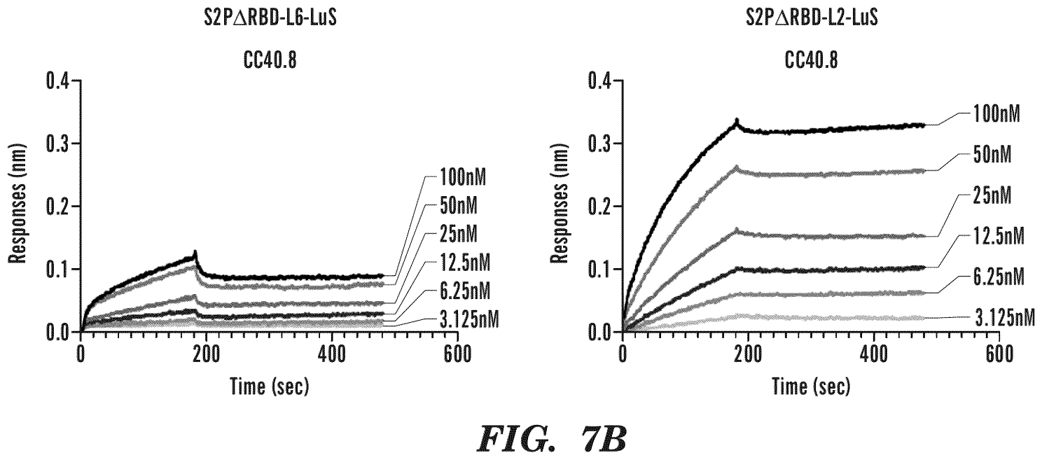

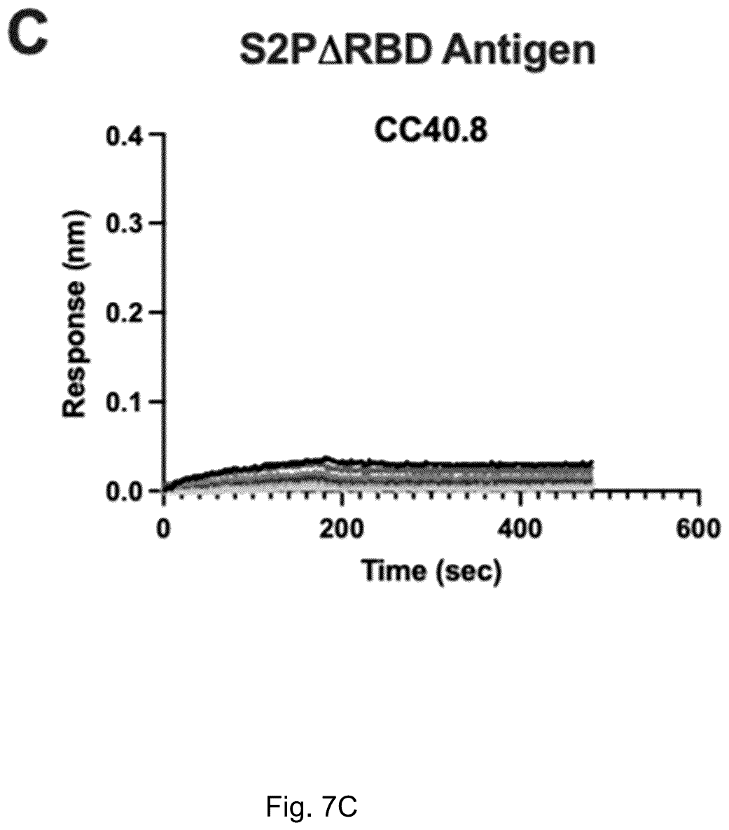

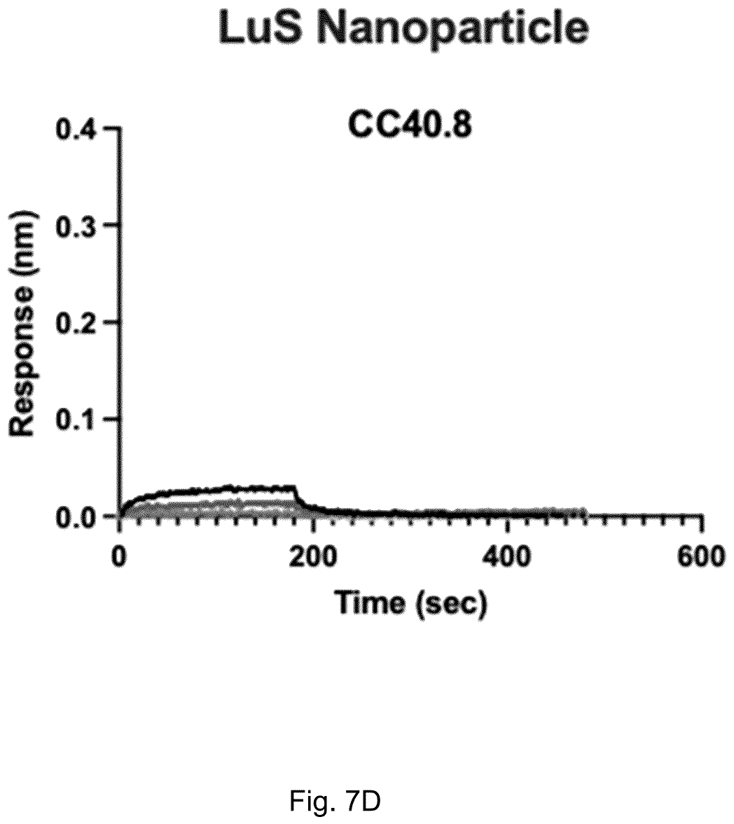

To investigate this possibility, the epitope accessibility or binding of the engineered spike S2PΔRBD-based nanoparticle immunogens containing linkers (L2 and L6) and the full spike antigen (S2PΔRBD) were compared to CC40.8. CC40.8 is a broadly neutralizing antibody that targets the conserved S2 stalk-helix peptide in the β-CoVs Spike protein (Zhou et al., 2022b). In the case of the MERS-CoV spike, this antibody can only bind to the short peptide, but not when the peptide epitope is available in the full spike protein. To discern the binding relationship between these engineered immunogens and CC40.8, BLI experiments were performed. S2PΔRBD-based nanoparticles with the flexible linker (L6) did not bind to CC40.8 (FIG. 7A, 7B). Also, the S2PΔRBD antigen had no detectable binding (FIG. 7C), similar to that reported for the MERS S2P spike using BLI (Zhou et al., 2022b). However, for S2PΔRBD displayed with the rigid/helical linker (L2), both nanoparticle immunogens had robust binding to CC40.8. The K_{D}s for S2PΔRBD-L2-Aferr and S2PΔRBD-L2-LuS were ˜4 pM and 3 pM, respectively (FIGS. 45A-45B). It is therefore possible that the rigid linker in S2PΔRBD-based nanoparticle immunogens permit better access to the S2 stem-helix peptide epitope targeted by CC40.8. This may have allowed a “mimicry” of spike placement or presentation on the virion surface, but needs to be structurally characterized, modelled, and confirmed using electron microscopy. To rule out non-specific binding, BLI was performed to assess the CC40.8 binding to the LuS nanoparticle only. It was confirmed that the CC40.8 did not bind to the LuS (FIG. 7D), supporting the notion that the rigid linker exposed this conserved epitope. Even though it has been shown that CC40.8 also does not bind to the MERS-CoV full spike (S2P) in ELISA assays (Zhou et al., 2022b), further ELISA experiments can be performed to validate the S2PΔRBD-based nanoparticle immunogen binding to antibody CC40.8.

Notwithstanding, it was also shown that CC40.8 binds to SS-based nanoparticle immunogens designed with rigid and flexible linkers but not to the SS alone (FIGS. 45A-45B). Therefore, nanoparticle presentation could also be a contributing factor to the exposure effect. Nonetheless, the rigid linker provides obvious benefits in the case of the full spike and should be used in future designs in order to expose certain conserved S2 stem-helix neutralizing epitopes that are inaccessible in the full spike protein.

To this end, the inventors contemplate that other multiple distinct epitopes targeted by neutralizing S2 (IgG22, IgG72, G4) and S1 (G2, A2) antibodies were exposed and accessible for binding in the context of the designed spike MERS S2PΔRBD nanoparticle immunogens.

### Model of Strong Antibody Binding, Epitope Exposure, and Possible B-Cell Activation by Nanoparticle Vaccine Candidates

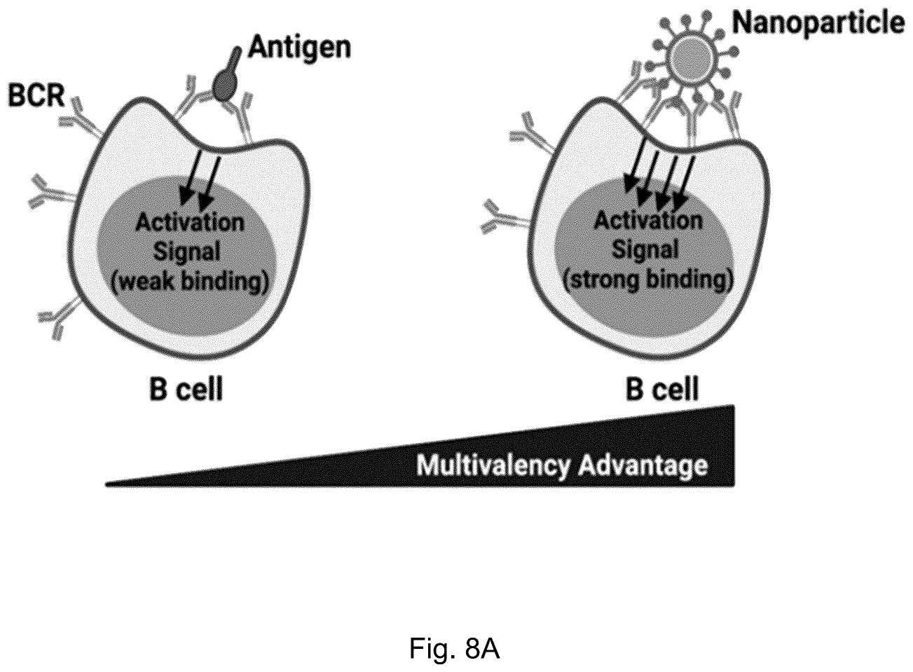

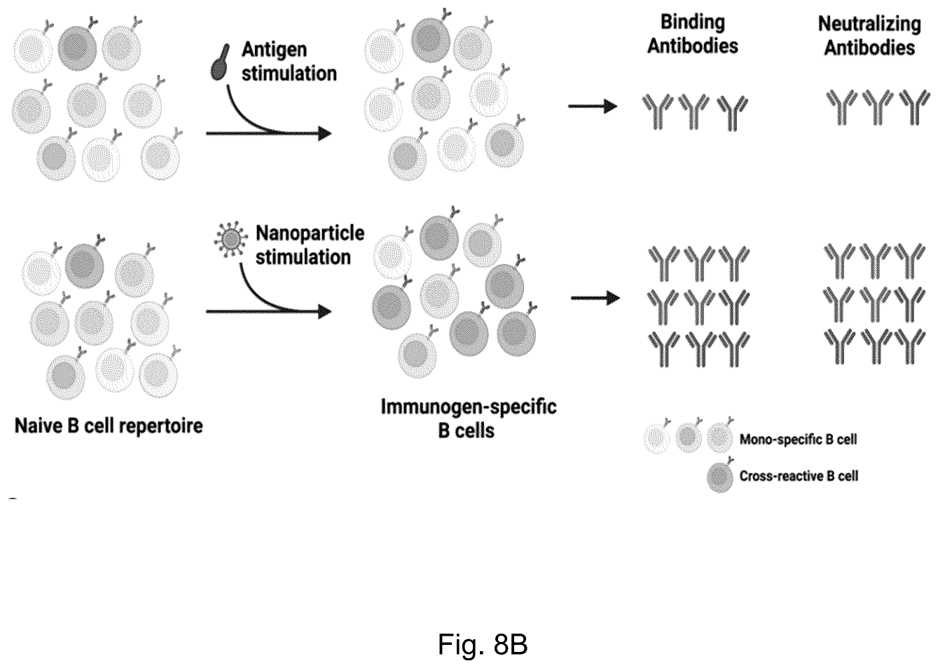

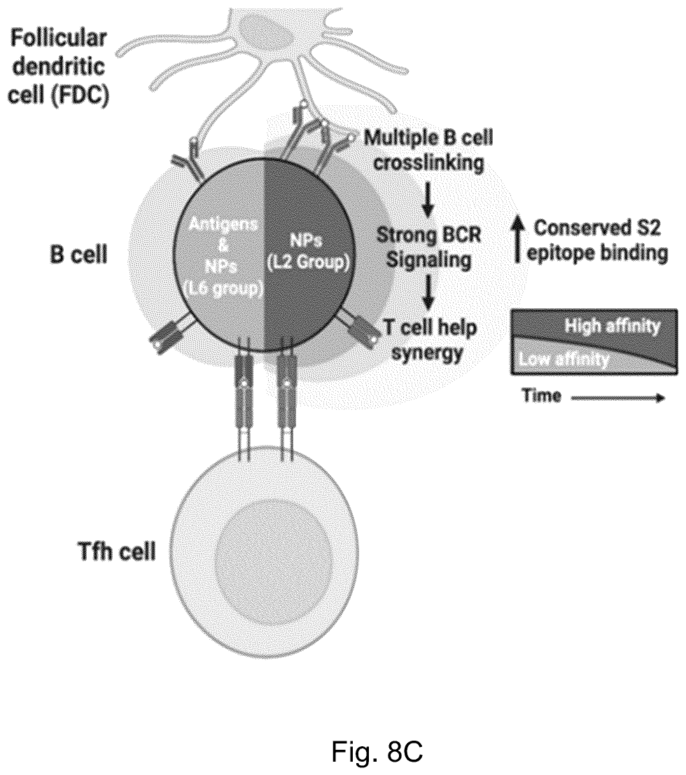

Based on the antigenic characterization findings, collective multiple epitope accessibility, and conserved S2 stem/stalk exposure, using FIGS. 6 and 7 as examples, it is contemplated herein that designed nanoparticle vaccine candidates (particularly the rigid linker group) at the same or lower dose could induce cross-reactive immune responses across multiple β-CoV strains compared to the engineered antigens (FIG. 8). In MERS NTD-A2 and MERS SS-G4 binding curves, the antibody-antigen interaction as evident by binding response (association) at different concentrations will likely result in weak B-cell activation and response, potentially failing to flourish or produce more antibodies and cross-reactive B-cells (FIG. 8A, left). In contrast, due to the multivalency/avidity advantage, the relatively stronger binding of antibody to the nanoparticle immunogen could facilitate strong B-cell activation via antigen crosslinking with multiple BCRs (FIG. 8A, right). Therefore, when the immune system encounters these nanoparticles, the outcome may be enormous (FIG. 8B). Compared to the antigen group, cross-reactive B-cells will be favored and expanded, leading to production of more potent binding and neutralizing antibodies. Lastly, MERS S2PΔRBD or SS based immunogens, nanoparticle groups, particularly those designed with rigid linker, would provide advantages to cross-reactive B-cells over strain-specific B-cells in a diverse manner during epitope binding, immunogen capture, and T-cell help as depicted in FIG. 8C. When presented on follicular dendritic cell (FDC) after the capture step, MERS SS or S2PΔRBD NPs (L2 group) can form multivalent interaction with cross-reactive B-cells but not with strain-specific B-cells, because a cross-reactive spike or S2 directed B-cell can engage a single immunogen with up to three or more BCRs (targeting NTD (G2 antibody) or S2 (IgG72 and exposed IgG22 and CC40.8 antibodies) epitopes). However, strain-specific B-cells can recognize a single immunogen (for S2PΔRBD/SS and L6 NPs group) with, at most, two BCRs (targeting NTD (A2, G2 antibodies) or S2 (IgG72 antibody) epitopes). Subsequently during selection for T cell help, cross-reactive B-cells that capture MERS SS or S2PΔRBD NP (L2 group) immunogens can present peptides from multiple potent cross-reactive antibody (G2, IgG72, IgG22, CC40.8) epitopes. In contrast, strain specific B-cells for S2PΔRBD/SS and L6 NPs groups can only present peptides/epitopes for the few monospecific antibodies (A2, G2, IgG72) to which they bind.

### Discussion

In the past two decades, multiple zoonotic $3-CoVs have spilled over into humans, including: SARS-CoV (Drosten et al., 2003; Menachery et al., 2016; Zhong et al., 2003); MERS-CoV (van Boheemen et al., 2012; Zaki, 2012; Zaki et al., 2012); endemic human CoV (HKU1,OC43 (Corman et al., 2018; Islam et al., 2021; Vijgen et al., 2005)); and SARS-CoV-2 (McCallum et al., 2021; Wang et al., 2021b; Wu et al., 2020; Zhu et al., 2020). These β-CoVs, especially SARS-CoV-2 variants such as omicron, have resulted in enormous global disease burden, severe hospitalization, and mortality. While an arsenal of vaccines has been developed or licensed (Callaway, 2023; Krammer, 2020; Tai et al., 2022), the antigenic discordance between the current vaccines and circulating variants has diminished their utility. This discordance is largely due to the rapid evolution of virus circulating on a pandemic scale and the numerous resulting escape mutations within the immunodominant receptor-binding domain (RBD) of the spike protein that decorates the virion surface. For instance, the SARS-CoV-2 omicron variant harbors over 15 amino acid mutations within RBD of the spike protein (Wu et al., 2022). Furthermore, the recent emergence of bat MERS-CoV variants (NeoCoV and PDF-2180) raises additional concerns about including RBD in $3-CoV vaccines (Menachery Vineet et al., 2020; Xiong et al., 2022). These MERS-CoVs were shown to strongly bind and use bat angiotensin-converting enzyme 2 (ACE2) as entry receptor, and evade MERS-CoV RBD-targeting specific nanobodies.

In addition, the conserved S2 subunit contains an exposed hypervariable immunogenic epitope or loop among closely related lineage C $3-CoVs, which upon immunization results in elicitation of an antibody G4 (Pallesen et al., 2017; Wang et al., 2018; Wang et al., 2015). In accordance, immunization with MERS stem antigens elicit about one-third G4-like neutralizing antibodies in mice (Hsieh et al., 2021). Notwithstanding, the G4 antibody exerts selective pressure, leading to resistant variants (Pallesen et al., 2017). Taken together, these problems highlight an urgent need for next-generation vaccines to broadly protect against β-CoVs.

Additionally, S2 stem stalk has been identified as target for broadly neutralizing antibodies and it is very conserved among β-CoVs (Dacon et al., 2022; Pinto et al., 2021; Sun et al., 2022; Zhou et al., 2022a). It was noted that these anti-S2 antibodies were effectively generated/isolated from convalescent spike vaccinated individuals (hybrid immunity) compared to infection or vaccination only (Dacon et al., 2022; Zhou et al., 2022a). A possible explanation for the paucity of these antibodies in the latter cases could be the difference in the spike conformation associated with the spike vaccines and the infectious virions. Such conformational differences might influence the exposure and accessibility of the S2 stalk bnAb site and subsequent B-cell responses.

It is demonstrated herein that rationally designed non-RBD immunogens displayed on precision nanoparticles using engineered linkers provide a next-generation vaccine strategy against $3-CoVs, and these classes of immunogens would induce broadly protective immune responses. The data reported in this work suggests that nanoparticle immunogens lacking escape mutation-prone epitopes and “mimicry” of spike presentation on virion surface might favor priming of S2 stem stalk bnAb B-cell precursors. The nanoparticle designed with the rigid linker increased antibody binding of antigen and facilitated access to key epitopes. Moreover, the nanoparticle/rigid linker design exposed the occluded S2 epitope, targeted by bnAbs such as IgG22 and CC40.8 on the native spike protein.

Other research groups have shown that CC40.8-like antibodies are rare in humans following infection with SARS-CoV-2, and the authors suggested their elicitation will require rational structure vaccine design (Dacon et al., 2022; Zhou et al., 2022b). The data reported in this thesis confirms this notion, as CC40.8 did not bind to the S2PΔRBD antigen. Although some work has reported an approach of grafting or transplanting these peptide epitopes onto scaffolds, these scaffolds may be distracting and could dampen the immune responses (Correia et al., 2014; Kapingidza et al., 2023). In addition, the instant nanoparticle designs exposed these regions and still retained antibody binding to other conserved and protective epitopes, such as G2 targeting the NTD and the IgG72 epitopes. Thus, the instant nanoparticle design provides a more promising approach, with potential to elicit a multi-epitope protective antibody repertoire after immunization.

In summary, described herein are engineered non-RBD immunogens and the characterization of their antigenicity using BLI. This work provides a detailed blueprint for the rational design and characterization of vaccine immunogens against other viral families.

With continued efforts and refinement, nanoparticle immunogens are becoming a mainstay in the battle against emerging viruses with high antigenic diversity such as CoVs, influenza, hepatitis C virus and HIV globally. However, despite great promise, these nanoparticle vaccines are mainly constrained by their inability to induce more targeted, potent, and cross-reactive immune responses (Aung et al., 2023). The multivalent nature of these nanoparticle vaccines has been implicated in attenuation and skewing the specificity of vaccine-elicited neutralizing antibodies (Powell et al., 2022). Moreover, the magnitude and quality of humoral responses induced by some previously described nanoparticle immunogens are minimal due to the inaccessibility of key conserved epitopes and steric crowding caused by the density of their multivalent antigen array (Brouwer et al., 2019; Hutchinson et al., 2022). These factors result in restricted access of the B-cell receptors to the conserved domains and skewed selection to the non-conserved immunodominant epitopes only. Lastly, a key limitation has been the inability of nanoparticle vaccines to induce certain rare and/or convergent potent antibodies (Dacon et al., 2022; Zhou et al., 2022b). Nevertheless, if immunogens (void of variable epitopes or rapidly mutating viral domains) could be designed and precisely displayed on nanoparticles to consistently induce broad immune responses, protection against these pathogens, particularly CoVs and their circulating variants might be achieved. This work began with the rational design of MERS-CoV antigens, their precision display on nanoparticles using designed linkers, followed by their in-depth antigenic characterization, and concluded with exploring their ability to expose epitopes in a manner that induces production of specific broadly protective antibodies, a feat inaccessible to native or stabilized spike glycoprotein. The significant scientific findings are summarized below.

Design, Expression, and Characterization of Nanoparticle Immunogens. Leveraging rational antigen designs, linkers, and CoV precision nanoparticle platforms, the inventors successfully engineered various nanoparticle vaccine candidates based on the MERS spike glycoprotein components with focus on targeting more conserved neutralizing epitopes/domains to induce broad β-CoV protection. It was demonstrated that nanoparticle immunogens recombinantly expressed at a decent level in mammalian (Expi293F) cells and separated to higher purity. In addition, nanoparticle immunogens purified with size-exclusion chromatography (SEC) confirmed predominant peaks corresponding to the predicted assemblies and sizes. In further biophysical or structural characterization using negative-stain electron microscopy (NS-TEM) or cryo-electron microscopy (Cryo-EM), nanoparticle immunogens were homogenous and monodispersed with engineered spike domains or components protruding from the surface of the nanoparticle core.

Antigenic Characterization of the Nanoparticle Vaccine Candidates. Following successful rational design, production, and structural characterization of the nanoparticle vaccine candidates, the inventors evaluated their antigenicity using BLI by comparing their binding affinities to the antigens. By using relevant monoclonal antibodies (A2, G2, F11, D12, IgG22, IgG72, G4) the inventors confirmed binding affinities for the nanoparticle vaccine candidates, thus establishing their antigenicity profiles. The antibody binding (evident by their equilibrium dissociation constants, K_{D}) for the nanoparticle candidates were comparable or better than the respective antigens. These data indicate that designed nanoparticle immunogens displayed the antigens in their native conformations and that their dense state does not sterically inhibit antibody access. On the contrary, compared to SS ΔG4loop, it was found that the SSAG4loop nanoparticle immunogens still bound to G4 antibody with similar K_{D} as MERS SS nanoparticles and will require further modifications prior to their future exploration in vivo. Nonetheless, these results show that nanoparticle platforms may generate vaccine candidates which are sufficiently robust to enable efficient B-cell receptor (BCR) cross-linking leading to substantial immunogenicity of the displayed antigens.

Moreover, in a detailed epitope exposure analysis, it was found that nanoparticle immunogens bound well to respective antibodies with highest binding signals by several order of magnitude (2-3-fold increase) compared to antigens at similar or higher concentrations (15× the NPs). In summary, these antigenic characterization data serve as a blueprint for selecting nanoparticle vaccine candidates for future animal immunization and exploration.

Precision Nanoparticle Display and Epitope Accessibility. The inventors recognized previously reported CoV spike nanoparticle vaccines that lacked antibody responses to the conserved S2 domain and/or produced weak antibody responses due to inaccessibility of key conserved neutralizing epitopes on spike antigens or steric crowding and restricted BCR access in the context of dense multivalent nanoparticle immunogens. In turn, the inventors engineered nanoparticle vaccine candidates with a rigid linker to successfully expose otherwise shielded epitopes of currently available rare potent neutralizing antibodies (isolated from convalescent patients) which were unattainable in reaction to the full spike antigens or nanoparticle vaccine candidates with flexible linkers. Spike displayed with L2 on respective nanoparticles (MERS S2PΔRBD-L2-Aferr/LuS) bound to antibody CC40.8 in a context that roughly mimics B-cell receptor-immunogen interaction (that is essential for B-cell activation). This may possibly lead to positive B-cell selection, induction, and elicitation of similar or even higher potency antibody responses later after immunization. The inventors understand this to be the first report of engineered nanoparticle vaccines with the ability to expose a potent neutralizing epitope of an antibody, which was not recognized in the full spike state. Hence it is possible that the rigid linker in the MERS S2PΔRBD-L2-Aferr/LuS enabled better access to the epitope targeted by CC40.8. In addition, other multiple distinct epitopes targeted by neutralizing S2 antibodies, such as IgG22, IgG72, and G4, were exposed and accessible for binding to the designed spike nanoparticle vaccine (for example, MERS S2PΔRBD-L2-Aferr/LuS) highlighting that these epitopes may be more accessible to BCRs than less neutralizing sub physiological epitopes, like foldon, after immunization. This conclusion further emphasizes the need for precision nanoparticle display with features such as size, shape, antigen number, and their spatial organization.

### Future Directions

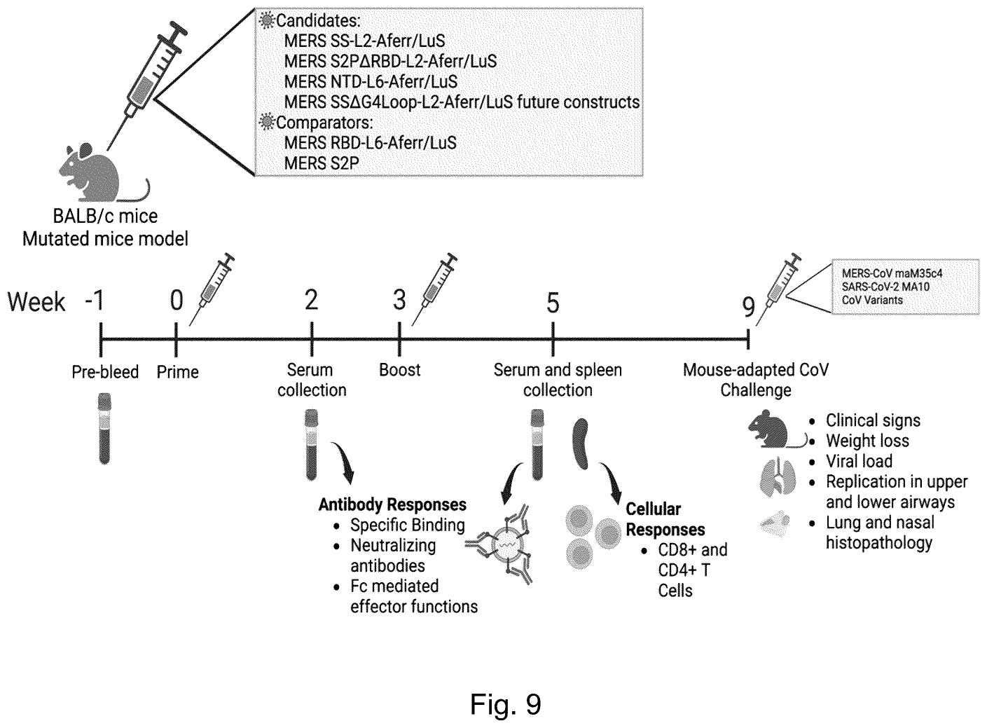

The outcomes of this work have highlighted the advantages of rationally designed nanoparticle vaccines against β-CoVs and established the fundamental understanding of the antigenic signatures of CoV spikes and their precise presentation on nanoparticles, exploring knowledge which is imperative for multi-epitope exposure, targeting, and pan-nanoparticle vaccine development. The work has built a selection blueprint to guide future exploration. Based on these accomplishments and antigenicity data, the following future directions (as summarized in FIG. 9) are recommended to explore the full potentials of the promising vaccine candidates to combat CoVs.

Engineering of SSAG4loop Nanoparticle vaccine candidates. Despite the initial deletion of antibody G4's variable immunogenic epitope, it still binds to the respective nanoparticle vaccine candidates and may elicit G4-like antibodies after vaccination. It is hypothesized, without wishing to be bound by theory, that this effect may be due to the avidity, where individual binding strength derived from possible G4's non-covalent interactions with hydrophobic S2 residues outside of the extended loop accumulated on nanoparticles due to their multimeric nature. Therefore, prior to animal immunization, it would be essential to further engineer regions around the G4 epitope for these nanoparticle vaccine candidates to prevent natural immunofocusing and elicitation of these G4-like antibodies after immunization. To this end, the inventors contemplate these strategies and their combination thereof: (i) further amino acid deletion around G4 epitope; (ii) glycan masking via addition of one or more specific N-linked glycosylation sites/motifs within and around the deleted G4 loop. Following these strategies, the engineered nanoparticle vaccine candidates must be rigorously characterized to confirm the complete blocking or shielding of the G4 antibody recognition and retention, or even improvement of the binding of the potent S2 or stalk-targeting antibodies. Finally, the best design strategy should be applied to the initial engineered SSAG4loop antigen.

Evaluation of Immunogenicity of Selected Nanoparticle Vaccines Candidates in Mouse Models. Based on these promising antigenicity data, some nanoparticle vaccine candidates should be selected to further investigate whether these findings translate or replicate in in vivo animal models. Particularly, animal models should seek to confirm that the multivalent presentation using nanoparticle platforms, such as Aferr and LuS, can enhance and shape the breadth, magnitude, and/or strength of NTD and cross-reactive stem responses in vivo across multiple CoV strains. To this end, the inventors contemplate the design and immunization studies in FIG. 9. First, the immunogenicity of these immunogens will be evaluated in the BALB/cJ mouse model. The animals will be immunized intramuscularly (IM) twice at weeks 0 and 3 with the selected nanoparticle vaccine candidates adjuvanted with Sigma Adjuvant System. In addition, two nanoparticle immunogens will be assessed in their unadjuvanted form and compared to the adjuvanted MERS-SS antigen to discern if the unadjuvanted nanoparticles elicit similar or even more binding and neutralizing titers. The serum will be collected two weeks after each injection and heat-inactivated to evaluate immune responses. The humoral responses will be measured/quantified by comparative amounts of binding and neutralizing antibody titers (via enzyme-linked immunosorbent assay (ELISA) and lentiviral-pseudotyped virus neutralization assay) using the MERS-S2P antigen and MERS-RBD nanoparticles as benchmark or comparators.

Most importantly, to assess the nanoparticle immunogens' cross-reactivity and broad protective capability, some CoV spikes and pseudoviruses from the β-CoVs clade will be evaluated. Besides humoral immunity, previous studies (Marcandalli et al., 2019; Moon et al., 2012) have shown that nanoparticle immunogens can enhance Germinal centre (GC) formation and T follicular helper cells (T_{fh}) expansion compared to soluble antigens. Moreover, Interferon-γ (IFN-γ)-producing T_{H}1 cells are vital for generating and inducing potent antibody response and cellular immunity, respectively (Laidlaw et al., 2016; Snell et al., 2016). Cellular immunity is essential for eliminating infected cells to control viral infections. Early T-cell response and memory have been demonstrated to protect against SARS-CoV-2 after vaccination (Grifoni et al., 2020; Peng et al., 2020). However, the CoV vaccines must induce CD4+T_{H}1, but not T_{H}2-type T cell response as the latter has been shown to compromise vaccine-associated enhancement of respiratory disease (Graham, 2020). Two weeks after the second immunization (week 5), mouse splenocytes, lymph nodes, and bone marrow tissues will be collected and/or isolated for assessment of T cell responses. Using flow cytometry, the total number or frequency of spike- and S2-specific T_{fh}, GC B-cells or antibody-secreting cells, IFN-γ-producing CD4 T_{H}1 and CD8+Tc (all stained with respective biomarkers or antibodies) will be analysed and compared between nanoparticle and antigen groups. The naïve samples will be used as a negative control.

Antibody (Ab) Fc-mediated effector functions play a vital role in protection in vivo by facilitating viral clearance and antiviral immune responses (Bournazos et al., 2020; Schafer et al., 2021). Following the second immunization (at week 3) and serum collection (at week 5), the ability of immune serum to trigger activation of Fc receptors, FcγRIIa and FcγRIIIa will be analysed and compared using a luciferase reporter assay (in vitro). Moreover, the ability of sera from immunized mice to exert or promote robust activation of Ab-dependent cell cytotoxicity (ADCC), Ab-dependent cellular phagocytosis (ADCP), and complement-dependent cytotoxicity (ECDC) will be examined.

Evaluation of Protective Efficacy of Selected Nanoparticle Vaccines Candidates in Mice. Following the immunogenicity study, the protective efficacy of the most potent nanoparticle vaccine candidate (compared to the MERS S2P) will be evaluated in wild-type (BALB/cJ) or mutated mouse models (288/330^{+/+} and K18-human ACE2 transgenic mice). The mice will be immunized (IM) similar to the immunogenicity schedule or design (FIG. 9) with appropriate vaccine doses. The mice will be challenged (with mouse-adapted MERS maM35c4 and SARS-CoV MA10) in the BSL3 facility for at least four weeks following the second immunization. Following the challenge, mice will be monitored for clinical signs, body weight loss, and survival, and euthanized to investigate viral replication, lung discoloration, and viral burden in the upper and lower airways (FIG. 9). Moreover, polyclonal immunoglobulin purified from collected immune sera will be used to passively immunize mice via the intraperitoneal (IP) route per the design and analysis of tissue discoloration and viral titers will be performed.

Molecular Mechanism, Plasma epitope mapping, and Antibody detection. The inventors contemplate that some selected nanoparticle vaccine candidates will induce a rapid or broad immune response after single or second immunization. Therefore, contingent on the immunogenicity study, these nanoparticle vaccines will be further characterized in a dose-ranging study using the same immunization design in FIG. 9. In addition, the antibody binding and neutralizing responses to MERS-CoV spike and S2 will be assessed in the same immunogenicity assays at all doses/concentrations.

To investigate the molecular mechanism for induced antibody responses and define the relative contributions of which known NTD-like or S2/stalk-like/G4-like antibodies (such as G2, A2, IgG22, IgG72,CC40.8,G4) have been elicited, these antibodies will be used as competitors in assays (ELISA and BLI) against serum/pooled plasma from immunized mice to permit determination of relative concentrations or fractions of similar antibodies in the polyclonal fractions. This will also permit confirmation of whether the antigens were intact with previously observed antigenicity upon immunization, or if the nanoparticle skewed the epitope specificity of elicited antibodies. Next, the competitive difference will be confirmed by directly visualizing digested polyclonal antibody fab fragments from the purified nanoparticle-elicited antibodies in complex with MERS-CoV and SARS-CoV-2 spike proteins using single-particle negative-stain and cryo-electron microscopy analysis (FIG. 5B). This will permit mapping of the epitope recognized by the polyclonal fab and their known or unknown conserved antigenic supersites on the spike S2.

### REFERENCES

Aung, A., Cui, A., Maiorino, L., Amini, A. P., Gregory, J. R., Bukenya, M., Zhang, Y., Lee, H., Cottrell, C. A., Morgan, D. M., et al. (2023). Low protease activity in B cell follicles promotes retention of intact antigens after immunization. Science 379, eabn8934.
Bachmann, M. F., and Jennings, G. T. (2010). Vaccine delivery: a matter of size, geometry, kinetics and molecular patterns. Nat Rev Immunol 10, 787-796.
Barnes, C. O., West, A. P., Jr., Huey-Tubman, K. E., Hoffmann, M. A. G., Sharaf, N. G., Hoffman, P. R., Koranda, N., Gristick, H. B., Gaebler, C., Muecksch, F., et al. (2020). Structures of Human Antibodies Bound to SARS-CoV-2 Spike Reveal Common Epitopes and Recurrent Features of Antibodies. Cell 182, 828-842.e816.
Bournazos, S., Corti, D., Virgin, H. W., and Ravetch, J. V. (2020). Fc-optimized antibodies elicit CD8 immunity to viral respiratory infection. Nature 588, 485-490.
Brouwer, P. J. M., Antanasijevic, A., Berndsen, Z., Yasmeen, A., Fiala, B., Bijl, T. P. L., Bontjer, I., Bale, J. B., Sheffler, W., Allen, J. D., et al. (2019). Enhancing and shaping the immunogenicity of native-like HIV-1 envelope trimers with a two-component protein nanoparticle. Nature Communications 10, 4272.
Callaway, E. (2023). The next generation of coronavirus vaccines: a graphical guide. Nature 614, 22-25.
Cao, Y., Wang, J., Jian, F., Xiao, T., Song, W., Yisimayi, A., Huang, W., Li, Q., Wang, P., An, R., et al. (2022). Omicron escapes the majority of existing SARS-CoV-2 neutralizing antibodies. Nature 602, 657-663.
Castón, J. R., and Carrascosa, J. L. (2013). The basic architecture of viruses. Subcell Biochem 68, 53-75.
CDC (2022). SARS-CoV-2 Variant Classifications and Definitions. In COVID-19 (Center for Disease Control and Prevention).
Chattopadhyay, S., Chen, J. Y., Chen, H. W., and Hu, C. J. (2017). Nanoparticle Vaccines Adopting Virus-like Features for Enhanced Immune Potentiation. Nanotheranostics 1, 244-260.
Cohen, A. A., Gnanapragasam, P. N. P., Lee, Y. E., Hoffman, P. R., Ou, S., Kakutani, L. M., Keeffe, J. R.,
Wu, H.-J., Howarth, M., West, A. P., et al. (2021). Mosaic nanoparticles elicit cross-reactive immune responses to zoonotic coronaviruses in mice. Science 371, 735-741.
Cohen, A. A., van Doremalen, N., Greaney, A. J., Andersen, H., Sharma, A., Starr, T. N., Keeffe, J. R.,
Fan, C., Schulz, J. E., Gnanapragasam, P. N. P., et al. (2022a). Mosaic RBD nanoparticles protect against challenge by diverse sarbecoviruses in animal models. Science 377, eabq0839.
Cohen, L. E., Spiro, D. J., and Viboud, C. (2022b). Projecting the SARS-CoV-2 transition from pandemicity to endemicity: Epidemiological and immunological considerations. PLOS Pathogens 18, e1010591.
Corman, V. M., Muth, D., Niemeyer, D., and Drosten, C. (2018). Hosts and Sources of Endemic Human Coronaviruses. Adv Virus Res 100, 163-188.
Correia, B. E., Bates, J. T., Loomis, R. J., Baneyx, G., Carrico, C., Jardine, J. G., Rupert, P., Correnti, C., Kalyuzhniy, O., Vittal, V., et al. (2014). Proof of principle for epitope-focused vaccine design. Nature 507, 201-206.
Cui, J., Li, F., and Shi, Z.-L. (2019). Origin and evolution of pathogenic coronaviruses. Nature Reviews Microbiology 17, 181-192.
Dacon, C., Peng, L., Lin, T.-H., Tucker, C., Lee, C.-C. D., Cong, Y., Wang, L., Purser, L., Cooper, A. J. R., Williams, J. K., et al. (2022). Rare, convergent antibodies targeting the stem helix broadly neutralize diverse betacoronaviruses. Cell Host & Microbe.
Dong, E., Du, H., and Gardner, L. (2020). An interactive web-based dashboard to track COVID-19 in real time. Lancet Infect Dis 20, 533-534.
Drosten, C., Günther, S., Preiser, W., van der Werf, S., Brodt, H. R., Becker, S., Rabenau, H., Panning, M., Kolesnikova, L., Fouchier, R. A., et al. (2003). Identification of a novel coronavirus in patients with severe acute respiratory syndrome. N Engl J Med 348, 1967-1976.
Duan, L., Zheng, Q., Zhang, H., Niu, Y., Lou, Y., and Wang, H. (2020). The SARS-CoV-2 Spike Glycoprotein Biosynthesis, Structure, Function, and Antigenicity: Implications for the Design of Spike-Based Vaccine Immunogens. Front Immunol 11, 576622.
ECDC (2022). Geographical distribution of confirmed cases of MERS-CoV by reporting country, April 2012-10 Nov. 2022. In Analysis and Guidance (ecdc.europa.eu/: European Centre for Disease Prevention and Control), pp. Since the disease was first identified in Saudi Arabia in April 2012, over 2012 2600 cases of Middle East respiratory syndrome coronavirus (MERS-CoV) have been detected in 2027 countries.
Elhazmi, A., Al-Tawfiq, J. A., Sallam, H., Al-Omari, A., Alhumaid, S., Mady, A., and Al Mutair, A. (2021). Severe acute respiratory syndrome coronavirus 2 (SARS-CoV-2) and Middle East Respiratory Syndrome Coronavirus (MERS-CoV) coinfection: A unique case series. Travel Med Infect Dis 41, 102026.
Goddard, T. D., Huang, C. C., Meng, E. C., Pettersen, E. F., Couch, G. S., Morris, J. H., and Ferrin, T. E. (2018). UCSF ChimeraX: Meeting modern challenges in visualization and analysis. Protein Sci 27, 14-25.
Graham, B. S. (2020). Rapid COVID-19 vaccine development. Science 368, 945-946.
Graham, B. S., and Sullivan, N. J. (2018). Emerging viral diseases from a vaccinology perspective: preparing for the next pandemic. Nature Immunology 19, 20-28.
Greaney, A. J., Starr, T. N., Barnes, C. O., Weisblum, Y., Schmidt, F., Caskey, M., Gaebler, C., Cho, A., Agudelo, M., Finkin, S., et al. (2021). Mapping mutations to the SARS-CoV-2 RBD that escape binding by different classes of antibodies. Nature Communications 12, 4196.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al. (2020). Targets of T Cell Responses to SARS-CoV-2 Coronavirus in Humans with COVID-19 Disease and Unexposed Individuals. Cell 181, 1489-1501.e1415.
Harvey, W. T., Carabelli, A. M., Jackson, B., Gupta, R. K., Thomson, E. C., Harrison, E. M., Ludden, C., Reeve, R., Rambaut, A., Peacock, S. J., et al. (2021). SARS-CoV-2 variants, spike mutations and immune escape. Nature Reviews Microbiology 19, 409-424.
Houser, K. V., Chen, G. L., Carter, C., Crank, M. C., Nguyen, T. A., Burgos Florez, M. C., Berkowitz, N. M., Mendoza, F., Hendel, C. S., Gordon, I. J., et al. (2022). Safety and immunogenicity of a ferritin nanoparticle H2 influenza vaccine in healthy adults: a phase 1 trial. Nature Medicine 28, 383-391.
Hsieh, C. L., Werner, A. P., Leist, S. R., Stevens, L. J., Falconer, E., Goldsmith, J. A., Chou, C. W., Abiona, O. M., West, A., Westendorf, K., et al. (2021). Stabilized coronavirus spike stem elicits a broadly protective antibody. Cell Rep 37, 109929.
Hu, J., Chen, X., Lu, X., Wu, L., Yin, L., Zhu, L., Liang, H., Xu, F., and Zhou, Q. (2022). A spike protein S2 antibody efficiently neutralizes the Omicron variant. Cellular & Molecular Immunology 19, 644-646.
Huang, Y., Yang, C., Xu, X.-f, Xu, W., and Liu, S.-w. (2020). Structural and functional properties of SARS-CoV-2 spike protein: potential antivirus drug development for COVID-19. Acta Pharmacologica Sinica 41, 1141-1149.
Hurlburt, N. K., Homad, L. J., Sinha, I., Jennewein, M. F., MacCamy, A. J., Wan, Y.-H., Boonyaratanakornkit, J., Sholukh, A. M., Jackson, A. M., Zhou, P., et al. (2022). Structural definition of a pan-sarbecovirus neutralizing epitope on the spike S2 subunit. Communications Biology 5, 342.
Hutchinson, G., Abiona, O., Ziwawo, C., Werner, A., Ellis, D., Tsybovsky, Y., Leist, S., Palandjian, C., West, A., Fritch, E., et al. (2022). Nanoparticle display of prefusion coronavirus spike elicits S1-focused cross-reactive protection across divergent subgroups. Res Sq.
Islam, A., Ferdous, J., Islam, S., Sayeed, M. A., Dutta Choudhury, S., Saha, O., Hassan, M. M., and Shirin, T. (2021). Evolutionary Dynamics and Epidemiology of Endemic and Emerging Coronaviruses in Humans, Domestic Animals, and Wildlife. Viruses 13.
Jardine, J., Julien, J. P., Menis, S., Ota, T., Kalyuzhniy, O., McGuire, A., Sok, D., Huang, P. S., MacPherson, S., Jones, M., et al. (2013). Rational HIV immunogen design to target specific germline B cell receptors. Science 340, 711-716.
Kanekiyo, M., Wei, C. J., Yassine, H. M., McTamney, P. M., Boyington, J. C., Whittle, J. R., Rao, S. S., Kong, W. P., Wang, L., and Nabel, G. J. (2013). Self-assembling influenza nanoparticle vaccines elicit broadly neutralizing H1N1 antibodies. Nature 499, 102-106.
Kapingidza, B., Marston, D. J., Harris, C., Wrapp, D., Winters, K., Rhodes, B., Vure, P., Woods, C. W., Petzold, E. A., Walter, E. B., et al. (2023). Engineered Immunogens to Expose Conserved Epitopes Targeted by Broad Coronavirus Antibodies. bioRxiv, 2023.2002.2027.530277.
Kissler, S. M., Tedijanto, C., Goldstein, E., Grad, Y. H., and Lipsitch, M. (2020). Projecting the transmission dynamics of SARS-CoV-2 through the postpandemic period. Science 368, 860-868.
Krammer, F. (2020). SARS-CoV-2 vaccines in development. Nature 586, 516-527.
Kurhade, C., Zou, J., Xia, H., Liu, M., Chang, H. C., Ren, P., Xie, X., and Shi, P. Y. (2023). Low neutralization of SARS-CoV-2 Omicron BA.2.75.2, BQ.1.1 and XBB.1 by parental mRNA vaccine or a BA.5 bivalent booster. Nature Medicine 29, 344-347.
Laidlaw, B. J., Craft, J. E., and Kaech, S. M. (2016). The multifaceted role of CD4(+) T cells in CD8(+) T cell memory. Nat Rev Immunol 16, 102-111.
Lamiable, A., Thévenet, P., Rey, J., Vavrusa, M., Derreumaux, P., and Tufféry, P. (2016). PEP-FOLD3: faster de novo structure prediction for linear peptides in solution and in complex. Nucleic Acids Research 44, W449-W454.
Lee, D. B., Kim, H., Jeong, J. H., Jang, U. S., Jang, Y., Roh, S., Jeon, H., Kim, E. J., Han, S. Y., Maeng,
J. Y., et al. (2023). Mosaic RBD nanoparticles induce intergenus cross-reactive antibodies and protect against SARS-CoV-2 challenge. Proceedings of the National Academy of Sciences 120, e2208425120.
Lee, L. A., and Wang, Q. (2006). Adaptations of nanoscale viruses and other protein cages for medical applications. Nanomedicine 2, 137-149.
Lin, L. C., Huang, C. Y., Yao, B. Y., Lin, J. C., Agrawal, A., Algaissi, A., Peng, B. H., Liu, Y. H., Huang,
P. H., Juang, R. H., et al. (2019). Viromimetic STING Agonist-Loaded Hollow Polymeric Nanoparticles for Safe and Effective Vaccination against Middle East Respiratory Syndrome Coronavirus. Adv Funct Mater 29, 1807616.
López-Sagaseta, J., Malito, E., Rappuoli, R., and Bottomley, M. J. (2016). Self-assembling protein nanoparticles in the design of vaccines. Comput Struct Biotechnol J 14, 58-68.
Ma, Y., Wang, Y., Dong, C., Gonzalez, G. X., Zhu, W., Kim, J., Wei, L., Kang, S. M., and Wang, B. Z. (2022). SARS-CoV-2 Spike Stem Protein Nanoparticles Elicited Broad ADCC and Robust Neutralization against Variants in Mice. Small 18, e2200836.
Mackay, I. M., and Arden, K. E. (2015). MERS coronavirus: diagnostics, epidemiology and transmission. Virology Journal 12, 222.
Marcandalli, J., Fiala, B., Ols, S., Perotti, M., de van der Schueren, W., Snijder, J., Hodge, E., Benhaim, M., Ravichandran, R., Carter, L., et al. (2019). Induction of Potent Neutralizing Antibody Responses by a Designed Protein Nanoparticle Vaccine for Respiratory Syncytial Virus. Cell 176, 1420-1431.e1417.
Mastronarde, D. N. (2005). Automated electron microscope tomography using robust prediction of specimen movements. Journal of Structural Biology 152, 36-51.
McCallum, M., Bassi, J., De Marco, A., Chen, A., Walls, A. C., Di Iulio, J., Tortorici, M. A., Navarro, M. J., Silacci-Fregni, C., Saliba, C., et al. (2021). SARS-CoV-2 immune evasion by the B.1.427/B.1.429 variant of concern. Science 373, 648-654.
Menachery, V. D., Yount, B. L., Jr., Sims, A. C., Debbink, K., Agnihothram, S. S., Gralinski, L. E., Graham, R. L., Scobey, T., Plante, J. A., Royal, S. R., et al. (2016). SARS-like WIV1-CoV poised for human emergence. Proc Natl Acad Sci USA 113, 3048-3053.
Menachery Vineet, D., Dinnon Kenneth, H., Yount Boyd, L., McAnarney Eileen, T., Gralinski Lisa, E., Hale, A., Graham Rachel, L., Scobey, T., Anthony Simon, J., Wang, L., et al. (2020). Trypsin Treatment Unlocks Barrier for Zoonotic Bat Coronavirus Infection. Journal of Virology 94, e01774-01719.
Montgomerie, I., Bird, T. W., Palmer, OR., Mason, N. C., Pankhurst, T. E., Lawley, B., Hernández, L. C., Harfoot, R., Authier-Hall, A., Anderson, D. E., et al. (2023). Incorporation of SARS-CoV-2 spike NTD to RBD Protein Vaccine Improves Immunity Against Viral Variants. iScience, 106256.
Moon, J. J., Suh, H., Polhemus, M. E., Ockenhouse, C. F., Yadava, A., and Irvine, D. J. (2012). Antigen-displaying lipid-enveloped PLGA nanoparticles as delivery agents for a Plasmodium vivax malaria vaccine. PLoS One 7, e31472.
Morens, D. M., Taubenberger, J. K., and Fauci, A. S. (2021). Universal Coronavirus Vaccines—An Urgent Need. New England Journal of Medicine 386, 297-299.
Ng, K. W., Faulkner, N., Finsterbusch, K., Wu, M., Harvey, R., Hussain, S., Greco, M., Liu, Y., Kjaer, S., Swanton, C., et al. (2022). SARS-CoV-2 S2& #x2013; targeted vaccination elicits broadly neutralizing antibodies. Science Translational Medicine 14, eabn3715.
Nguyen, B., and Tolia, N. H. (2021). Protein-based antigen presentation platforms for nanoparticle vaccines. npj Vaccines 6, 70.
Noy-Porat, T., Alcalay, R., Mechaly, A., Peretz, E., Makdasi, E., Rosenfeld, R., and Mazor, O. (2021). Characterization of antibody-antigen interactions using biolayer interferometry. STAR Protocols 2, 100836.
Okba, N. M. A., Widjaja, I., van Dieren, B., Aebischer, A., van Amerongen, G., de Waal, L., Stittelaar, K. J., Schipper, D., Martina, B., van den Brand, J. M. A., et al. (2020). Particulate multivalent presentation of the receptor binding domain induces protective immune responses against MERS-CoV. Emerg Microbes Infect 9, 1080-1091.
Pallesen, J., Wang, N., Corbett, K. S., Wrapp, D., Kirchdoerfer, R. N., Turner, H. L., Cottrell, C. A., Becker, M. M., Wang, L., Shi, W., et al. (2017). Immunogenicity and structures of a rationally designed prefusion MERS-CoV spike antigen. Proc Natl Acad Sci USA 114, E7348-e7357.
Paules, C. I., Marston, H. D., and Fauci, A. S. (2020). Coronavirus Infections-More Than Just the Common Cold. JAMA 323, 707-708.
Peiris, J. S., Lai, S. T., Poon, L. L., Guan, Y., Yam, L. Y., Lim, W., Nicholls, J., Yee, W. K., Yan, W. W., Cheung, M. T., et al. (2003). Coronavirus as a possible cause of severe acute respiratory syndrome. Lancet 361, 1319-1325.
Peng, Y., Mentzer, A. J., Liu, G., Yao, X., Yin, Z., Dong, D., Dejnirattisai, W., Rostron, T., Supasa, P., Liu, C., et al. (2020). Broad and strong memory CD4(+) and CD8(+) T cells induced by SARS-CoV-2 in UK convalescent individuals following COVID-19. Nat Immunol 21, 1336-1345.
Pinto, D., Sauer, M. M., Czudnochowski, N., Low, J. S., Tortorici, M. A., Housley, M. P., Noack, J., Walls, A. C., Bowen, J. E., Guarino, B., et al. (2021). Broad betacoronavirus neutralization by a stem helix& #x2013; specific human antibody. Science 373, 1109-1116.
Poon, C., and Patel, A. A. (2020). Organic and inorganic nanoparticle vaccines for prevention of infectious diseases. Nano Express 1, 012001.
Powell, A. E., Xu, D., Roth, G. A., Zhang, K., Chiu, W., Appel, E. A., and Kim, P. S. (2022). Multimerization of Ebola GPΔmucin on protein nanoparticle vaccines has minimal effect on elicitation of neutralizing antibodies. Front Immunol 13, 942897.
Punjani, A., Rubinstein, J. L., Fleet, D. J., and Brubaker, M. A. (2017). cryoSPARC: algorithms for rapid unsupervised cryo-EM structure determination. Nat Methods 14, 290-296.
Rodon, J., Mykytyn, A. Z., Cantero, G., Albulescu, I. C., Bosch, B.-J., Brix, A., Audonnet, J.-C., Bensaid, A., Vergara-Alert, J., Haagmans, B. L., et al. (2022). Protective efficacy of an RBD-based Middle East respiratory syndrome coronavirus (MERS-CoV) particle vaccine in llamas. One Health Outlook 4, 12.
Sauer, M. M., Tortorici, M. A., Park, Y.-J., Walls, A. C., Homad, L., Acton, O. J., Bowen, J. E., Wang, C., Xiong, X., de van der Schueren, W., et al. (2021). Structural basis for broad coronavirus neutralization. Nature Structural & Molecular Biology 28, 478-486.
Schäfer, A., Muecksch, F., Lorenzi, J. C. C., Leist, S. R., Cipolla, M., Bournazos, S., Schmidt, F., Maison, R. M., Gazumyan, A., Martinez, D. R., et al. (2021). Antibody potency, effector function, and combinations in protection and therapy for SARS-CoV-2 infection in vivo. J Exp Med 218.
Snell, L. M., Osokine, I., Yamada, D. H., De la Fuente, J. R., Elsaesser, H. J., and Brooks, D. G. (2016). Overcoming CD4 Th1 Cell Fate Restrictions to Sustain Antiviral CD8 T Cells and Control Persistent Virus Infection. Cell Rep 16, 3286-3296.
Song, G., He, W.-t., Callaghan, S., Anzanello, F., Huang, D., Ricketts, J., Torres, J. L., Beutler, N., Peng, L., Vargas, S., et al. (2021). Cross-reactive serum and memory B-cell responses to spike protein in SARS-CoV-2 and endemic coronavirus infection. Nature Communications 12, 2938.
Su, S., Li, W., and Jiang, S. (2022). Developing pan-β-coronavirus vaccines against emerging SARS-CoV-2 variants of concern. Trends Immunol 43, 170-172.
Sun, X., Yi, C., Zhu, Y., Ding, L., Xia, S., Chen, X., Liu, M., Gu, C., Lu, X., Fu, Y., et al. (2022). Neutralization mechanism of a human antibody with pan-coronavirus reactivity including SARS-CoV-2. Nature Microbiology 7, 1063-1074.
Tai, W., Zhang, X., Yang, Y., Zhu, J., and Du, L. (2022). Advances in mRNA and other vaccines against MERS-CoV. Transl Res 242, 20-37.
van Boheemen, S., de Graaf, M., Lauber, C., Bestebroer, T. M., Raj, V. S., Zaki, A. M., Osterhaus, A. D., Haagmans, B. L., Gorbalenya, A. E., Snijder, E. J., et al. (2012). Genomic characterization of a newly discovered coronavirus associated with acute respiratory distress syndrome in humans. mBio 3.
Vijgen, L., Keyaerts, E., Moës, E., Thoelen, I., Wollants, E., Lemey, P., Vandamme, A. M., and Van Ranst, M. (2005). Complete genomic sequence of human coronavirus OC43: molecular clock analysis suggests a relatively recent zoonotic coronavirus transmission event. J Virol 79, 1595-1604.
Vu, M. N., Kelly, H. G., Kent, S. J., and Wheatley, A. K. (2021). Current and future nanoparticle vaccines for COVID-19. eBioMedicine 74, 103699.
Walls, A. C., Fiala, B., Schäfer, A., Wrenn, S., Pham, M. N., Murphy, M., Tse, L. V., Shehata, L., O'Connor, M. A., Chen, C., et al. (2020a). Elicitation of Potent Neutralizing Antibody Responses by Designed Protein Nanoparticle Vaccines for SARS-CoV-2. Cell 183, 1367-1382.e1317.
Walls, A. C., Park, Y. J., Tortorici, M. A., Wall, A., McGuire, A. T., and Veesler, D. (2020b). Structure, Function, and Antigenicity of the SARS-CoV-2 Spike Glycoprotein. Cell 181, 281-292.e286.
Wang, C., van Haperen, R., Gutiérrez-Álvarez, J., Li, W., Okba, N. M. A., Albulescu, I., Widjaja, I., van Dieren, B., Fernandez-Delgado, R., Sola, I., et al. (2021a). A conserved immunogenic and vulnerable site on the coronavirus spike protein delineated by cross-reactive monoclonal antibodies. Nature Communications 12, 1715.
Wang, L., Shi, W., Chappell, J. D., Joyce, M. G., Zhang, Y., Kanekiyo, M., Becker, M. M., van Doremalen, N., Fischer, R., Wang, N., et al. (2018). Importance of Neutralizing Monoclonal Antibodies Targeting Multiple Antigenic Sites on the Middle East Respiratory Syndrome Coronavirus Spike Glycoprotein To Avoid Neutralization Escape. J Virol 92.
Wang, L., Shi, W., Joyce, M. G., Modjarrad, K., Zhang, Y., Leung, K., Lees, C. R., Zhou, T., Yassine, H. M., Kanekiyo, M., et al. (2015). Evaluation of candidate vaccine approaches for MERS-CoV. Nature Communications 6, 7712.
Wang, N., Rosen, O., Wang, L., Turner, H. L., Stevens, L. J., Corbett, K. S., Bowman, C. A., Pallesen, J., Shi, W., Zhang, Y., et al. (2019). Structural Definition of a Neutralization-Sensitive Epitope on the MERS-CoV S1-NTD. Cell Rep 28, 3395-3405.e3396.
Wang, P., Nair, M. S., Liu, L., Iketani, S., Luo, Y., Guo, Y., Wang, M., Yu, J., Zhang, B., Kwong, P. D., et al. (2021b). Antibody resistance of SARS-CoV-2 variants B.1.351 and B.1.1.7. Nature 593, 130-135.
Wang, Q., Sun, L., and Jiang, S. (2023). Potential recombination between SARS-CoV-2 and MERS-CoV: calls for the development of Pan-CoV vaccines. Signal Transduction and Targeted Therapy 8, 122.
Wang, Z., Muecksch, F., Cho, A., Gaebler, C., Hoffmann, H.-H., Ramos, V., Zong, S., Cipolla, M.,
Johnson, B., Schmidt, F., et al. (2022). Analysis of memory B cells identifies conserved neutralizing epitopes on the N-terminal domain of variant SARS-Cov-2 spike proteins. Immunity 55, 998-1012.e1018.
Woo, P. C., Lau, S. K., Huang, Y., and Yuen, K. Y. (2009). Coronavirus diversity, phylogeny and interspecies jumping. Exp Biol Med (Maywood) 234, 1117-1127.
Wu, F., Zhao, S., Yu, B., Chen, Y.-M., Wang, W., Song, Z.-G., Hu, Y., Tao, Z.-W., Tian, J.-H., Pei,
Y.-Y., et al. (2020). A new coronavirus associated with human respiratory disease in China. Nature 579, 265-269.
Wu, L., Zhou, L., Mo, M., Liu, T., Wu, C., Gong, C., Lu, K., Gong, L., Zhu, W., and Xu, Z. (2022). SARS-CoV-2 Omicron RBD shows weaker binding affinity than the currently dominant Delta variant to human ACE2. Signal Transduction and Targeted Therapy 7, 8.
Xiong, Q., Cao, L., Ma, C., Tortorici, M. A., Liu, C., Si, J., Liu, P., Gu, M., Walls, A. C., Wang, C., et al. (2022). Close relatives of MERS-CoV in bats use ACE2 as their functional receptors. Nature 612, 748-757.
Zaki, A. M. (2012). Novel coronavirus—Saudi Arabia: human isolate. International Society for Infectious Diseases ProMED-mail.
Zaki, A. M., van Boheemen, S., Bestebroer, T. M., Osterhaus, A. D. M. E., and Fouchier, R. A. M. (2012). Isolation of a Novel Coronavirus from a Man with Pneumonia in Saudi Arabia. New England Journal of Medicine 367, 1814-1820.
Zhang, A.-R., Shi, W.-Q., Liu, K., Li, X.-L., Liu, M.-J., Zhang, W.-H., Zhao, G.-P., Chen, J.-J., Zhang, X.-A., Miao, D., et al. (2021). Epidemiology and evolution of Middle East respiratory syndrome coronavirus, 2012-2020. Infectious Diseases of Poverty 10, 66.
Zhong, N. S., Zheng, B. J., Li, Y. M., Poon, Xie, Z. H., Chan, K. H., Li, P. H., Tan, S. Y., Chang, Q., Xie, J. P., et al. (2003). Epidemiology and cause of severe acute respiratory syndrome (SARS) in Guangdong, People's Republic of China, in February, 2003. Lancet 362, 1353-1358.
Zhou, P., Song, G., He, W. T., Beutler, N., Tse, L. V., Martinez, D. R., Schäfer, A., Anzanello, F., Yong, P., Peng, L., et al. (2022a). Broadly neutralizing anti-S2 antibodies protect against all three human betacoronaviruses that cause severe disease. bioRxiv.
Zhou, P., Yuan, M., Song, G., Beutler, N., Shaabani, N., Huang, D., He, W.-t., Zhu, X., Callaghan, S., Yong, P., et al. (2022b). A human antibody reveals a conserved site on beta-coronavirus spike proteins and confers protection against SARS-CoV-2 infection. Science Translational Medicine 14, eabi9215.
Zhu, N., Zhang, D., Wang, W., Li, X., Yang, B., Song, J., Zhao, X., Huang, B., Shi, W., Lu, R., et al. (2020). A Novel Coronavirus from Patients with Pneumonia in China, 2019. N Engl J Med 382, 727-733.

### Example 3

Described herein are vaccine immunogens that provide broad coronavirus protection. These immunogens comprise engineered non-RBD spike protein antigens. In some embodiments, the immunogens comprise full spike ectodomain void of the RBD and hypervariable immunogenic epitope. In some embodiments, the immunogens comprise the engineered stem (S2), void of hypervariable immunogenic epitope and joined to a trimerization domain. In some embodiments, the immunogens further comprising nanoparticles displaying a polypeptide described herein. The immunogens described herein can be used to vaccinate individuals against, e.g., MERS-CoV and coronaviruses (CoV) that are antigenically divergent from the CoV from which the immunogens were obtained. Further described herein are nucleic acids encoding the immunogens described herein.

Coronaviruses remain a global health threat. A zoonotic reservoir exists, rendering coronaviruses constantly poised to jump into humans. Endemic CoVs are now up to 30% of common colds seasonally.

The SARS-CoV Clade demonstrated pandemic potential, with SARS-CoV-2 variants able to evade immune responses. Immune responses also wane over time and endemicity raises need for additional protection.

The MERS-CoV Clade has steady-state transmission from camels to humans with potential for outbreak. This clade has a high case-fatality rate of approximately 35%.

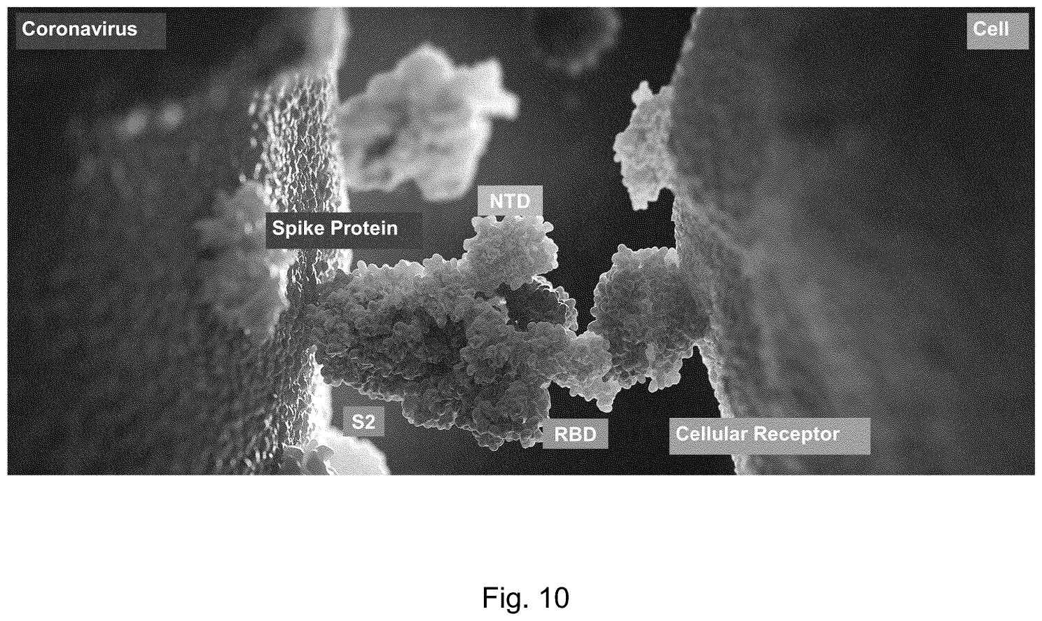

The structure of coronavirus spike proteins is shown in FIG. 10. The escape mutations of MERS are known, and understood to alter the receptor usage. See, e.g., Xiong et al. Nature 2022 612:748-757; which is incorporated by reference herein in its entirety.

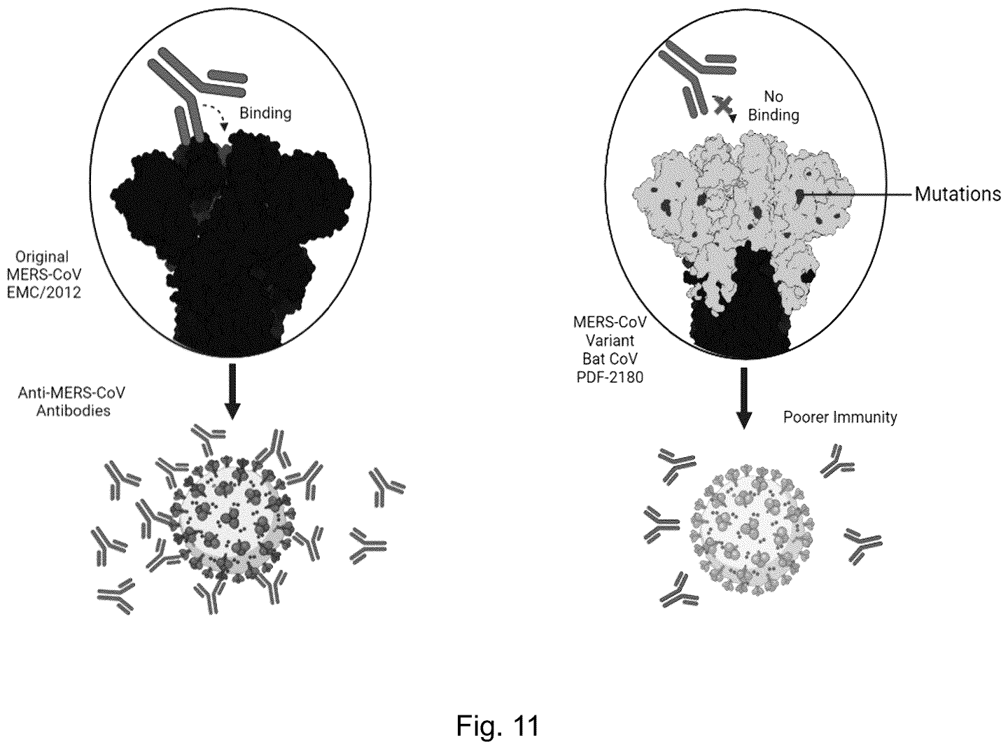

MERS-CoV mutations also elude immunity (FIG. 11). S2 conservation and variable epitope sequences among lineage C betacoronaviruses is shown in FIGS. 12A-12B. The G4 loop or epitope is variable in both sequence and length.

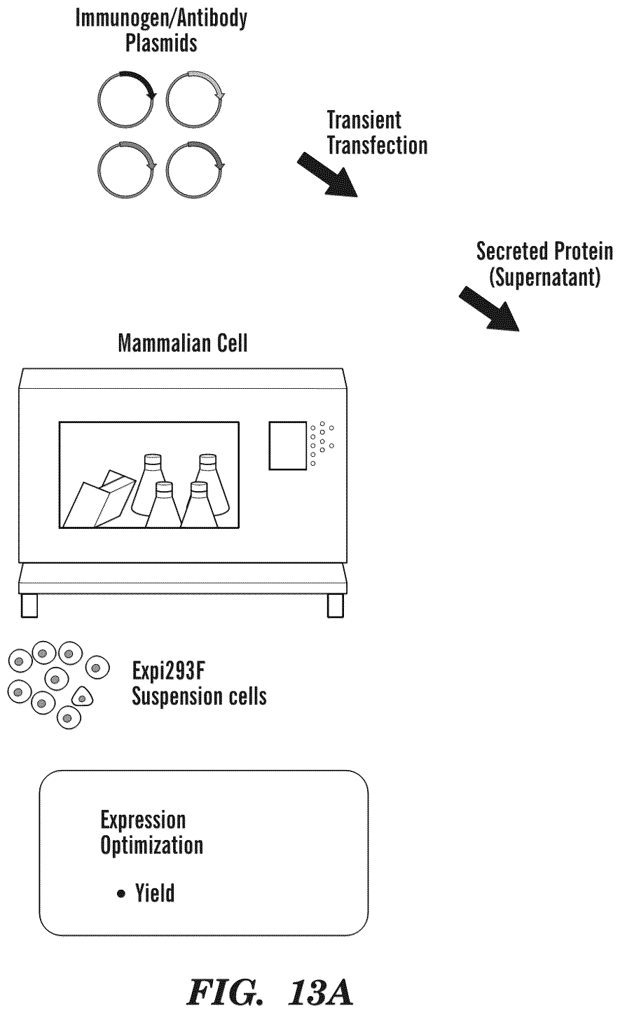

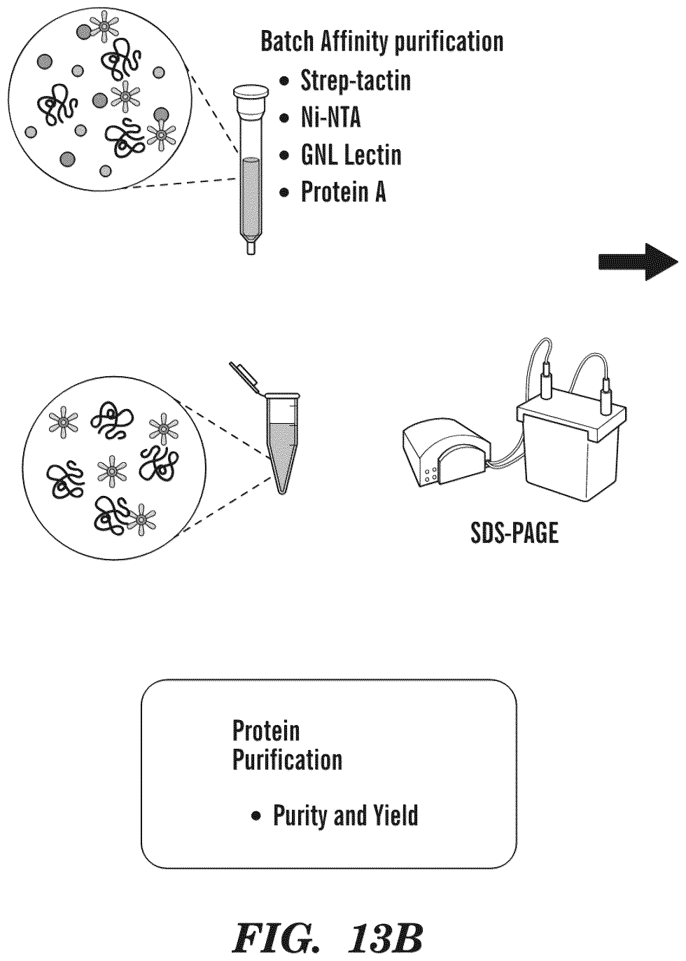

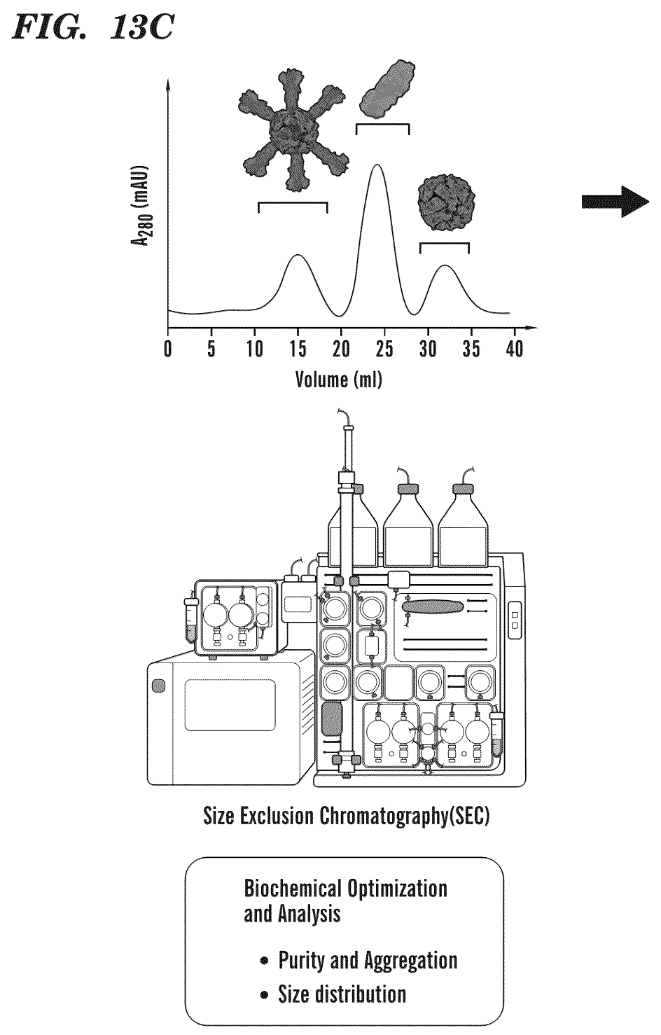

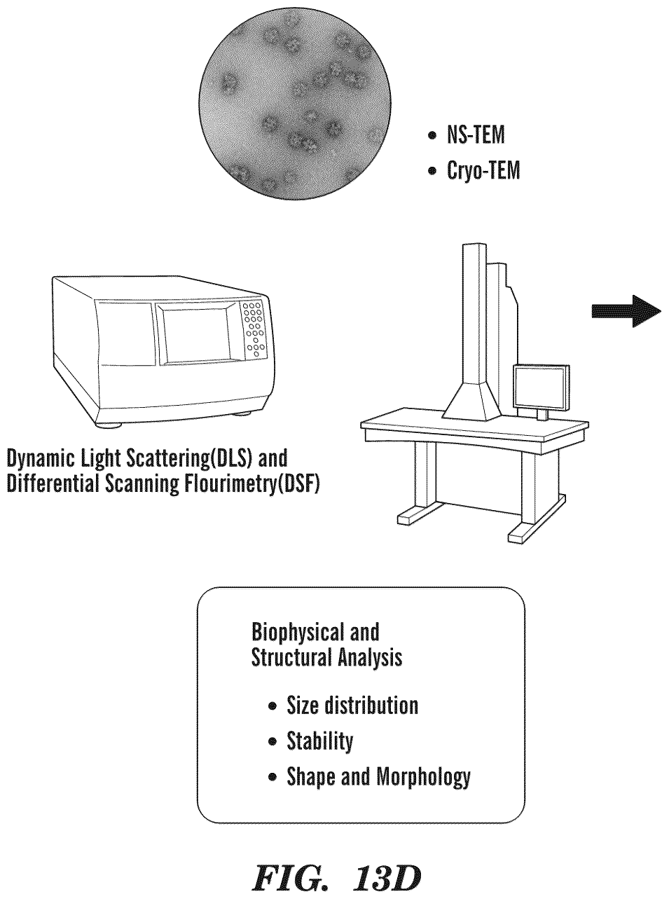

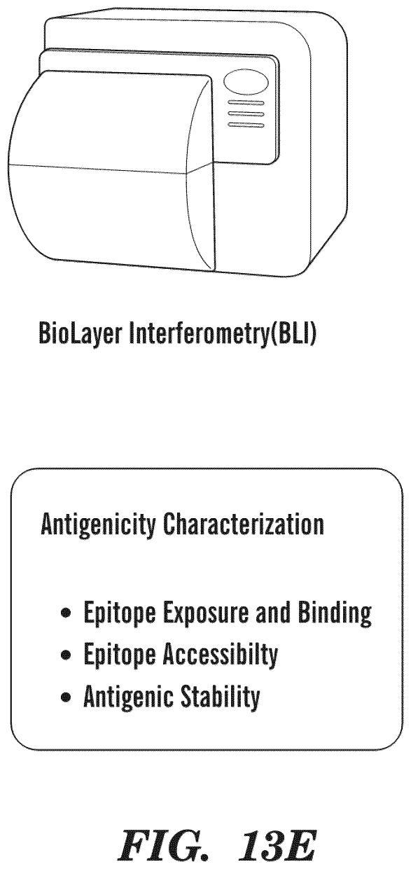

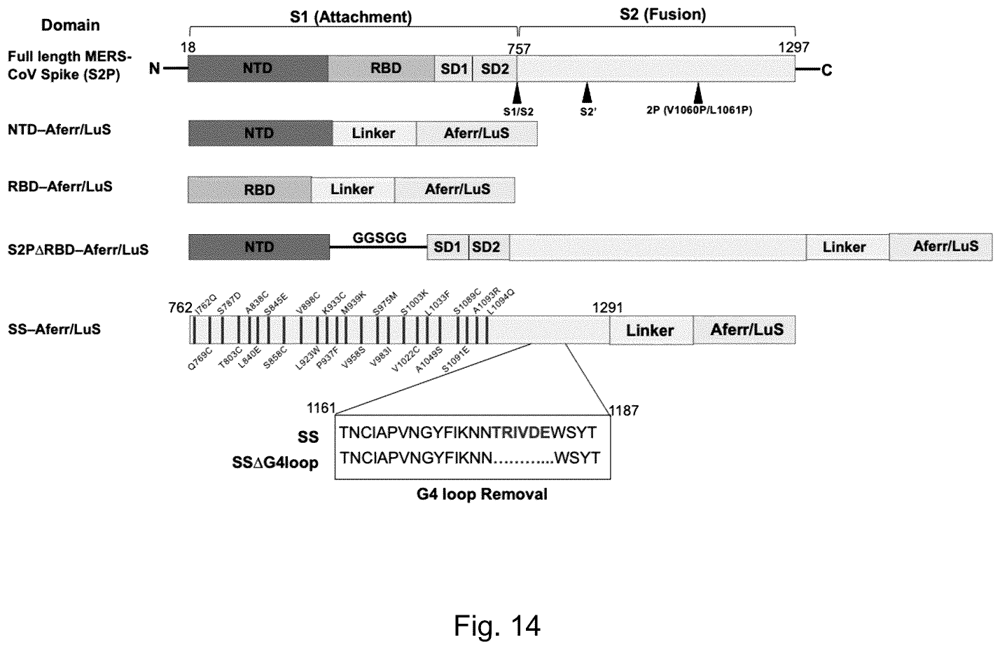

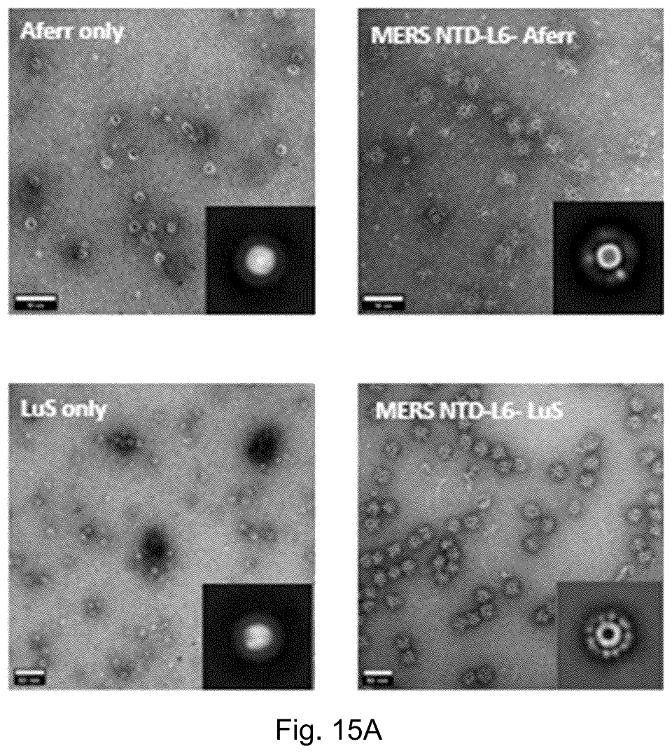

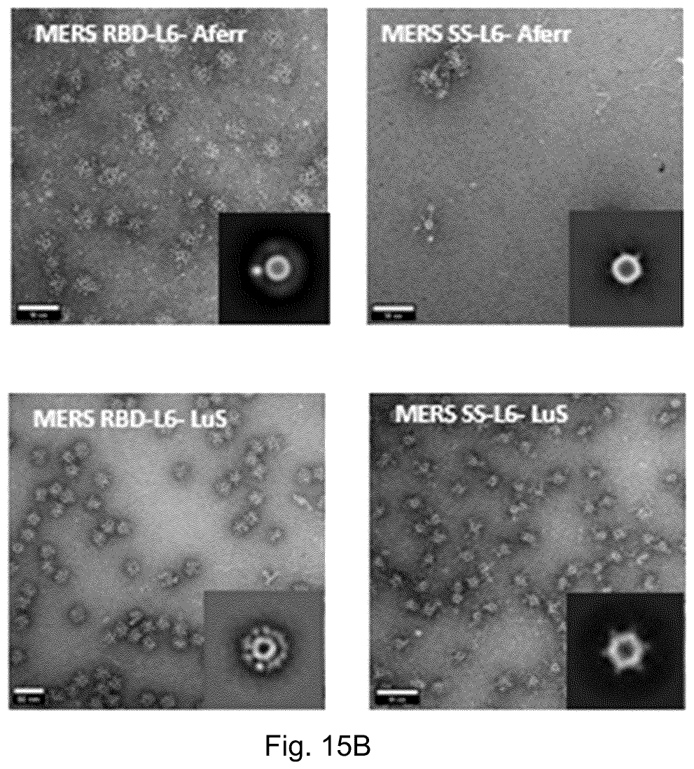

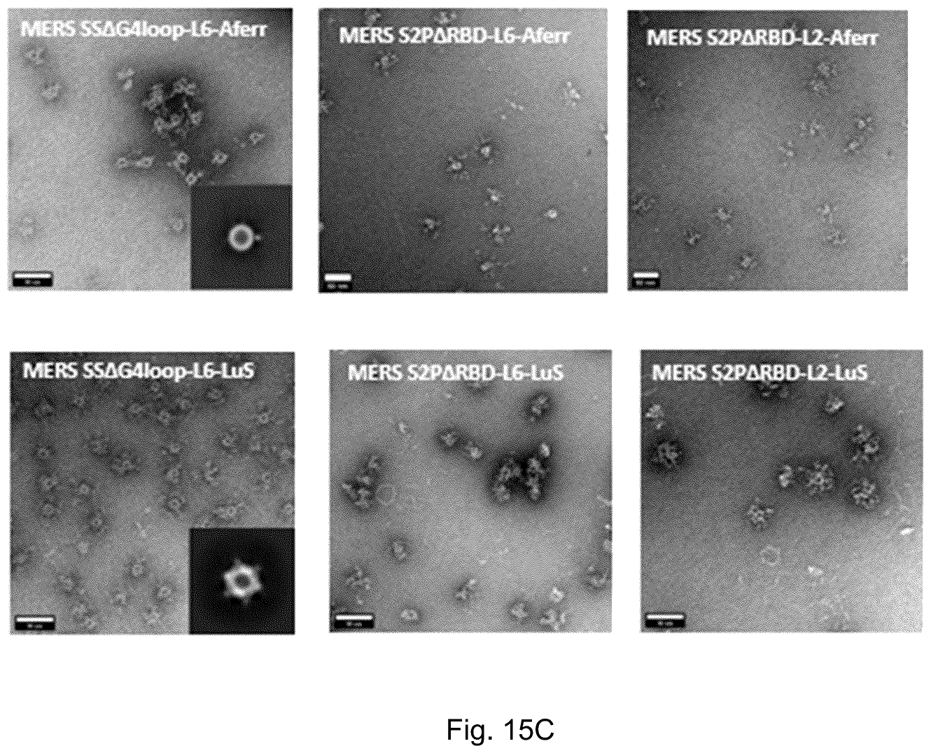

The pan-coronavirus vaccine development trajectory described herein involves: 1) antigen design to hone more cross-reactive responses, 2) nanoparticle display to optimize antigen display and maximize immunogenicity towards more cross-reactive responses, and 3) gene-based delivery to shorten manufacturing timeline. A workflow is depicted in FIGS. 13A-13E. Rational immunogen designs are depicted in FIGS. 3A-3C and 14. Structural characterization of nanoparticle immunogens is depicted in FIGS. 15A-15C.

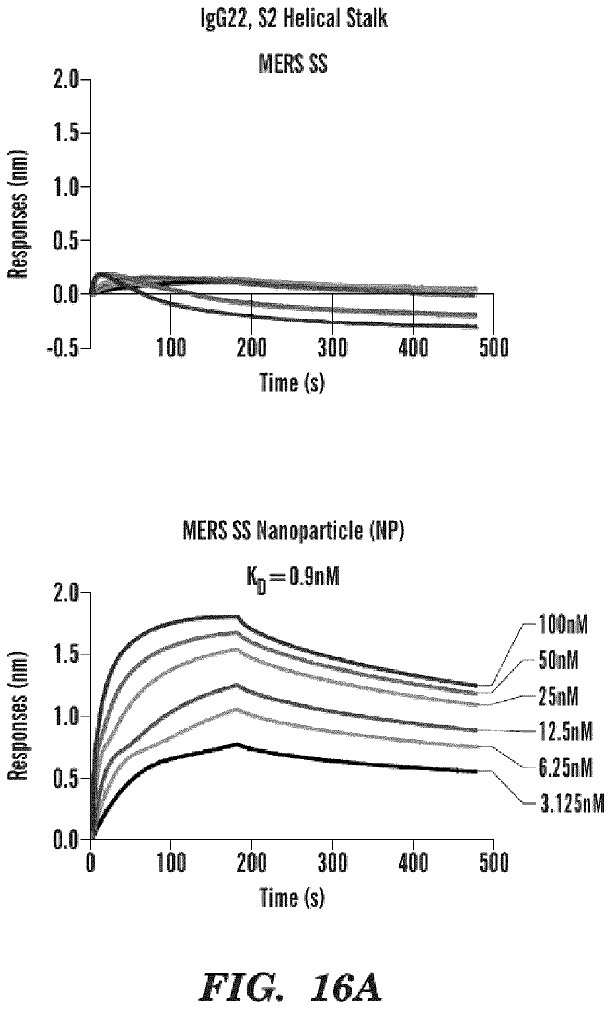

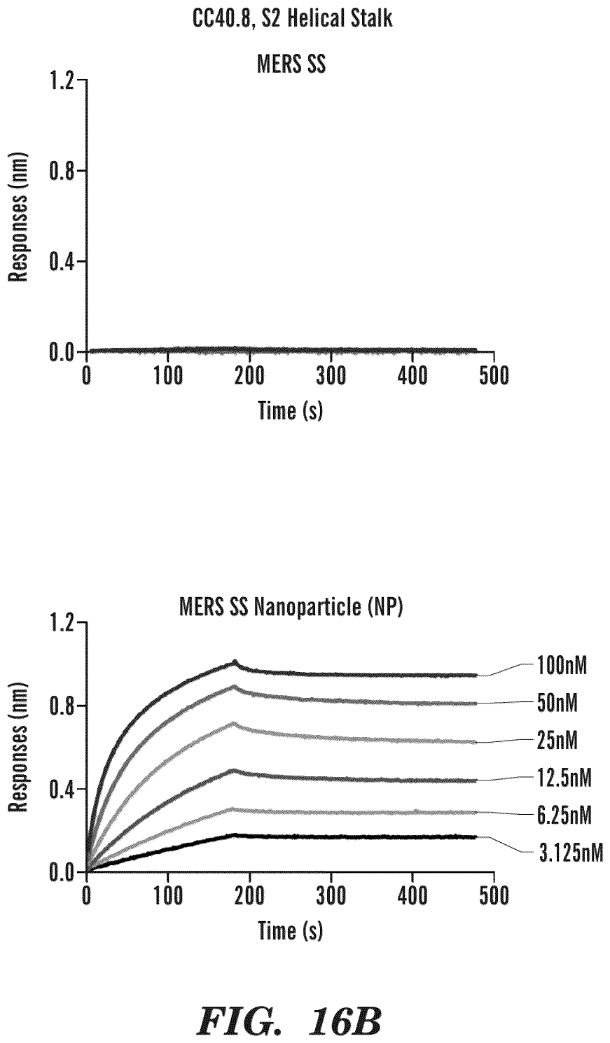

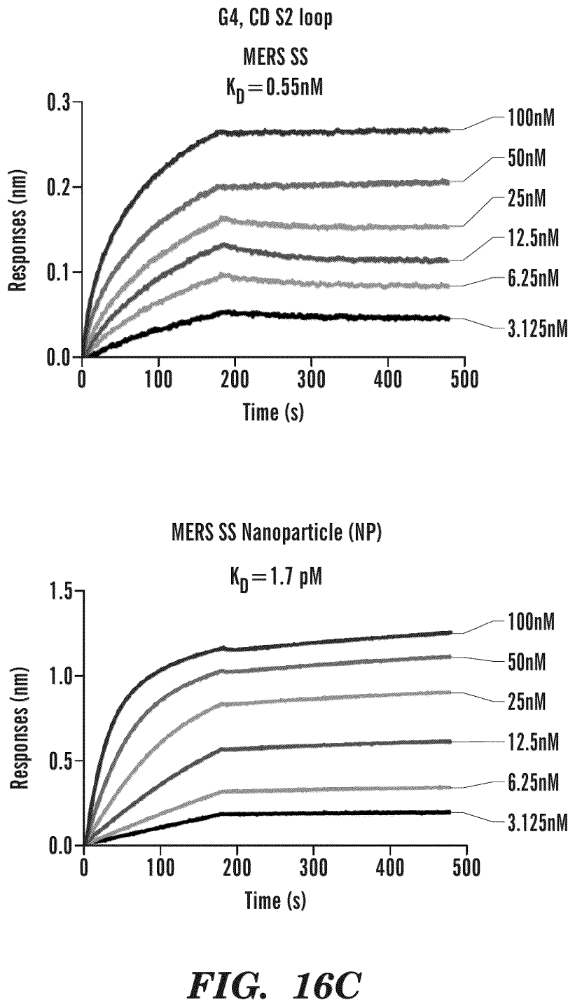

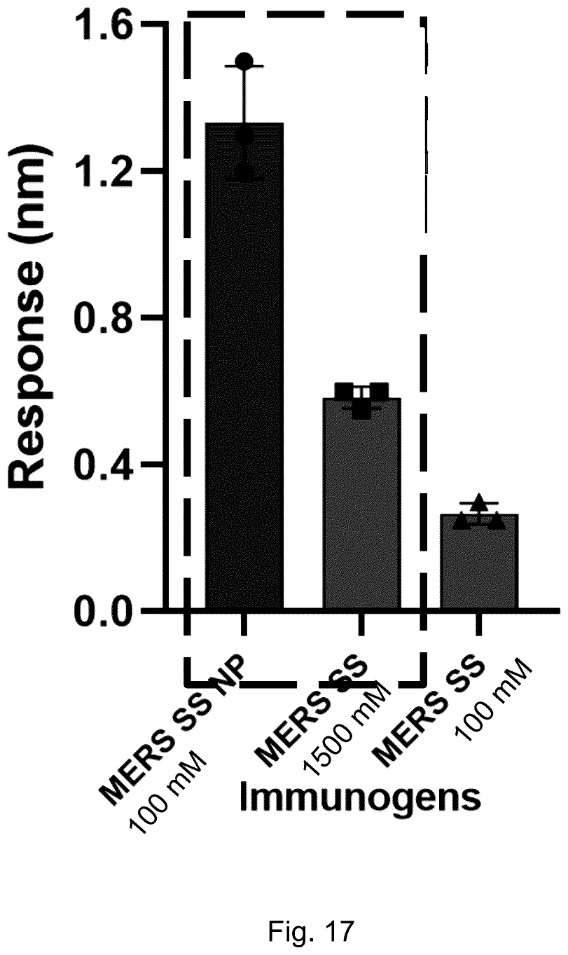

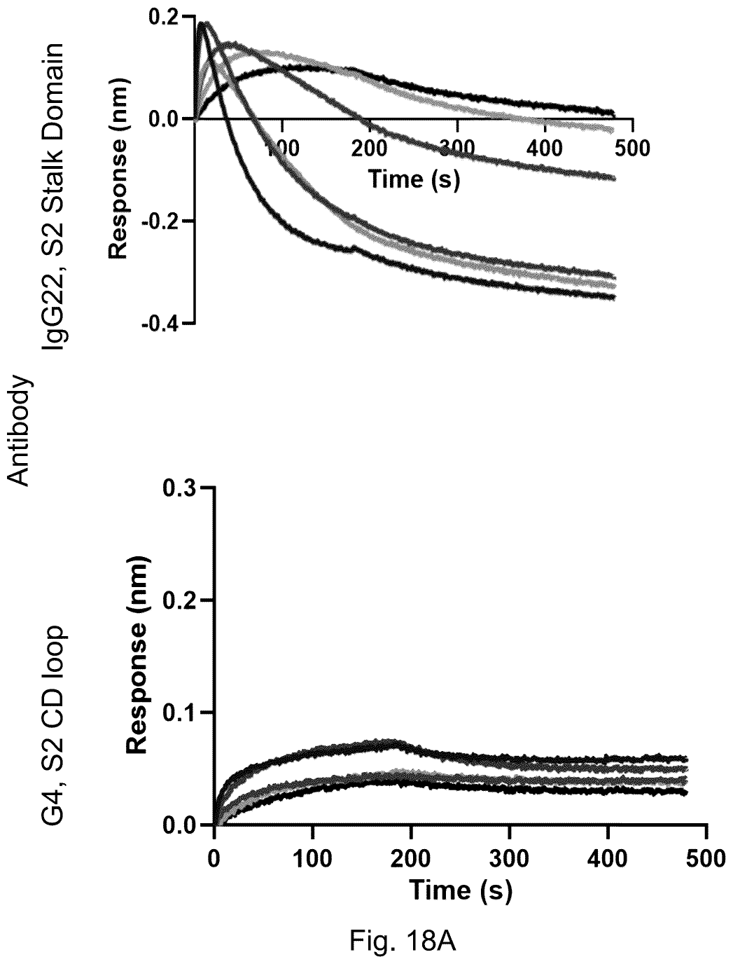

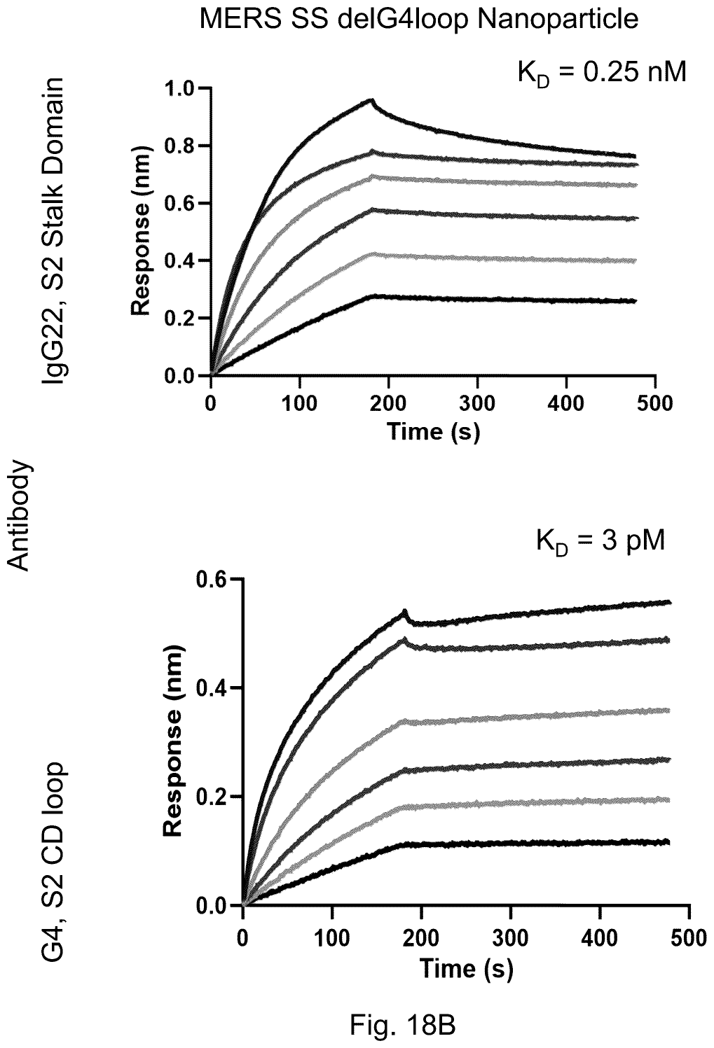

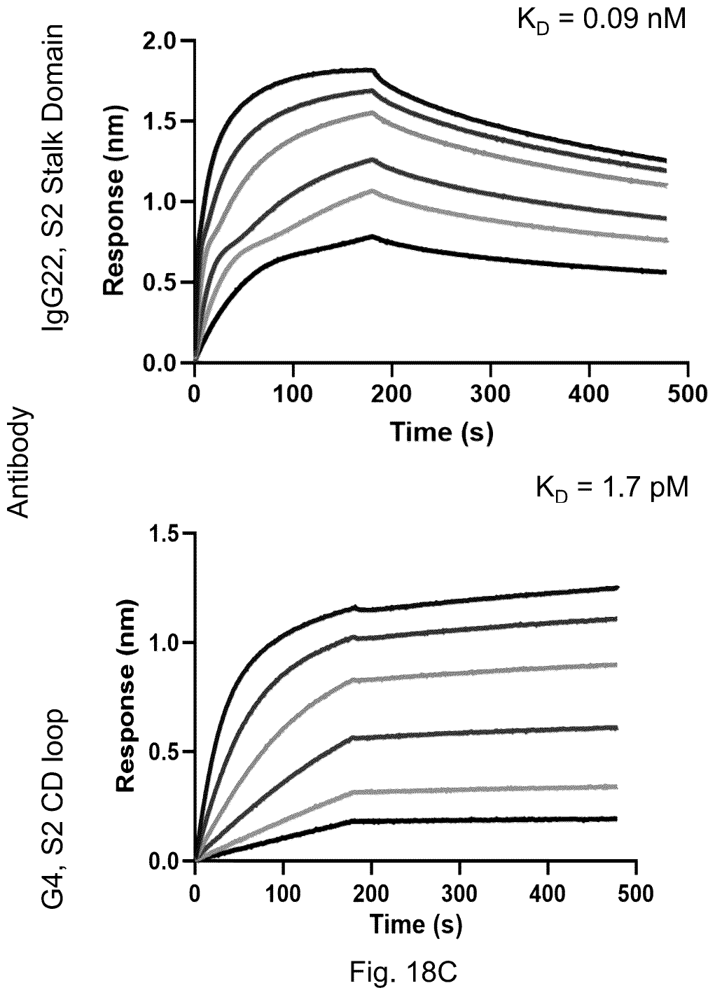

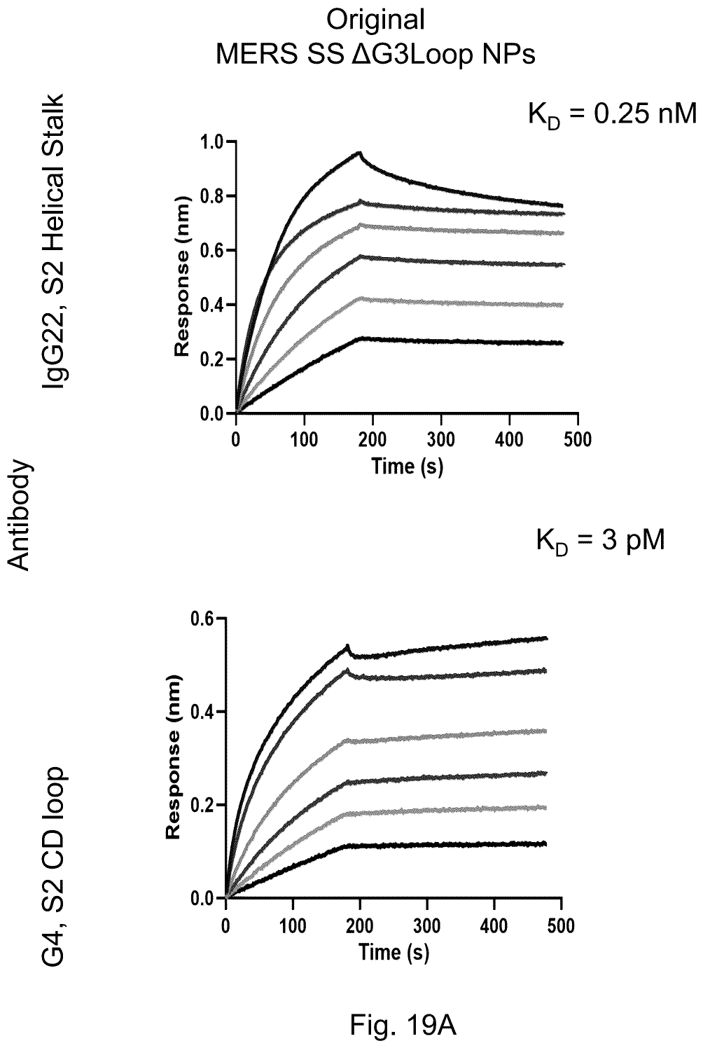

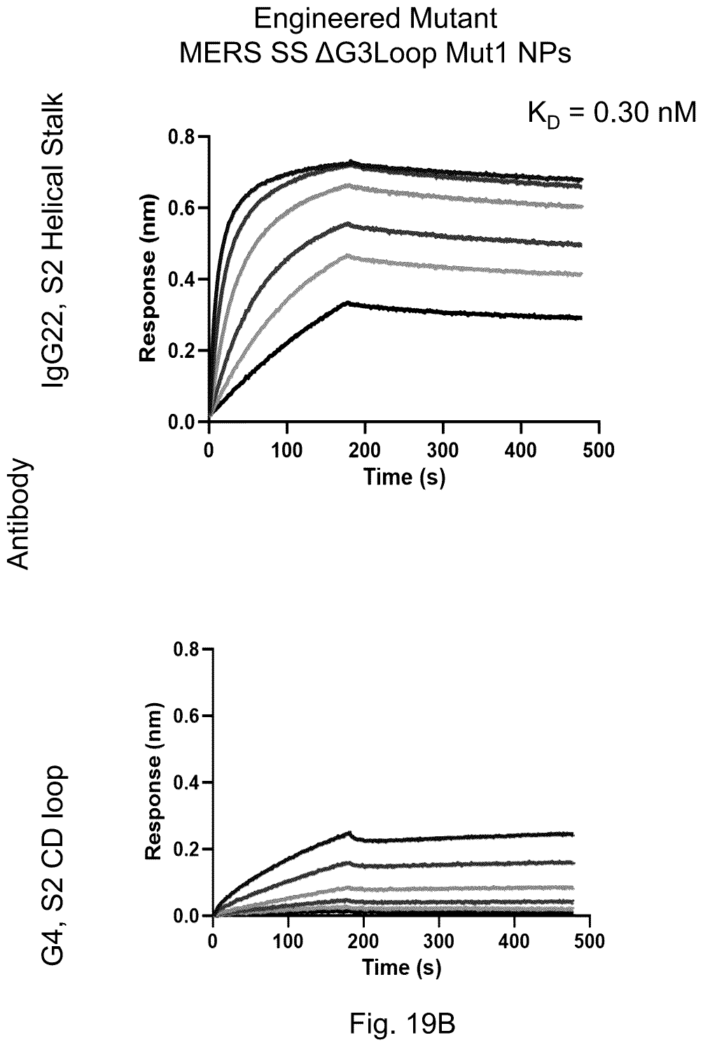

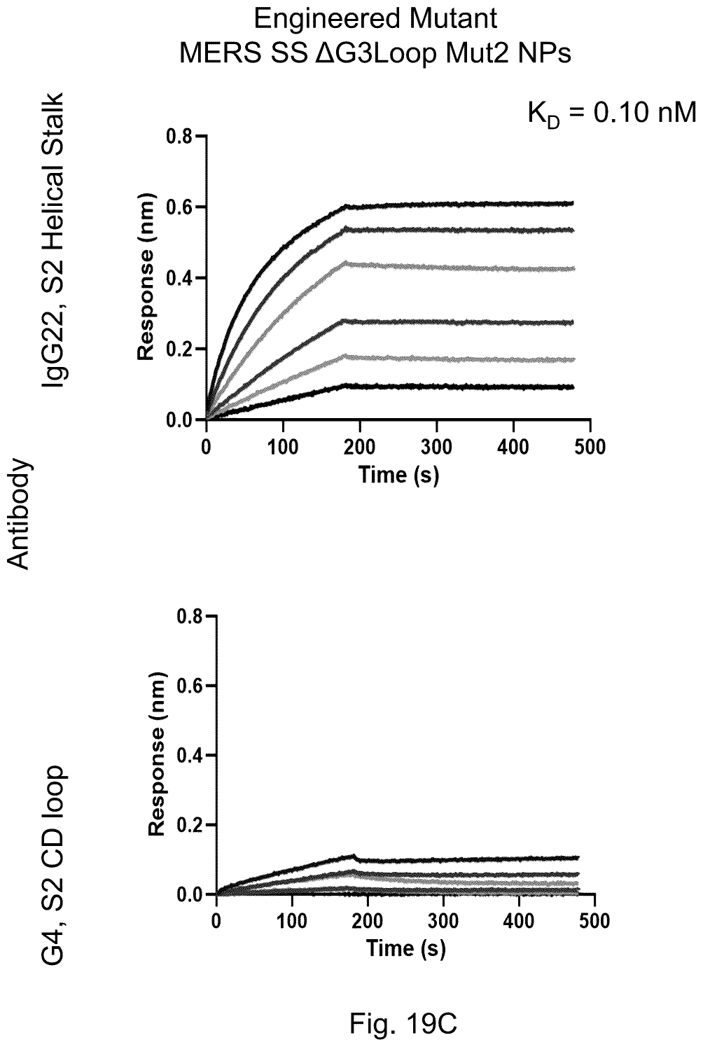

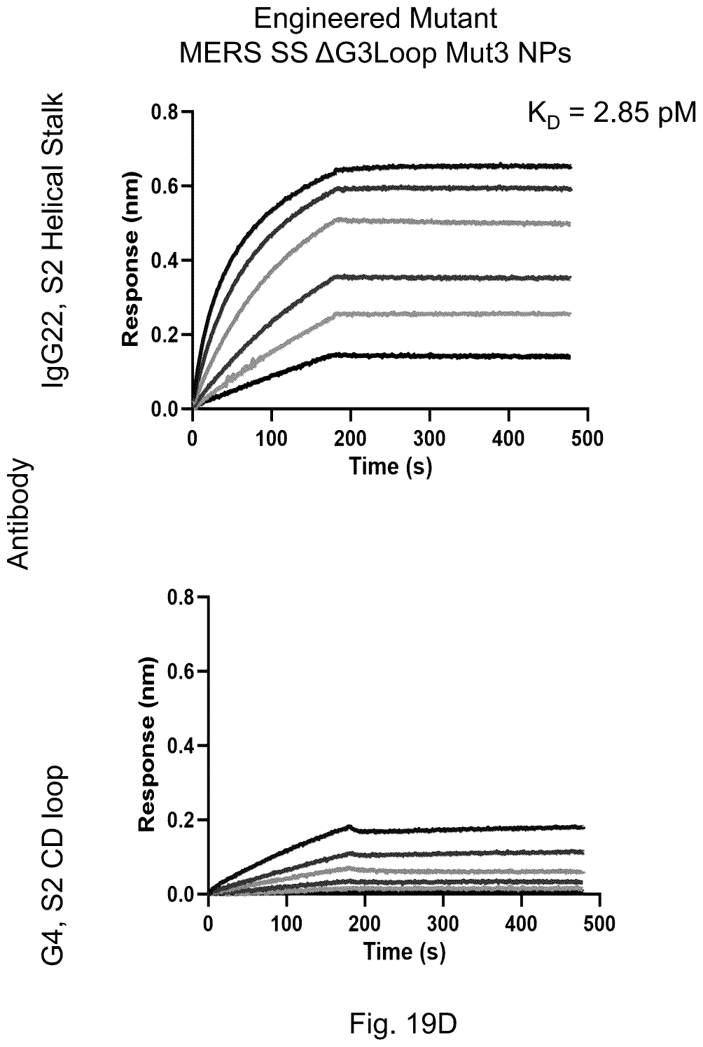

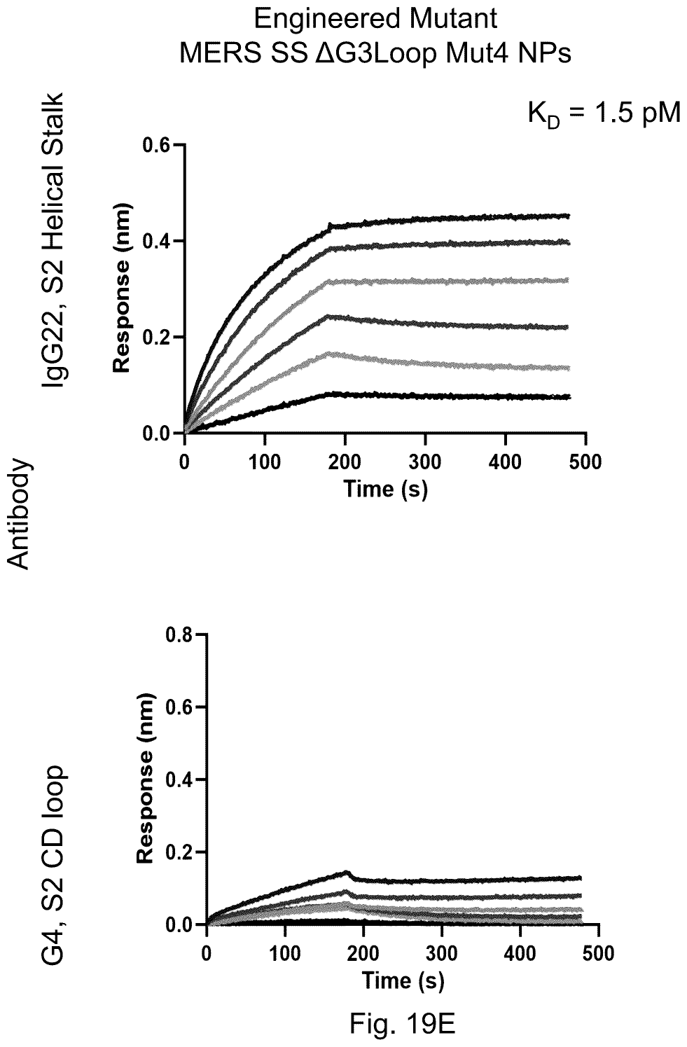

MERS SS nanoparticle vaccines were subjected to antigenic characterization (FIGS. 16, 17, 18, 19).

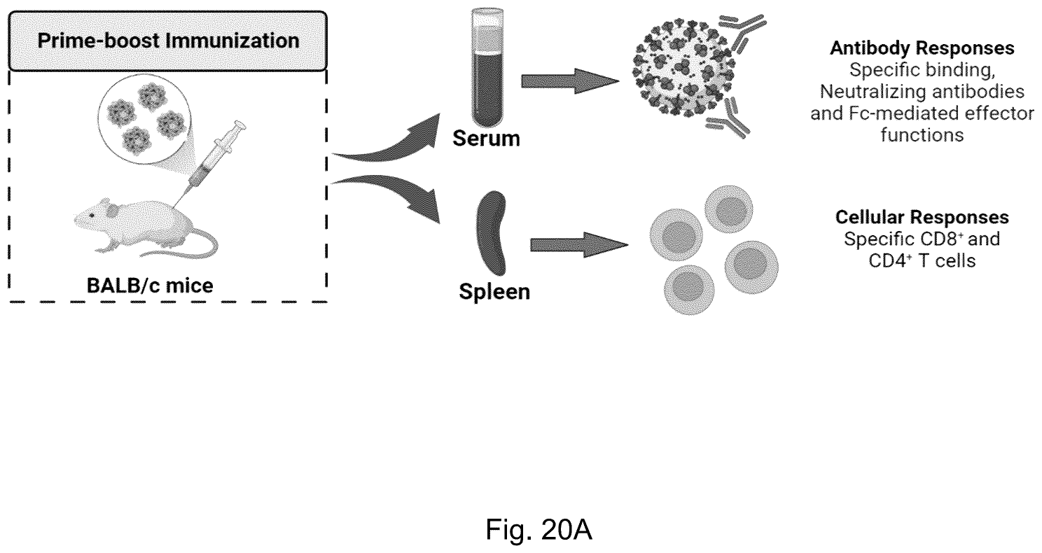

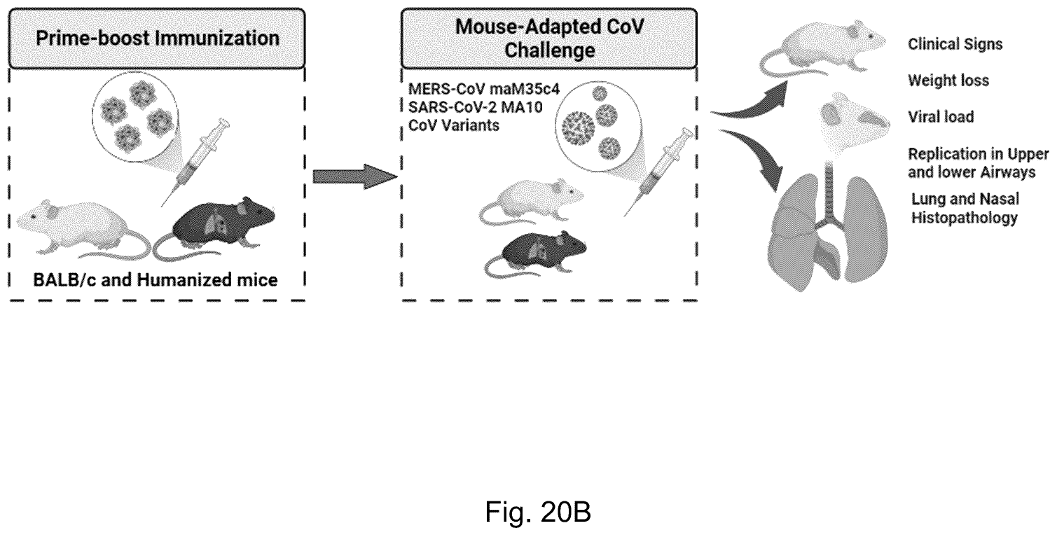

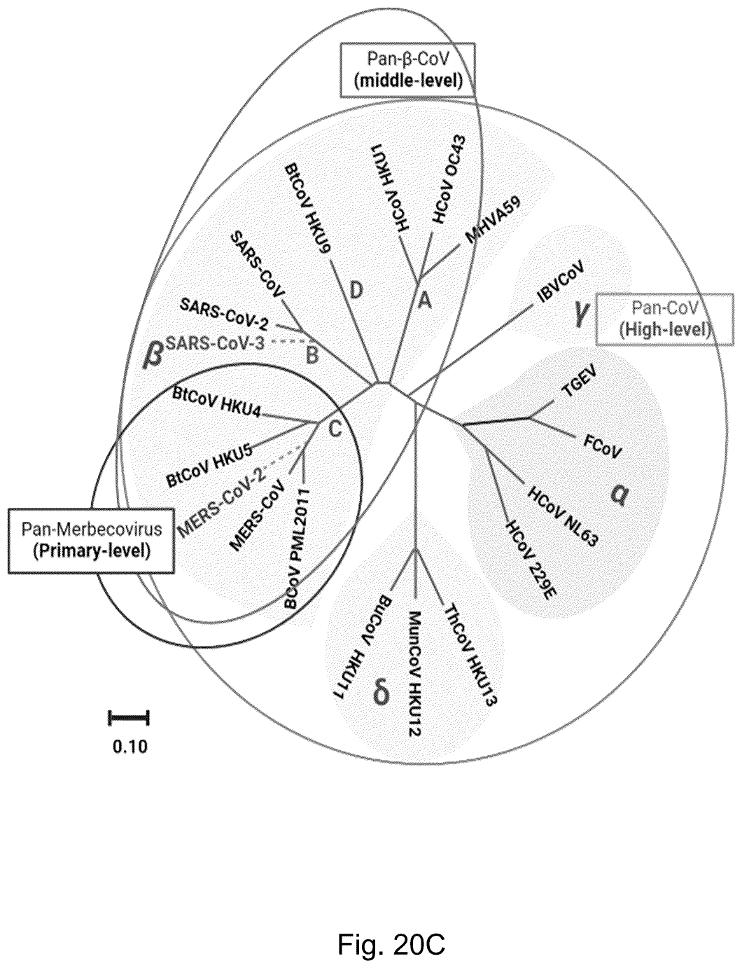

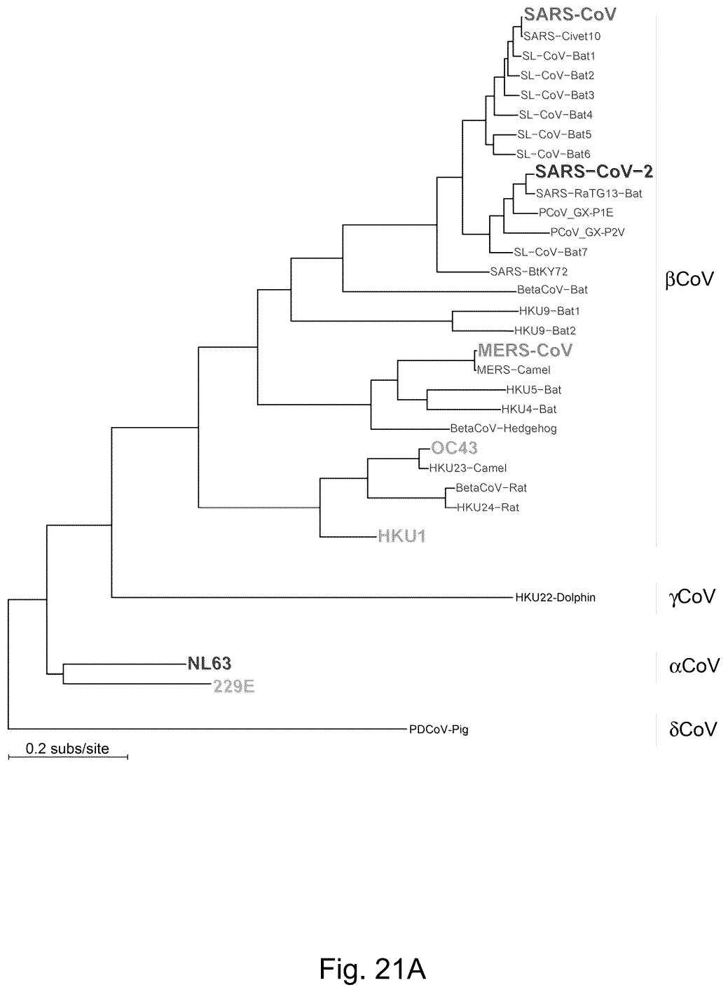

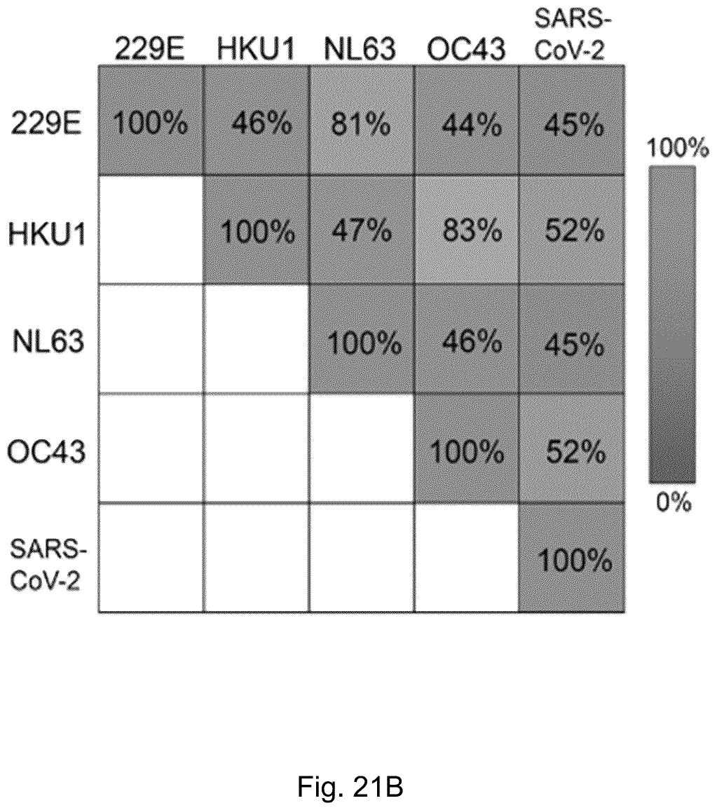

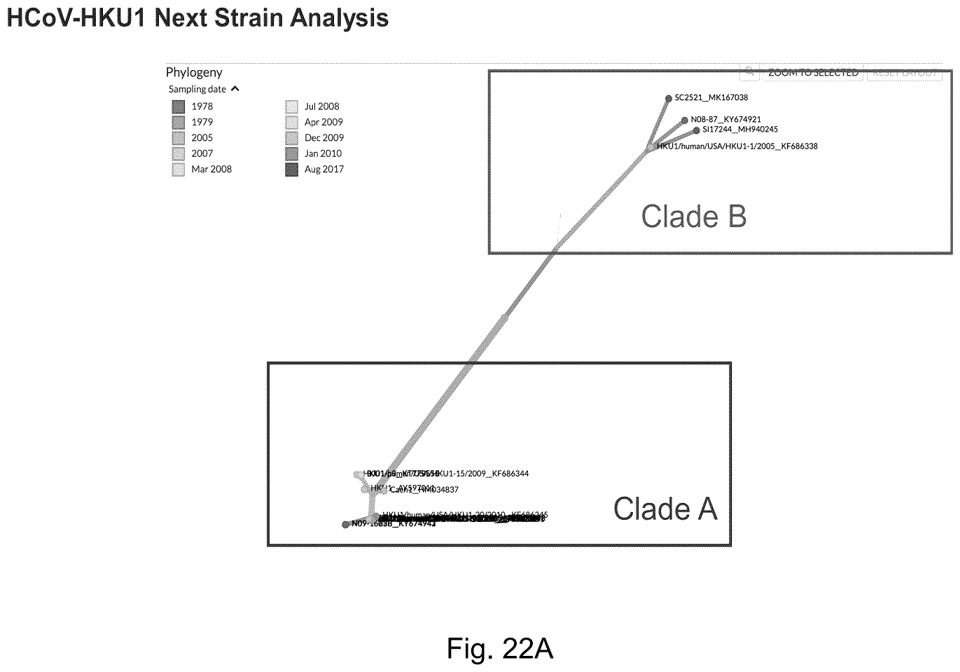

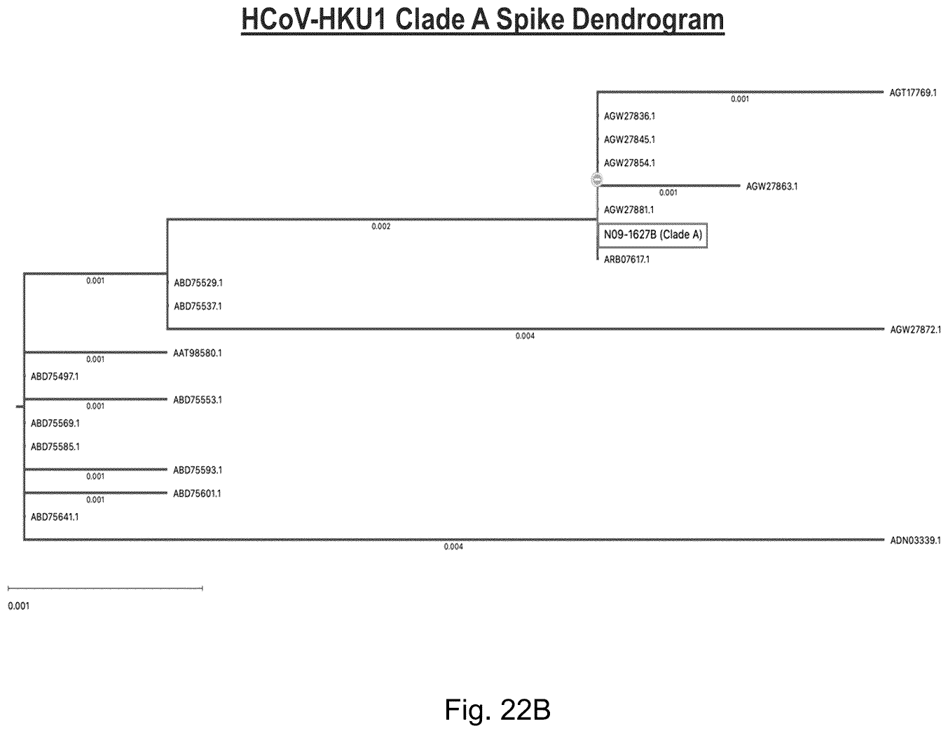

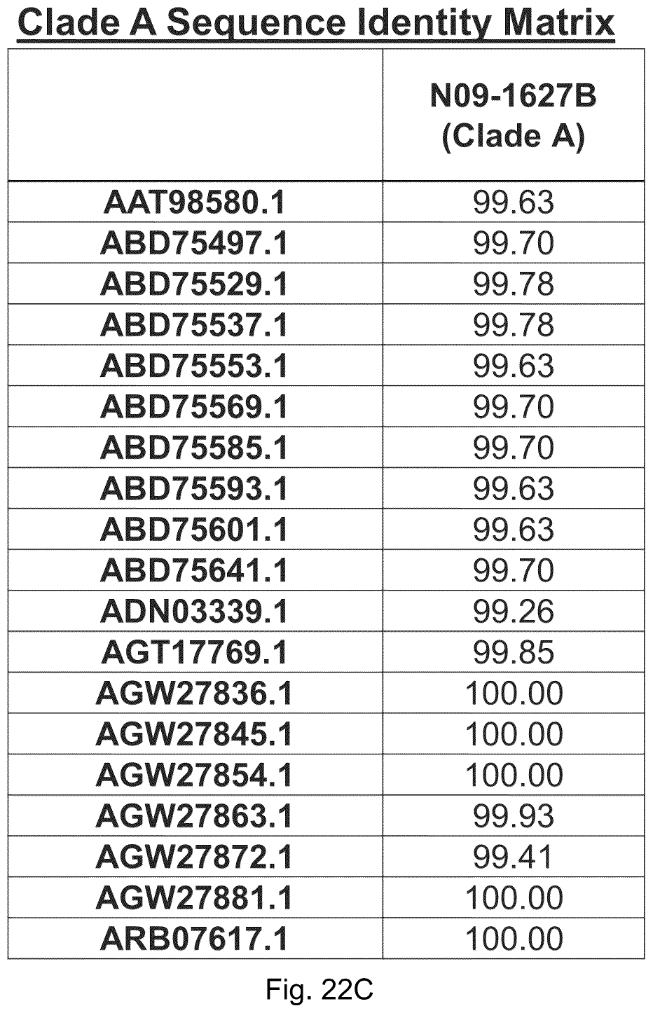

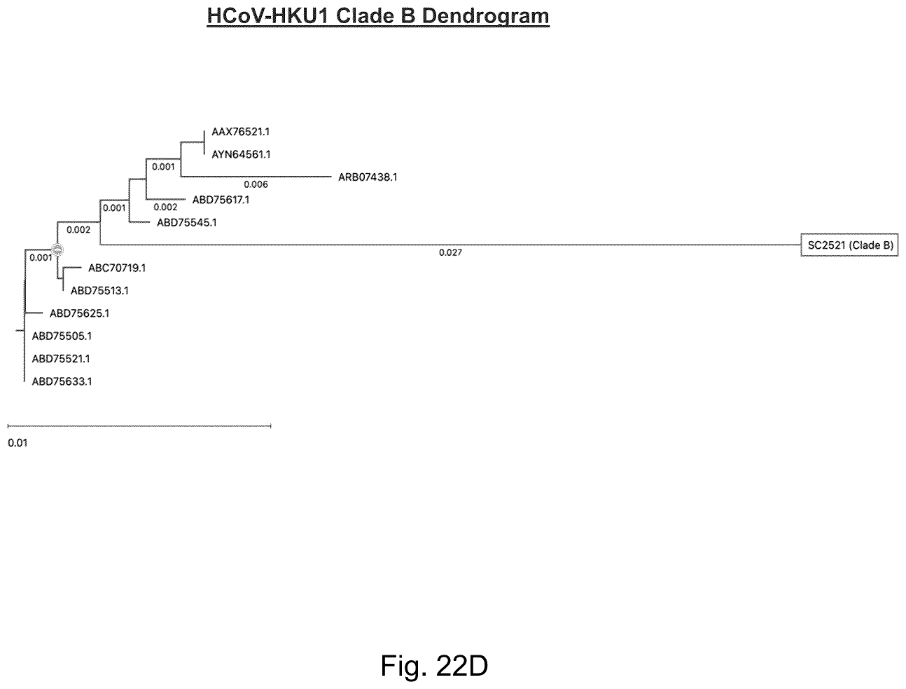

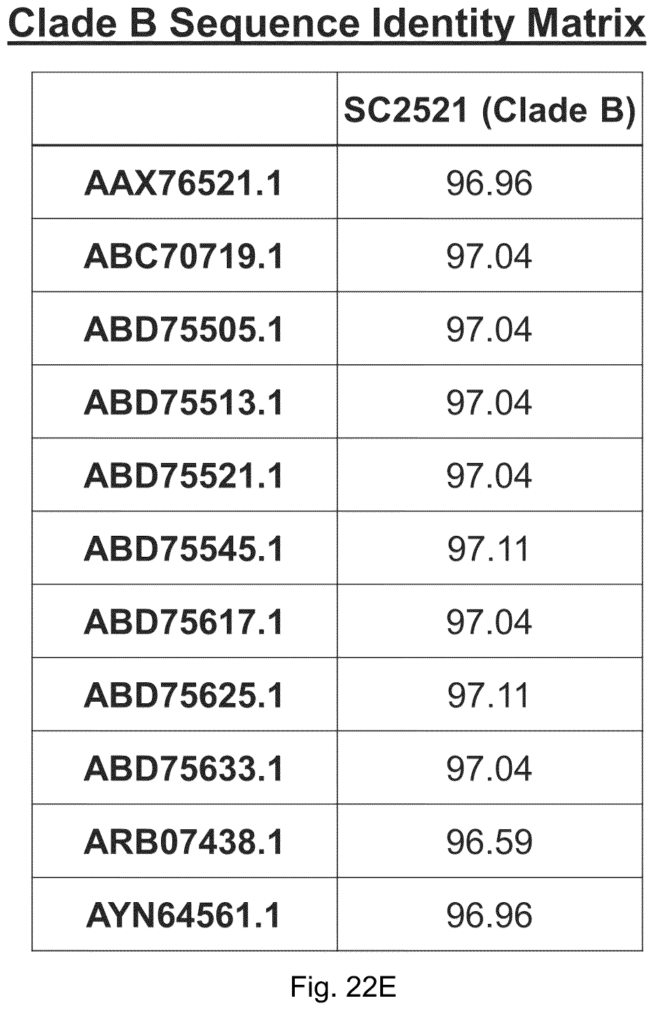

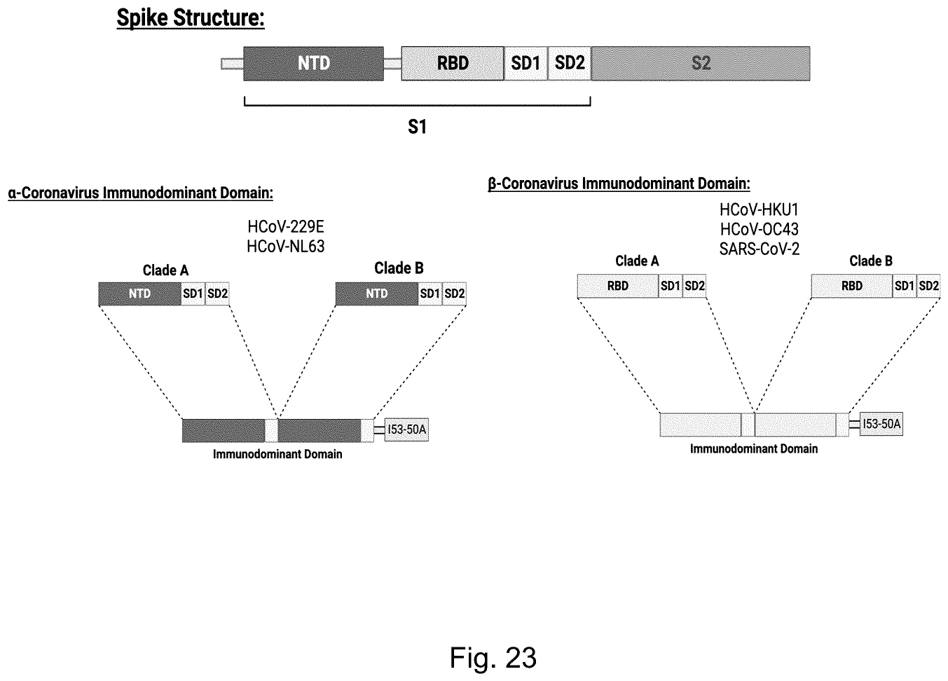

Vaccine candidates can be further characterized by evaluating pan-nanoparticle vaccines' immunogenicity in mouse models (FIG. 20A), evaluating pan-nanoparticle vaccines' protective efficacy in mouse models (FIG. 20B), and establishing pan-CoV assessment levels (FIG. 20C).

### Example 4: Evaluation of a Novel Linker to Display Antigens on Nanoparticles Towards Exposure of Cross-Reactive Antibody Epitopes

Current coronavirus (CoV) vaccines primarily induce antibody responses directed against the immunodominant spike (S) receptor binding domain (RBD). RBD-specific antibody responses are readily escapable as CoVs mutate due to genetic drift and shift, thus vaccines that target RBD are restricted in breadth against divergent CoV strains. The CoV S stem is highly conserved across the seven human CoVs. To focus antibody responses towards conserved S stem, stabilized S stem (SS) immunogens were developed and displayed on self-assembling protein nanoparticles (NPs), using novel linkers. These linkers were designed to optimally project SS from nanoparticle cores and expose occluded conserved epitopes. Structural determination and antigenic characterization of CoV-SS-NPs confirmed exposure of cross-reactive SS epitopes, illustrating the potential of these vaccines to protect against current and emerging CoVs. Described herein is the evaluation of CoV-SS-NPs to validate immunogenicity and broad protection and cross-reactive antibody responses in mice.

Coronaviruses (CoVs) cause severe morbidity and mortality in humans, as poignantly exemplified by the COVID-19 pandemic. In the past two decades, three highly pathogenic coronaviruses (CoVs), severe acute respiratory syndrome coronavirus (SARS-CoV), Middle East respiratory syndrome coronavirus (MERS-CoV), and, most recently, SARS-CoV-2, have spilt over into humans. In addition, four human coronaviruses (hCoVs), namely hCoV-HKU1, hCoV-229E, hCoV-NL63, and hCoV-OC43, circulate endemically causing relatively mild respiratory disease. Notwithstanding, novel CoVs are constantly poised for human emergence [1-3]. While an arsenal of COVID-19 vaccines has been developed, the antigenic discordance between current vaccines and circulating variants has threatened their efficacy. Although strategies of updating vaccines with spike (S) proteins of circulating strains have been employed, this is not an optimal solution for universal protection against the coronaviridae family [4-7].

CoVs engage with host cell receptors using a trimeric spike (S) protein of S1-S2 heterodimers—an interaction that facilitates membrane fusion, viral entry, and subsequent viral replication [8]. Current CoV vaccines primarily induce immune responses to the hypervariable S1 (head) region. These responses provide protection against the vaccinating strain but little to no cross-reactivity to other CoV strains or subtypes. Conversely, the immunosubdominant yet conserved CoV S2 (stem) harbors cross-reactive supersites representing the prime target for universal vaccines. Such cross-reactive S2 epitopes are either in the S2 apex [9] or the membrane proximal region; both areas are occluded by the S1 head [10]. Compared to vaccination only, it was recently observed that SARS-CoV-2-infected individuals vaccinated with mRNA or vector-based SARS-CoV-2 vaccines were effective at generating and augmenting unique S2-targeted antibodies [11]. These antibodies and B-cells are rare, only ˜5-10% of the human population produce them at significant levels, and their elicitation requires unmet rational vaccine design strategies.

### Summary of Nanoparticle Linker Designs.

| Linker Type | Sequence | Reference | SEQ ID NO |
|---|---|---|---|
| Short Flexible | GSG; SGG | [12] [13, 14] [15] [16] | |
| [symbol] | | | |
| Long Flexible (L6) | GGGSGGGGSGGGGSGLS | [17] | 9 |
| [symbol] | K | | |
| Helical Rigid (L2) | GGGGSAEAAAKEAAAKE | | 2 |
| [symbol] | AAAKAGGGGS | | |

Immunodominance of CoV S head in CoV vaccines that are licensed or under-development impedes the induction of cross-reactive protective stem-directed antibodies. New technologies are required to improve CoV vaccines, enabling cross-reactive immunity induction [18]. Such technologies come in two flavors: (1) antigen design optimizations to focus on cross-reactive antibody epitopes and (2) nanoparticle display to augment immune responses, enable optimal spatial organization for B-cell engagement, and allow multivalent display of multiple antigens [19, 20]. Nanoparticles displaying other viral proteins, including the inventor's own influenza SapNP with a ferritin core, have utilized short flexible linkers to protrude antigens [15]. Current CoV nanoparticle vaccines consist of antigens displayed on nanoparticles with flexible, random-coiled linkers (Table above). Neither of the aforementioned linkers allow for targeted antigen presentation thus rarely inducing high-level antibody responses toward conserved S2 epitopes [9, 10, 21-24].

To that end, described herein are structural helical linkers (e.g., SEQ ID NO: 2) that will permit optimal orientation of CoV S antigens displayed from nanoparticles allowing for targeting of cross-reactive antibody epitopes. As proof of concept, stabilized CoV S stem (SS) immunogens lacking the immunodominant head were utilized to focus immune responses on subdominant but conserved S stem on self-assembling protein nanoparticles (SapNs).

These novel vaccine candidates possess homogeneous conformation, morphology, and cross-reactive antigenicity profiles. The immunogenicity of the helical rigid linker (L2) can be assessed in comparison to the field standard long flexible linker (L6). The studies will determine whether the novel L2 linkers improve the immunogenicity and cross-reactivity of CoV SS SapNPs compared to soluble SS protein and SS SapNPs made with classical L2 linkers.

Evaluate the immunogenicity of CoV stem SapNPs containing various linker technology. Humoral antibody responses are sufficient and correlative to protection against CoVs [25, 26]. Mice will be immunized with soluble CoV SS, SS L2 SapNPs, or SS L6 SapNPs to compare antibody responses. Cross-reactivity will be assessed using S antigens from all seven human CoVs and relevant zoonotic CoVs, such as WIV1, which has been deemed poised for emergence [27]. Cross-reactivity will be screened using binding and neutralizing antibody assays. Additional antibody functionality will be assessed, such as Fc-mediated functions, which are characteristic of previously characterized S2 responses [9]. The antibody responses will be further dissected for quantity of cross-reactive epitopes using competition assays.

Evaluate B-cell responses following immunization with CoV SapNPs containing novel linker technology. Driving optimal CoV vaccine-induced antibody responses is dependent on optimal B-cell responses, though sometimes B-cell signatures are not apparent in serum antibody assessments. To truly gauge the ability of the instant novel linker technology to excite optimal B-cell responses, antibody production will be quantified at the cellular level and antibody responses of particular B-cell subtypes characterized using ELISpot and flow cytometry. Finally, vaccine-elicited monoclonal antibodies will be isolated and characterized to reveal novel epitopes. While these linkers have been preliminary assessed for antigenicity relevant to known cross-reactive epitopes, in addition to known epitopes, these novel linkers expose unexplored epitopes. Thus, B-cells of L2 CoV SS SapNP-immunized mice will be probed to isolate and characterize monoclonal antibody responses. This exercise will not only further validate the linker as superior over others but also inform future linker design iterations to improve antibody epitope targeting.

Beyond vaccines, these nanoparticles using the instant linkers can be used as tools to decipher CoV immune responses and isolate novel monoclonal antibodies for therapeutic interventions. There are currently CoV nanoparticles in clinical trial [28] and many companies, such as GlaxoSmithKline and Vaccines, Inc., are pre-clinically evaluating CoV nanoparticles, highlighting the immediate utility of this linker technology. Nanoparticles displaying other viral fusion proteins, such as HIV Env, would benefit from L2 linker technology as cross-reactive HIV antibodies are occluded on the N-terminal region of the HIV-1 fusion peptide [29]. In all, L2 linker technology has broad applicability across viral families and perhaps even bacterial nanoparticle vaccines and therapeutics. The L2 linker is effectively the missing link to universal vaccine development and discovery of broadly protective monoclonal antibodies.

### REFERENCE LIST

1. Menachery, V. D., et al., A SARS-like cluster of circulating bat coronaviruses shows potential for human emergence. Nature medicine, 2015. 21(12): p. 1508-1513.
2. Menachery, V. D., et al., SARS-like WIV1-CoV poised for human emergence. Proceedings of the National Academy of Sciences, 2016. 113(11): p. 3048-3053.
3. Xiong, Q., et al., Close relatives of MERS-CoV in bats use ACE2 as their functional receptors. Nature, 2022: p. 1-10.
4. Cao, Y., et al., Imprinted SARS-CoV-2 humoral immunity induces convergent Omicron RBD evolution. Nature, 2023. 614(7948): p. 521-529.
5. Kurhade, C., et al., Low neutralization of SARS-CoV-2 Omicron BA. 2.75. 2, BQ. 1.1 andXBB. 1 by parental mRNA vaccine or a BA. 5 bivalent booster. Nature medicine, 2023. 29(2): p. 344-347.
6. Yue, C., et al., ACE2 binding and antibody evasion in enhanced transmissibility of XBB. 1.5. The Lancet Infectious Diseases, 2023. 23(3): p. 278-280.
7. Khoury, D. S., et al., Predicting the efficacy ofvariant-modified COVID-19 vaccine boosters. Nature Medicine, 2023. 29(3): p. 574-578.
8. Li, F., Structure, function, and evolution of coronavirus spike proteins. Annual review of virology, 2016. 3: p. 237-261.
9. Johnson, N. V., et al., Discovery and Characterization of a Pan-betacoronavirus S2-binding antibody. bioRxiv, 2024: p. 2024.01.15.575741.
10. Hsieh, C. L., et al., Stabilized coronavirus spike stem elicits a broadly protective antibody. Cell Rep, 2021. 37(5): p. 109929.
11. Kaplonek, P., et al., Hybrid immunity expands the functional humoral footprint of both mRNA and vector-based SARS-CoV-2 vaccines. Cell Reports Medicine, 2023. 4(5).
12. Kanekiyo, M., et al., Self-assembling influenza nanoparticle vaccines elicit broadly neutralizing H1N1 antibodies. Nature, 2013. 499(7456): p. 102-106.
13. Yassine, H. M., et al., Hemagglutinin-stem nanoparticles generate heterosubtypic influenza protection. Nature Medicine, 2015. 21(9): p. 1065-1070.
14. Kanekiyo, M., et al., Mosaic nanoparticle display of diverse influenza virus hemagglutinins elicits broad B cell responses. Nature Immunology, 2019. 20(3): p. 362-372.
15. Corbett, K. S., et al., Design of Nanoparticulate Group 2 Influenza Virus Hemagglutinin Stem Antigens That Activate Unmutated Ancestor B Cell Receptors of Broadly Neutralizing Antibody Lineages. mBio, 2019. 10(1).
16. Moin, S. M., et al., Co-immunization with hemagglutinin stem immunogens elicits cross-group neutralizing antibodies and broad protection against influenza A viruses. Immunity, 2022. 55(12): p. 2405-2418.e7.
17. Mu, Z., et al., mRNA-encoded HIV-1 Env trimer ferritin nanoparticles induce monoclonal antibodies that neutralize heterologous HIV-1 isolates in mice. Cell Rep, 2022. 38(11): p. 110514.
18. Dolgin, E., Pan-coronavirus vaccine pipeline takes form. Nat. Rev. Drug Discov, 2022. 21(5): p. 324-326.
19. López-Sagaseta, J., et al., Self-assembling protein nanoparticles in the design of vaccines. Computational and structural biotechnology journal, 2016. 14: p. 58-68.
20. Nguyen, B. and N. H. Tolia, Protein-based antigen presentation platforms for nanoparticle vaccines. NPJ vaccines, 2021. 6(1): p. 70-70.
21. Zhang, B., et al., A platform incorporating trimeric antigens into self-assembling nanoparticles reveals SARS-CoV-2-spike nanoparticles to elicit substantially higher neutralizing responses than spike alone. Scientific reports, 2020. 10(1): p. 18149.
22. Hutchinson, G., et al., Nanoparticle display of prefusion coronavirus spike elicits S1-focused cross-reactive protection across divergent subgroups. 2022.
23. Carreño, J. M., et al., An inactivated NDV-HXP-S COVID-19 vaccine elicits a higher proportion of neutralizing antibodies in humans than mRNA vaccination. Science Translational Medicine, 2023. 15(683): p. eabo2847.
24. Brinkkemper, M., et al., Co-display of diverse spike proteins on nanoparticles broadens sarbecovirus neutralizing antibody responses. Iscience, 2022. 25(12): p. 105649.
25. Perry, J., et al., Does a humoral correlate of protection exist for SARS-CoV-2?A systematic review. PLoS One, 2022. 17(4): p. e0266852.
26. Qi, H., et al., The humoral response and antibodies against SARS-CoV-2 infection. Nature Immunology, 2022. 23(7): p. 1008-1020.
27. Menachery, V. D., et al., SARS-like WIVJ-CoV poised for human emergence. Proc Natl Acad Sci USA, 2016. 113(11): p. 3048-53.
28. Rauf, A., et al., Nanoparticles in clinical trials of COVID-19: An update. Int J Surg, 2022. 104: p. 106818.
29. Xu, K., et al., Epitope-based vaccine design yields fusion peptide-directed antibodies that neutralize diverse strains of HIV-1. Nature Medicine, 2018. 24(6): p. 857-867.

### Example 5

Recombinant spike protein sequences derived from the indicated coronavirus prefix. S-2P suffix indicates that the construct is a soluble prefusion-stabilized spike protein using two-proline mutations. The _dn5b suffix indicates that the prefusion-stabilized spikes are displayed on the I53_dn5 scaffold through genetic fusion to the I53_dn5b component. Linker indicates a short flexible linker between the spike and I53_dn5b scaffold component.

MERS_S-2P (SEQ ID NO: 78):
MIHSVFLLMFLLTPTESYVDVGPDSVKSACIEVDIQQTFFDKTWPRPIDVSKADGIIYPQGRTYSNITITYQGLFP
YQGDHGDMYVYSAGHATGTTPQKLFVANYSQDVKQFANGFVVRIGAAANSTGTVIISPSTSATIRKIYPAFML
GSSVGNFSDGKMGRFFNHTLVLLPDGCGTLLRAFYCILEPRSGNHCPAGNSYTSFATYHTPATDCSDGNYNRN
ASLNSFKEYFNLRNCTFMYTYNITEDEILEWFGITQTAQGVHLFSSRYVDLYGGNMFQFATLPVYDTIKYYSIIPH
SIRSIQSDRKAWAAFYVYKLQPLTFLLDFSVDGYIRRAIDCGFNDLSQLHCSYESFDVESGVYSVSSFEAKPSGSV
VEQAEGVECDFSPLLSGTPPQVYNFKRLVFTNCNYNLTKLLSLFSVNDFTCSQISPAAIASNCYSSLILDYFSYPLS
MKSDLSVSSAGPISQFNYKQSFSNPTCLILATVPHNLTTITKPLKYSYINKCSRFLSDDRTEVPQLVNANQYSPCV
SIVPSTVWEDGDYYRKQLSPLEGGGWLVASGSTVAMTEQLQMGFGITVQYGTDTNSVCPKLEFANDTKIASQ
LGNCVEYSLYGVSGRGVFQNCTAVGVRQQRFVYDAYQNLVGYYSDDGNYYCLRACVSVPVSVIYDKETKTHAT
LFGSVACEHISSTMSQYSRSTRSMLKRRDSTYGPLQTPVGCVLGLVNSSLFVEDCKLPLGQSLCALPDTPSTLTP
ASVGSVPGEMRLASIAFNHPIQVDQLNSSYFKLSIPTNFSFGVTQEYIQTTIQKVTVDCKQYVCNGFQKCEQLL
REYGQFCSKINQALHGANLRQDDSVRNLFASVKSSQSSPIIPGFGGDFNLTLLEPVSISTGSRSARSAIEDLLFDK
VTIADPGYMQGYDDCMQQGPASARDLICAQYVAGYKVLPPLMDVNMEAAYTSSLLGSIAGVGWTAGLSSFA
AIPFAQSIFYRLNGVGITQQVLSENQKLIANKFNQALGAMQTGFTTTNEAFHKVQDAVNNNAQALSKLASEL
SNTFGAISASIGDIIQRLDPPEQDAQIDRLINGRLTTLNAFVAQQLVRSESAALSAQLAKDKVNECVKAQSKRSG
FCGQGTHIVSFVVNAPNGLYFMHVGYYPSNHIEVVSAYGLCDAANPTNCIAPVNGYFIKTNNTRIVDEWSYT
GSSFYAPEPITSLNTKYVAPQVTYQNISTNLPPPLLGNSTGIDFQDELDEFFKNVSTSIPNFGSLTQINTTLLDLTY
EMLSLQQVVKALNESYIDLKELGNYTYGSGYIPEAPRDGQAYVRKDGEWVLLSTFLGRSLEVLFQGPGHHHH
HHHHSAWSHPQFEKGGGSGGGGSGGSAWSHPQFEK
SARS_S-2P (SEQ ID NO: 79):
MFIFLLFLTLTSGSDLDRCTTFDDVQAPNYTQHTSSMRGVYYPDEIFRSDTLYLTQDLFLPFYSNVTGFHTINHTF
GNPVIPFKDGIYFAATEKSNVVRGWVFGSTMNNKSQSVIIINNSTNVVIRACNFELCDNPFFAVSKPMGTQTH
TMIFDNAFNCTFEYISDAFSLDVSEKSGNFKHLREFVFKNKDGFLYVYKGYQPIDVVRDLPSGFNTLKPIFKLPL
GINITNFRAILTAFSPAQDIWGTSAAAYFVGYLKPTTFMLKYDENGTITDAVDCSQNPLAELKCSVKSFEIDKGIY
QTSNFRVVPSGDVVRFPNITNLCPFGEVFNATKFPSVYAWERKKISNCVADYSVLYNSTFFSTFKCYGVSATKLN
DLCFSNVYADSFVVKGDDVRQIAPGQTGVIADYNYKLPDDFMGCVLAWNTRNIDATSTGNYNYKYRYLRHG
KLRPFERDISNVPFSPDGKPCTPPALNCYWPLNDYGFYTTTGIGYQPYRVVVLSFELLNAPATVCGPKLSTDLIK
NQCVNFNFNGLTGTGVLTPSSKRFQPFQQFGRDVSDFTDSVRDPKTSEILDISPCAFGGVSVITPGTNASSEVA
VLYQDVNCTDVSTAIHADQLTPAWRIYSTGNNVFQTQAGCLIGAEHVDTSYECDIPIGAGICASYHTVSLLRSTS
QKSIVAYTMSLGADSSIAYSNNTIAIPTNFSISITTEVMPVSMAKTSVDCNMYICGDSTECANLLLQYGSFCTQL
NRALSGIAAEQDRNTREVFAQVKQMYKTPTLKYFGGFNFSQILPDPLKPTKRSFIEDLLFNKVTLADAGFMKQ
YGECLGDINARDLICAQKFNGLTVLPPLLTDDMIAAYTAALVSGTATAGWTFGAGAALQIPFAMQMAYRENGI
GVTQNVLYENQKQIANQFNKAISQIQESLTTTSTALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGKGYHLMSFPQAA
PHGVVFLHVTYVPSQERNFTTAPAICHEGKAYFPREGVFVFNGTSWFITQRNFFSPQIITTDNTFVSGNCDVVI
GIINNTVYDPLQPELDSFKEELDKYFKNHTSPDVDLGDISGINASVVNIQKEIDRLNEVAKNLNESLIDLQELGKY
EQGSGYIPEAPRDGQAYVRKDGEWVLLSTFLGRSLEVLFQGPGHHHHHHHHSAWSHPQFEK
SARS-2_S-2P (SEQ ID NO: 80):
MFVFLVLLPLVSSQCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCNDPFLGVYYHKN
NKSWMESEFRVYSSANNCTFEYVSQPFLMDLEGKQGNFKNLREFVFKNIDGYFKIYSKHTPINLVRDLPQGFS
ALEPLVDLPIGINITRFQTLLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQPYRVVVLSFELLHA
PATVCGPKKSTNLVKNKCVNFNFNGLTGTGVLTESNKKFLPFQQFGRDIADTTDAVRDPQTLEILDITPCSFGG
VSVITPGTNTSNQVAVLYQDVNCTEVPVAIHADQLTPTWRVYSTGSNVFQTRAGCLIGAEHVNNSYECDIPIG
AGICASYQTQTNSPRRARSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFNFSQILPDPSKPSKRSFIE
DLLFNKVTLADAGFIKQYGDCLGDIAARDLICAQKFNGLTVLPPLLTDEMIAQYTSALLAGTITSGWTFGAGAA
LQIPFAMQMAYRFNGIGVTQNVLYENQKLIANQFNSAIGKIQDSLSSTASALGKLQDVVNQNAQALNTLVKQ
LSSNFGAISSVLNDILSRLDPPEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISGINASVVNIQKEIDRLN
EVAKNLNESLIDLQELGKYEQGSGYIPEAPRDGQAYVRKDGEWVLLSTFLGRSLEVLFQGPGHHHHHHHHSA
WSHPQFEK
HKU1_S-2P (SEQ ID NO: 81):
MFLIIFILPTTLAVIGDFNCTNSFINDYNKTIPRISEDVVDVSLGLGTYYVLNRVYLNTTLLFTGYFPKSGANFRDL
ALKGSIYLSTLWYKPPFLSDFNNGIFSKVKNTKLYVNNTLYSEFSTIVIGSVFVNTSYTIVVQPHNGILEITACQYT
MCEYPHTVCKSKGSIRNESWHIDSSEPLCLFKKNFTYNVSADWLYFHFYQERGVFYAYYADVGMPTTFLFSLYL
GTILSHYYVMPLTCNAISSNTDNETLEYWVTPLSRRQYLLNFDEHGVITNAVDCSSSFLSEIQCKTQSFAPNTGV
YDLSGFTVKPVATVYRRIPNLPDCDIDNWLNNVSVPSPLNWERRIFSNCNFNLSTLLRLVHVDSFSCNNLDKSK
IFGSCFNSITVDKFAIPNRRRDDLQLGSSGFLOSSNYKIDISSSSCQLYYSLPLVNVTINNFNPSSWNRRYGFGSF
NLSSYDVVYSDHCFSVNSDFCPCADPSVVNSCAKSKPPSAICPAGTKYRHCDLDTTLYVKNWCRCSCLPDPIST
YSPNTCPQKKVVVGIGEHCPGLGINEEKCGTQLNHSSCFCSPDAFLGWSFDSCISNNRCNIFSNFIFNGINSGT
TCSNDLLYSNTEISTGVCVNYDLYGITGQGIFKEVSAAYYNNWQNLLYDSNGNIIGFKDFLINKTYTILPCYSGRV
SAAFYQNSSSPALLYRNLKCSYVLNNISFISQPFYFDSYLGCVLNAVNLTSYSVSSCDLRMGSGFCIDYALPSSGG
SGSGISSPYRFVTFEPFNVSFVNDSVETVGGLFEIQIPTNFTIAGHEEFIQTSSPKVTIDCSAFVCSNYAACHDLLS
EYGTFCDNINSILNEVNDLLDITQLQVANALMQGVTLSSNLNTNLHSDVDNIDFKSLLGCLGSQCGSSSRSLLE
DLLFNKVKLSDVGFVEAYNNCTGGSEIRDLLCVQSFNGIKVLPPILSETQISGYTTAATVAAMFPPWSAAAGVP
FSLNVQYRINGLGVTMDVLNKNQKLIANAFNKALLSIQNGFTATNSALAKIQSVVNANAQALNSLLQQLFNKF
GAISSSLQEILSRLDPPEAQVQIDRLINGRLTALNAYVSQQLSDITLIKAGASRAIEKVNECVKSQSPRINFCGNG
NHILSLVQNAPYGLLFIHFSYKPTSFKTVLVSPGLCLSGDRGIAPKQGYFIKQNDSWMFTGSSYYYPEPISDKNV
VFMNSCSVNFTKAPFIYLNNSIPNLSDFEAELSLWFKNHTSIAPNLTENSHINATFLDLYYEMNVIQESIKSLNSG
RLEVLFQGPGGYIPEAPRDGQAYVRKDGEWVLLSTFLGHHHHHHHHSAWSHPQFEK
229E_S-2P (SEQ ID NO: 82):
MFVLLVAYALLHIAGCQTTNGTNTSHSVCNGCVGHSENVFAVESGGYIPSNFAFNNWFLLTNTSSVVDGVVRS
FQPLLLNCLWSVSGSQFTTGFVYFNGTGRGACKGFYSNASSDVIRYNINFEENLRRGTILFKTSYGAVVFYCTN
NTLVSGDAHIPSGTVLGNFYCFVNTTIGNETTSAFVGALPKTVREFVISRTGHFYINGYRYFSLGDVEAVNFNVT
NAATTVCTVALASYADVLVNVSQTAIANIIYCNSVINRLRCDQLSFDVPDGFYSTSPIQPVELPVSIVSLPVYHKH
TFIVLYVNFEHRRGPGKCYNCRPAVINITLANFNETKGPLCVDTSHFTTQFVDNVKLARWSASINTGNCPFSFG
KVNNFVKFGSVCFSLKDIPGGCAMPIMANLVNSKSHNIGSLYVSWSDGDVITGVPKPVEGVSSFMNVTLNKC
TKYNIYDVSGVGVIRISNDTFLNGITYTSTSGNLLGFKDVTNGTIYSITPCNPPDQLVVYQQAVVGAMLSENFTS
YGFSNVVEMPKFFYASNGTYNCTDAVLTYSSFGVCADGSIIAVQPRNVSYDSVSAIVTANLSIPFNWTTSVQVE
YLQITSTPIVVDCSTYVCNGNVRCVELLKQYTSACKTIEDALRNSAMLESADVSEMLTFDKKAFTLANVSSFGDY
NLSSVIPSLPRSGSRVAGRSAIEDILFSKLVTSGLGTVDADYKKCTKGLSIADLACAQYYNGIMVLPGVADAERMAM
YTGSLIGGIALGGLTSAASIPFSLAIQSRLNYVALQTDVLQENQRILAASFNKAMTNIVDAFTGVNDAITQTS
QALQTVATALNKIQDVVNQQGNSLNHLTSQLRQNFQAISSSIQAIYDRLDPPQADQQVDRLITGRLAALNVFV
SHTLTKYTEVRASRQLAQQKVNECVKSQSKRYGFCGNGTHIFSLVNAAPEGLVFLHTVLLPTQYKDVEAWSGL
CVDGINGYVLRQPNLALYKEGNYYRITSRIMFEPRIPTIADFVQIENCNVTFVNISRSELQTIVPEYIDVNKTLQEL
SYKLPNYTVPDLVVEQYNQTILNLTSEISTLENKSAELNYTVQKLQTLIDNINSTLVDLKWLNRVETGSGYIPEAP
RDGQAYVRKDGEWVLLSTFLGRSLEVLFQGPGHHHHHHHHSAWSHPQFEKGGGSGGGGSGGSAWSHPQ
FEK
OC43_S-2P (SEQ ID NO: 83):
MFLILLISLPTAFAVIGDLKCPLDSRTGSLNNIDTGPPSISTATVDVTNGLGTYYVLDRVYLNTTLFLNGYYPTSGS
TYRNMALKGTDKLSTLWFKPPFLSDFINGIFAKVKNTKVFKDGVMYSEFPAITIGSTFVNTSYSVVVQPRTINST
QDGVNKLQGLLEVSVCQYNMCEYPHTICHPKLGNHFKELWHMDTGVVSCLYKRNFTYDVNATYLYFHFYQE
GGTFYAYFTDTGVVTKFLFNVYLGMALSHYYVMPLTCISRRDIGFTLEYWVTPLTSRQYLLAFNQDGIIFNAVD
CMSDFMSEIKCKTQSIAPPTGVYELNGYTVQPIADVYRRKPDLPNCNIEAWLNDKSVPSPLNWERKTFSNCN
FNMSSLMSFIQADSFTCNNIDAAKIYGMCFSSITIDKFAIPNGRKVDLOLGNLGYLQSFNYRIDTTATSCQLYYN
LPAANVSVSRFNPSTWNKRFGFIENSVFKPQPAGVLTNHDVVYAQHCFKAPKNFCPCKLNSSLCVGSGPGKN
NGIGTCPAGTNYLTCHNLCNPDPITFTGPYKCPQTKSLVGIGEHCSGLAVKSDYCGGNPCTCQPQAFLGWSAD
SCLQGDKCNIFANLILHDVNSGLTCSTDLQKANTDIKLGVCVNYDLYGISGQGIFVEVNATYYNSWQNLLYDSN
GNLYGFRDYITNRTFMIRSCYSGRVSAAFHANSSEPALLFRNIKCNYVFNNSLIRQLQPINYFDSYLGCVVNAYN
STAISVQTCDLTVGSGYCVDYSKNRRSRRAITTGYRFTNFEPFTVNSVNDSLEPVGGLYEIQIPSEFTIGNMEEFI
QTSSPKVTIDCAAFVCGDYAACKSQLVEYGSFCDNINAILTEVNELLDTTQLQVANSLMNGVTLSTKLKDGVNF
NVDDINFSSVLGCLGSECSKASSRSAIEDLLFDKVKLSDVGFVAAYNNCTGGAEIRDLICVQSYKGIKVLPPLLSE
NQISGYTLAATSASLFPPWTAAAGVPFYLNVQYRINGLGVTMDVLSQNQKLIANAFNNALDAIQEGFDATNS
ALVKIQAVVNANAEALNNLLQQLSNRFGAISSSLQEILSRLDPPEAEAQIDRLINGRLTALNAYVSQQLSDSTLVK
FSAAQAMEKVNECVKSQSSRINFCGNGNHIISLVQNAPYGLYFIHFSYVPTKYVTAKVSPGLCIAGDRGIAPKSG
YFVNVNNTWMYTGSGYYYPEPITENNVVVMSTCAVNYTKAPYVMLNTSTPNLPDFREELDQWFKNQTSVA
PDLSLDYINVTFLDLQVEMNRLQEAIKVLNGSGYIPEAPRDGQAYVRKDGEWVLLSTFLGRSLEVLFQGPGHH
HHHHHHSAWSHPQFEKGGGSGGGGSGGSAWSHPQFEK
NL63_S-2P (SEQ ID NO: 84):
MKLFLILLVLPLASCFFTCNSNANLSMLQLGVPDNSSTIVTGLLPTHWFCANQSTSVYSANGFFYIDVGNHRSA
FALHTGYYDANQYYIYVTNEIGLNASVTLKICKFSRNTTFDFLSNASSSFDCIVNLLFTEQLGAPLGITISGETVRL
HLYNVTRTFYVPAAYKLTKLSVKCYFNYSCVFSVVNATVTVNVTTHNGRVVNYTVCDDCNGYTDNIFSVQQDG
RIPNGFPFNNWFLLTNGSTLVDGVSRLYQPLRLTCLWPVPGLKSSTGFVYFNATGSDVNCNGYQHNSVVDVM
RYNLNFSANSLDNLKSGVIVFKTLQYDVLFYCSNSSSGVLDTTIPFGPSSQPYYCFINSTINTTHVSTFVGILPPTV
REIVVARTGQFYINGFKYFDLGFIEAVNFNVTTASATDFWTVAFATFVDVLVNVSATNIQNLLYCDSPFEKLQCE
HLQFGLQDGFYSANFLDDNVLPETYVALPIYYQHTDINFTATASFGGSCYVCKPHQVNISLNGNTSVCVRTSHF
SIRYIYNRVKSGSPGDSSWHIYLKSGTCPFSFSKLNNFQKFKTICFSTVEVPGSCNFPLEATWHYTSYTIVGALYV
TWSEGNSITGVPYPVSGIREFSNLVLNNCTKYNIYDYVGTGIIRSSNQSLAGGITYVSNSGNLLGFKNVSTGNIFI
VTPCNQPDQVAVYQQSIIGAMTAVNESRYGLQNLLQLPNFYYVSNGGNNCTTAVMTYSNFGICADGSLIPVR
PRNSSDNGISAIITANLSIPSNWTTSVQVEYLQITSTPIVVDCATYVCNGNPRCKNLLKQYTSACKTIEDALRLSA
HLETNDVSSMLTFDSNAFSLANVTSFGDYNLSSVLPQRNIRSSRIAGRSALEDLLFSKVVTSGLGTVDVDYKSCT
KGLSIADLACAQYYNGIMVLPGVADAERMAMYTGSLIGGMVLGGLTSAAAIPFSLALQARLNYVALQTDVLQ
ENQKILAASFNKAINNIVASFSSVNDAITQTAEAIHTVTIALNKIQDVVNQQGSALNHLTSQLRHNFQAISNSIQ
AIYDRLDPPQADQQVDRLITGRLAALNAFVSQVLNKYTEVRGSRRLAQQKINECVKSQSNRYGFCGNGTHIFS
IVNSAPDGLLFLHTVLLPTDYKNVKAWSGICVDGIYGYVLRQPNLVLYSDNGVFRVTSRIMFQPRLPVLSDFVQIYN
CNVTFVNISRVELHTVIPDYVDVNKTLQEFAQNLPKYVKPNFDLTPFNLTYLNLSSELKOLEAKTASLFQTTV
ELQGLIDQINSTYVDLKLLNRFENGSGYIPEAPRDGQAYVRKDGEWVLLSTFLGRSLEVLFQGPGHHHHHHH
HSAWSHPQFEKGGGSGGGGSGGSAWSHPQFEK
SARS_Linker_dn5b (SEQ ID NO: 85):
MFIFLLFLTLTSGSDLDRCTTFDDVQAPNYTQHTSSMRGVYYPDEIFRSDTLYLTQDLFLPFYSNVTGFHTINHTF
GNPVIPFKDGIYFAATEKSNVVRGWVFGSTMNNKSQSVIIINNSTNVVIRACNFELCDNPFFAVSKPMGTQTH
TMIFDNAFNCTFEYISDAFSLDVSEKSGNFKHLREFVFKNKDGFLYVYKGYQPIDVVRDLPSGFNTLKPIFKLPL
GINITNFRAILTAFSPAQDIWGTSAAAYFVGYLKPTTFMLKYDENGTITDAVDCSQNPLAELKCSVKSFEIDKGIY
QTSNFRVVPSGDVVRFPNITNLCPFGEVFNATKFPSVYAWERKKISNCVADYSVLYNSTFFSTFKCYGVSATKLN
DLCFSNVYADSFVVKGDDVRQIAPGQTGVIADYNYKLPDDFMGCVLAWNTRNIDATSTGNYNYKYRYLRHG
KLRPFERDISNVPFSPDGKPCTPPALNCYWPLNDYGFYTTTGIGYQPYRVVVLSFELLNAPATVCGPKLSTDLIK
NQCVNFNFNGLTGTGVLTPSSKRFQPFQQFGRDVSDFTDSVRDPKTSEILDISPCAFGGVSVITPGTNASSEVA
VLYQDVNCTDVSTAIHADQLTPAWRIYSTGNNVFQTQAGCLIGAEHVDTSYECDIPIGAGICASYHTVSLLRSTS
QKSIVAYTMSLGADSSIAYSNNTIAIPTNFSISITTEVMPVSMAKTSVDCNMYICGDSTECANLLLQYGSFCTQL
NRALSGIAAEQDRNTREVFAQVKQMYKTPTLKYFGGFNFSQILPDPLKPTKRSFIEDLLFNKVTLADAGFMKQ
YGECLGDINARDLICAQKFNGLTVLPPLLTDDMIAAYTAALVSGTATAGWTFGAGAALQIPFAMQMAYRENGI
GVTQNVLYENQKQIANQFNKAISQIQESLTTTSTALGKLQDVVNQNAQALNTLVKQLSSNFGAISSVLNDILSR
LDPPEAEVQIDRLITGRLQSLQTYVTQQLIRAAEIRASANLAATKMSECVLGQSKRVDFCGKGYHLMSFPQAA
PHGVVFLHVTYVPSQERNFTTAPAICHEGKAYFPREGVFVFNGTSWFITQRNFFSPQIITTDNTFVSGNCDVVI
GIINNTVYDPLQPELDSFKEELDKYFKNHTSPDVDLGDISGINASVVNIQKEIDRLNEVAKNLNESLIDLQELGKY
EQGGPGGEEAELAYLLGELAYKLGEYRIAIRAYRIALKRDPNNAEAWYNLGNAYYKQGRYREAIEYYQKALELD
PNNAEAWYNLGNAYYERGEYEEAIEYYRKALRLDPNNADAMQNLLNAKMREEGGWELQHHHHHH
HKU1_Linker_dn5b (SEQ ID NO: 86):
MFLIIFILPTTLAVIGDFNCTNSFINDYNKTIPRISEDVVDVSLGLGTYYVLNRVYLNTTLLFTGYFPKSGANFRDL
ALKGSIYLSTLWYKPPFLSDFNNGIFSKVKNTKLYVNNTLYSEFSTIVIGSVFVNTSYTIVVQPHNGILEITACQYT
MCEYPHTVCKSKGSIRNESWHIDSSEPLCLFKKNFTYNVSADWLYFHFYQERGVFYAYYADVGMPTTFLFSLYL
GTILSHYYVMPLTCNAISSNTDNETLEYWVTPLSRRQYLLNFDEHGVITNAVDCSSSFLSEIQCKTQSFAPNTGV
YDLSGFTVKPVATVYRRIPNLPDCDIDNWLNNVSVPSPLNWERRIFSNCNFNLSTLLRLVHVDSFSCNNLDKSK
IFGSCFNSITVDKFAIPNRRRDDLQLGSSGFLQSSNYKIDISSSSCQLYYSLPLVNVTINNFNPSSWNRRYGFGSF
NLSSYDVVYSDHCFSVNSDFCPCADPSVVNSCAKSKPPSAICPAGTKYRHCDLDTTLYVKNWCRCSCLPDPIST
YSPNTCPQKKVVVGIGEHCPGLGINEEKCGTQLNHSSCFCSPDAFLGWSFDSCISNNRCNIFSNFIFNGINSGT
TCSNDLLYSNTEISTGVCVNYDLYGITGQGIFKEVSAAYYNNWQNLLYDSNGNIIGFKDFLINKTYTILPCYSGRV
SAAFYQNSSSPALLYRNLKCSYVLNNISFISQPFYFDSYLGCVLNAVNLTSYSVSSCDLRMGSGFCIDYALPSSGG
SGSGISSPYRFVTFEPFNVSFVNDSVETVGGLFEIQIPTNFTIAGHEEFIQTSSPKVTIDCSAFVCSNYAACHDLLS
EYGTFCDNINSILNEVNDLLDITQLQVANALMQGVTLSSNLNTNLHSDVDNIDFKSLLGCLGSQCGSSSRSLLE
DLLFNKVKLSDVGFVEAYNNCTGGSEIRDLLCVQSFNGIKVLPPILSETQISGYTTAATVAAMFPPWSAAAGVP
FSLNVQYRINGLGVTMDVLNKNQKLIANAFNKALLSIQNGFTATNSALAKIQSVVNANAQALNSLLQQLFNKF
GAISSSLQEILSRLDPPEAQVQIDRLINGRLTALNAYVSQQLSDITLIKAGASRAIEKVNECVKSQSPRINFCGNG
NHILSLVQNAPYGLLFIHFSYKPTSFKTVLVSPGLCLSGDRGIAPKQGYFIKQNDSWMFTGSSYYYPEPISDKNV
VFMNSCSVNFTKAPFIYLNNSIPNLSDFEAELSLWFKNHTSIAPNLTENSHINATFLDLYYEMNVIQESIKSLNGE
EAELAYLLGELAYKLGEYRIAIRAYRIALKRDPNNAEAWYNLGNAYYKQGRYREAIEYYQKALELDPNNAEAWY
NLGNAYYERGEYEEAIEYYRKALRLDPNNADAMQNLLNAKMREEGGWELQHHHHHH
OC43_Linker_dn5b (SEQ ID NO: 87):
MFLILLISLPTAFAVIGDLKCPLDSRTGSLNNIDTGPPSISTATVDVTNGLGTYYVLDRVYLNTTLFLNGYYPTSGS
TYRNMALKGTDKLSTLWFKPPFLSDFINGIFAKVKNTKVFKDGVMYSEFPAITIGSTFVNTSYSVVVQPRTINST
QDGVNKLQGLLEVSVCQYNMCEYPHTICHPKLGNHFKELWHMDTGVVSCLYKRNFTYDVNATYLYFHFYQE
GGTFYAYFTDTGVVTKFLFNVYLGMALSHYYVMPLTCISRRDIGFTLEYWVTPLTSRQYLLAFNQDGIIFNAVD
CMSDFMSEIKCKTQSIAPPTGVYELNGYTVQPIADVYRRKPDLPNCNIEAWLNDKSVPSPLNWERKTFSNCN
FNMSSLMSFIQADSFTCNNIDAAKIYGMCFSSITIDKFAIPNGRKVDLOLGNLGYLQSFNYRIDTTATSCQLYYN
LPAANVSVSRFNPSTWNKRFGFIENSVFKPQPAGVLTNHDVVYAQHCFKAPKNFCPCKLNSSLCVGSGPGKN
NGIGTCPAGTNYLTCHNLCNPDPITFTGPYKCPQTKSLVGIGEHCSGLAVKSDYCGGNPCTCQPQAFLGWSAD
SCLQGDKCNIFANLILHDVNSGLTCSTDLQKANTDIKLGVCVNYDLYGISGQGIFVEVNATYYNSWQNLLYDSN
GNLYGFRDYITNRTFMIRSCYSGRVSAAFHANSSEPALLFRNIKCNYVFNNSLIRQLQPINYFDSYLGCVVNAYN
STAISVQTCDLTVGSGYCVDYSKNRRSRRAITTGYRFTNFEPFTVNSVNDSLEPVGGLYEIQIPSEFTIGNMEEFI
QTSSPKVTIDCAAFVCGDYAACKSQLVEYGSFCDNINAILTEVNELLDTTQLQVANSLMNGVTLSTKLKDGVNF
NVDDINFSSVLGCLGSECSKASSRSAIEDLLFDKVKLSDVGFVAAYNNCTGGAEIRDLICVQSYKGIKVLPPLLSE
NQISGYTLAATSASLFPPWTAAAGVPFYLNVQYRINGLGVTMDVLSQNQKLIANAFNNALDAIQEGFDATNS
ALVKIQAVVNANAEALNNLLQQLSNRFGAISSSLQEILSRLDPPEAEAQIDRLINGRLTALNAYVSQQLSDSTLVK
FSAAQAMEKVNECVKSQSSRINFCGNGNHIISLVQNAPYGLYFIHFSYVPTKYVTAKVSPGLCIAGDRGIAPKSG
YFVNVNNTWMYTGSGYYYPEPITENNVVVMSTCAVNYTKAPYVMLNTSTPNLPDFREELDQWFKNQTSVA
PDLSLDYINVTFLDLQVEMNRLQEAIKVLNGGEEAELAYLLGELAYKLGEYRIAIRAYRIALKRDPNNAEAWYNL
GNAYYKQGRYREAIEYYQKALELDPNNAEAWYNLGNAYYERGEYEEAIEYYRKALRLDPNNADAMQNLLNAK
MREEGGWELQHHHHHH
MERS_Linker_dn5b (SEQ ID NO: 88):
MIHSVFLLMFLLTPTESYVDVGPDSVKSACIEVDIQQTFFDKTWPRPIDVSKADGIIYPQGRTYSNITITYQGLFP
YQGDHGDMYVYSAGHATGTTPQKLFVANYSQDVKQFANGFVVRIGAAANSTGTVIISPSTSATIRKIYPAFML
GSSVGNFSDGKMGRFFNHTLVLLPDGCGTLLRAFYCILEPRSGNHCPAGNSYTSFATYHTPATDCSDGNYNRN
ASLNSFKEYFNLRNCTFMYTYNITEDEILEWFGITQTAQGVHLFSSRYVDLYGGNMFQFATLPVYDTIKYYSIIPH
SIRSIQSDRKAWAAFYVYKLQPLTFLLDFSVDGYIRRAIDCGFNDLSQLHCSYESFDVESGVYSVSSFEAKPSGSV
VEQAEGVECDFSPLLSGTPPQVYNFKRLVFTNCNYNLTKLLSLFSVNDFTCSQISPAAIASNCYSSLILDYFSYPLS
MKSDLSVSSAGPISQFNYKQSFSNPTCLILATVPHNLTTITKPLKYSYINKCSRFLSDDRTEVPQLVNANQYSPCV
SIVPSTVWEDGDYYRKQLSPLEGGGWLVASGSTVAMTEQLQMGFGITVQYGTDTNSVCPKLEFANDTKIASQ
LGNCVEYSLYGVSGRGVFQNCTAVGVRQQRFVYDAYQNLVGYYSDDGNYYCLRACVSVPVSVIYDKETKTHAT
LFGSVACEHISSTMSQYSRSTRSMLKRRDSTYGPLQTPVGCVLGLVNSSLFVEDCKLPLGQSLCALPDTPSTLTP
ASVGSVPGEMRLASIAFNHPIQVDQLNSSYFKLSIPTNFSFGVTQEYIQTTIQKVTVDCKQYVCNGFQKCEQLL
REYGQFCSKINQALHGANLRQDDSVRNLFASVKSSQSSPIIPGFGGDFNLTLLEPVSISTGSRSARSAIEDLLFDK
VTIADPGYMQGYDDCMQQGPASARDLICAQYVAGYKVLPPLMDVNMEAAYTSSLLGSIAGVGWTAGLSSFA
AIPFAQSIFYRLNGVGITQQVLSENQKLIANKFNQALGAMQTGFTTTNEAFHKVQDAVNNNAQALSKLASEL
SNTFGAISASIGDIIQRLDPPEQDAQIDRLINGRLTTLNAFVAQQLVRSESAALSAQLAKDKVNECVKAQSKRSG
FCGQGTHIVSFVVNAPNGLYFMHVGYYPSNHIEVVSAYGLCDAANPTNCIAPVNGYFIKTNNTRIVDEWSYT
GSSFYAPEPITSLNTKYVAPQVTYQNISTNLPPPLLGNSTGIDFQDELDEFFKNVSTSIPNFGSLTQINTTLLDLTY
EMLSLQQVVKALNESYIDLKELGNYTYGSGEEAELAYLLGELAYKLGEYRIAIRAYRIALKRDPNNAEAWYNLGN
AYYKQGRYREAIEYYQKALELDPNNAEAWYNLGNAYYERGEYEEAIEYYRKALRLDPNNADAMQNLLNAKM
REEGGWELQHHHHHH
SARS-2_Linker_dn5b (SEQ ID NO: 89):
MFVFLVLLPLVSSQCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCNDPFLGVYYHKNNKS
WMESEFRVYSSANNCTFEYVSQPFLMDLEGKQGNFKNLREFVFKNIDGYFKIYSKHTPINLVRDLPQGFS
ALEPLVDLPIGINITRFQTLLALHRSYLTPGDSSSGWTAGAAAYYVGYLQPRTFLLKYNENGTITDAVDCALDPLS
ETKCTLKSFTVEKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNSNNLDSKVG
GNYNYLYRLFRKSNLKPFERDISTEIYQAGSTPCNGVEGFNCYFPLQSYGFQPTNGVGYQPYRVVVLSFELLHA
PATVCGPKKSTNLVKNKCVNFNFNGLTGTGVLTESNKKFLPFQQFGRDIADTTDAVRDPQTLEILDITPCSFGG
VSVITPGTNTSNQVAVLYQDVNCTEVPVAIHADQLTPTWRVYSTGSNVFQTRAGCLIGAEHVNNSYECDIPIG
AGICASYQTQTNSPGSASSVASQSIIAYTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTPPIKDFGGFNFSQILPDPSKPSKRSFIE
DLLFNKVTLADAGFIKQYGDCLGDIAARDLICAQKFNGLTVLPPLLTDEMIAQYTSALLAGTITSGWTFGAGAA
LQIPFAMQMAYRFNGIGVTQNVLYENQKLIANQFNSAIGKIQDSLSSTASALGKLQDVVNQNAQALNTLVKQ
LSSNFGAISSVLNDILSRLDPPEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVSNGTHWFVTQRNFYEP
QIITTDNTFVSGNCDVVIGIVNNTVYDPLQPELDSFKEELDKYFKNHTSPDVDLGDISGINASVVNIQKEIDRLN
EVAKNLNESLIDLQELGKYEQGSGEEAELAYLLGELAYKLGEYRIAIRAYRIALKRDPNNAEAWYNLGNAYYKQG
RYREAIEYYQKALELDPNNAEAWYNLGNAYYERGEYEEAIEYYRKALRLDPNNADAMQNLLNAKMREEGGW
ELQHHHHHH

### Example 6

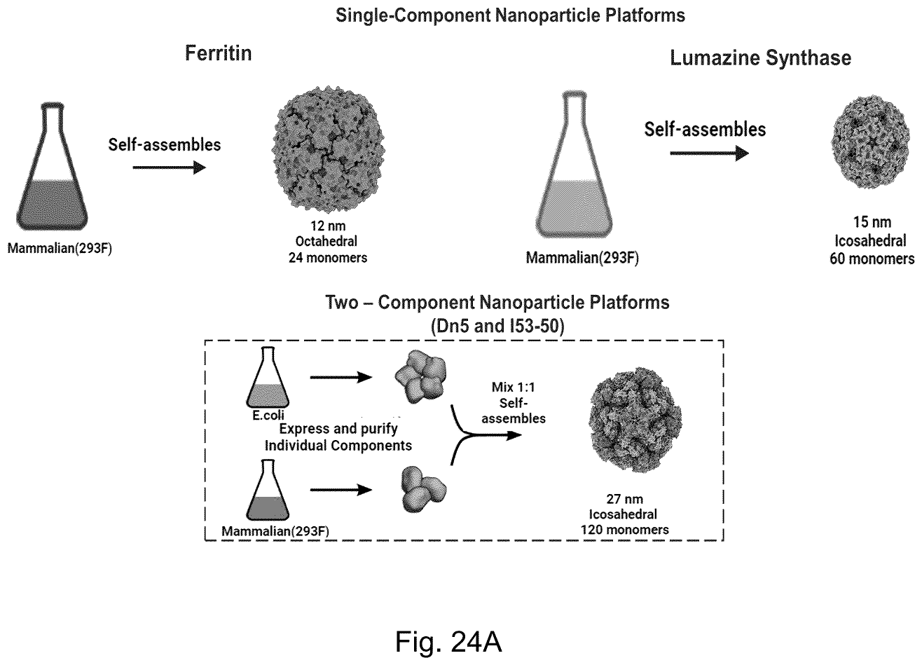

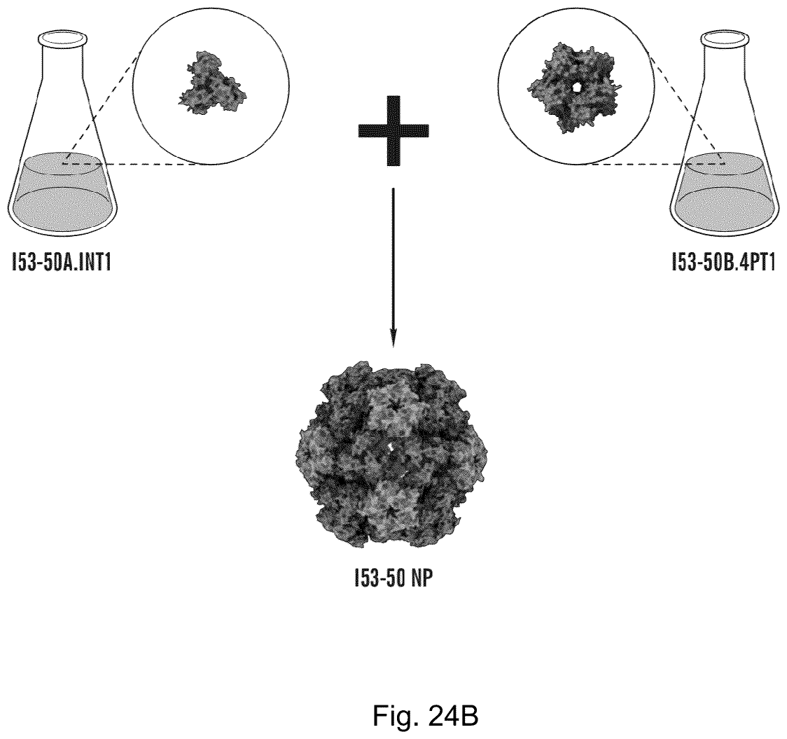

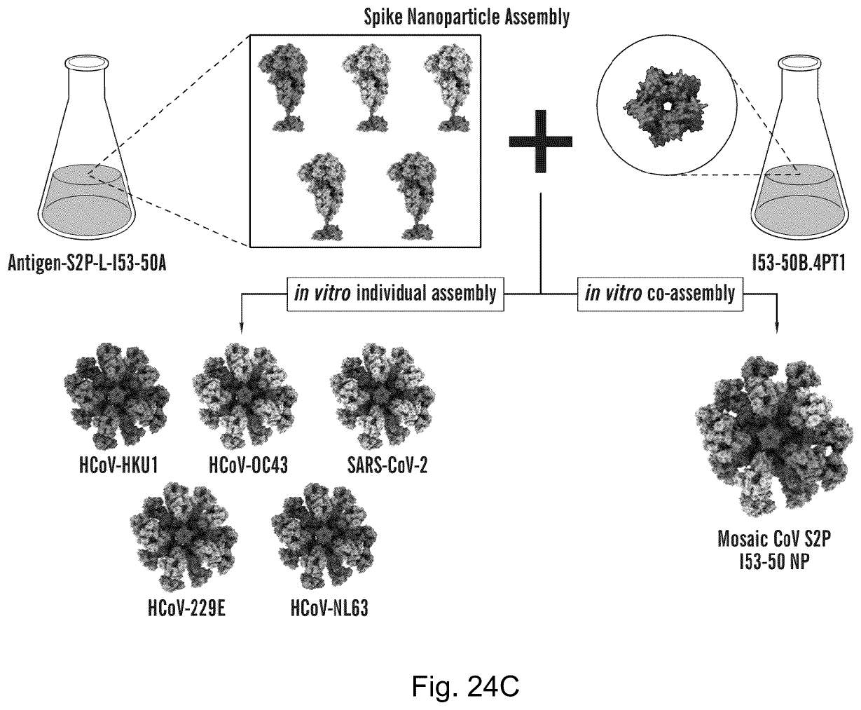

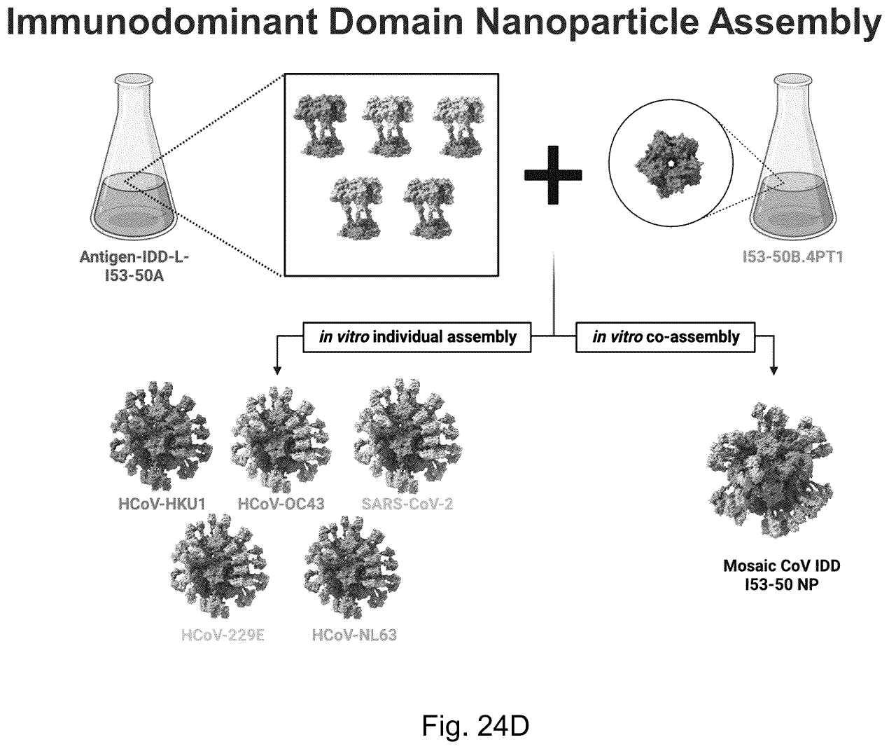

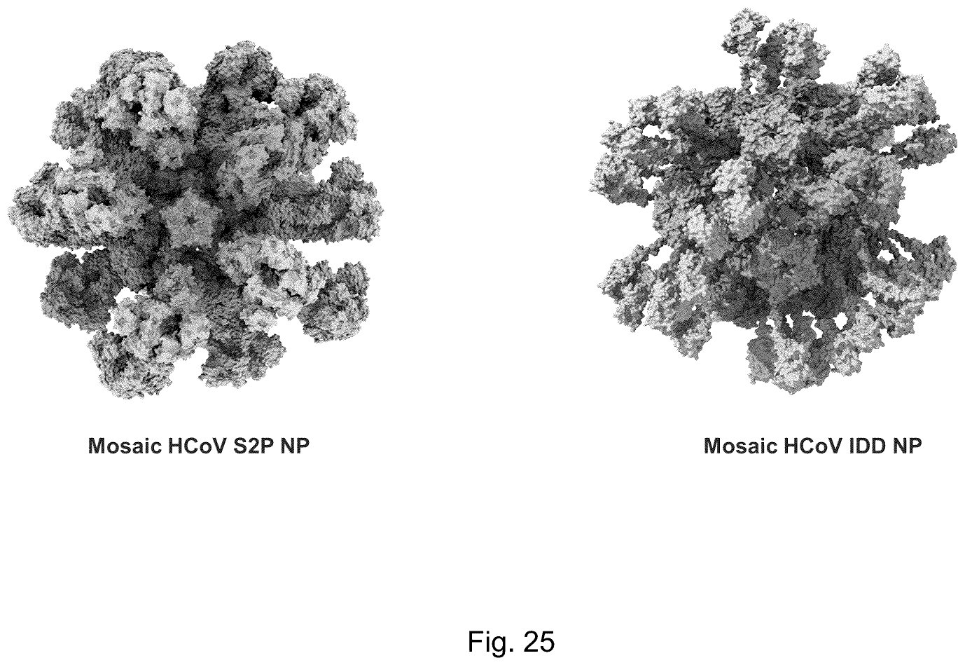

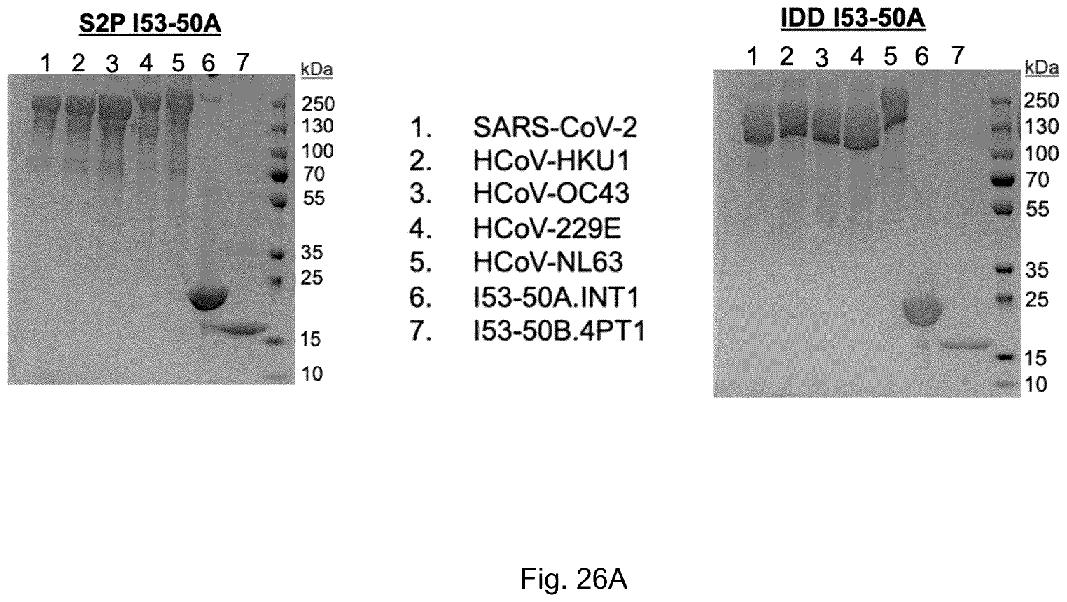

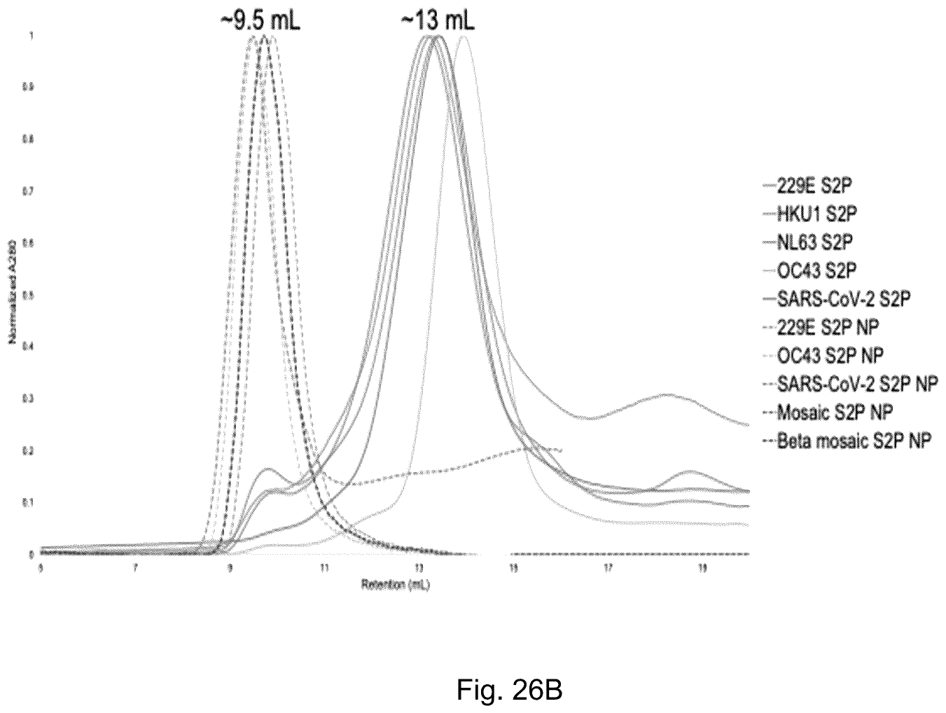

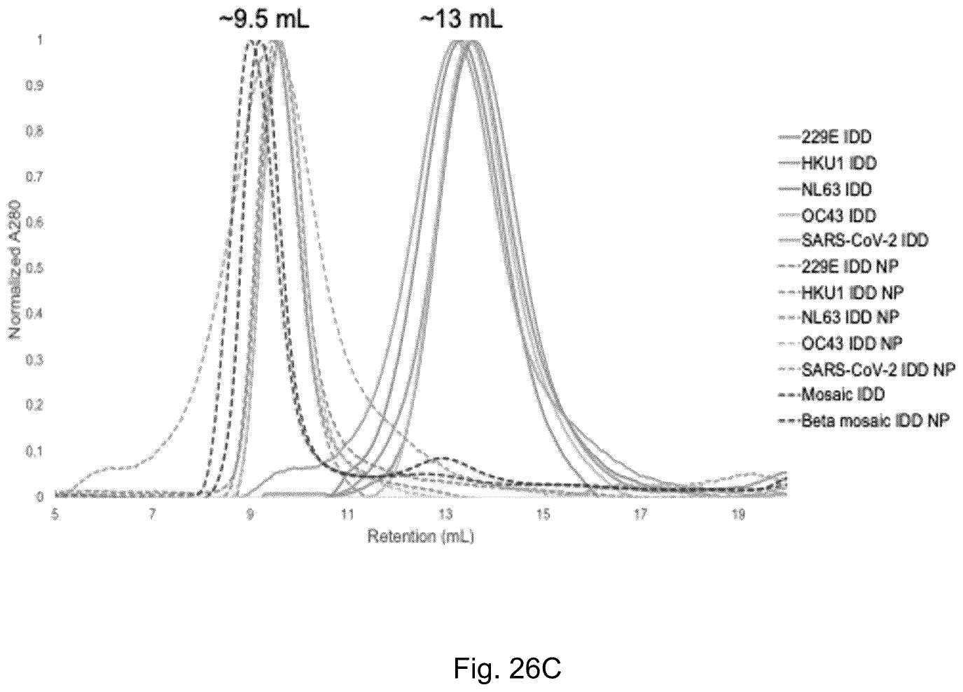

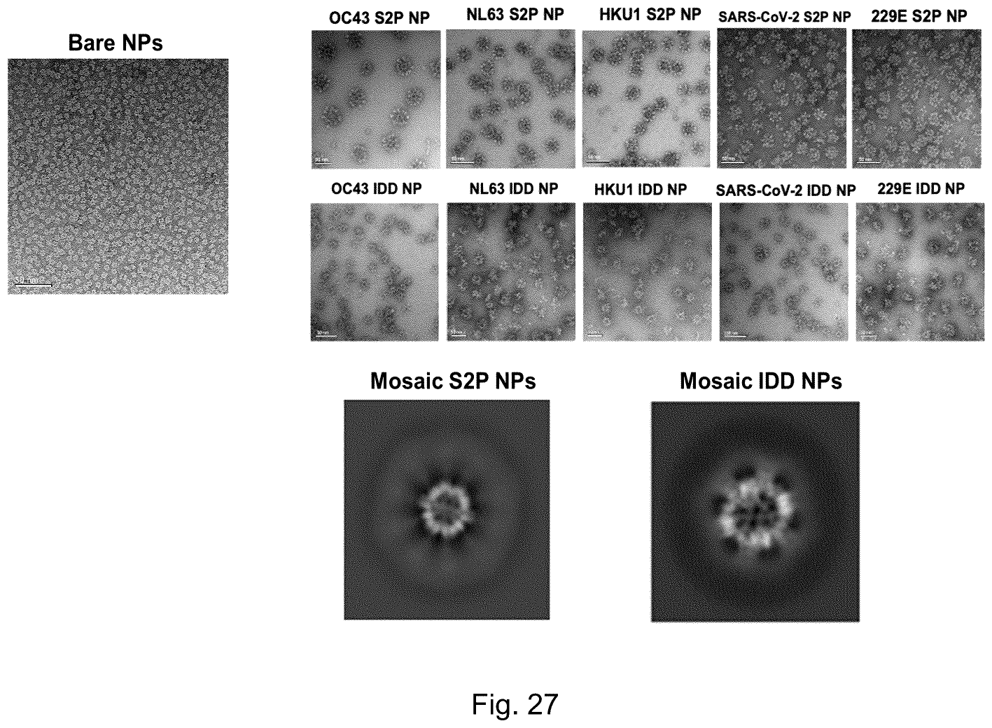

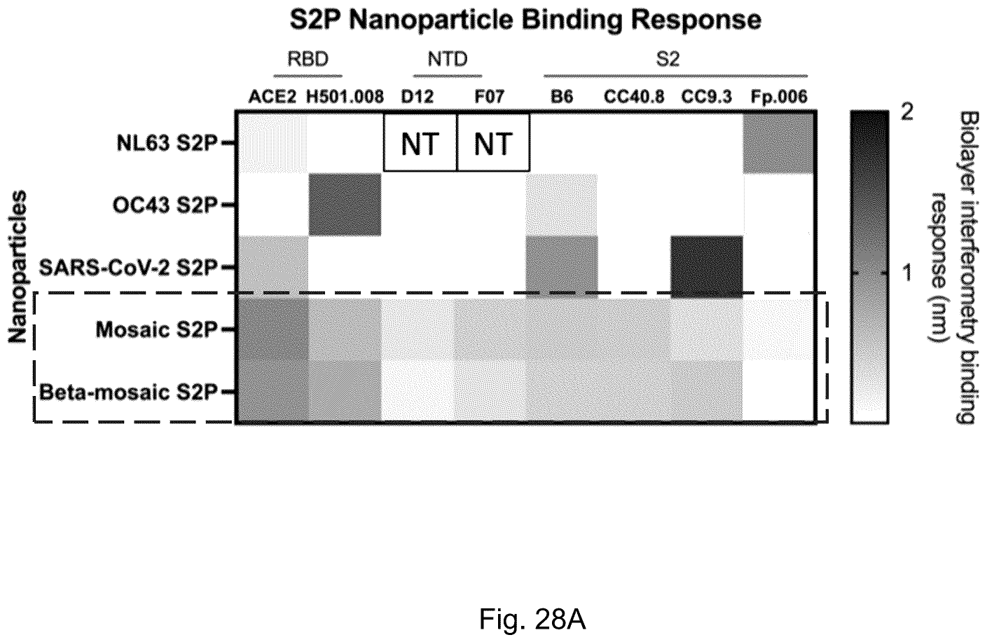

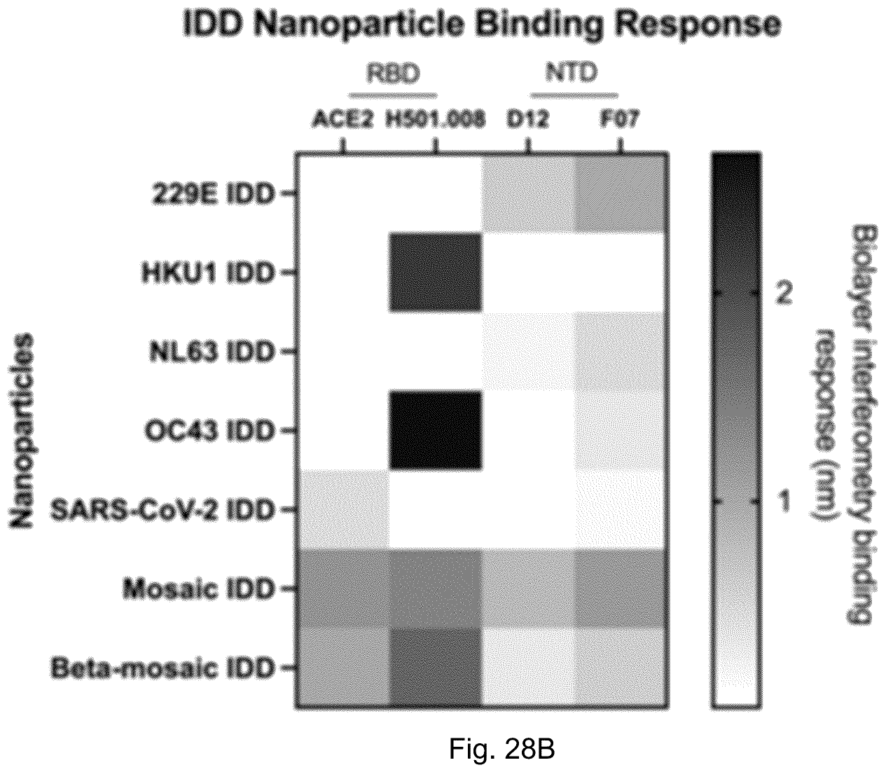

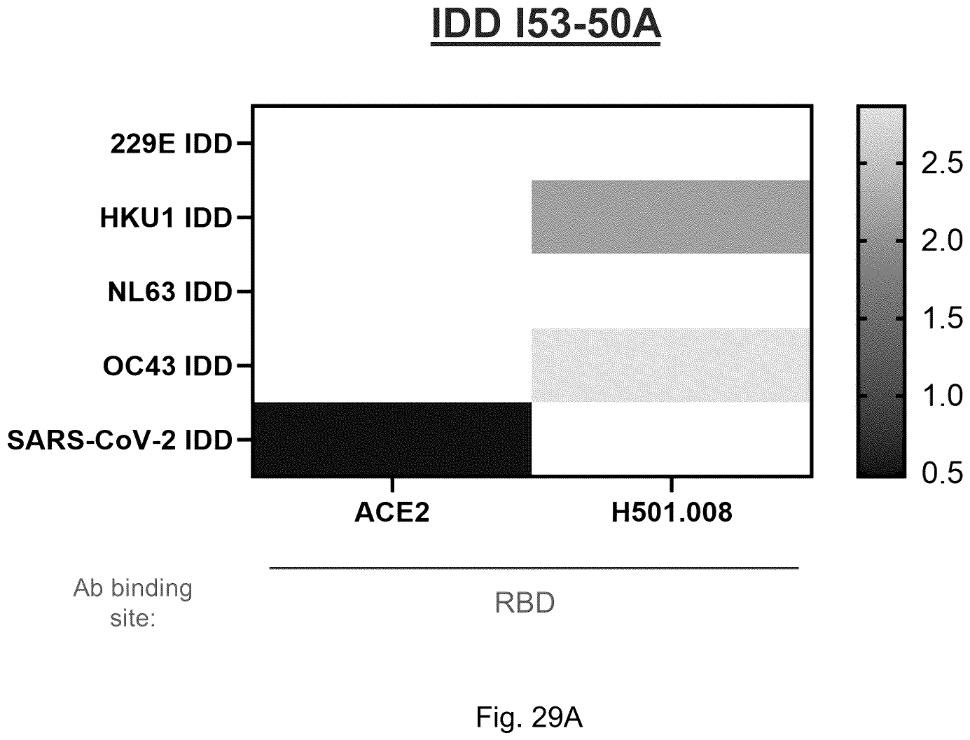

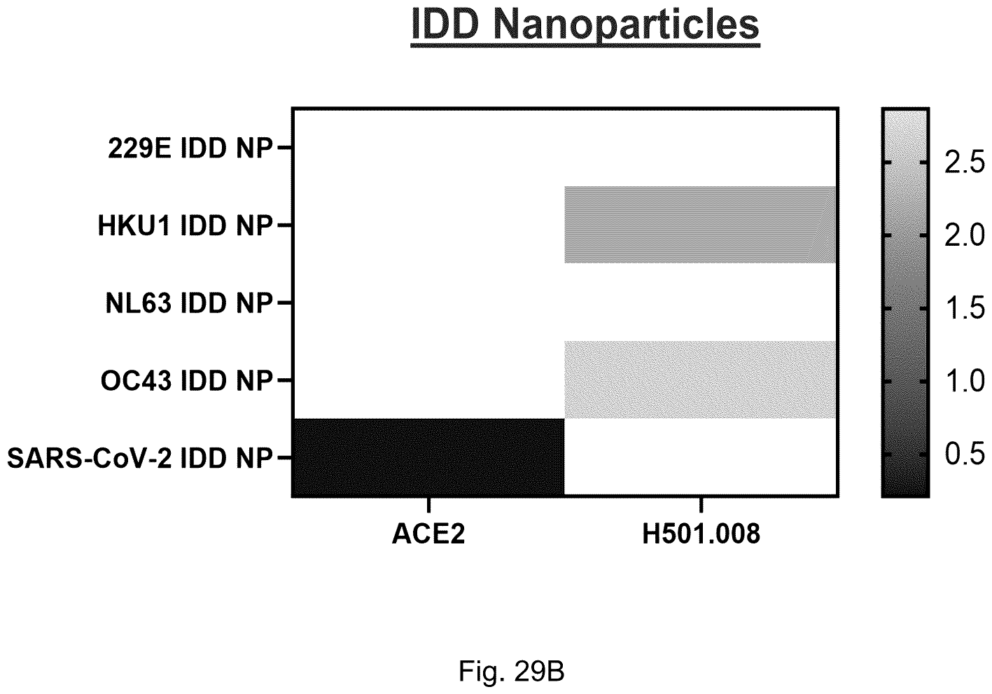

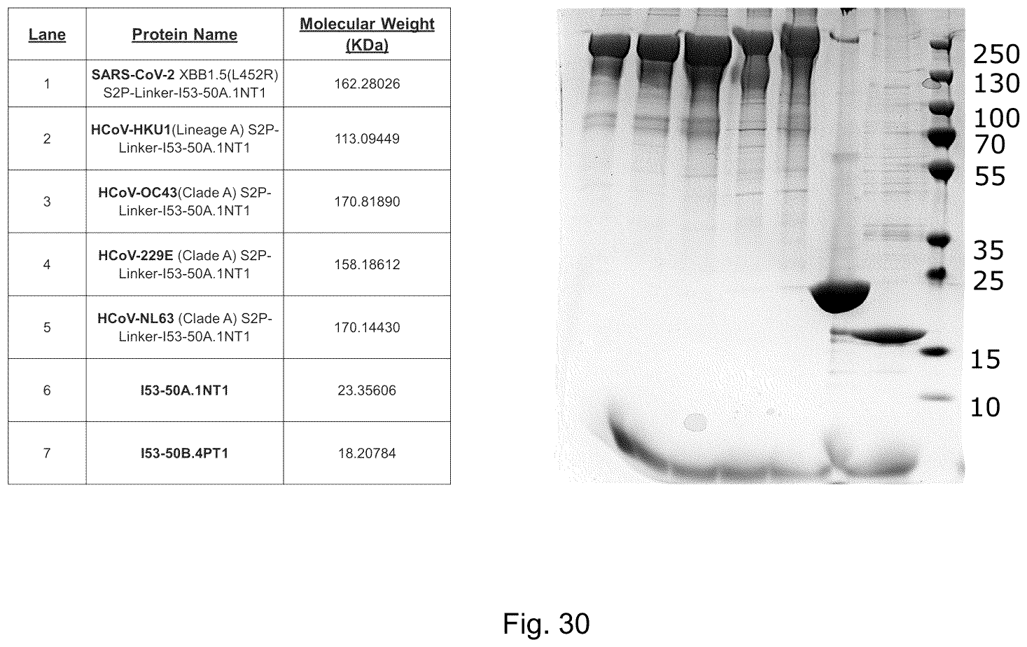

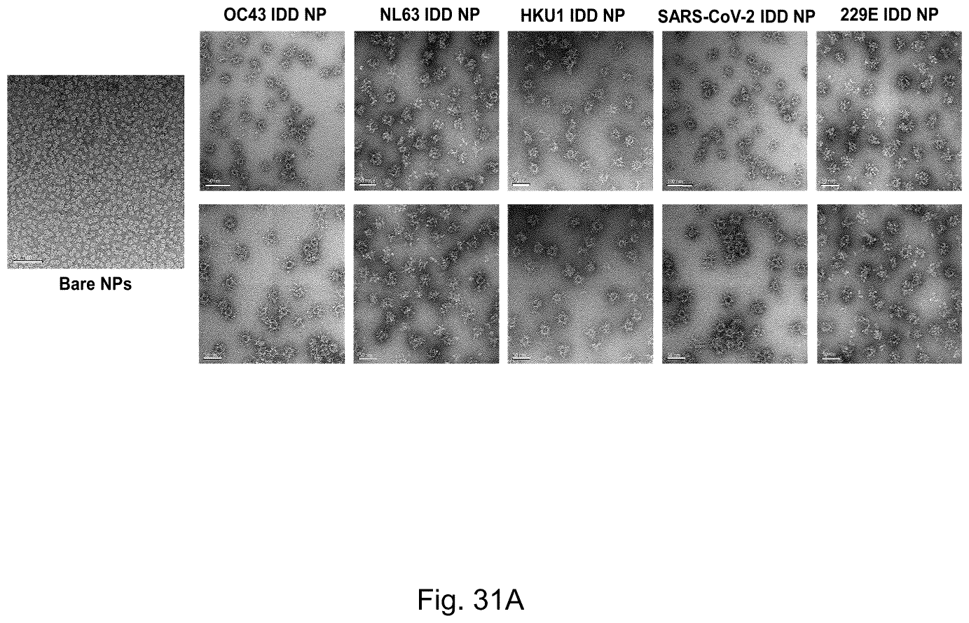

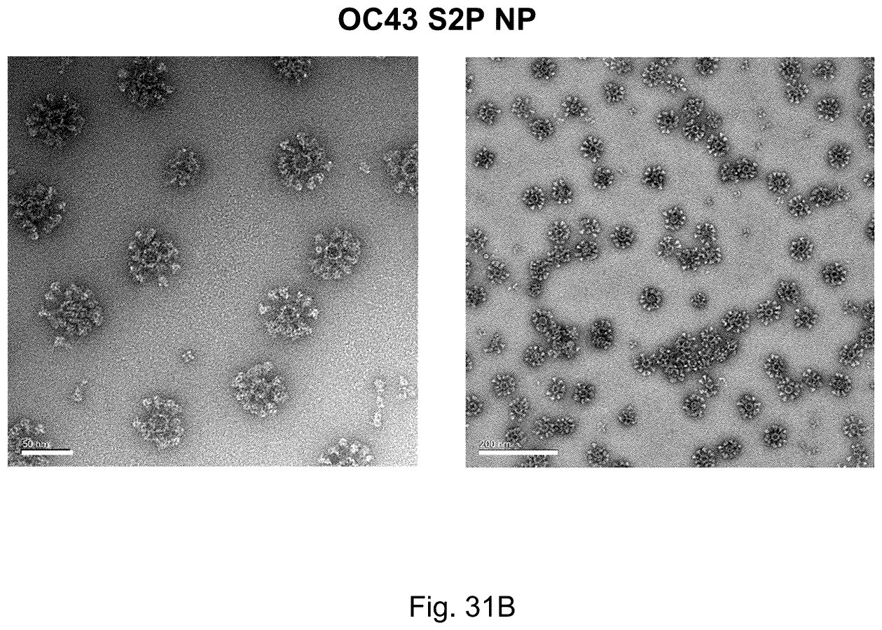

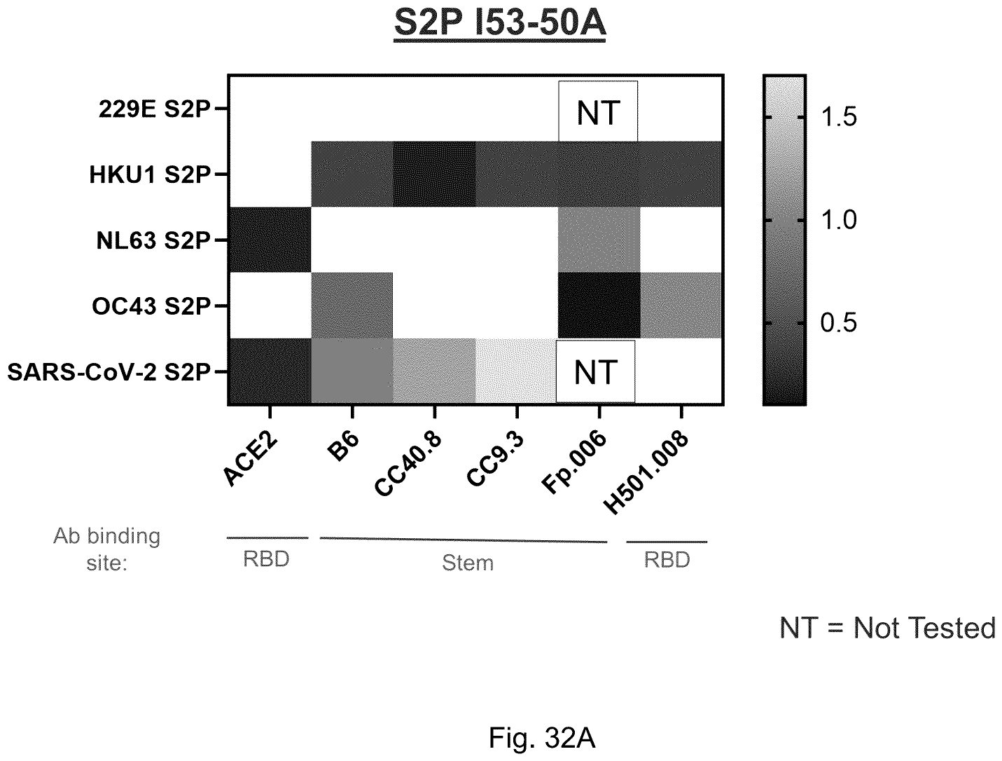

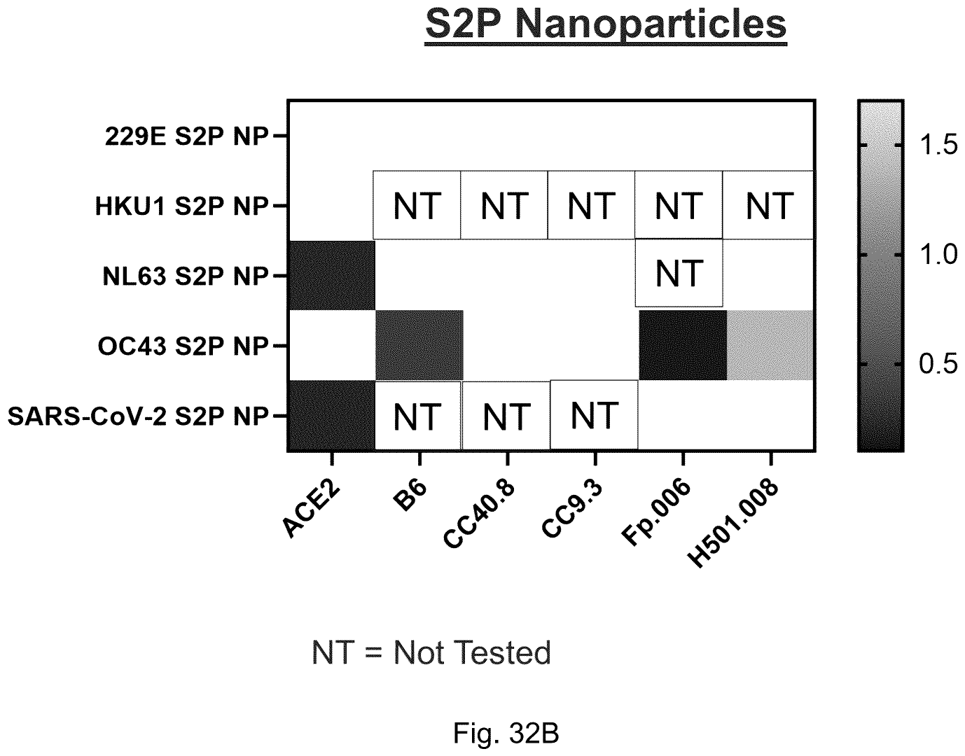

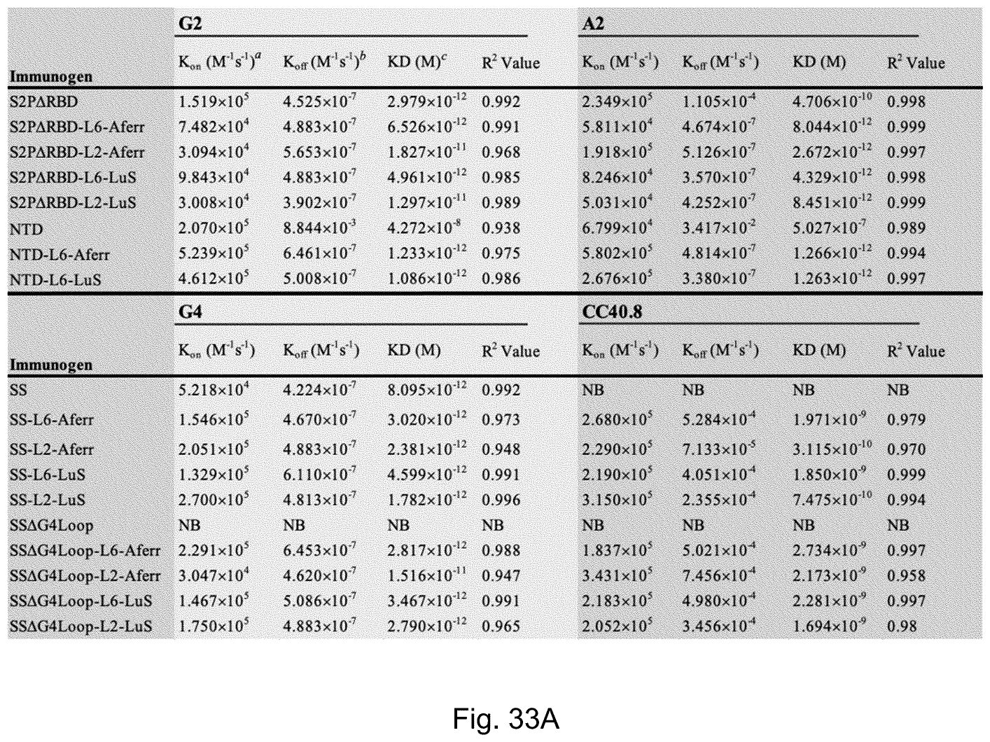

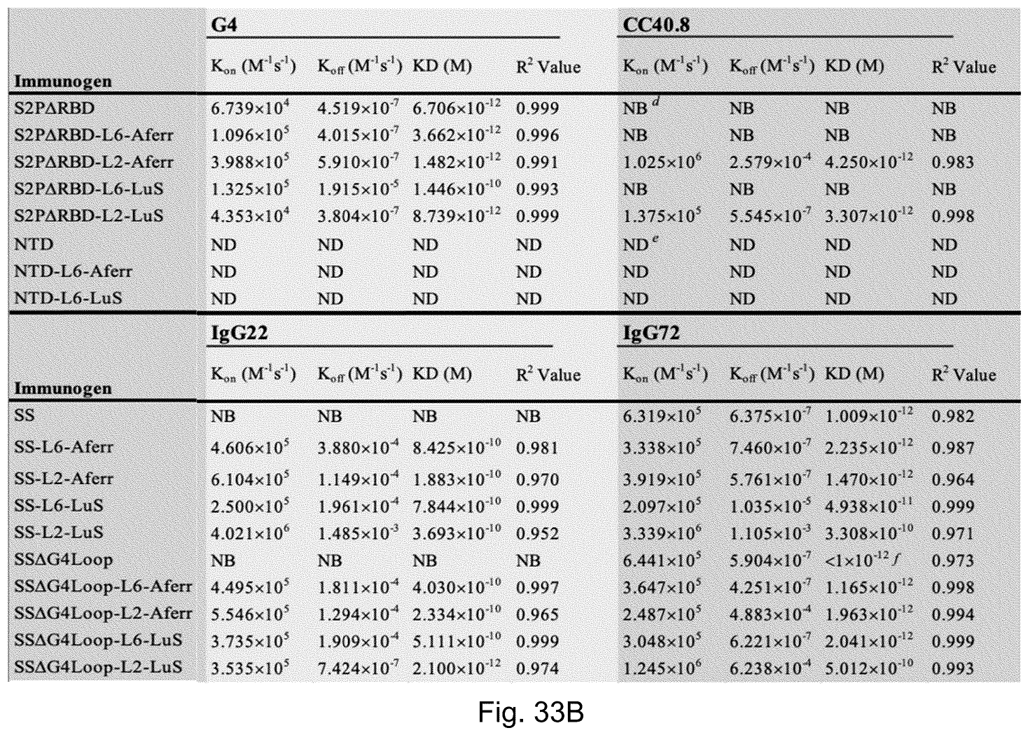

Antigens/display domains were prepared as nanoparticles as depicted in FIGS. 23 and 24A-24D. The resulting polypeptides and nanoparticles were expressed and purified, then characterized (FIGS. 26A-26B, 27, 28A-28B, 29A-29B, 30, 31A-31B, and 32A-32B).

Animals can be immunized with these compositions and the immunogenicity and breadth of protection determines. Resulting monoclonal antibodies can be isolated and characterized.

### Example 7

Nucleic acid sequences for the polypeptides described herein can be readily determined by one of skill in the art, Genes can be codon-optimized for mammalian expression (HEK293 or Expi293F). The genes can be cloned into HindIII and EcoRI in pCDNA 3.1, e.g., pCDNA 3.1(+) vector. Exemplary amino acid sequences are provided below.

SARS-CoV-2 XBB 1.5(L452R) S2P -Linker-153-50A.1NT1 (SEQ ID NO: 109):
MDAMKRGLCCVLLLCGAVFVSPSAS VNLIT RTQSYTNSFT RGVYYPDKVF RSSVLHSTQD
LFLPFFSNVT
WFHAIHVSGT NGTKRFDNPA LPFNDGVYFA STEKSNIIRG WIFGTTLDSK TQSLLIVNNA
TNVVIK VCEF QFCNDPFLDV YQKNNKSWME SEFRVYSSAN NCTFEYVSQP FLMDLEGKEG
NFKNLREFVF KNIDGYFKIY SKHTPINLER DLPQGFSALE PLVDLPIGIN ITRFQTLLAL
HRSYLTPVDS SSGWTAGAAA YYVGYLQPRT FLLKYNENGT ITDAVDCALD PLSETKCTLK
SFTVEKGIYQ TSNFRVQPTE SIVRFPNITN LCPFHEVFNA TTFASVYAWN RKRISNCVAD
YSVIYNFAPF FAFKCYGVSP TKLNDLCFTN VYADSFVIRG NEVSQIAPGQ TGNIADYNYK
LPDDFTGCVI AW NSNKLDSK PSGNYNYRYR LFRKSKLKPF ERDISTEIYQ AGNKPCNGVA
GPNCYSPLQS YGFRPTYGVG HQPY RVVVLS FELLHAPATV CGPKKSTNLV KNKCVNFNFN
GLTGTGVLTE SNKKFLPFQQ FGRDIADTTD AVRDPQTLEI LDITPCSFGG VSVITPGTNT
SNQVAVLYQG VNCTEVPVAI HADQLTPTWR VYSTGSNVFQ TRAGCLIGAE YVNNSYECDI
PIGAGICASY QTQTKSHGSA SSVASQSIIA YTMSLGAENS VAYSNNSIAI PTNFTISVTT
EILPVSMTKT SVDCTMYICG DSTECSNLLL QYGSFCTQLK RALTGIAVEQ DKNTQEVFAQ
VKQIYKTPPI KYFGGFNFSQ ILPDPSKPSK RSFIEDLLFN KVTLADAGFI KQYGDCLGDI
AARDLICAQK FNGLTVLPPL LTDEMIAQYT SALLAGTITS GWTFGAGAAL QIPFAMQMAY
RFNGIGVTQN VL YENQKLIA NQFNSAIGKI QDSLFSTASA LGKLQDVVNH NAQALNTL VK
QLSSKFGAIS SVLNDILSRL DPPEAEVQID RLITGRLQSL QTYVTQQLIR AAEIRASANL
AATKMSECVL GQSKRVDFCG KGYHLMSFPQ SAPHGVVFLH VTYVPAQEKN FTTAPAICHD
GKAHFPREGV FVSNGTHWFV TQRNFYEPQI ITTDNTFVSG NCDVVIGIVN NTVYDPLQPE
LDSFKEELDK YFKNHTSPDV DLGDISGINA SVVNIQKEID RLNEVAKNLN ESLIDLQELG
KYEQGGGGSAEAAAKASSAEAAAKEAAAKEAAAKEEAARKMEELFKKHKIVAVLRANSVEEAIEK
AVAVFAGGVHLIEITFTVPDADTVIKALSVLKEKGAIIGAGTVTSVEQCRKAVESGAEFIVSPHLDEEISQ
FCKEKGVFYMPGVMTPTEL VKAMKLGHDILKLFPGEVVGPEFVKAMKGPFPNVKFVPTGGVDLDNVC
EWFDAGVLAVGVGDALVEG DPDEVREKAKEFVEKIRGCTEGSLE WSHPQFEKGSGHHHHHHHH
SARS-CoV-2 IID LMoS-Linker-153-50A.1NT1 (SEQ ID NO: 110):
MRGLGTCLATLAGLLLTAAG RFP NITNLCPFGE VFNATRFASV YAWNRKRISN CVADYSVLYN
SASFSTFKCY GVSPTKLNDL CFTNVYADSF VIRGDEVRQI APGQTGTIAD YNYKLPDDFT
GCVIAWNSNN LDSK VGGNYN YRYRLFRKSN LKPFERDIST EIYQAGSKPC NGVKGFNCYF
PLQSYGFQPT YGVGYQPYRV VVLSFELLHA PATVCGPKKS TN LVKNKCVN FNFNGLTGTG
VLTESNKKFL PFQQFGRDIA DTTDA VRDPQ TLEILDITPC SFGG GGS RFPNITN LCPFHEVFNA
TTFASVYAWN RKRISNCVAD
YSVIYNFAPF FAFKCYGVSP TKLNDLCFTN VYADSFVIRG NEVSQIAPGQ TGNIADYNYK
LPDDFTGCVI AWNSNKLDST PSGNYNYRYR LFRKSKLKPF ERDISTEIYQ AGNKPCNGVA
GPNCYSPLQS YGFRPTYGVG HQPYRVVVLS FELLHAPATV CGPKKSTNL V KNKCVNFNFN
GLTGTGVLTE SNKKFLPFQQ FGRDIADTTD AVRDPQTLEI LDITPCSFGG
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDALVEG DPDEVREKAKEFVEKIRGCTEGSLE WSHPQFEKGSGHHHHHHHH
HCoV-OC43 S2P(Clade A)-Linker-153-50A.1NT1 (SEQ ID NO: 111):
MPMGSLQPLATLYLLGMLVASVLA VIGDLN CTLDPRLKGS FNNRDTGPPS ISIDTVDVTN
GLGTYYVLDRVYLNTTLFLN GYYPTSGSTY RNMALKGTDL LSTLWFKPPF LSDFINGIFA
KVKNTKVFKDGVMYSEFPAI TIGSTFVNTS YSVVVQPRTI NSTQDGVNKL QGLLEVSVCQ
YNMCEYPHTICHPNLGNHFK ELWHYDTGVV SCLYKRNFTY DVNATYLYFH
FYQEGGTFYA YFTDTGFVTKFLFNVYLGMA LSHYYVMPLT CIRRSKDGFS PEYWVTPLTP
RQYLLAFNQD GIIFNAVDCMSDFMSEIKCK TQSIAPPTGV YELNGYTVQP IADVYRRKPD
LPNCNIEAWL NDKSVPSPLN
WERKTFSNCN FNMSSLMSFI QADSFTCNNI DAAKIYGMCF SSITIDKFAI PNRRKVDLQL
GNLGYLQSSN YRIDTTATSC QLYYNLPAAN VSVSRFNPST WNKRFGFIED AVFKPQPAGV
LTNHDVVYAQ HCFKAPKNFC PCSSCSGKNN GIGTCPAGTN SLTCDNLCTL DPITLKAPDT
YKCPQSKSLV GIGEHCSGLA VKSDYCGNNS CTCQPQAFLG WSADSCLQGD KCNIFANFIL
HDVNNGLTCS TDLQKANTEI ELGVCVNYDL YGISGQGIFV EVNATYYNSW
QNLLYDSNGN
LYGFRDYITN RTFMIHSCYS GRVSAAYHAN SSEPALLFRN IKCNYVFNNS LTRQLQPINY
SFDSYLGCVV NAYNSTAISV QTCDLTVGSG YCVDYFKNGG SGGAITTGYR FTNFEPFTVN
SVNDSLEPVG GLYEIQIPSE FTIGNMEEFI QTSSPKVTID CAAFVCGDYA ACKLQLVEYG
SFCDNINAIL TEVNELLDTT QLQVANSLMN GVTLSTKLKD GVNFNVDDIN FAPVLGCLGS
ECSKASSRSA IEDLLFDKVK LSDVGFVEAY NNCTGGAEIR DLICVQSYKG IKVLPPLLSE
NQISGYTLAA TSASLFPPWT AAAGVPFYLN VQYRINGLGV TMDVLSQNQK LIANAFNNAL
HAIQQGFDAT NSALVKIQAV VNANSEALNN LLQQLSNRFG AISASLQEIL SRLDPPEAEA
QIDRLINGRL TALNAYVSQQ LSDSTLVKFS AAQAMEKVNE CVKSQSSRIN FCGNGNHIIS
LVQNAPYGLY FIHFNYVPTK YVTAKVSPGL CIAGNRGIAP KSGYFVNVNN
TWMYTGSGYY
YPEPITENNV VVMSTCAVNY TKAPYVMLNT SIPNLPDFKE ELDQWFKNQT SVAPDLSLDY
INV TFLDLQV EMNRLQEAIK
VLNGGGGSAEAAAKASSAEAAAKEAAAKEAAAKEEAARKMEELFKKHKIVAVLRANSVEEAIEKA
VAVFAGGVHLIEITFTVPDADTVIKALSVLKEKGAIIGAGTVTSVEQCRKAVESGAEFIVSPHLDEEISQF
CKEKGVFYMPGVMTPTELVKAMKLGHDILKLFPGEVVGPEFVKAMKGPFPNVKFVPTGGVDLDNVCE
WFDAGVLAVGVGDALVEG DPDEVREKAKEFVEKIRGCTEGSLE WSHPQFEKGSGHHHHHHHH
HCoV-OC43 IID LMoS-Linker-153-50A.1NT1 (SEQ ID NO: 112):
MPMGSLQPLATLYLLGMLVASVLA IADVYRRKPD LPNCNIEAWL NDKSVPSPLN
WERKTFSNCN FNMSSLMSFI QADSFTCNNI DAAKIYGMCF SSITIDKFAI PNRRKVDLQL
GNLGYLQSSN YRIDTTATSC QLYYNLPAAN VSVSRFNPST WNKRFGFIED AVFKPQPAGV
LTNHDVVYAQ HCFKAPKNFC PCSSCSGKNN GIGTCPAGTN SLTCDNLCTL DPITLKAPDT
YKCPQSKSLV GIGEHCSGLA VKSDYCGNNS CTCQPQAFLG WSADSCLQGD KCNIFANFIL
HDVNNGLTCS TDLQKANTEI ELGVCVNYDL YGISGQGIFV EVNATYYNSW
QNLLYDSNGN
LYGFRDYITN RTFMIHSCYS GGGSGVY ELNGYTVQPI ADVYRRKPDL PNCNIEAWLN
DKSVPSPLNWERKTFSNCNF NMSSLMSFIQ ADSFTCNNID AAKIYGMCFS SITIDKFAIP
NGRK VDLQLGNLGYLQSFNY RIDTTATSCQ LYYNLPAANV SVSRFNPSTW NKRFGFIEDS
VFKPQPAGVLTDHDVVYAQH CFKAPKNFCP CKSNSSLCVG SGPGKNNGIG TCPAGTNYLT
CHNLCNPDPITFTGPYKCPQ TKSLVGIGEH CSGLAVKSDY CGGNPCTCQP QAFLGWSADS
CLQGDKCNIFANLILHDVNS GLTCSTDLQK ANTDIKLGVC VNYDLYGISG QGIFVEVNAT
YYNSWQNLL YDSNGNLYGFR DYITNRTFMI
RSCYSGGGGSAEAAAKASSAEAAAKEAAAKEAAAKEEAARKMEELFKKHKIVAVLRANSVEEAIEK
AVAVFAGGVHLIEITFTVPDADTVIKALSVLKEKGAIIGAGTVTSVEQCRKAVESGAEFIVSPHLDEEISQ
FCKEKGVFYMPGVMTPTEL VKAMKLGHDILKLFPGEVVGPEFVKAMKGPFPNVKFVPTGGVDLDNVC
EWFDAGVLAVGVGDALVEG DPDEVREKAKEFVEKIRGCTEGSLE WSHPQFEKGSGHHHHHHHH
HRV-3C-OC43 IDD (NTD)-Linker-153-50A.1NT1 SSmodA (SEQ ID NO: 113)
MDAMKRGLCCVLLLCGAVFVSAGSVIGDLN CTLDPRLKGS FNNRDTGPPS ISIDTVDVTN
GLGTYYVLDRVYLNTTLFLN GYYPTSGSTY RNMALKGTDL LSTLWFKPPF LSDFINGIFA
KVKNTKVFKDGVMYSEFPAI TIGSTFVNTS YSVVVQPRTI NSTQDGVNKL QGLLEVSVCQ
YNMCEYPHTICHPNLGNHFK ELWHYDTGVV SCLYKRNFTY DVNATYLYFH
FYQEGGTFYA YFTDTGFVTKFLFNVYLGMA LSHYYVMPLT CIRRSKDGFS PEYWVTPLTP
RQYLLAFNQD GIIFNAGGS VIGDLK CPLDSRTGSL NNIDTGPPSI STATVDVTNG
LGTYYVLDRVYLNTTLFLNG YYPTSGSTYL NMALKGTDKL STLWFKPPFL SDFINGIFAK
VKNTKVFKDGVMYSEFPAIT IGSTFVNTSY SVVVQPRTIN STQDGVNKLQ GLLEVSVCQY
NMCEYPHTICHPNLGNHFKE LWHMDTGVVS CLYKRNFTYD VNATYLYFHF
YQEGGTFYAY FTDTGVVTKFLFNVYLGMAL SHYYVMPLTC ISRRNIGFTL EYWVTPLTSR
QYLLAFNQDG
IIFNAVDCMSDFMSGGGGSAEAAAKASSAEAAAKEAAAKEAAAKEEAARKMEELFKKHKIVAVL
RANSVEEAIEKAVAVFAGGVHLIEITFTVPDADTVIKALSVLKEKGAIIGAGTVTSVEQCRKAVESGAEF
IVSPHLDEEISQFCKEKGVFYMPGVMTPTELVKAMKLGHDILKLFPGEVVGPEFVKAMKGPFPNVKFV
PTGGVDLDNVCEWFDAGVLAVGVGDALVEG
DPDEVREKAKEFVEKIRGCTEGSLEGRSLEVLFQGPG WSHPQFEKGSGHHHHHHHH
HCoV-HKU1(Lineage A) S2P-Linker-153-50A.1NT1 (SEQ ID NO: 114):
MLLIIFILPT TLAVIGDENC TNFAINDKNT TVPRISEYVV DVSYGLGTYY ILDRVYLNTT
ILFTGYFPKS GANFRDLSLK GTTYLSTLWY QKPFLSDENN GIFSRVKNTK
LYVNKTLYSEFSTIVIGSVF INNSYTIVVQ PHNGVLEITA CQYTMCEYPH TICKSKGSSR
NESWHFDKSE PLCLFKKNFT YNVSTDFLYF HFYQERGTFY AYYADSGMPT TFLFSLYLGT
LLSHYYVLPL TCNAISSNTD NETLQYWVTP LSKRQYLLKF DNRGVITNAV DCSSSFFSEI
QCKTKSLLPN TGVYDLSGFT VKPVATVHRR IPDLPDCDID KWLNNFNVPS PLNWERKIFS
NCNFNLSTLL RLVHTDSFSC NNFDESKIYG SCFKSIVLDK FAIPNSRRSD LQLGSSGFLQ
SSNYKIDTTS SSCQLYYSLP AINVTINNYN PSSWNRRYGF NNFNLSSHSV VYSRYCFSVN
NTFCPCAKPS
FASSCKSHKP PSASCPIGTN YRSCESTTVL DHTDWCRCSC LPDPITAYDP RSCSQKKSLV
GVGEHCAGFG VDEEKCGVLD GSYNVSCLCS TDAFLGWSYD TCVSNNRCNI FSNFILNGIN
SGTTCS NDLL QPNTEVYTDV CVDYDLYGIT GQGIFKEVSA VYYNSWQNLL YDSNGNIIGF
KDFVTNKTYN IFPCYAGRVS AAFHQNASSL ALLYRNLKCS YVLNNISLAT QPYFDSYLGC
VFNADNLTDY SVSSCALRMG SGFCVDYNSP SFSSS GGSGS SISASYRFVT FEPFNVSFVN
DSIESVGGLY EIKIPTNFTI VGQEEFIQTN SPKVTIDCSL FVCSNYAACH DLLSEYGTFC
DNINSILDEV NGLLDTTQLH VADTLMQGVT LSSNLNTNLH FDVDNINFKS LVGCLGPHCG
SSSRSFFEDL LFDKVKLSDV GFVEAYNNCT GGSEIRDLLC VQSFNGIKVL PPILSESQIS
GYTTAATVAA MFPPWSAAAG IPFSLNVQYR INGLGVTMDV LNKNQKLIAT AFNNALLSIQ
NGFSATNSAL AKIQSVVNSN AQALNSLLQQ LFNKFGAISS SLQEILSRLD PPEAQVQIDR
LINGRLTALN AYVSQQLSDI SLVKFGAALA MEKVNECVKS QSPRINFCGN GNHILSLVQN
APYGLLFMHF SYKPISFKTV LVSPGLCISG DVGIAP
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDAL VEG DPDEVREKAKEFVEKIRGCTEGSLE WSHPQFEKGSGHHHHHHHH
HCoV-HKU1 IDD (LMoS)-Linker-153-50A.1NT1 (SEQ ID NO: 115):
MDAMKRGLCCVLLLCGAVFVSPSAS GS T VKPVATVHRR IPDLPDCDID KWLNNFNVPS
PLNWERKIFS NCNFNLSTLL RLVHTDSFSC NNFDESKIYG SCFKSIVLDK FAIPNSRRSD
LQLGSSGFLQ SSNYKIDTTS SSCQLYYSLP AINVTINNYN PSSWNRRYGF NNFNLSSHSV
VYSRYCFSVN NTFCPCAKPS FASSCKSHKP PSASCPIGTN YRSCESTTVL DHTDWCRCSC
LPDPITAYDP RSCSQKKSLV GVGEHCAGFG VDEEKCGVLD GSYNVSCLCS TDAFLGWSYD
TCVSNNRCNI FSNFILNGIN SGTTCSNDLL QPNTEVYTDV CVDYDLYGIT GQGIFKEVSA
VYYNSWQNLL YDSNGNIIGF KDFVTNKTYN IFPCYAG
GGS T VKPVATVYRR IPNLPDCDID NWLNNVSVPS PLNWERRIFS NCNFNLSTLL RLVHVDSFSC
NNLDKSKIFG SCFNSITVDK FAIPNRRRDD LQLGSSGFLQ SSNYKIDTTSSSCQLYYSLP AINVTINNYN
PSSWNRRYGF NNFNLSSHSV VYSRYCFSVN NTFCPCAKPSFASSCKSHKP PSASCPIGTK
YRHCDLDTTL YVKNWCRCSC LPDPISTYSP NTCPQKKVVVGIGEHCPGLG INEEKCGTQL
NHSSCSCSPD AFLGWSFDSC ISNNRCNIFS NFIFNGINSGTTCSNDLLYS NTDVSTGVCV
NYDLYGITGQ GIFKEVSAAY YNDWQNLLYD SNGNIIGFKDFLINKTYTIL PCYSGRVS
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTELVKAMKLGHDILKLFPGEVVGPEFVKAMKGPFPNVKFVPTGGVDLDNVCEWF
DAGVLAVGVGDALVEG DPDEVREKAKEFVEKIRGCTEGSLE WSHPQFEKGSGHHHHHHHH
HRV-3C-HKU1 IDD (NTD)-Linker-153-50A.1NT1 (SEQ ID NO: 116)
MPMGSLQPLATLYLLGMLVASVLAVIGDENC TNFAINDKNT TVPRISEYVV DVSYGLGTYY
ILDRVYLNTT ILFTGYFPKS GANFRDLSLK GTTYLSTLWY QKPFLSDFNN GIFSRVKNTK
LYVNKTLYSEFSTIVIGSVF INNSYTIVVQ PHNGVLEITA CQYTMCEYPH TICKSKGSSR
NESWHFDKSE PLCLFKKNFT YNVSTDFLYF HFYQERGTFY AYYADSGMPT TFLFSLYLGT
LLSHYYVLPL TCNAISSNTD NETLQYWVTP LSKRQYLLKF DNRGVITNAV DCSSSFFSEI QCKTKS
GGS VIGDFNC TNSFINHYNK TIPRISEDGV DVSLGLGTYY VLNRVYLNTTLLFTGYFPKS
GANFTDLALK GSTYLSTLWY KPPFLSDFNN GIFSKVKNTK LYVNNILYSEFSTIVIGSVF
VNTSYTIVVQ PHNGILEITA CQYTMCEYPH TVCKSKGSIR NESWHIDSSEPLCLFKKNFT
YNVSADWLYF HFYQERGVFY AYYADVGMPT TFLFSLYLGT ILSHYYVMPLTCNAISPKTD
KETLEYWVTP LSRRQYLLNF DEHGVITKAV DCSSSFLSEI
QCKTQSGGGGSAEAAAKASSAEAAAKEAAAKEAAAKEEAARKMEELFKKHKIVAVLRANSVEEAIEK
AVAVFAGGVHLIEITFTVPDADTVIKALSVLKEKGAIIGAGTVTSVEQCRKAVESGAEFIVSPHLDEEISQ
FCKEKGVFYMPGVMTPTEL VKAMKLGHDILKLFPGEVVGPEFVKAMKGPFPNVKFVPTGGVDLDNVC
EWFDAGVLAVGVGDALVEG DPDEVREKAKEFVEKIRGCTEGSLE GRSLEVLFQGPG
WSHPQFEKGSGHHHHHHHH
HCoV-229E (Clade A)S2P-Linker-153-50A.1NT1 (SEQ ID NO: 117):
MFVLLVAYAL LHIAGCQTTN GTNTSHAVCN GCVGHSENVF AVESGGYIPS NFAFNNWFLL
TNTSSVVDGV VRSFQPLLLN CLWSVSGSQV ITGFVYFNGT GRGACKGFYS NASSDVIRYN
INFEENLRRG TILFKTSYGA VVFYCTNNTL VSGDAHIPSG TVLGNFYCFV NTTIGNETTS
AFVGALPKTV REFVISRTGH FYINGYRYFS LGNVEAVNFN VTNAATTVCT VALASYADVL
VNVSQTAIAN IIYCNSVINR LRCDQLSFDV PDGFYSTSPI QPVELPVSIV SLPVYHKHTF
IVLYVNFEHQ RGPGKCYNCR PAVINITLAN FNETKGPLCV DTSHFTTQFV DNVKLPRWSA
SIDTGNCPFS FGKVINFVKF GSVCFSLKYI PGGCAMPIMA NLVNYKSHNI GSLYVSWSDG
DVITGVPKPV EGVSSFMNVT LNKCTKYNIY DVSGVGVIRI SNDTFLNGIT YTSTSGNLLG
FKDVTNGTIY SITPCNPPDQ LVVYQQAVVG AMLSENFTSY GFSNVVEMPK FFYASNGTYN
CTDAVLTYSS FGVCADGSII AVQPRNVSYD SVSAIVTANL SIPSNWTTSV QVEYLQITST
PIVVDCSTYV CNGNVRCVEL LKQYTSACKT IEDALRNSAM LESADVSEML TFDKKAFTLA
NVSSFGDYNL SSVIPSLPRS GSRVAGRSAI EDILFSKLVT SGLGTVDADY KKCTKGLSIA
DLACAQYYNG IMVLPGAADA ERMAMYTGSL IGGIALGGLT SAASIPFSLA IQSRLNYVAL
QTDVLQENQR ILAASFNKAM TNIVDAFTGV NDAITQTSQA LOTVATALNK IQDVVNQQGN
SLNHLTSQLR QNFQAISSSI QAIYDRLDPP QADQQVDRLI TGRLAALNVF VSHTLTKYTE
VRASRQLAQQ KVNECVKSQS KRYGFCGNGT HIFSL VNAAP EGLVFLHTVL LPTQYKDVEA
WSGLCVDGIN GYVLRQPNLA LYKEGNYYRI TSRIMFEPRI PTIADFVQIE NCNVTFVNIS
RSELQTIVPE YIDVNKTLQE LSYKLPNYTV PDLVVEQYNQ TILNLTSEIS TLENKSAELN
YTVQKLQTLI DNINSTLVDL KWLNRVETYI KWPWW
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTELVKAMKLGHDILKLFPGEVVGPEFVKAMKGPFPNVKFVPTGGVDLDNVCEWF
DAGVLAVGVGDALVEG DPDEVREKAKEFVEKIRGCTEGSLE WSHPQFEKGSGHHHHHHHH
HCoV-229E (LMoS)IDD -Linker-153-50A.1NT1 (SEQ ID NO: 118):
MFVLLVAYAL LHIAGCQTTN GTNTSHAVCN GCVGHSENVF AVESGGYIPS NFAFNNWFLL
TNTSSVVDGV VRSFQPLLLN CLWSVSGSQV ITGFVYFNGT GRGACKGFYS NASSDVIRYN
INFEENLRRG TILFKTSYGA VVFYCTNNTL VSGDAHIPSG TVLGNFYCFV NTTIGNETTS
AFVGALPKTV REFVISRTGH FYINGYRYFS LGNVEAVNFN VTNAATTVCT VALASYADVL
VNVSQTAIAN IIYCNSVINR LRCDQLS GGS QTTN GMNTSHSVCN GCVGHSENVF AVESGGYIPS
NFAFNNWFLL
TNTSSVVDGV VRSFQPLLLN CLWSVSGSRL TTGFVYFNGT GRGDCKGFYS NASSDVIRYN
INFEENLRRG TILFKTSYGA VVFYCTNNTL VSGDAHIPSG TVLGNFYCFV NTTIGNETTS
AFVGALPKTV REFVISRTGH FYINGYRYFS LGNVEAVNFN VTNAATTVCT VALASYADVL
VNVSQTAIAN IIYCNSVINR LRCDQLS
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDALVEG DPDEVREKAKEFVEKIRGCTEGSLE WSHPQFEKGSGHHHHHHHH
HCoV-NL63 (Clade A)S2P-Linker-153-50A.1NT1 (SEQ ID NO: 119):
MKLFLILLIL PLVSCFSTCN SNASISMLQL GVPDNSSTIV TGLLPVHWIC ANQSTSTYPA
NGFFYIDVGK HRSAFALHSG YYDANQYYIY LTNNISLNAP VTLKICKFGN TSFACLSNVS
TSHHCIVNSS FTEQLGVPLG ITISGETVRL HLYNATRTFY VPAAYKLTKL SVKCYFSESC
VFSVVNATVT VNVTTLNGRI VNYTVCDDCN GYTDNIFSVQ QDGRIPNGFS FNNWFLLTNG
STLVDGVSRL YQPLRLTCLW PVPGLKSSTG FVYFNATGSD VNCNGYQHHS VADVMRYNLN
FSSNSVDNLK SGVIVFKTLQ YDVLFYCSNS SSGVLDTTIP FGPSSQPYYC FINSTINTTH
VSTFVGVLPP IVREIVVART GQFYINGFKY FDLGFIEAVN FNVTTASATD FWTVAFATFV
DVLVNVSATN IQNLLYCDSP FEKLQCEHLQ FGLQDGFYSA NFLDDNVLPD TYVALPIYYQ
HTDINFTATA SFGGSCYVCK PHQVNISLNG NTSVCVRTSH FSIRYIYNRV KSGSPGDSSW
HIYLKSGTCP FSFSKLNNFQ KFKTICFSTV AVPGSCNFPL EATWHYTSYT IVGALYVTWS
EGNSITGVPY PVSGIREFSN LVLNNCTKYN IYDYVGTGII RSSNQSLAGG ITYVSNSGNL
LGFKNVSTGN IFIVTPCNQP DQVAVYQQSI IGAMTAVNES RYGLQNLLQL PNFYYVSNGG
NNCTTAVMTY SNFGICADGS LIPVRPRNSS DNGISAIITA NLSIPSNWTT SVQVEYLQIT
STPIVVDCAT YVCNGNPRCK NLLKQYTSAC KTIEDALRLS AHLETNDVSS MLTFDSNAFS
LANVTSFGDY NLSSVLPQRN IRSSRIAGRS ALEDLLFSKV VTSGLGTVDV DYKSCTKGLS
IADLACAQYY NGIMVLPGVA DAERMAMYTG SLIGGMVLGG LTSAAAIPFS LALQARLNYV
ALQTDVLQEN QKILAASFNK AINNIVASFS SVNDAITQTA EAIHTVTIAL NKIQDVVNQQ
GSALNHLTSQ LRHNFQAISN SIQAIYDRLD PPQADQQVDR LITGRLAALN AFVSQVLNKY
TEVRSSRRLA QQKINECVKS QSNRYGFCGN GTHIFSIVNS APDGLLFLHT VLLPTDYKNV
KAWSGICVDG IYGYVLRQPN LVLYSDNGVF RVTSRVMFQP RLPVLSDFVQ IYNCNVTFVN
ISRVKLHTVI PDYVDVNKTL QEFAQNLPKY VKPNFDLTPF NLTYLNFSFE LLQLRTQTAS
LFQTTVELQG LIDQINSTYV DLKLLNRFEN Y
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDALVEG DPDEVREKAKEFVEKIRGCTEGSLE WSHPQFEKGSGHHHHHHHH
HCoV-NL63 (LMoS) IDD-Linker-153-50A.1NT1 (SEQ ID NO: 120):
MKLFLILLIL PL VSCFSTCN SNASISMLQL GVPDNSSTIV TGLLPVHWIC ANQSTSTYPA
NGFFYIDVGK HRSAFALHSG YYDANQYYIY LTNNISLNAP VTLKICKFGN TSFACLSNVS
TSHHCIVNSS FTEQLGVPLG ITISGETVRL HLYNATRTFY VPAAYKLTKL SVKCYFSESC
VFSVVNATVT VNVTTLNGRI VNYTVCDDCN GYTDNIFSVQ QDGRIPNGFS FNNWFLLTNG
STLVDGVSRL YQPLRLTCLW PVPGLKSSTG FVYFNATGSD VNCNGYQHHS VADVMRYNLN
FSSNSVDNLK SGVIVFKTLQ YDVLFYCSNS SSGVLDTTIP FGPSSQPYYC FINSTINTTH
VSTFVGVLPP IVREIVVART GQFYINGFKY FDLGFIEAVN FNVTTASATD FWTVAFATFV
DVLVNVSATN IQNLLYCDSP FEKLQCEHLQ FGLQDGFYSA NFLDDNVLPDGGSSNANLSMLQL
GVPDNSSTIV TGLLPTHWIC ANQSTSVYSA
NGFFFIDVGN HRSAFALHTG YYDVNQYYIY VTNEIGLNAS VTLKICKFGN TTFDFLSNSS
SSFDCIVNLL FTEQLGAPLG ITISGETVRL HLYNVTRTFY VPAAYKLTKL SVKCYFNYSC
VFSVVNATVT VNVTTHNGRV VNYTVCDDCN GYTDNIFSVQ QDGRIPNGFP FNNWFLLTNG
STLVDGVSRL YQPLRLTCLW PVPGLKSSTG FVYFNATGSD VNCNGYQHNS VADVMRYNLN
FSANSVDNLK SGVIVFKTLQ YDVLFYCSNS SSGVLDTTIP FGPSSQPYYC FINSTINTTH
VSTFVGVLPP TVREIVVART GQFYINGFKY FDLGFIEAVN FNVTTASATD FWTVAFATFV
DVLVNVSATK IQNLLYCDSP FEKLQCEHLQ FGLQDGFYSA NFLDDN
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDALVEG DPDEVREKAKEFVEKIRGCTEGSLE WSHPQFEKGSGHHHHHHHH

Bacterial Vector (pET29b)

The I53-50A.1PT1 and I53-50B.4PT1 can be cloned individually into pET29b using the NdeI and XhoI restriction sites.

153-50B.4PT1 (SEQ ID NO:121):
MNQHSHKDHETVRIAVVRARWHAEIVDACVSAFEAAMRDIGGDRFAVDVFDVPGAYEIPLH
ARTLAETGRYGAVLGTAFVVNGGIYRHEFVASAVINGMMNVQLNTGVPVLSAVLTPHNYD
KSKAHTLLFLALFAVKGMEAARACVEILAAREKIAAGSLEHHHHHH
153-50A.1NT1 (SEQ ID NO: 122):
MKMEELFKKHKIVAVLRANSVEEAIEKAVAVFAGGVHLIEITFTVPDADTVIKALSVLKEKG
AIIGAGTVTSVEQCRKAVESGAEFIVSPHLDEEISQFCKEKGVFYMPGVMTPTELVKAMKLG
HDILKLFPGEVVGPEFVKAMKGPFPNVKFVPTGGVDLDNVCEWFDAGVLAVGVGDALVEG
DPDEVREKAKEFVEKIRGCTEGSLEHHHHHH

TABLE 4
Exemplary coronaviruses and sequences

| Endemic Coronaviruses | Identifier Sequence |
|---|---|
| SARS-CoV-2 [Clade A(Delta) | GenBank: WEV00574.1; Gene Accession Number: OQ691663.1 |
| and Clade B(Omicron | Clade A(Delta : available on the world wide web at |
| XBB.1.5)] | nextstrain.org/ncov/gisaid/global/6m?1-unrooted&s=hCoV- |
| | 19/Shanxi/IVDC-579/2022 |
| HCoV-HKU1 | HKU1 bifurcates immediately after the root and is split into |
| | lineage A and lineage B. |
| | Lineage A |
| | Accession KY674942 |
| | N09-1627B_KY674942 |
| | Spike - ARB07608: GenBank: ARB07608.1 |
| | The furin cleavage site at the S1/S2 junction can be mutated |
| | from 755 RRKRR (SEQ ID NO: 229) 760 to GGSGS (SEQ ID NO: |
| | 230) to avert cleavage of the spike protein |
| | Lineage B |
| | SC2521_MK167038 |
| | Accession MK167038 |
| | Spike protein - AZS52618 |
| HCoV-OC43 | Genbank Accession number (Clade A Spike- QEG03794.1) |
| | oc43/MN306053/HCoV_OC43/Seattle/USA/SC9430/2018/2019 |
| | Accession MN306053 |
| | The furin cleavage site 759 RRSRR (SEQ ID NO: 231) 763 at |
| | S1/S2 junction can be mutated to GGSGG (SEQ ID NO: 232). 2P |
| | Stabilizing mutation was introduced at 1075 and 1076. |
| | Genbank Accession number (Clade B Spike - QEG03776/ |
| | QEG03776.1) |
| | oc43/MN306043/HCoV_OC43/Seattle/USA/SC0841/2019/2019 |
| | Accession MN306043 |
| HCoV-NL63 | ChinaGD04_MK334047 |
| | Accession MK334047 |
| | Spike protein (QED88040 - GenBank: QED88040.1) |
| | 2P Stabilizing mutation can be introduced at 1051 and 1052. |
| | SC0768_MN306040 |
| | Accession MN306040 |
| | QEG03748/ GenBank: QEG03748.1 |
| HCoV-229E | 229e/MN306046/HCoV_229E/Seattle/USA/SC0865/2019/2019 |
| | Accession MN306046 |
| | Spike (Genbank No: QEG03785.1) |
| | 229e/KY674919/N08 434B/2016 |
| | Accession KY674919 |
| | GenBank: ARB07425.1 |

### Exemplary Sequences of an at Least One Viral Spike Protein Domain

SARS-CoV-2 XBB 1.5(L452R) S2P (SEQ ID NO: 123)
VNLIT RTQSYTNSFT RGVYYPDK VF RSSVLHSTQD LFLPFFSNVT
WFHAIHVSGT NGTKRFDNPA LPFNDGVYFA STEKSNIIRG WIFGTTLDSK TQSLLIVNNA
TNVVIKVCEF QFCNDPFLDV YQKNNKSWME SEFRVYSSAN NCTFEYVSQP FLMDLEGKEG
NFKNLREFVF KNIDGYFKIY SKHTPINLER DLPQGFSALE PLVDLPIGIN ITRFQTLLAL
HRSYLTPVDS SSGWTAGAAA YYVGYLQPRT FLLKYNENGT ITDAVDCALD PLSETKCTLK
SFTVEKGIYQ TSNFRVQPTE SIVRFPNITN LCPFHEVFNA TTFASVYAWN RKRISNCVAD
YSVIYNFAPF FAFKCYGVSP TKLNDLCFTN VYADSFVIRG NEVSQIAPGQ TGNIADYNYK
LPDDFTGCVI AW NSNKLDSK PSGNYNYRYR LFRKSKLKPF ERDISTEIYQ AGNKPCNGVA
GPNCYSPLQS YGFRPTYGVG HQPY RVVVLS FELLHAPATV CGPKKSTNLV KNKCVNFNFN
GLTGTGVLTE SNKKFLPFQQ FGRDIADTTD AVRDPQTLEI LDITPCSFGG VSVITPGTNT
SNQVAVLYQG VNCTEVPVAI HADQLTPTWR VYSTGSNVFQ TRAGCLIGAE YVNNSYECDI
PIGAGICASY QTQTKSHGSA SSVASQSIIA YTMSLGAENS VAYSNNSIAI PTNFTISVTT
EILPVSMTKT SVDCTMYICG DSTECSNLLL QYGSFCTQLK RALTGIAVEQ DKNTQEVFAQ
VKQIYKTPPI KYFGGFNFSQ ILPDPSKPSK RSFIEDLLFN KVTLADAGFI KQYGDCLGDI
AARDLICAQK FNGLTVLPPL LTDEMIAQYT SALLAGTITS GWTFGAGAAL QIPFAMQMAY
RFNGIGVTQN VLYENQKLIA NQFNSAIGKI QDSLFSTASA LGKLQDVVNH NAQALNTLVK
QLSSKFGAIS SVLNDILSRL DPPEAEVQID RLITGRLQSL QTYVTQQLIR AAEIRASANL
AATKMSECVL GQSKRVDFCG KGYHLMSFPQ SAPHGVVFLH VTYVPAQEKN FTTAPAICHD
GKAHFPREGV FVSNGTHWFV TORNFYEPQI ITTDNTFVSG NCDVVIGIVN NTVYDPLQPE
LDSFKEELDK YFKNHTSPDV DLGDISGINA SVVNIQKEID RLNEVAKNLN ESLIDLQELG
KYEQ
SARS-CoV-2 IID LMOS (SEQ ID NO: 124):
MRGLGTCLATLAGLLLTAAG RFP NITNLCPFGE VFNATRFASV YAWNRKRISN CVADYSVLYN
SASFSTFKCY GVSPTKLNDL CFTNVYADSF VIRGDEVRQI APGQTGTIAD YNYKLPDDFT
GCVIAWNSNN LDSKVGGNYN YRYRLFRKSN LKPFERDIST EIYQAGSKPC NGVKGFNCYF
PLQSYGFQPT YGVGYQPYRV VVLSFELLHA PATVCGPKKS TN LVKNKCVN FNFNGLTGTG
VLTESNKKFL PFQQFGRDIA DTTDAVRDPQ TLEILDITPC SFGG GGS RFPNITN LCPFHEVFNA
TTFASVYAWN RKRISNCVAD
YSVIYNFAPF FAFKCYGVSP TKLNDLCFTN VYADSFVIRG NEVSQIAPGQ TGNIADYNYK
LPDDFTGCVI AWNSNKLDST PSGNYNYRYR LFRKSKLKPF ERDISTEIYQ AGNKPCNGVA
GPNCYSPLQS YGFRPTYGVG HQPYRVVVLS FELLHAPATV CGPKKSTNLV KNKCVNFNFN
GLTGTGVLTE SNKKFLPFQQ FGRDIADTTD AVRDPQTLEI LDITPCSFGG
HcoV-OC43 S2P(Clade A) (SEQ ID NO: 125):
VIGDLN CTLDPRLKGS FNNRDTGPPS ISIDTVDVTN GLGTYYVLDRVYLNTTLFLN
GYYPTSGSTY RNMALKGTDL LSTLWFKPPF LSDFINGIFA KVKNTKVFKDGVMYSEFPAI
TIGSTFVNTS YSVVVQPRTI NSTQDGVNKL QGLLEVSVCQ YNMCEYPHTICHPNLGNHFK
ELWHYDTGVV SCLYKRNFTY DVNATYLYFH FYQEGGTFYA
YFTDTGFVTKFLFNVYLGMA LSHYYVMPLT CIRRSKDGFS PEYWVTPLTP RQYLLAFNQD
GIIFNAVDCMSDFMSEIKCK TQSIAPPTGV YELNGYTVQP IADVYRRKPD LPNCNIEAWL
NDKSVPSPLN
WERKTFSNCN FNMSSLMSFI QADSFTCNNI DAAKIYGMCF SSITIDKFAI PNRRKVDLQL
GNLGYLQSSN YRIDTTATSC QLYYNLPAAN VSVSRFNPST WNKRFGFIED AVFKPQPAGV
LTNHDVVYAQ HCFKAPKNFC PCSSCSGKNN GIGTCPAGTN SLTCDNLCTL DPITLKAPDT
YKCPQSKSLV GIGEHCSGLA VKSDYCGNNS CTCQPQAFLG WSADSCLQGD KCNIFANFIL
HDVNNGLTCS TDLQKANTEI ELGVCVNYDL YGISGQGIFV EVNATYYNSW
QNLLYDSNGN
LYGFRDYITN RTFMIHSCYS GRVSAAYHAN SSEPALLFRN IKCNYVFNNS LTRQLQPINY
SFDSYLGCVV NAYNSTAISV QTCDLTVGSG YCVDYFKNGG SGGAITTGYR FTNFEPFTVN
SVNDSLEPVG GLYEIQIPSE FTIGNMEEFI QTSSPKVTID CAAFVCGDYA ACKLQLVEYG
SFCDNINAIL TEVNELLDTT QLQVANSLMN GVTLSTKLKD GVNFNVDDIN FAPVLGCLGS
ECSKASSRSA IEDLLFDKVK LSDVGFVEAY NNCTGGAEIR DLICVQSYKG IKVLPPLLSE
NQISGYTLAA TSASLFPPWT AAAGVPFYLN VQYRINGLGV TMDVLSQNQK LIANAFNNAL
HAIQQGFDAT NSALVKIQAV VNANSEALNN LLQQLSNRFG AISASLQEIL SRLDPPEAEA
QIDRLINGRL TALNAYVSQQ LSDSTLVKFS AAQAMEKVNE CVKSQSSRIN FCGNGNHIIS
LVQNAPYGLY FIHFNYVPTK YVTAKVSPGL CIAGNRGIAP KSGYFVNVNN
TWMYTGSGYY
YPEPITENNV VVMSTCAVNY TKAPYVMLNT SIPNLPDFKE ELDQWFKNQT SVAPDLSLDY
INV TFLDLQV EMNRLQEAIK VLN
HCoV-OC43 IID LMOS (SEQ ID NO: 126):
IADVYRRKPD LPNCNIEAWL NDKSVPSPLN
WERKTFSNCN FNMSSLMSFI QADSFTCNNI DAAKIYGMCF SSITIDKFAI PNRRKVDLQL
GNLGYLQSSN YRIDTTATSC QLYYNLPAAN VSVSRFNPST WNKRFGFIED AVFKPQPAGV
LTNHDVVYAQ HCFKAPKNFC PCSSCSGKNN GIGTCPAGTN SLTCDNLCTL DPITLKAPDT
YKCPQSKSLV GIGEHCSGLA VKSDYCGNNS CTCQPQAFLG WSADSCLQGD KCNIFANFIL
HDVNNGLTCS TDLQKANTEI ELGVCVNYDL YGISGQGIFV EVNATYYNSW
QNLLYDSNGN
LYGFRDYITN RTFMIHSCYS GGGSGVY ELNGYTVQPI ADVYRRKPDL PNCNIEAWLN
DKSVPSPLNWERKTFSNCNF NMSSLMSFIQ ADSFTCNNID AAKIYGMCFS SITIDKFAIP
NGRK VDLQLGNLGYLQSFNY RIDTTATSCQ LYYNLPAANV SVSRFNPSTW NKRFGFIEDS
VFKPQPAGVLTDHDVVYAQH CFKAPKNFCP CKSNSSLCVG SGPGKNNGIG TCPAGTNYLT
CHNLCNPDPITFTGPYKCPQ TKSL VGIGEH CSGLAVKSDY CGGNPCTCQP QAFLGWSADS
CLQGDKCNIFANLILHDVNS GLTCSTDLQK ANTDIKLGVC VNYDLYGISG QGIFVEVNAT
YYNSWQNLL YDSNGNLYGFR DYITNRTFMI RSCYS
HRV-3C-OC43 IDD (NTD) (SEQ ID NO: 127):
LN CTLDPRLKGS FNNRDTGPPS ISIDTVDVTN GLGTYYVLDRVYLNTTLFLN GYYPTSGSTY
RNMALKGTDL LSTLWFKPPF LSDFINGIFA KVKNTKVFKDGVMYSEFPAI TIGSTFVNTS
YSVVVQPRTI NSTQDGVNKL QGLLEVSVCQ YNMCEYPHTICHPNLGNHFK ELWHYDTGVV
SCLYKRNFTY DVNATYLYFH FYQEGGTFYA YFTDTGFVTKFLFNVYLGMA LSHYYVMPLT
CIRRSKDGFS PEYWVTPLTP RQYLLAFNQD GIIFNAGGS VIGDLK CPLDSRTGSL
NNIDTGPPSI STATVDVTNG LGTYYVLDRVYLNTTLFLNG YYPTSGSTYL NMALKGTDKL
STLWFKPPFL SDFINGIFAK VKNTKVFKDGVMYSEFPAIT IGSTFVNTSY SVVVQPRTIN
STQDGVNKLQ GLLEVSVCQY NMCEYPHTICHPNLGNHFKE LWHMDTGVVS
CLYKRNFTYD VNATYLYFHF YQEGGTFYAY FTDTGVVTKFLFNVYLGMAL
SHYYVMPLTC ISRRNIGFTL EYWVTPLTSR QYLLAFNQDG IIFNAVDCMSDFMS
HCoV-HKU1(Lineage A) S2P (SEQ ID NO: 128):
MLLIIFILPT TLAVIGDENC TNFAINDKNT TVPRISEYVV DVSYGLGTYY ILDRVYLNTT
ILFTGYFPKS GANFRDLSLK GTTYLSTLWY QKPFLSDFNN GIFSRVKNTK
LYVNKTLYSEFSTIVIGSVF INNSYTIVVQ PHNGVLEITA CQYTMCEYPH TICKSKGSSR
NESWHFDKSE PLCLFKKNFT YNVSTDFLYF HFYQERGTFY AYYADSGMPT TFLFSLYLGT
LLSHYYVLPL TCNAISSNTD NETLQYWVTP LSKRQYLLKF DNRGVITNAV DCSSSFFSEI
QCKTKSLLPN TGVYDLSGFT VKPVATVHRR IPDLPDCDID KWLNNFNVPS PLNWERKIFS
NCNFNLSTLL RLVHTDSFSC NNFDESKIYG SCFKSIVLDK FAIPNSRRSD LQLGSSGFLQ
SSNYKIDTTS SSCQLYYSLP AINVTINNYN PSSWNRRYGF NNFNLSSHSV VYSRYCFSVN
NTFCPCAKPS
FASSCKSHKP PSASCPIGTN YRSCESTTVL DHTDWCRCSC LPDPITAYDP RSCSQKKSLV
GVGEHCAGFG VDEEKCGVLD GSYNVSCLCS TDAFLGWSYD TCVSNNRCNI FSNFILNGIN
SGTTCS NDLL QPNTEVYTDV CVDYDLYGIT GQGIFKEVSA VYYNSWQNLL YDSNGNIIGF
KDFVTNKTYN IFPCYAGRVS AAFHQNASSL ALLYRNLKCS YVLNNISLAT QPYFDSYLGC
VFNADNLTDY SVSSCALRMG SGFCVDYNSP SFSSS GGSGS SISASYRFVT FEPFNVSFVN
DSIESVGGLY EIKIPTNFTI VGQEEFIQTN SPKVTIDCSL FVCSNYAACH DLLSEYGTFC
DNINSILDEV NGLLDTTQLH VADTLMQGVT LSSNLNTNLH FDVDNINFKS LVGCLGPHCG
SSSRSFFEDL LFDKVKLSDV GFVEAYNNCT GGSEIRDLLC VQSFNGIKVL PPILSESQIS
GYTTAATVAA MFPPWSAAAG IPFSLNVQYR INGLGVTMDV LNKNQKLIAT AFNNALLSIQ
NGFSATNSAL AKIQSVVNSN AQALNSLLQQ LFNKFGAISS SLQEILSRLD PPEAQVQIDR
LINGRLTALN AYVSQQLSDI SLVKFGAALA MEKVNECVKS QSPRINFCGN GNHILSLVQN
APYGLLFMHF SYKPISFKTV LVSPGLCISG DVGIAP
HCoV-HKU1 IDD (LMoS) (SEQ ID NO: 129):
GS T VKPVATVHRR IPDLPDCDID KWLNNFNVPS PLNWERKIFS NCNFNLSTLL RLVHTDSFSC
NNFDESKIYG SCFKSIVLDK FAIPNSRRSD LQLGSSGFLQ SSNYKIDTTS SSCQLYYSLP AINVTINNYN
PSSWNRRYGF NNFNLSSHSV VYSRYCFSVN NTFCPCAKPS FASSCKSHKP PSASCPIGTN
YRSCESTTVL DHTDWCRCSC LPDPITAYDP RSCSQKKSLV GVGEHCAGFG VDEEKCGVLD
GSYNVSCLCS TDAFLGWSYD TCVSNNRCNI FSNFILNGIN SGTTCSNDLL QPNTEVYTDV
CVDYDLYGIT GQGIFKEVSA VYYNSWQNLL YDSNGNIIGF KDFVTNKTYN IFPCYAG
GGS T VKPVATVYRR IPNLPDCDID NWLNNVSVPS PLNWERRIFS NCNFNLSTLL RLVHVDSFSC
NNLDKSKIFG SCFNSITVDK FAIPNRRRDD LQLGSSGFLQ SSNYKIDTTSSSCQLYYSLP AINVTINNYN
PSSWNRRYGF NNFNLSSHSV VYSRYCFSVN NTFCPCAKPSFASSCKSHKP PSASCPIGTK
YRHCDLDTTL YVKNWCRCSC LPDPISTYSP NTCPQKK VVVGIGEHCPGLG INEEKCGTQL
NHSSCSCSPD AFLGWSFDSC ISNNRCNIFS NFIFNGINSGTTCSNDLLYS NTDVSTGVCV
NYDLYGITGQ GIFKEVSAAY YNDWQNLLYD SNGNIIGFKDFLINKTYTIL PCYSGRVS
HRV-3C-HKU1 IDD (NTD) (SEQ ID NO: 130):
VIGDFNC TNFAINDKNT TVPRISEYVV DVSYGLGTYY ILDRVYLNTT ILFTGYFPKS GANFRDLSLK
GTTYLSTLWY QKPFLSDFNN GIFSRVKNTK LYVNKTLYSEFSTIVIGSVF INNSYTIVVQ
PHNGVLEITA CQYTMCEYPH TICKSKGSSR NESWHFDKSE PLCLFKKNFT YNVSTDFLYF
HFYQERGTFY AYYADSGMPT TFLFSLYLGT LLSHYYVLPL TCNAISSNTD NETLQYWVTP
LSKRQYLLKF DNRGVITNAV DCSSSFFSEI QCKTKS GGS VIGDFNC TNSFINHYNK TIPRISEDGV
DVSLGLGTYY VLNRVYLNTTLLFTGYFPKS GANFTDLALK GSTYLSTLWY KPPFLSDFNN
GIFSKVKNTK LYVNNILYSEFSTIVIGSVF VNTSYTIVVQ PHNGILEITA CQYTMCEYPH TVCKSKGSIR
NESWHIDSSEPLCLFKKNFT YNVSADWLYF HFYQERGVFY AYYADVGMPT TFLFSLYLGT
ILSHYYVMPLTCNAISPKTD KETLEYWVTP LSRRQYLLNF DEHGVITKAV DCSSSFLSEIQCKTQS
HCoV-229E (Clade A)S2P (SEQ ID NO: 131):
MFVLLVAYAL LHIAGCQTTN GTNTSHAVCN GCVGHSENVF AVESGGYIPS NFAFNNWFLL
TNTSSVVDGV VRSFQPLLLN CLWSVSGSQV ITGFVYFNGT GRGACKGFYS NASSDVIRYN
INFEENLRRG TILFKTSYGA VVFYCTNNTL VSGDAHIPSG TVLGNFYCFV NTTIGNETTS
AFVGALPKTV REFVISRTGH FYINGYRYFS LGNVEAVNFN VTNAATTVCT VALASYADVL
VNVSQTAIAN IIYCNSVINR LRCDQLSFDV PDGFYSTSPI QPVELPVSIV SLPVYHKHTF
IVLYVNFEHQ RGPGKCYNCR PAVINITLAN FNETKGPLCV DTSHFTTQFV DNVKLPRWSA
SIDTGNCPFS FGKVINFVKF GSVCFSLKYI PGGCAMPIMA NLVNYKSHNI GSLYVSWSDG
DVITGVPKPV EGVSSFMNVT LNKCTKYNIY DVSGVGVIRI SNDTFLNGIT YTSTSGNLLG
FKDVTNGTIY SITPCNPPDQ LVVYQQAVVG AMLSENFTSY GFSNVVEMPK FFYASNGTYN
CTDAVLTYSS FGVCADGSII AVQPRNVSYD SVSAIVTANL SIPSNWTTSV QVEYLQITST
PIVVDCSTYV CNGNVRCVEL LKQYTSACKT IEDALRNSAM LESADVSEML TFDKKAFTLA
NVSSFGDYNL SSVIPSLPRS GSRVAGRSAI EDILFSKLVT SGLGTVDADY KKCTKGLSIA
DLACAQYYNG IMVLPGAADA ERMAMYTGSL IGGIALGGLT SAASIPFSLA IQSRLNYVAL
QTDVLQENQR ILAASFNKAM TNIVDAFTGV NDAITQTSQA LQTVATALNK IQDVVNQQGN
SLNHLTSQLR QNFQAISSSI QAIYDRLDPP QADQQVDRLI TGRLAALNVF VSHTLTKYTE
VRASRQLAQQ KVNECVKSQS KRYGFCGNGT HIFSLVNAAP EGLVFLHTVL LPTQYKDVEA
WSGLCVDGIN GYVLRQPNLA LYKEGNYYRI TSRIMFEPRI PTIADFVQIE NCNVTFVNIS
RSELQTIVPE YIDVNKTLQE LSYKLPNYTV PDLVVEQYNQ TILNLTSEIS TLENKSAELN
YTVQKLQTLI DNINATLVDL KWLNRVETYIKWPWW
HCoV-229E (LMoS)IDD (SEQ ID NO: 132):
MFVLLVAYAL LHIAGCQTTN GTNTSHAVCN GCVGHSENVF AVESGGYIPS NFAFNNWFLL
TNTSSVVDGV VRSFQPLLLN CLWSVSGSQV ITGFVYFNGT GRGACKGFYS NASSDVIRYN
INFEENLRRG TILFKTSYGA VVFYCTNNTL VSGDAHIPSG TVLGNFYCFV NTTIGNETTS
AFVGALPKTV REFVISRTGH FYINGYRYFS LGNVEAVNFN VTNAATTVCT VALASYADVL
VNVSQTAIAN IIYCNSVINR LRCDQLS GGS QTTN GMNTSHSVCN GCVGHSENVF AVESGGYIPS
NFAFNNWFLL
TNTSSVVDGV VRSFQPLLLN CLWSVSGSRL TTGFVYFNGT GRGDCKGFYS NASSDVIRYN
INFEENLRRG TILFKTSYGA VVFYCTNNTL VSGDAHIPSG TVLGNFYCFV NTTIGNETTS
AFVGALPKTV REFVISRTGH FYINGYRYFS LGNVEAVNFN VTNAATTVCT VALASYADVL
VNVSQTAIAN IIYCNSVINR LRCDQLS
HCoV-NL63 (Clade A)S2P (SEQ ID NO: 133):
MKLFLILLIL PLVSCFSTCN SNASISMLQL GVPDNSSTIV TGLLPVHWIC ANQSTSTYPA
NGFFYIDVGK HRSAFALHSG YYDANQYYIY LTNNISLNAP VTLKICKFGN TSFACLSNVS
TSHHCIVNSS FTEQLGVPLG ITISGETVRL HLYNATRTFY VPAAYKLTKL SVKCYFSESC
VFSVVNATVT VNVTTLNGRI VNYTVCDDCN GYTDNIFSVQ QDGRIPNGFS FNNWFLLTNG
STLVDGVSRL YQPLRLTCLW PVPGLKSSTG FVYFNATGSD VNCNGYQHHS VADVMRYNLN
FSSNSVDNLK SGVIVFKTLQ YDVLFYCSNS SSGVLDTTIP FGPSSQPYYC FINSTINTTH
VSTFVGVLPP IVREIVVART GQFYINGFKY FDLGFIEAVN FNVTTASATD FWTVAFATFV
DVLVNVSATN IQNLLYCDSP FEKLQCEHLQ FGLQDGFYSA NFLDDNVLPD TYVALPIYYQ
HTDINFTATA SFGGSCYVCK PHQVNISLNG NTSVCVRTSH FSIRYIYNRV KSGSPGDSSW
HIYLKSGTCP FSFSKLNNFQ KFKTICFSTV AVPGSCNFPL EATWHYTSYT IVGALYVTWS
EGNSITGVPY PVSGIREFSN LVLNNCTKYN IYDYVGTGII RSSNQSLAGG ITYVSNSGNL
LGFKNVSTGN IFIVTPCNQP DQVAVYQQSI IGAMTAVNES RYGLQNLLQL PNFYYVSNGG
NNCTTAVMTY SNFGICADGS LIPVRPRNSS DNGISAIITA NLSIPSNWTT SVQVEYLQIT
STPIVVDCAT YVCNGNPRCK NLLKQYTSAC KTIEDALRLS AHLETNDVSS MLTFDSNAFS
LANVTSFGDY NLSSVLPQRN IRSSRIAGRS ALEDLLFSKV VTSGLGTVDV DYKSCTKGLS
IADLACAQYY NGIMVLPGVA DAERMAMYTG SLIGGMVLGG LTSAAAIPFS LALQARLNYV
ALQTDVLQEN QKILAASFNK AINNIVASFS SVNDAITQTA EAIHTVTIAL NKIQDVVNQQ
GSALNHLTSQ LRHNFQAISN SIQAIYDRLD PPQADQQVDR LITGRLAALN AFVSQVLNKY
TEVRSSRRLA QQKINECVKS QSNRYGFCGN GTHIFSIVNS APDGLLFLHT VLLPTDYKNV
KAWSGICVDG IYGYVLRQPN LVLYSDNGVF RVTSRVMFQP RLPVLSDFVQ IYNCNVTFVN
ISRVKLHTVI PDYVDVNKTL QEFAQNLPKY VKPNFDLTPF NLTYLNFSFE LLQLRTQTAS
LFQTTVELQG LIDQINSTYV DLKLLNRFEN Y
HCoV-NL63 (LMoS) IDD (SEQ ID NO: 134):
MKLFLILLIL PLVSCFSTCN SNASISMLQL GVPDNSSTIV TGLLPVHWIC ANQSTSTYPA
NGFFYIDVGK HRSAFALHSG YYDANQYYIY LTNNISLNAP VTLKICKFGN TSFACLSNVS
TSHHCIVNSS FTEQLGVPLG ITISGETVRL HLYNATRTFY VPAAYKLTKL SVKCYFSESC
VFSVVNATVT VNVTTLNGRI VNYTVCDDCN GYTDNIFSVQ QDGRIPNGFS FNNWFLLTNG
STLVDGVSRL YQPLRLTCLW PVPGLKSSTG FVYFNATGSD VNCNGYQHHS VADVMRYNLN
FSSNSVDNLK SGVIVFKTLQ YDVLFYCSNS SSGVLDTTIP FGPSSQPYYC FINSTINTTH
VSTFVGVLPP IVREIVVART GQFYINGFKY FDLGFIEAVN FNVTTASATD FWTVAFATFV
DVLVNVSATN IQNLLYCDSP FEKLQCEHLQ FGLQDGFYSA NFLDDNVLPDGGSSNANLSMLQL
GVPDNSSTIV TGLLPTHWIC ANQSTSVYSA
NGFFFIDVGN HRSAFALHTG YYDVNQYYIY VTNEIGLNAS VTLKICKFGN TTFDFLSNSS
SSFDCIVNLL FTEQLGAPLG ITISGETVRL HLYNVTRTFY VPAAYKLTKL SVKCYFNYSC
VFSVVNATVT VNVTTHNGRV VNYTVCDDCN GYTDNIFSVQ QDGRIPNGFP FNNWFLLTNG
STLVDGVSRL YQPLRLTCLW PVPGLKSSTG FVYFNATGSD VNCNGYQHNS VADVMRYNLN
FSANSVDNLK SGVIVFKTLQ YDVLFYCSNS SSGVLDTTIP FGPSSQPYYC FINSTINTTH
VSTFVGVLPP TVREIVVART GQFYINGFKY FDLGFIEAVN FNVTTASATD FWTVAFATFV
DVLVNVSATK IQNLLYCDSP FEKLQCEHLQ FGLQDGFYSA NFLDDN

### Example 8

Nucleic acid sequences for the polypeptides described herein can be readily determined by one of skill in the art. Genes can be codon-optimized for mammalian expression (HEK293 or Expi293F). The genes can be cloned into HindIII and EcoR1 in pCDNA 3.1, e.g., pCDNA 3.1+) vector. Exemplary amino acid sequences are provided below.

TABLE 5
Exemplary coronaviruses and sequences

| MERBECOVIRUS | |
|---|---|
| (CLADES) | ACCESSION NO AND ID |
| CLADE 1 | SINGLE(MONO): HKU4/SM3A (MW218395: SPIKE ID: |
| MULTISPECIES(DPP4) | GENBANK: QQD78083.1) |
| | CHIMERA OF |
| | MERS/EMC12 (JX869059 - SPIKE ID: GENBANK: AFS88936.1) |
| | AND MJIHKU4R-COV-1(OQ786862- SPIKE ID: GENBANK: |
| | WFQ83186 1) |
| CLADE 2 | SINGLE(MONO): HKU5-19S(AGP04932: SPIKE ID: GENBANK: |
| SPECIES- | AGP04932.1) |
| SPECIFIC(ACE2) | CHIMERA OF BT-COV-SC2013(KJ473821: SPIKE ID: |
| | GENBANK: AHY61337.1) AND HKU5-17S(AGP04930: SPIKE |
| | ID: GENBANK: AGP04930.1) |
| CLADE 3 | SINGLE(MONO): PDF2180(KX574227: SPIKE ID: GENBANK: |
| MULTISPECIES- | ARJ34226.1) |
| SPECIFIC(ACE2) | CHIMERA OF PDF2180(KX574227: SPIKE ID: GENBANK: |
| | ARJ34226.1) AND PML-PHE1-2011(KC869678: SPIKE ID: |
| | GENBANK: AGY29650.2) |
| CLADE 4 | SINGLE(MONO): ERINCOV-15-19(MW246802: SPIKE ID: |
| UNKNOWN | GENBANK: QRN68101.1) |
| | CHIMERA OF ERINCOV-19-18(MW246796: GENBANK: |
| | QRN68031.1) AND ERINCOV-11-19(MW246799 GENBANK: |
| | QRN68066.1) |

MERS CHIRBD-L-153-50A(CLADE 1)
(SEQ ID NO: 135)
ATMGILPSPGMPALLSLVSLLSVLLMGCVAETGTECDFSPLLSGTPPQVYNFKRL VFT
NCNYNLTKLLSLFSVNDFTCSQISPAAIASNCYSSLILDYFSYPLSMKSDLSVSSAGPI
SQFNYKQSFSNPTCLILATVPHNLTTITKPLKYSYINKCSRLLSDDRTEVPQL VNANQY
SPCVSIVPSTVWEDGDYYRKQLSPLEGGGWLVASGSTVAMTEQLQMGFGITVQYG
TDTNSVCPKLEFANDTKI ASQLGNCVEY SLYGVSGROV FONCTAVGVR QQRFVYDAYQ NLVGY
GGS EAAATG TFIEQPKSKE CDFTPMLVGV PPQVYNFKRL VFTNCNYNLT
KLLSLFMVNE FSCNGISPDA IARGCYSSLT VDYFAYPLSM RSYIQPGSAG DISL YNYKQS
FANPTCRVLA TAPANLTLTK PSAYGYFQKC SRVSGEHNSV ETPL YINPGE YSICRSFSPY
GFSEDGEVFR RQLTQYEGGG ILVGVGAKLA MTDKLEMGFI ISVQYGTDTN SVCPMLDLGN
SSTITHYLGK CVDYSLYGVT GRGVFQNCTA VGIRQQRFVY DSFDNL VGYY SDDGN
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDAL VEG DPDEVREKAKEFVEKIRGCTEGSLE GRSLEVLFQGPG
WSHPQFEKGSGHHHHHHHH
MERS MONORBD-L-153-50A(CLADE 1)
(SEQ ID NO: 136)
ATMGILPSPGMPALLSLVSLLSVLLMGCVAETGTASATGTFI EQPNATECDF SPMLTGVAPQ
VYNFKRLVFS NCNYNLTKLLSLFAVDEFSC NGISPDAIAR GCYSTLTVDY FAYPLSMKSY
IRPGSAGNIP LYNYKQSFAN
PTCRVMASVP ANVTITKPDA YGYISKCSRL TGANQDVETP LYINPGEYSI CRDFSPGGFS
EDGQVFKRTL TQFEGGGLLI GVGTRVPMTD NLQMSFIIFV QYGTGTDSVC PMLDLGDSLT
ITNRLGKCVD YSLYGVTGRG VFQNCSAVGV KQQRFVYDSF DNL VGYYSDD GNYYC
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDAL VEG DPDEVREKAKEFVEKIRGCTEGSLE GRSLEVLFQGPG
WSHPQFEKGSGHHHHHHHH
MERS CHIRBD-L-153-50A(CLADE 2)
(SEQ ID NO: 137)
ATMGILPSPGMPALLSLVSLLSVLLMGCVAETGT EAQPRGSFI EQAQGTECDF
SPLLKDEPPQ VYNFSRLVFT NCNYNLTKLL ALFQVSQFSC HQVSPSALAS GCYSSLTVDY
FAYPTYMSSY LQQGSTGEIS QFNYKQDFSN PTCRILATVP ANLSASGLLP KPSNYVWLSE
CYQNSFTGKN FQYVKAGQYT PCLGLAANGF EKSYQTHRDP VSKLAVTGVV TPMTSALQMA
FIISVQYGTD SNSVCPMQAV RNDTSVDDKL GLCIDYSLYG ITGRGVFQNC TAVGIRQQRF
VYDSFDNLVG YHADNGNYYC VRPCVSVPVS VVFDRSTNTH ATLFGS GGS EASPRG MFVEQSTSEE
CDFTKMLTGT PPQVYNFNRM VFTNCNYNLT
KLLNLFQVSE FSCYQVSPSS LATGCYSSLT VDSFAFPLEL SSYMQTGSAG PIRQFNYNQD
FSKPTCRVLA SVPSNYTALS KPSNYVHLTE CYTLGYSAKQ YHNTQPGQYT PCLQLANQGF
SRSGQSNSVE LPSGEFKTAT GYIIPMTDQL QMAFVISVQY GTDTNSVCPM QALRNDTSIQ
DKLDVCVEYS LHGVTGRGVF HNCTSVGLRN QRFVYDTFDN L VG YHSDNGN YYCVR
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTELVKAMKLGHDILKLFPGEVVGPEFVKAMKGPFPNVKFVPTGGVDLDNVCEWF
DAGVLAVGVGDALVEG DPDEVREKAKEFVEKIRGCTEGSLE GRSLEVLFQGPG
WSHPQFEKGSGHHHHHHHH
MERS MONORBD-L-153-50A(CLADE 2)
(SEQ ID NO: 138)
ATMGILPSPGMPALLSLVSLLSVLLMGCVAETGT EASPR GEFIEQATTQ ECDFTPMLTG
TPPPIYNFKR LVFTNCNYNLTKLLSLFQVS EFSCHQVSPS SLATGCYSSL TVDYFAYSTD
MSSYLQPGSA GEIVQFNYKQ
DFSNPTCRVL ATVPQNLTTI TKPSNYAYLT ECYKTSAYGK NYLYNAPGGY TPCLSLASRG
FSTK YQSHSD GELTTTGYIY PVTGNLQMAF IISVQYGTDT NSVCPMQALR NDTSIEDKLD
VCVEYSLHGI TGRGVFHNCT SVGLRNQRFV YDTFDNLVGY HSYNGNYYCV RPCVS
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDALVEG DPDEVREKAKEFVEKIRGCTEGSLE GRSLEVLFQGPG
WSHPQFEKGSGHHHHHHHH
MERS CHIRBD-L-153-50A(CLADE 3)
(SEQ ID NO: 139)
ATMGILPSPGMPALLSLVSLLSVLLMGCVAETGT
ATMGILPSPGMPALLSLVSLLSVLLMGCVAETGT E AHVYPDCNFT DLFRENAPTI MQYKRQVFTR
CNYNLTLLLS
LVQVDEFVCD KITPEALATG CYSSLTVDWF AFPYAWKSYL AIGSADRIVR FNYNQDYSNP
SCRIHSKVNS SVGISYSGLY SYITNCNYGG FNKDDVVKPG GRASQPCVTG ALNSPTNGQV
WSFNFGGVPY RTSRLTYTDH LKNPLDMVYV ITVKYEPGAE TVCPKQVRPD YSTNITGLLG
SCISYDIYGI TGTGVFQLCN ATGIPQQKFV YDKFDNIIGF HSDDGNYYCV APCVS GGS S
AKPVGAYYEA HVYPDCNFTE LFRERAPTIM QYKREVFTRC NYNLSLLLSL
VQVDEFVCDK ITPEALATGC YSSLTVDWFA FPYAWKSYLA IGSADRIVRF NYNQDYSNPS
CRIHSK VNSS IGISYAGAYS YITNCNYGAT NKDDVVKPGG RASQQCITGA LNSPTTGQLW
AYNFGGVPYR VSRLTYTDHL SDPLDMVYVI TVK YEPGAET VCPKQIRPDY STNITHLLGS
CISYDIYGIT GTGVFQLCNA TGIRQQKFVY DKFDNIIGFH SDDGNYYCVA PCVSV
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTELVKAMKLGHDILKLFPGEVVGPEFVKAMKGPFPNVKFVPTGGVDLDNVCEWF
DAGVLAVGVGDAL VEG DPDEVREKAKEFVEKIRGCTEGSLE GRSLEVLFQGPG
WSHPQFEKGSGHHHHHHHH
MERS MONORBD-L-153-50A(CLADE 3)
(SEQ ID NO: 140)
ATMGILPSPGMPALLSLVSLLSVLLMGCVAETGT E AHVYPDCNFT DLFRENAPTI MQYKRQVFTR
CNYNLTLLLS
LVQVDEFVCD KITPEALATG CYSSLTVDWF AFPYAWKSYL AIGSADRIVR FNYNQDYSNP
SCRIHSKVNS SVGISYSGLY SYITNCNYGG FNKDDVVKPG GRASQPCVTG ALNSPTNGQV
WSFNFGGVPY RTSRLTYTDH LKNPLDMVYV ITVKYEPGAE TVCPKQVRPD YSTNITGLLG
SCISYDIYGI TGTGVFQLCN ATGIPQQKFV YDKFDNIIGF HSDDGNYYCV APCVS
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDAL VEG DPDEVREKAKEFVEKIRGCTEGSLE GRSLEVLFQGPG
WSHPQFEKGSGHHHHHHHH
MERS CHIRBD-L-153-50A(CLADE 4)
(SEQ ID NO: 141)
ATMGILPSPGMPALLSLVSLLSVLLMGCVAETGT ECELDVLFKN DAPIIANYSR RVFTNCNYNL
TKLLSL VEVD EFICHKTTPE
ALATGCYSSL TVDWFALPFS MKSTLAIGSA EAISMFNYKQ DYSNPTCRIH AAVTGNVSTA
LNFTANANYA YISRCQGTDG KPILLQPGQM TNVACRSGVL ALPSDADYFG YSFQGRTYYV
GRKSYIPKTS EGDIQMVYVI TPNYNKGPDT VCPLKDVSAA ATSLDSLLGQ CIDYDIHG VT
GRGVFQVCNS TGIASQIFVY DGFGNIIGFH SKNGTYYCMA PCVSVPVSVI YDKSS GGS
ECALDVLFKG NAPIIANYSR RVFTNCNYNL TKLLSLVQVD EFVCHKTTPE
ALATGCYSSL TVDWFALPLS MKSTLAIGSA EAISMFNYNQ DYSNPTCRIH ATVTANVSSV
MNFTADNNYA YISRCQGTDG KPILLQPGQM TNIACRSGVL GMQNDADYFG YSFQGRTYYI
GRKSYKPKIS DDVRSGDIQM VYVITPNYNK GPDTVCPLKD VSAASSSLDS LLGQCIDYDI
HGVTGRGVFQ VCNSTGIASQ IFVYDGFGNI IGFHSKNGTY YCMAPCVSVP VSVIYDKS
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDALVEG DPDEVREKAKEFVEKIRGCTEGSLE GRSLEVLFQGPG
WSHPQFEKGSGHHHHHHHH
MERS MONORBD-L-153-50A(CLADE 4)
(SEQ ID NO: 142)
ATMGILPSPGMPALLSLVSLLSVLLMGCVAETGT ECALDVLFKG NAPIIANYSR RVFTNCNYNL
TKLLSL VQVD EFVCHKTTPE
ALATGCYSSL TVDWFALPFS MKSTLAIGSA EAISMFNYNQ DYSNPTCRIH ATVTANVSTA
MNFTAGANYA YISRCQGIDG KPILLQPGQT TNIACRSGVL GMKKDVDYFG YSFQGRIYYV
GRKSYLPKTS EGDIQMVYVI TPNYNKGPDT VCPLKDVSTA STSLDSLLGQ CIDYDIHGVT
GRGVFQVCNS TGIASQIFVY DGFGNIIGFH SKNGTYYCMA PCVSVPVSVI YDKNS
GGGGSAEAAAKASSAEAAAKEAAAKEAAAKEEAARKMEELFKKHKIVAVLRANSVEEAIEKAVAV
FAGGVHLIEITFTVPDADTVIKALSVLKEKGAIIGAGTVTSVEQCRKAVESGAEFIVSPHLDEEISQFCKE
KGVFYMPGVMTPTEL VKAMKLGHDILKLFPGEVVGPEFVKAMKGPFPNVKFVPTGGVDLDNVCEWF
DAGVLAVGVGDAL VEG DPDEVREKAKEFVEKIRGCTEGSLE GRSLEVLFQGPG
WSHPQFEKGSGHHHHHHHH
MERS CHIRBD (CLADE 1)
(SEQ ID NO: 143)
ECDFSPLLSGTPPQVYNFKRL VFT
NCNYNLTKLLSLFSVNDFTCSQISPAAIASNCYSSLILDYFSYPLSMKSDLSVSSAGPI
SQFNYKQSFSNPTCLILATVPHNLTTITKPLKYSYINKCSRLLSDDRTEVPQL VNANQY
SPCVSIVPSTVWEDGDYYRKQLSPLEGGGWLVASGSTVAMTEQLQMGFGITVQYG
TDTNSVCPKLEFANDTKI ASQLGNCVEY SLYGVSGRGV FONCTAVGVR QQRFVYDAYQ NL VGY
GGS EAAATG TFIEQPKSKE CDFTPML VGV PPQVYNFKRL VFTNCNYNLT
KLLSLFMVNE FSCNGISPDA IARGCYSSLT VDYFAYPLSM RSYIQPGSAG DISLYNYKQS
FANPTCRVLA TAPANLTLTK PSAYGYFQKC SRVSGEHNSV ETPL YINPGE YSICRSFSPY
GFSEDGEVFR RQLTQYEGGG IL VGVGAKLA MTDKLEMGFI ISVQYGTDTN SVCPMLDLGN
SSTITHYLGK CVDYSLYGVT GRGVFQNCTA VGIRQQRFVY DSFDNL VGYY SDDGN
MERS MONORBD (CLADE 1)
(SEQ ID NO: 144)
ASATGTFI EQPNATECDF SPMLTGVAPQ VYNFKRLVFS NCNYNLTKLLSLFAVDEFSC NGISPDAIAR
GCYSTLTVDY FAYPLSMKSY IRPGSAGNIP LYNYKQSFAN
PTCRVMASVP ANVTITKPDA YGYISKCSRL TGANQDVETP LYINPGEYSI CRDFSPGGFS
EDGQVFKRTL TQFEGGGLLI GVGTRVPMTD NLQMSFIIFV QYGTGTDSVC PMLDLGDSLT
ITNRLGKCVD YSLYGVTGRG VFQNCSAVGV KQQRFVYDSF DNLVGYYSDD GNYYC
MERS CHIRBD (CLADE 2)
(SEQ ID NO: 145)
EAQPRGSFI EQAQGTECDF
SPLLKDEPPQ VYNFSRLVFT NCNYNLTKLL ALFQVSQFSC HQVSPSALAS GCYSSLTVDY
FAYPTYMSSY LQQGSTGEIS QFNYKQDFSN PTCRILATVP ANLSASGLLP KPSNYVWLSE
CYQNSFTGKN FQYVKAGQYT PCLGLAANGF EKSYQTHRDP VSKLAVTGVV TPMTSALQMA
FIISVQYGTD SNSVCPMQAV RNDTSVDDKL GLCIDYSLYG ITGRGVFQNC TAVGIRQQRF
VYDSFDNLVG YHADNGNYYC VRPCVSVPVS VVFDRSTNTH ATLFGS GGS EASPRG MFVEQSTSEE
CDFTKMLTGT PPQVYNFNRM VFTNCNYNLT
KLLNLFQVSE FSCYQVSPSS LATGCYSSLT VDSFAFPLEL SSYMQTGSAG PIRQFNYNQD
FSKPTCRVLA SVPSNYTALS KPSNYVHLTE CYTLGYSAKQ YHNTQPGQYT PCLQLANQGF
SRSGQSNSVE LPSGEFKTAT GYIIPMTDQL QMAFVISVQY GTDTNSVCPM QALRNDTSIQ
DKLDVCVEYS LHGVTGRGVF HNCTSVGLRN QRFVYDTFDN LVG YHSDNGN YYCVR
MERS MONORBD (CLADE 2)
(SEQ ID NO: 146)
EASPR GEFIEQATTQ ECDFTPMLTG TPPPIYNFKR LVFTNCNYNLTKLLSLFQVS EFSCHQVSPS
SLATGCYSSL TVDYFAYSTD MSSYLQPGSA GEIVQFNYKQ
DFSNPTCRVL ATVPQNLTTI TKPSNYAYLT ECYKTSAYGK NYLYNAPGGY TPCLSLASRG
FSTKYQSHSD GELTTTGYIY PVTGNLQMAF IISVQYGTDT NSVCPMQALR NDTSIEDKLD
VCVEYSLHGI TGRGVFHNCT SVGLRNQRFV YDTFDNLVGY HSYNGNYYCV RPCVS
MERS CHIRBD (CLADE 3)
(SEQ ID NO: 147)
E AHVYPDCNFT DLFRENAPTI MQYKRQVFTR CNYNLTLLLS
LVQVDEFVCD KITPEALATG CYSSLTVDWF AFPYAWKSYL AIGSADRIVR FNYNQDYSNP
SCRIHSKVNS SVGISYSGLY SYITNCNYGG FNKDDVVKPG GRASQPCVTG ALNSPINGQV
WSFNFGGVPY RTSRLTYTDH LKNPLDMVYV ITVKYEPGAE TVCPKQVRPD YSTNITGLLG
SCISYDIYGI TGTGVFQLCN ATGIPQQKFV YDKEDNIIGF HSDDGNYYCV APCVS GGS S
AKPVGAYYEA HVYPDCNFTE LFRERAPTIM QYKREVFTRC NYNLSLLLSL
VQVDEFVCDK ITPEALATGC YSSLTVDWFA FPYAWKSYLA IGSADRIVRF NYNQDYSNPS
CRIHSKVNSS IGISYAGAYS YITNCNYGAT NKDDVVKPGG RASQQCITGA LNSPTTGQLW
AYNFGGVPYR VSRLTYTDHL SDPLDMVYVI TVKYEPGAET VCPKQIRPDY STNITHLLGS
CISYDIYGIT GTGVFQLCNA TGIRQQKFVY DKFDNIIGFH SDDGNYYCVA PCVSV
MERS MONORBD (CLADE 3)
(SEQ ID NO: 148)
E AHVYPDCNFT DLFRENAPTI MQYKRQVFTR CNYNLTLLLS
LVQVDEFVCD KITPEALATG CYSSLTVDWF AFPYAWKSYL AIGSADRIVR FNYNQDYSNP
SCRIHSKVNS SVGISYSGLY SYITNCNYGG FNKDDVVKPG GRASQPCVTG ALNSPTNGQV
WSFNFGGVPY RTSRLTYTDH LKNPLDMVYV ITVK YEPGAE TVCPKQVRPD YSTNITGLLG
SCISYDIYGI TGTGVFQLCN ATGIPQQKFV YDKFDNIIGF HSDDGNYYCV APCVS
MERS CHIRBD (CLADE 4)
(SEQ ID NO: 149)
ECELDVLFKN DAPIIANYSR RVFTNCNYNL TKLLSL VEVD EFICHKTTPE
ALATGCYSSL TVDWFALPFS MKSTLAIGSA EAISMFNYKQ DYSNPTCRIH AAVTGNVSTA
LNFTANANYA YISRCQGTDG KPILLQPGQM TNVACRSGVL ALPSDADYFG YSFQGRTYYV
GRKSYIPKTS EGDIQMVYVI TPNYNKGPDT VCPLKDVSAA ATSLDSLLGQ CIDYDIHGVT
GRGVFQVCNS TGIASQIFVY DGFGNIIGFH SKNGTYYCMA PCVSVPVSVI YDKSS GGS
ECALDVLFKG NAPIIANYSR RVFTNCNYNL TKLLSLVQVD EFVCHKTTPE
ALATGCYSSL TVDWFALPLS MKSTLAIGSA EAISMFNYNQ DYSNPTCRIH ATVTANVSSV
MNFTADNNYA YISRCQGTDG KPILLQPGQM TNIACRSGVL GMQNDADYFG YSFQGRTYYI
GRKSYKPKIS DDVRSGDIQM VYVITPNYNK GPDTVCPLKD VSAASSSLDS LLGQCIDYDI
HGVTGRGVFQ VCNSTGIASQ IFVYDGFGNI IGFHSKNGTY YCMAPCVSVP VSVIYDKS
MERS MONORBD (CLADE 4)
(SEQ ID NO: 150)
ECALDVLFKG NAPIIANYSR RVFTNCNYNL TKLLSLVQVD EFVCHKTTPE
ALATGCYSSL TVDWFALPFS MKSTLAIGSA EAISMFNYNQ DYSNPTCRIH ATVTANVSTA
MNFTAGANYA YISRCQGIDG KPILLQPGQT TNIACRSGVL GMKKDVDYFG YSFQGRIYYV
GRKSYLPKTS EGDIQMVYVI TPNYNKGPDT VCPLKDVSTA STSLDSLLGQ CIDYDIHGVT
GRGVFQVCNS TGIASQIFVY DGFGNIIGFH SKNGTYYCMA PCVSVPVSVI YDKNS

What is claimed herein is:

1. A polypeptide comprising:
a) a display domain comprising
i) a concatenation of at least a first spike protein sequence and a second spike protein sequence; or
ii) a concatenation of at least a first spike protein sequence from a first virus and a second spike protein sequence from a second virus;
b) a linker domain; and
c) a multimerization domain.

2. A polypeptide comprising:
i) a concatenation of at least a first spike protein sequence and a second spike protein sequence; or
ii) a concatenation of at least a first spike protein sequence from a first virus and a second spike protein sequence from a second virus.

3. The polypeptide of claim 1, wherein the first spike protein sequence and/or second spike protein sequence comprises a spike protein ectodomain.

4. The polypeptide of claim 1, wherein the first spike protein sequence and/or second spike protein sequence comprises a spike protein stalk domain.

5. The polypeptide of claim 1, wherein the first spike protein sequence and/or second spike protein sequence comprises:
a virus stalk domain from which the hypervariable immunogenic epitope is absent,
and
does not comprise the subunit 1 (S1) region of virus spike protein.

6. The polypeptide of claim 1, wherein the polypeptide comprises a concatenation of at least a first spike protein sequence and a second spike protein sequence, and the concatenation comprises:
a concatenation of at least a first N-Terminal Domain (NTD) sequence and a second NTD sequence.

7. The polypeptide of claim 1, wherein the polypeptide comprises a concatenation of at least a first spike protein sequence and a second spike protein sequence, and the concatenation comprises:
a concatenation of at least a first RBD sequence and a second RBD sequence.

8. The polypeptide of claim 1, wherein the polypeptide comprises a concatenation of at least a first spike protein sequence and a second spike protein sequence, and the concatenation comprises:
a concatenation of at least:
a) a first NTD sequence, a first Subdomain 1 (SD1) sequence, and a first Subdomain 2 (SD2) sequence; and
b) a second NTD sequence, a second SD1 sequence, and a second SD2 sequence.

9. The polypeptide of claim 1, wherein the polypeptide comprises a concatenation of at least a first spike protein sequence and a second spike protein sequence, and the concatenation comprises:
a concatenation of at least:
a) a first RBD sequence, a first SD1 sequence, and a first SD2 sequence; and
b) a second RBD sequence, a second SD1 sequence, and a second SD2 sequence.

10. The polypeptide of claim 1, wherein the polypeptide comprises a concatenation of at least a first spike protein sequence and a second spike protein sequence, and the concatenation comprises:
a concatenation of at least:
a) a first fusion sequence; and
b) a second fusion sequence.

11. The polypeptide of claim 1, wherein the polypeptide comprises a concatenation of at least a first spike protein sequence from a first virus and a second spike protein sequence from a second virus, and the concatenation comprises:
i) a concatenation of at least a first N-Terminal Domain (NTD) sequence from the first virus and a second NTD sequence from the second virus;
ii) a concatenation of at least a first RBD sequence from the first virus and a second RBD sequence from the second virus;
iii)
a) a first NTD sequence from the first virus, a first SD1 sequence from the first virus, and a first SD2 sequence from the first virus; and
b) a second NTD sequence from the second virus, a second SD1 sequence from the second virus, and a second SD2 sequence from the second virus;
iv)
a) a first RBD sequence from the first virus, a first SD1 sequence from the first virus, and a first SD2 sequence from the first virus; and
b) a second RBD sequence from the second virus, a second SD1 sequence from the second virus, and a second SD2 sequence from the second virus; or
v) a concatenation of at least:
a) a first fusion sequence from the first virus; and
b) a second fusion sequence from the second virus.

12. The polypeptide of claim 1, wherein the spike protein is a coronavirus spike protein, an orthomyxovirus spike protein, a paramyxovirus spike protein, a rhabdovirus spike protein, a filovirus spike protein, a bunyavirus spike protein, an arenavirus spike protein, or a retrovirus spike protein.

13. The polypeptide of claim 1, wherein the first virus is the member of a first clade of a genus and the second virus is the member of a second clade of the virus genus.

14. The polypeptide of claim 1, wherein the first spike protein sequence and/or second spike protein sequence comprises a sequence with at least 80% sequence identity to the sequence of one of SEQ ID NOs: 21-27, 78-84, 90-96, 123-134, and 143-150.

15. The polypeptide of claim 1, further comprising a multimerization domain.

16. The polypeptide of claim 1, wherein the linker domain comprises the sequence of one of SEQ ID NOs: 1-9.

17. The polypeptide of claim 1, wherein the linker domain comprises the sequence of SEQ ID NO: 2.

18. A polypeptide complex comprising a multimer of:
a) a first polypeptide of claim 1; and
b) at least a second polypeptide of claim 1.

19. A nanoparticle comprising at least one polypeptide of claim 1.

20. A method of immunizing a subject, the method comprising administering a polypeptide of claim 1.


SEQUENCE LISTING (243 sequences)

SEQ ID NO: 1 - protein, 25 aa, synthetic construct
GGGGSGGGGSGGGGSGGGGSGGGGS

SEQ ID NO: 2 - protein, 27 aa, synthetic construct
GGGGSAEAAAKEAAAKEAAAKAGGGGS

SEQ ID NO: 3 - protein, 37 aa, synthetic construct
GGGGSAEAAAKEAAAKEAAAKEAAAKEAAAKAGGGGS

SEQ ID NO: 4 - protein, 18 aa, synthetic construct
GSSSSSGSSSSSGSSSSS

SEQ ID NO: 5 - protein, 43 aa, synthetic construct
GSAEAAAKEAAAKEAAAKAGGGGSAEAAAKEAAAKEAAAKAGS

SEQ ID NO: 6 - protein, 15 aa, synthetic construct
GGGSGGGGSGGGGSG

SEQ ID NO: 7 - protein, 29 aa, synthetic construct
GSSSSSAEAAAKEAAAKEAAAKAGSSSSS

SEQ ID NO: 8 - skipped

SEQ ID NO: 9 - protein, 18 aa, synthetic construct
GGGSGGGGSGGGGSGLSK

SEQ ID NO: 10 - protein, 35 aa, synthetic construct
MDAMKRGLCCVLLLCGAVFVSPSQEIHARFRRGAR

SEQ ID NO: 11 - protein, 30 aa, synthetic construct
MDSKGSSQKGSRLLLLLVVSNLLLPQGVLA

SEQ ID NO: 12 - protein, 23 aa, synthetic construct
MRPTWAWWLFLVLLLALWAPARG

SEQ ID NO: 13 - protein, 17 aa, synthetic construct
MIHSVFLLMFLLTPTES

SEQ ID NO: 14 - protein, 24 aa, synthetic construct
MPMGSLQPLATLYLLGMLVASVLA

SEQ ID NO: 15 - protein, 20 aa, synthetic construct
MRGLGTCLATLAGLLLTAAG

SEQ ID NO: 16 - protein, 15 aa, synthetic construct
MFVFLVLLPLVSSQC

SEQ ID NO: 17 - protein, 175 aa, synthetic construct
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 18 - protein, 167 aa, synthetic construct
MLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 19 - protein, 154 aa, synthetic construct
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 20 - protein, 154 aa, synthetic construct; identical to SEQ ID NO: 19

SEQ ID NO: 21 - protein, 587 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGTFIKGGSGGNSTGGSGGWNYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLG

SEQ ID NO: 22 - protein, 540 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSGGSGGSGGSGGSPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLG

SEQ ID NO: 23 - protein, 761 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGTGGGGSGGGGSNATGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 24 - protein, 630 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGTGGGGSGGGGSNATGSSFYAPENITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 25 - protein, 1104 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGGGSGGKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 26 - protein, 1123 aa, synthetic construct
MDSKGSSQKGSRLLLLLVVSNLLLPQGVLASSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGGGSGGKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 27 - protein, 1275 aa, synthetic construct
MDSKGSSQKGSRLLLLLVVSNLLLPQGVLASSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGGGSGGKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGGGGGSAEAAAKEAAAKEAAAKAGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 28 - protein, 593 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGGSGGGGSGGGGSGGGGSGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 29 - protein, 595 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGGSAEAAAKEAAAKEAAAKAGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 30 - protein, 605 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGGSAEAAAKEAAAKEAAAKEAAAKEAAAKAGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 31 - protein, 586 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGSSSSSGSSSSSGSSSSSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 32 - protein, 611 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGSAEAAAKEAAAKEAAAKAGGGGSAEAAAKEAAAKEAAAKAGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 33 - protein, 586 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGSGGGGSGGGGSGLSKMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 34 - protein, 597 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGSSSSSAEAAAKEAAAKEAAAKAGSSSSSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 35 - protein, 585 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGGSGGGGSGGGGSGGGGSGGGGSMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 36 - protein, 572 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGGSGGGGSGGGGSGGGGSGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 37 - protein, 587 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGGSAEAAAKEAAAKEAAAKAGGGGSMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 38 - protein, 589 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGSSSSSAEAAAKEAAAKEAAAKAGSSSSSMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 39 - protein, 578 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGSGGGGSGGGGSGLSKMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 40 - protein, 574 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 41 - protein, 576 aa, synthetic construct
MDAMKRGLCCVLLLCGAVFVSPSQEIHARFRRGARWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGSSSSSAEAAAKEAAAKEAAAKAGSSSSSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 42 - protein, 563 aa, synthetic construct
MPMGSLQPLATLYLLGMLVASVLAWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGGSAEAAAKEAAAKEAAAKAGGGGSMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLAQLSLELRKPITFGVITADTLEQAIERAGTKHGNKGWEAALSAIEMANLFKSLR

SEQ ID NO: 43 - protein, 554 aa, synthetic construct
MPMGSLQPLATLYLLGMLVASVLAWSHPQFEKHHHHHHHHLEVLFQGPYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYGGGSGGGGSGGGGSGLSKMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLAQLSLELRKPITFGVITADTLEQAIERAGTKHGNKGWEAALSAIEMANLFKSLR

SEQ ID NO: 44 - protein, 764 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 45 - protein, 755 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGSSSSSGSSSSSGSSSSSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 46 - protein, 755 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 47 - protein, 766 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGSSSSSAEAAAKEAAAKEAAAKAGSSSSSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 48 - protein, 736 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 49 - protein, 757 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGGGGSAEAAAKEAAAKEAAAKAGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 50 - protein, 785 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 51 - protein, 776 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 52 - protein, 777 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 53 - protein, 768 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 54 - protein, 787 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLRLEVLFQGPGHHHHHHHHSAWSHPQFEK

SEQ ID NO: 55 - protein, 760 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 56 - protein, 732 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 57 - protein, 752 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGGGGSAEAAAKEAAAKEAAAKAGGGGSSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 58 - protein, 781 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 59 - protein, 772 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 60 - protein, 773 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 61 - protein, 764 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 62 - protein, 751 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 63 - protein, 751 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKGGSGGWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGSSSSSGSSSSSGSSSSSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 64 - protein, 768 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGTFIKGGSGGNSTGGSGGWNYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 65 - protein, 721 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSGGSGGSGGSGGSPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 66 - protein, 761 aa, synthetic construct; identical to SEQ ID NO: 23

SEQ ID NO: 67 - protein, 761 aa, synthetic construct
MRPTWAWWLFLVLLLALWAPARGQAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGTGGGGSGGGGSNATGSSFYAPENITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 68 - protein, 756 aa, synthetic construct
MRGLGTCLATLAGLLLTAAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 69 - protein, 756 aa, synthetic construct
MRGLGTCLATLAGLLLTAAGSVASQSIIAYTMSLGAENSVACSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTICSGWTFGAGPALQIPFPMQMAYRFNGIGVTQNVLYENQKLIANQFNSAIGKIQDSLSSTPSALGKLQDVVNQNAQALNTLVKQLSSNCGAISSVLNDILSRLDKPEAEVQIDRLITCRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 70 - protein, 1300 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGGSGGKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 71 - protein, 1291 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGGSGGKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 72 - protein, 1272 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGGSGGKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGGGGSAEAAAKEAAAKEAAAKAGGGGSMQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLANLSLELRKPITFGVITADTLEQAIERAGTKHGNKGWEAALSAIEMANLFKSLR

SEQ ID NO: 73 - protein, 1321 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGGSGGKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 74 - protein, 1312 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGGSGGKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 75 - protein, 1293 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGGSGGKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGGGGSAEAAAKEAAAKEAAAKAGGGGSMSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 76 - protein, 1313 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGGSGGKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGGSAEAAAKEAAAKEAAAKAGGGGSMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 77 - protein, 1304 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGGSGGKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGGGGSGGGGSGGGGSGLSKMLSKDIIKLLNEQVNKEMNSSNLYMSMSSWCYTHSLDGAGLFLFDHAAEEYEHAKKLIVFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 78 - protein, 1371 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 79 - protein, 1249 aa, synthetic construct
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEK

SEQ ID NO: 80 - protein, 1267 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEK

SEQ ID NO: 81 - protein, 1334 aa, synthetic construct
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSGGSGSGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSLLEDLLFNKVKLSDVGFVEAYNNCTGGSEIRDLLCVQSFNGIKVLPPILSETQISGYTTAATVAAMFPPWSAAAGVPFSLNVQYRINGLGVTMDVLNKNQKLIANAFNKALLSIQNGFTATNSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNSGRLEVLFQGPGGYIPEAPRDGQAYVRKDGEWVLLSTFLGHHHHHHHHSAWSHPQFEK

SEQ ID NO: 82 - protein, 1188 aa, synthetic construct
MFVLLVAYALLHIAGCQTTNGTNTSHSVCNGCVGHSENVFAVESGGYIPSNFAFNNWFLLTNTSSVVDGVVRSFQPLLLNCLWSVSGSQFTTGFVYFNGTGRGACKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGDVEAVNFNVTNAATTVCTVALASYADVLVNVSQTAIANIIYCNSVINRLRCDQLSFDVPDGFYSTSPIQPVELPVSIVSLPVYHKHTFIVLYVNFEHRRGPGKCYNCRPAVINITLANFNETKGPLCVDTSHFTTQFVDNVKLARWSASINTGNCPFSFGKVNNFVKFGSVCFSLKDIPGGCAMPIMANLVNSKSHNIGSLYVSWSDGDVITGVPKPVEGVSSFMNVTLNKCTKYNIYDVSGVGVIRISNDTFLNGITYTSTSGNLLGFKDVTNGTIYSITPCNPPDQLVVYQQAVVGAMLSENFTSYGFSNVVEMPKFFYASNGTYNCTDAVLTYSSFGVCADGSIIAVQPRNVSYDSVSAIVTANLSIPFNWTTSVQVEYLQITSTPIVVDCSTYVCNGNVRCVELLKQYTSACKTIEDALRNSAMLESADVSEMLTFDKKAFTLANVSSFGDYNLSSVIPSLPRSGSRVAGRSAIEDILFSKLVTSGLGTVDADYKKCTKGLSIADLACAQYYNGIMVLPGVADAERMAMYTGSLIGGIALGGLTSAASIPFSLAIQSRLNYVALQTDVLQENQRILAASFNKAMTNIVDAFTGVNDAITQTSQALQTVATALNKIQDVVNQQGNSLNHLTSQLRQNFQAISSSIQAIYDRLDPPQADQQVDRLITGRLAALNVFVSHTLTKYTEVRASRQLAQQKVNECVKSQSKRYGFCGNGTHIFSLVNAAPEGLVFLHTVLLPTQYKDVEAWSGLCVDGINGYVLRQPNLALYKEGNYYRITSRIMFEPRIPTIADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 83 - protein, 1367 aa, synthetic construct
MFLILLISLPTAFAVIGDLKCPLDSRTGSLNNIDTGPPSISTATVDVTNGLGTYYVLDRVYLNTTLFLNGYYPTSGSTYRNMALKGTDKLSTLWFKPPFLSDFINGIFAKVKNTKVFKDGVMYSEFPAITIGSTFVNTSYSVVVQPRTINSTQDGVNKLQGLLEVSVCQYNMCEYPHTICHPKLGNHFKELWHMDTGVVSCLYKRNFTYDVNATYLYFHFYQEGGTFYAYFTDTGVVTKFLFNVYLGMALSHYYVMPLTCISRRDIGFTLEYWVTPLTSRQYLLAFNQDGIIFNAVDCMSDFMSEIKCKTQSIAPPTGVYELNGYTVQPIADVYRRKPDLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIENSVFKPQPAGVLTNHDVVYAQHCFKAPKNFCPCKLNSSLCVGSGPGKNNGIGTCPAGTNYLTCHNLCNPDPITFTGPYKCPQTKSLVGIGEHCSGLAVKSDYCGGNPCTCQPQAFLGWSADSCLQGDKCNIFANLILHDVNSGLTCSTDLQKANTDIKLGVCVNYDLYGISGQGIFVEVNATYYNSWQNLLYDSNGNLYGFRDYITNRTFMIRSCYSGRVSAAFHANSSEPALLFRNIKCNYVFNNSLIRQLQPINYFDSYLGCVVNAYNSTAISVQTCDLTVGSGYCVDYSKNRRSRRAITTGYRFTNFEPFTVNSVNDSLEPVGGLYEIQIPSEFTIGNMEEFIQTSSPKVTIDCAAFVCGDYAACKSQLVEYGSFCDNINAILTEVNELLDTTQLQVANSLMNGVTLSTKLKDGVNFNVDDINFSSVLGCLGSECSKASSRSAIEDLLFDKVKLSDVGFVAAYNNCTGGAEIRDLICVQSYKGIKVLPPLLSENQISGYTLAATSASLFPPWTAAAGVPFYLNVQYRINGLGVTMDVLSQNQKLIANAFNNALDAIQEGFDATNSALVKIQAVVNANAEALNNLLQQLSNRFGAISSSLQEILSRLDPPEAEAQIDRLINGRLTALNAYVSQQLSDSTLVKFSAAQAMEKVNECVKSQSSRINFCGNGNHIISLVQNAPYGLYFIHFSYVPTKYVTAKVSPGLCIAGDRGIAPKSGYFVNVNNTWMYTGSGYYYPEPITENNVVVMSTCAVNYTKAPYVMLNTSTPNLPDFREELDQWFKNQTSVAPDLSLDYINVTFLDLQVEMNRLQEAIKVLNGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 84 - protein, 1371 aa, synthetic construct
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDPPQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRIMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 85 - protein, 1326 aa, synthetic construct
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQGGPGGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 86 - protein, 1408 aa, synthetic construct
MFLIIFILPTTLAVIGDFNCTNSFINDYNKTIPRISEDVVDVSLGLGTYYVLNRVYLNTTLLFTGYFPKSGANFRDLALKGSIYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNLSSYDVVYSDHCFSVNSDFCPCADPSVVNSCAKSKPPSAICPAGTKYRHCDLDTTLYVKNWCRCSCLPDPISTYSPNTCPQKKVVVGIGEHCPGLGINEEKCGTQLNHSSCFCSPDAFLGWSFDSCISNNRCNIFSNFIFNGINSGTTCSNDLLYSNTEISTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSGGSGSGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSLLEDLLFNKVKLSDVGFVEAYNNCTGGSEIRDLLCVQSFNGIKVLPPILSETQISGYTTAATVAAMFPPWSAAAGVPFSLNVQYRINGLGVTMDVLNKNQKLIANAFNKALLSIQNGFTATNSALAKIQSVVNANAQALNSLLQQLFNKFGAISSSLQEILSRLDPPEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAELSLWFKNHTSIAPNLTFNSHINATFLDLYYEMNVIQESIKSLNGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 87 - protein, 1420 aa, synthetic construct
MFLILLISLPTAFAVIGDLKCPLDSRTGSLNNIDTGPPSISTATVDVTNGLGTYYVLDRVYLNTTLFLNGYYPTSGSTYRNMALKGTDKLSTLWFKPPFLSDFINGIFAKVKNTKVFKDGVMYSEFPAITIGSTFVNTSYSVVVQPRTINSTQDGVNKLQGLLEVSVCQYNMCEYPHTICHPKLGNHFKELWHMDTGVVSCLYKRNFTYDVNATYLYFHFYQEGGTFYAYFTDTGVVTKFLFNVYLGMALSHYYVMPLTCISRRDIGFTLEYWVTPLTSRQYLLAFNQDGIIFNAVDCMSDFMSEIKCKTQSIAPPTGVYELNGYTVQPIADVYRRKPDLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIENSVFKPQPAGVLTNHDVVYAQHCFKAPKNFCPCKLNSSLCVGSGPGKNNGIGTCPAGTNYLTCHNLCNPDPITFTGPYKCPQTKSLVGIGEHCSGLAVKSDYCGGNPCTCQPQAFLGWSADSCLQGDKCNIFANLILHDVNSGLTCSTDLQKANTDIKLGVCVNYDLYGISGQGIFVEVNATYYNSWQNLLYDSNGNLYGFRDYITNRTFMIRSCYSGRVSAAFHANSSEPALLFRNIKCNYVFNNSLIRQLQPINYFDSYLGCVVNAYNSTAISVQTCDLTVGSGYCVDYSKNRRSRRAITTGYRFTNFEPFTVNSVNDSLEPVGGLYEIQIPSEFTIGNMEEFIQTSSPKVTIDCAAFVCGDYAACKSQLVEYGSFCDNINAILTEVNELLDTTQLQVANSLMNGVTLSTKLKDGVNFNVDDINFSSVLGCLGSECSKASSRSAIEDLLFDKVKLSDVGFVAAYNNCTGGAEIRDLICVQSYKGIKVLPPLLSENQISGYTLAATSASLFPPWTAAAGVPFYLNVQYRINGLGVTMDVLSQNQKLIANAFNNALDAIQEGFDATNSALVKIQAVVNANAEALNNLLQQLSNRFGAISSSLQEILSRLDPPEAEAQIDRLINGRLTALNAYVSQQLSDSTLVKFSAAQAMEKVNECVKSQSSRINFCGNGNHIISLVQNAPYGLYFIHFSYVPTKYVTAKVSPGLCIAGDRGIAPKSGYFVNVNNTWMYTGSGYYYPEPITENNVVVMSTCAVNYTKAPYVMLNTSTPNLPDFREELDQWFKNQTSVAPDLSLDYINVTFLDLQVEMNRLQEAIKVLNGGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 88 - protein, 1425 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPASVGSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDPPEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 89 - protein, 1342 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 90 - protein, 564 aa, Middle East respiratory syndrome-related coronavirus
QAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGTFIKGGSGGNSTGGSGGWNYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLG

SEQ ID NO: 91 - protein, 517 aa, Middle East respiratory syndrome-related coronavirus
QAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSGGSGGSGGSGGSPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLG

SEQ ID NO: 92 - protein, 557 aa, Middle East respiratory syndrome-related coronavirus
QAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGTGGGGSGGGGSNATGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLG

SEQ ID NO: 93 - protein, 559 aa, Middle East respiratory syndrome-related coronavirus
QAFNHPIQVDQLNSSYFKLSIPTNFSFGVTQEYIQTTIQKVCVDCKQYVCNGFQKCEQLLREYGQFCSKINQALHGCNLRQDDSVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFHKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGTGGGGSGGGGSNATGSSFYAPENITSLNTKYVAPQVTYQNISTNLPPPLLGNSTGIDFQDELDEFFKNVSTSIPNFGSLTQINTTLLDLTYEMLSLQQVVKALNESYIDLKELGNYTYGSGYIPEAPRDGQAYVRKDGEWVLLSTFLGRS

SEQ ID NO: 94 - protein, 1093 aa, Severe acute respiratory syndrome coronavirus 2
SSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGGGSGGKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 95 - protein, 1093 aa, Severe acute respiratory syndrome coronavirus 2; identical to SEQ ID NO: 94

SEQ ID NO: 96 - protein, 1043 aa, Severe acute respiratory syndrome coronavirus 2
SSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGGGSGGKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLG

SEQ ID NO: 97 - protein, 27 aa, synthetic construct
GYIPEAPRDGQAYVRKDGEWVLLSTFL

SEQ ID NO: 98 - protein, 8 aa, synthetic construct
WSHPQFEK

SEQ ID NO: 99 - protein, 24 aa, synthetic construct
MPMGSLQPLATLYLLGMLVASVLA

SEQ ID NO: 100 - protein, 1353 aa, unidentified
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 101 - protein, 524 aa, synthetic construct
IAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTY

SEQ ID NO: 102 - skipped

SEQ ID NO: 103 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 104 - protein, 42 aa, Severe acute respiratory syndrome-related coronavirus
SFIEDLLFNKVTLADAGFIKQYGDCLGDIAARDLICAQKFNG

SEQ ID NO: 105 - protein, 42 aa, Severe acute respiratory syndrome-related coronavirus
SFIEDLLFNKVTLADAGFIKQYGDCLGDVAARDLICAQKFNG

SEQ ID NO: 106 - protein, 524 aa, synthetic construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 107 - protein, 529 aa, synthetic construct
AFNHPIQVDQLNSSYFKLSIPTNFSFGVTQEYIQTTIQKVCVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFQKVQDAVNNNAQALSKLASELSNTFGAISASIGDIIQRLDPPEQDAQIDRLINGRLTTLNAFVAQQLVRSEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTY

SEQ ID NO: 108 - protein, 527 aa, synthetic construct
AFNHPIQVDQLNSSYFKLSIPTNFSFGVTQEYIQTTIQKVCVDCKQYVCNGFQKCEQLLREYGQFCSKINQALHGCNLRQDVRNLFASVKSSQSSPIIPGFGGDFNLTLLEPVSISTGSRSARSAIEDLLFDKCTIADPGYMQGYDDCMQQGPASARDLICAQYVAGYCVLPPLMDVNMEAAYTSSLLGSIAGSGWTAGLSSFAAIPFAQMIFYRLNGIGITQQVLSENQKLIANKFNQALGAMQTGFTTTNEAFQKCQDAVNNNAQALSKLASELSNTFGAISASIGDIIQRLDPPEQDAQIDRLINGRLTTLNAFVAQQLVRCEEAAQSAQLAKDKVNECVKAQSKRSGFCGQGTHIVSFVVNAPYGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTY

SEQ ID NO: 109 - protein, 1476 aa, synthetic construct
MDAMKRGLCCVLLLCGAVFVSPSASVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYRYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 110 - protein, 819 aa, synthetic construct
MRGLGTCLATLAGLLLTAAGRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYRYRLFRKSNLKPFERDISTEIYQAGSKPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGGGSRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSTPSGNYNYRYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 111 - protein, 1555 aa, synthetic construct
MPMGSLQPLATLYLLGMLVASVLAVIGDLNCTLDPRLKGSFNNRDTGPPSISIDTVDVTNGLGTYYVLDRVYLNTTLFLNGYYPTSGSTYRNMALKGTDLLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHYDTGVVSCLYKRNFTYDVNATYLYFHFYQEGGTFYAYFTDTGFVTKFLFNVYLGMALSHYYVMPLTCIRRSKDGFSPEYWVTPLTPRQYLLAFNQDGIIFNAVDCMSDFMSEIKCKTQSIAPPTGVYELNGYTVQPIADVYRRKPDLPNCNIEAWLNDKSVPSPLNWERKTFSNCNFNMSSLMSFIQADSFTCNNIDAAKIYGMCFSSITIDKFAIPNRRKVDLQLGNLGYLQSSNYRIDTTATSCQLYYNLPAANVSVSRFNPSTWNKRFGFIEDAVFKPQPAGVLTNHDVVYAQHCFKAPKNFCPCSSCSGKNNGIGTCPAGTNSLTCDNLCTLDPITLKAPDTYKCPQSKSLVGIGEHCSGLAVKSDYCGNNSCTCQPQAFLGWSADSCLQGDKCNIFANFILHDVNNGLTCSTDLQKANTEIELGVCVNYDLYGISGQGIFVEVNATYYNSWQNLLYDSNGNLYGFRDYITNRTFMIHSCYSGRVSAAYHANSSEPALLFRNIKCNYVFNNSLTRQLQPINYSFDSYLGCVVNAYNSTAISVQTCDLTVGSGYCVDYFKNGGSGGAITTGYRFTNFEPFTVNSVNDSLEPVGGLYEIQIPSEFTIGNMEEFIQTSSPKVTIDCAAFVCGDYAACKLQLVEYGSFCDNINAILTEVNELLDTTQLQVANSLMNGVTLSTKLKDGVNFNVDDINFAPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALHAIQQGFDATNSALVKIQAVVNANSEALNNLLQQLSNRFGAISASLQEILSRLDPPEAEAQIDRLINGRLTALNAYVSQQLSDSTLVKFSAAQAMEKVNECVKSQSSRINFCGNGNHIISLVQNAPYGLYFIHFNYVPTKYVTAKVSPGLCIAGNRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 112 - protein, 1008 aa, synthetic construct
MPMGSLQPLATLYLLGMLVASVLAIADVYRRKPDLPNCNIEAWLNDKSVPSPLNWERKTFSNCNFNMSSLMSFIQADSFTCNNIDAAKIYGMCFSSITIDKFAIPNRRKVDLQLGNLGYLQSSNYRIDTTATSCQLYYNLPAANVSVSRFNPSTWNKRFGFIEDAVFKPQPAGVLTNHDVVYAQHCFKAPKNFCPCSSCSGKNNGIGTCPAGTNSLTCDNLCTLDPITLKAPDTYKCPQSKSLVGIGEHCSGLAVKSDYCGNNSCTCQPQAFLGWSADSCLQGDKCNIFANFILHDVNNGLTCSTDLQKANTEIELGVCVNYDLYGISGQGIFVEVNATYYNSWQNLLYDSNGNLYGFRDYITNRTFMIHSCYSGGGSGVYELNGYTVQPIADVYRRKPDLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPQPAGVLTDHDVVYAQHCFKAPKNFCPCKSNSSLCVGSGPGKNNGIGTCPAGTNYLTCHNLCNPDPITFTGPYKCPQTKSLVGIGEHCSGLAVKSDYCGGNPCTCQPQAFLGWSADSCLQGDKCNIFANLILHDVNSGLTCSTDLQKANTDIKLGVCVNYDLYGISGQGIFVEVNATYYNSWQNLLYDSNGNLYGFRDYITNRTFMIRSCYSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 113 - protein, 873 aa, synthetic construct
MDAMKRGLCCVLLLCGAVFVSAGSVIGDLNCTLDPRLKGSFNNRDTGPPSISIDTVDVTNGLGTYYVLDRVYLNTTLFLNGYYPTSGSTYRNMALKGTDLLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHYDTGVVSCLYKRNFTYDVNATYLYFHFYQEGGTFYAYFTDTGFVTKFLFNVYLGMALSHYYVMPLTCIRRSKDGFSPEYWVTPLTPRQYLLAFNQDGIIFNAGGSVIGDLKCPLDSRTGSLNNIDTGPPSISTATVDVTNGLGTYYVLDRVYLNTTLFLNGYYPTSGSTYLNMALKGTDKLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHMDTGVVSCLYKRNFTYDVNATYLYFHFYQEGGTFYAYFTDTGVVTKFLFNVYLGMALSHYYVMPLTCISRRNIGFTLEYWVTPLTSRQYLLAFNQDGIIFNAVDCMSDFMSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 114 - protein, 1438 aa, synthetic construct
MLLIIFILPTTLAVIGDFNCTNFAINDKNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDFLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVYTDVCVDYDLYGITGQGIFKEVSAVYYNSWQNLLYDSNGNIIGFKDFVTNKTYNIFPCYAGRVSAAFHQNASSLALLYRNLKCSYVLNNISLATQPYFDSYLGCVFNADNLTDYSVSSCALRMGSGFCVDYNSPSFSSSGGSGSSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDPPEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 115 - protein, 1029 aa, synthetic construct
MDAMKRGLCCVLLLCGAVFVSPSASGSTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVYTDVCVDYDLYGITGQGIFKEVSAVYYNSWQNLLYDSNGNIIGFKDFVTNKTYNIFPCYAGGGSTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTKYRHCDLDTTLYVKNWCRCSCLPDPISTYSPNTCPQKKVVVGIGEHCPGLGINEEKCGTQLNHSSCSCSPDAFLGWSFDSCISNNRCNIFSNFIFNGINSGTTCSNDLLYSNTDVSTGVCVNYDLYGITGQGIFKEVSAAYYNDWQNLLYDSNGNIIGFKDFLTNKTYTILPCYSGRVSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 116 - protein, 867 aa, synthetic construct
MPMGSLQPLATLYLLGMLVASVLAVIGDFNCTNFAINDKNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDFLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSGGSVIGDFNCTNSFINHYNKTIPRISEDGVDVSLGLGTYYVLNRVYLNTTLLFTGYFPKSGANFTDLALKGSTYLSTLWYKPPFLSDFNNGIFSKVKNTKLYVNNILYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISPKTDKETLEYWVTPLSRRQYLLNFDEHGVITKAVDCSSSFLSEIQCKTQSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 117 - protein, 1377 aa, synthetic construct
MFVLLVAYALLHIAGCQTTNGTNTSHAVCNGCVGHSENVFAVESGGYIPSNFAFNNWFLLTNTSSVVDGVVRSFQPLLLNCLWSVSGSQVITGFVYFNGTGRGACKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGNVEAVNFNVTNAATTVCTVALASYADVLVNVSQTAIANIIYCNSVINRLRCDQLSFDVPDGFYSTSPIQPVELPVSIVSLPVYHKHTFIVLYVNFEHQRGPGKCYNCRPAVINITLANFNETKGPLCVDTSHFTTQFVDNVKLPRWSASIDTGNCPFSFGKVINFVKFGSVCFSLKYIPGGCAMPIMANLVNYKSHNIGSLYVSWSDGDVITGVPKPVEGVSSFMNVTLNKCTKYNIYDVSGVGVIRISNDTFLNGITYTSTSGNLLGFKDVTNGTIYSITPCNPPDQLVVYQQAVVGAMLSENFTSYGFSNVVEMPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSAMLESADVSEMLTFDKKAFTLANVSSFGDYNLSSVIPSLPRSGSRVAGRSAIEDILFSKLVTSGLGTVDADYKKCTKGLSIADLACAQYYNGIMVLPGAADAERMAMYTGSLIGGIALGGLTSAASIPFSLAIQSRLNYVALQTDVLQENQRILAASFNKAMTNIVDAFTGVNDAITQTSQALQTVATALNKIQDVVNQQGNSLNHLTSQLRQNFQAISSSIQAIYDRLDPPQADQQVDRLITGRLAALNVFVSHTLTKYTEVRASRQLAQQKVNECVKSQSKRYGFCGNGTHIFSLVNAAPEGLVFLHTVLLPTQYKDVEAWSGLCVDGINGYVLRQPNLALYKEGNYYRITSRIMFEPRIPTIADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 118 - protein, 783 aa, synthetic construct
MFVLLVAYALLHIAGCQTTNGTNTSHAVCNGCVGHSENVFAVESGGYIPSNFAFNNWFLLTNTSSVVDGVVRSFQPLLLNCLWSVSGSQVITGFVYFNGTGRGACKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGNVEAVNFNVTNAATTVCTVALASYADVLVNVSQTAIANIIYCNSVINRLRCDQLSGGSQTTNGMNTSHSVCNGCVGHSENVFAVESGGYIPSNFAFNNWFLLTNTSSVVDGVVRSFQPLLLNCLWSVSGSRLTTGFVYFNGTGRGDCKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGNVEAVNFNVTNAATTVCTVALASYADVLVNVSQTAIANIIYCNSVINRLRCDQLSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 119 - protein, 1553 aa, synthetic construct
MKLFLILLILPLVSCFSTCNSNASISMLQLGVPDNSSTIVTGLLPVHWICANQSTSTYPANGFFYIDVGKHRSAFALHSGYYDANQYYIYLTNNISLNAPVTLKICKFGNTSFACLSNVSTSHHCIVNSSFTEQLGVPLGITISGETVRLHLYNATRTFYVPAAYKLTKLSVKCYFSESCVFSVVNATVTVNVTTLNGRIVNYTVCDDCNGYTDNIFSVQQDGRIPNGFSFNNWFLLTNGSTLVDGVSRLYQPLRLTCLWPVPGLKSSTGFVYFNATGSDVNCNGYQHHSVADVMRYNLNFSSNSVDNLKSGVIVFKTLQYDVLFYCSNSSSGVLDTTIPFGPSSQPYYCFINSTINTTHVSTFVGVLPPIVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPDTYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDPPQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVKLHTVIPDYVDVNKTLQEFAQNLPKYVKPNFDLTPFNLTYLNFSFELLQLRTQTASLFQTTVELQGLIDQINSTYVDLKLLNRFENYGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 120 - protein, 1181 aa, synthetic construct
MKLFLILLILPLVSCFSTCNSNASISMLQLGVPDNSSTIVTGLLPVHWICANQSTSTYPANGFFYIDVGKHRSAFALHSGYYDANQYYIYLTNNISLNAPVTLKICKFGNTSFACLSNVSTSHHCIVNSSFTEQLGVPLGITISGETVRLHLYNATRTFYVPAAYKLTKLSVKCYFSESCVFSVVNATVTVNVTTLNGRIVNYTVCDDCNGYTDNIFSVQQDGRIPNGFSFNNWFLLTNGSTLVDGVSRLYQPLRLTCLWPVPGLKSSTGFVYFNATGSDVNCNGYQHHSVADVMRYNLNFSSNSVDNLKSGVIVFKTLQYDVLFYCSNSSSGVLDTTIPFGPSSQPYYCFINSTINTTHVSTFVGVLPPIVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPDGGSSNANLSMLQLGVPDNSSTIVTGLLPTHWICANQSTSVYSANGFFFIDVGNHRSAFALHTGYYDVNQYYIYVTNEIGLNASVTLKICKFGNTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDNGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEWSHPQFEKGSGHHHHHHHH

SEQ ID NO: 121 - protein, 167 aa, synthetic construct
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAAGSLEHHHHHH

SEQ ID NO: 122 - protein, 215 aa, synthetic construct
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEHHHHHH

SEQ ID NO: 123 - protein, 1189 aa, Severe acute respiratory syndrome coronavirus 2
VNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYRYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 124 - protein, 557 aa, Severe acute respiratory syndrome coronavirus 2
MRGLGTCLATLAGLLLTAAGRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYRYRLFRKSNLKPFERDISTEIYQAGSKPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGGGSRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSTPSGNYNYRYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGG

SEQ ID NO: 125 - protein, 1269 aa, unidentified
VIGDLNCTLDPRLKGSFNNRDTGPPSISIDTVDVTNGLGTYYVLDRVYLNTTLFLNGYYPTSGSTYRNMALKGTDLLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHYDTGVVSCLYKRNFTYDVNATYLYFHFYQEGGTFYAYFTDTGFVTKFLFNVYLGMALSHYYVMPLTCIRRSKDGFSPEYWVTPLTPRQYLLAFNQDGIIFNAVDCMSDFMSEIKCKTQSIAPPTGVYELNGYTVQPIADVYRRKPDLPNCNIEAWLNDKSVPSPLNWERKTFSNCNFNMSSLMSFIQADSFTCNNIDAAKIYGMCFSSITIDKFAIPNRRKVDLQLGNLGYLQSSNYRIDTTATSCQLYYNLPAANVSVSRFNPSTWNKRFGFIEDAVFKPQPAGVLTNHDVVYAQHCFKAPKNFCPCSSCSGKNNGIGTCPAGTNSLTCDNLCTLDPITLKAPDTYKCPQSKSLVGIGEHCSGLAVKSDYCGNNSCTCQPQAFLGWSADSCLQGDKCNIFANFILHDVNNGLTCSTDLQKANTEIELGVCVNYDLYGISGQGIFVEVNATYYNSWQNLLYDSNGNLYGFRDYITNRTFMIHSCYSGRVSAAYHANSSEPALLFRNIKCNYVFNNSLTRQLQPINYSFDSYLGCVVNAYNSTAISVQTCDLTVGSGYCVDYFKNGGSGGAITTGYRFTNFEPFTVNSVNDSLEPVGGLYEIQIPSEFTIGNMEEFIQTSSPKVTIDCAAFVCGDYAACKLQLVEYGSFCDNINAILTEVNELLDTTQLQVANSLMNGVTLSTKLKDGVNFNVDDINFAPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALHAIQQGFDATNSALVKIQAVVNANSEALNNLLQQLSNRFGAISASLQEILSRLDPPEAEAQIDRLINGRLTALNAYVSQQLSDSTLVKFSAAQAMEKVNECVKSQSSRINFCGNGNHIISLVQNAPYGLYFIHFNYVPTKYVTAKVSPGLCIAGNRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLN

SEQ ID NO: 126 - protein, 722 aa, unidentified
IADVYRRKPDLPNCNIEAWLNDKSVPSPLNWERKTFSNCNFNMSSLMSFIQADSFTCNNIDAAKIYGMCFSSITIDKFAIPNRRKVDLQLGNLGYLQSSNYRIDTTATSCQLYYNLPAANVSVSRFNPSTWNKRFGFIEDAVFKPQPAGVLTNHDVVYAQHCFKAPKNFCPCSSCSGKNNGIGTCPAGTNSLTCDNLCTLDPITLKAPDTYKCPQSKSLVGIGEHCSGLAVKSDYCGNNSCTCQPQAFLGWSADSCLQGDKCNIFANFILHDVNNGLTCSTDLQKANTEIELGVCVNYDLYGISGQGIFVEVNATYYNSWQNLLYDSNGNLYGFRDYITNRTFMIHSCYSGGGSGVYELNGYTVQPIADVYRRKPDLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPQPAGVLTDHDVVYAQHCFKAPKNFCPCKSNSSLCVGSGPGKNNGIGTCPAGTNYLTCHNLCNPDPITFTGPYKCPQTKSLVGIGEHCSGLAVKSDYCGGNPCTCQPQAFLGWSADSCLQGDKCNIFANLILHDVNSGLTCSTDLQKANTDIKLGVCVNYDLYGISGQGIFVEVNATYYNSWQNLLYDSNGNLYGFRDYITNRTFMIRSCYS

SEQ ID NO: 127 - protein, 571 aa, unidentified
LNCTLDPRLKGSFNNRDTGPPSISIDTVDVTNGLGTYYVLDRVYLNTTLFLNGYYPTSGSTYRNMALKGTDLLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHYDTGVVSCLYKRNFTYDVNATYLYFHFYQEGGTFYAYFTDTGFVTKFLFNVYLGMALSHYYVMPLTCIRRSKDGFSPEYWVTPLTPRQYLLAFNQDGIIFNAGGSVIGDLKCPLDSRTGSLNNIDTGPPSISTATVDVTNGLGTYYVLDRVYLNTTLFLNGYYPTSGSTYLNMALKGTDKLSTLWFKPPFLSDFINGIFAKVKNTKVFKDGVMYSEFPAITIGSTFVNTSYSVVVQPRTINSTQDGVNKLQGLLEVSVCQYNMCEYPHTICHPNLGNHFKELWHMDTGVVSCLYKRNFTYDVNATYLYFHFYQEGGTFYAYFTDTGVVTKFLFNVYLGMALSHYYVMPLTCISRRNIGFTLEYWVTPLTSRQYLLAFNQDGIIFNAVDCMSDFMS

SEQ ID NO: 128 - protein, 1176 aa, Human coronavirus HKU1
MLLIIFILPTTLAVIGDFNCTNFAINDKNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDFLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVYTDVCVDYDLYGITGQGIFKEVSAVYYNSWQNLLYDSNGNIIGFKDFVTNKTYNIFPCYAGRVSAAFHQNASSLALLYRNLKCSYVLNNISLATQPYFDSYLGCVFNADNLTDYSVSSCALRMGSGFCVDYNSPSFSSSGGSGSSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDPPEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAP

SEQ ID NO: 129 - protein, 742 aa, Human coronavirus HKU1
GSTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVYTDVCVDYDLYGITGQGIFKEVSAVYYNSWQNLLYDSNGNIIGFKDFVTNKTYNIFPCYAGGGSTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTKYRHCDLDTTLYVKNWCRCSCLPDPISTYSPNTCPQKKVVVGIGEHCPGLGINEEKCGTQLNHSSCSCSPDAFLGWSFDSCISNNRCNIFSNFIFNGINSGTTCSNDLLYSNTDVSTGVCVNYDLYGITGQGIFKEVSAAYYNDWQNLLYDSNGNIIGFKDFLTNKTYTILPCYSGRVS

SEQ ID NO: 130 - protein, 569 aa, unidentified
VIGDFNCTNFAINDKNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDFLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSGGSVIGDFNCTNSFINHYNKTIPRISEDGVDVSLGLGTYYVLNRVYLNTTLLFTGYFPKSGANFTDLALKGSTYLSTLWYKPPFLSDFNNGIFSKVKNTKLYVNNILYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCNAISPKTDKETLEYWVTPLSRRQYLLNFDEHGVITKAVDCSSSFLSEIQCKTQS

SEQ ID NO: 131 - protein, 1115 aa, Human coronavirus 229E
MFVLLVAYALLHIAGCQTTNGTNTSHAVCNGCVGHSENVFAVESGGYIPSNFAFNNWFLLTNTSSVVDGVVRSFQPLLLNCLWSVSGSQVITGFVYFNGTGRGACKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGNVEAVNFNVTNAATTVCTVALASYADVLVNVSQTAIANIIYCNSVINRLRCDQLSFDVPDGFYSTSPIQPVELPVSIVSLPVYHKHTFIVLYVNFEHQRGPGKCYNCRPAVINITLANFNETKGPLCVDTSHFTTQFVDNVKLPRWSASIDTGNCPFSFGKVINFVKFGSVCFSLKYIPGGCAMPIMANLVNYKSHNIGSLYVSWSDGDVITGVPKPVEGVSSFMNVTLNKCTKYNIYDVSGVGVIRISNDTFLNGITYTSTSGNLLGFKDVTNGTIYSITPCNPPDQLVVYQQAVVGAMLSENFTSYGFSNVVEMPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSAMLESADVSEMLTFDKKAFTLANVSSFGDYNLSSVIPSLPRSGSRVAGRSAIEDILFSKLVTSGLGTVDADYKKCTKGLSIADLACAQYYNGIMVLPGAADAERMAMYTGSLIGGIALGGLTSAASIPFSLAIQSRLNYVALQTDVLQENQRILAASFNKAMTNIVDAFTGVNDAITQTSQALQTVATALNKIQDVVNQQGNSLNHLTSQLRQNFQAISSSIQAIYDRLDPPQADQQVDRLITGRLAALNVFVSHTLTKYTEVRASRQLAQQKVNECVKSQSKRYGFCGNGTHIFSLVNAAPEGLVFLHTVLLPTQYKDVEAWSGLCVDGINGYVLRQPNLALYKEGNYYRITSRIMFEPRIPTIADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWW

SEQ ID NO: 132 - protein, 521 aa, Human coronavirus 229E
MFVLLVAYALLHIAGCQTTNGTNTSHAVCNGCVGHSENVFAVESGGYIPSNFAFNNWFLLTNTSSVVDGVVRSFQPLLLNCLWSVSGSQVITGFVYFNGTGRGACKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGNVEAVNFNVTNAATTVCTVALASYADVLVNVSQTAIANIIYCNSVINRLRCDQLSGGSQTTNGMNTSHSVCNGCVGHSENVFAVESGGYIPSNFAFNNWFLLTNTSSVVDGVVRSFQPLLLNCLWSVSGSRLTTGFVYFNGTGRGDCKGFYSNASSDVIRYNINFEENLRRGTILFKTSYGAVVFYCTNNTLVSGDAHIPSGTVLGNFYCFVNTTIGNETTSAFVGALPKTVREFVISRTGHFYINGYRYFSLGNVEAVNFNVTNAATTVCTVALASYADVLVNVSQTAIANIIYCNSVINRLRCDQLS

SEQ ID NO: 133 - protein, 1291 aa, Human coronavirus NL63
MKLFLILLILPLVSCFSTCNSNASISMLQLGVPDNSSTIVTGLLPVHWICANQSTSTYPANGFFYIDVGKHRSAFALHSGYYDANQYYIYLTNNISLNAPVTLKICKFGNTSFACLSNVSTSHHCIVNSSFTEQLGVPLGITISGETVRLHLYNATRTFYVPAAYKLTKLSVKCYFSESCVFSVVNATVTVNVTTLNGRIVNYTVCDDCNGYTDNIFSVQQDGRIPNGFSFNNWFLLTNGSTLVDGVSRLYQPLRLTCLWPVPGLKSSTGFVYFNATGSDVNCNGYQHHSVADVMRYNLNFSSNSVDNLKSGVIVFKTLQYDVLFYCSNSSSGVLDTTIPFGPSSQPYYCFINSTINTTHVSTFVGVLPPIVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPDTYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVAVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDPPQADQQVDRLITGRLAALNAFVSQVLNKYTEVRSSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVKLHTVIPDYVDVNKTLQEFAQNLPKYVKPNFDLTPFNLTYLNFSFELLQLRTQTASLFQTTVELQGLIDQINSTYVDLKLLNRFENY

SEQ ID NO: 134 - protein, 919 aa, Human coronavirus NL63
MKLFLILLILPLVSCFSTCNSNASISMLQLGVPDNSSTIVTGLLPVHWICANQSTSTYPANGFFYIDVGKHRSAFALHSGYYDANQYYIYLTNNISLNAPVTLKICKFGNTSFACLSNVSTSHHCIVNSSFTEQLGVPLGITISGETVRLHLYNATRTFYVPAAYKLTKLSVKCYFSESCVFSVVNATVTVNVTTLNGRIVNYTVCDDCNGYTDNIFSVQQDGRIPNGFSFNNWFLLTNGSTLVDGVSRLYQPLRLTCLWPVPGLKSSTGFVYFNATGSDVNCNGYQHHSVADVMRYNLNFSSNSVDNLKSGVIVFKTLQYDVLFYCSNSSSGVLDTTIPFGPSSQPYYCFINSTINTTHVSTFVGVLPPIVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPDGGSSNANLSMLQLGVPDNSSTIVTGLLPTHWICANQSTSVYSANGFFFIDVGNHRSAFALHTGYYDVNQYYIYVTNEIGLNASVTLKICKFGNTTFDFLSNSSSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVADVMRYNLNFSANSVDNLKSGVIVFKTLQYDVLFYCSNSSSGVLDTTIPFGPSSQPYYCFINSTINTTHVSTFVGVLPPTVREIVVARTGQFYINGFKYFDLGFIEAVNFNVTTASATDFWTVAFATFVDVLVNVSATKIQNLLYCDSPFEKLQCEHLQFGLQDGFYSANFLDDN

SEQ ID NO: 135 - protein, 852 aa, synthetic construct
ATMGILPSPGMPALLSLVSLLSVLLMGCVAETGTECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYGGSEAAATGTFIEQPKSKECDFTPMLVGVPPQVYNFKRLVFTNCNYNLTKLLSLFMVNEFSCNGISPDAIARGCYSSLTVDYFAYPLSMRSYIQPGSAGDISLYNYKQSFANPTCRVLATAPANLTLTKPSAYGYFQKCSRVSGEHNSVETPLYINPGEYSICRSFSPYGFSEDGEVFRRQLTQYEGGGILVGVGAKLAMTDKLEMGFIISVQYGTDTNSVCPMLDLGNSSTITHYLGKCVDYSLYGVTGRGVFQNCTAVGIRQQRFVYDSFDNLVGYYSDDGNGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 136 - protein, 591 aa, synthetic construct
ATMGILPSPGMPALLSLVSLLSVLLMGCVAETGTASATGTFIEQPNATECDFSPMLTGVAPQVYNFKRLVFSNCNYNLTKLLSLFAVDEFSCNGISPDAIARGCYSTLTVDYFAYPLSMKSYIRPGSAGNIPLYNYKQSFANPTCRVMASVPANVTITKPDAYGYISKCSRLTGANQDVETPLYINPGEYSICRDFSPGGFSEDGQVFKRTLTQFEGGGLLIGVGTRVPMTDNLQMSFIIFVQYGTGTDSVCPMLDLGDSLTITNRLGKCVDYSLYGVTGRGVFQNCSAVGVKQQRFVYDSFDNLVGYYSDDGNYYCGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 137 - protein, 897 aa, synthetic construct
ATMGILPSPGMPALLSLVSLLSVLLMGCVAETGTEAQPRGSFIEQAQGTECDFSPLLKDEPPQVYNFSRLVFTNCNYNLTKLLALFQVSQFSCHQVSPSALASGCYSSLTVDYFAYPTYMSSYLQQGSTGEISQFNYKQDFSNPTCRILATVPANLSASGLLPKPSNYVWLSECYQNSFTGKNFQYVKAGQYTPCLGLAANGFEKSYQTHRDPVSKLAVTGVVTPMTSALQMAFIISVQYGTDSNSVCPMQAVRNDTSVDDKLGLCIDYSLYGITGRGVFQNCTAVGIRQQRFVYDSFDNLVGYHADNGNYYCVRPCVSVPVSVVFDRSTNTHATLFGSGGSEASPRGMFVEQSTSEECDFTKMLTGTPPQVYNFNRMVFTNCNYNLTKLLNLFQVSEFSCYQVSPSSLATGCYSSLTVDSFAFPLELSSYMQTGSAGPIRQFNYNQDFSKPTCRVLASVPSNYTALSKPSNYVHLTECYTLGYSAKQYHNTQPGQYTPCLQLANQGFSRSGQSNSVELPSGEFKTATGYIIPMTDQLQMAFVISVQYGTDTNSVCPMQALRNDTSIQDKLDVCVEYSLHGVTGRGVFHNCTSVGLRNQRFVYDTFDNLVGYHSDNGNYYCVRGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 138 - protein, 588 aa, synthetic construct
ATMGILPSPGMPALLSLVSLLSVLLMGCVAETGTEASPRGEFIEQATTQECDFTPMLTGTPPPIYNFKRLVFTNCNYNLTKLLSLFQVSEFSCHQVSPSSLATGCYSSLTVDYFAYSTDMSSYLQPGSAGEIVQFNYKQDFSNPTCRVLATVPQNLTTITKPSNYAYLTECYKTSAYGKNYLYNAPGGYTPCLSLASRGFSTKYQSHSDGELTTTGYIYPVTGNLQMAFIISVQYGTDTNSVCPMQALRNDTSIEDKLDVCVEYSLHGITGRGVFHNCTSVGLRNQRFVYDTFDNLVGYHSYNGNYYCVRPCVSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 139 - protein, 907 aa, synthetic construct
ATMGILPSPGMPALLSLVSLLSVLLMGCVAETGTATMGILPSPGMPALLSLVSLLSVLLMGCVAETGTEAHVYPDCNFTDLFRENAPTIMQYKRQVFTRCNYNLTLLLSLVQVDEFVCDKITPEALATGCYSSLTVDWFAFPYAWKSYLAIGSADRIVRFNYNQDYSNPSCRIHSKVNSSVGISYSGLYSYITNCNYGGFNKDDVVKPGGRASQPCVTGALNSPTNGQVWSFNFGGVPYRTSRLTYTDHLKNPLDMVYVITVKYEPGAETVCPKQVRPDYSTNITGLLGSCISYDIYGITGTGVFQLCNATGIPQQKFVYDKFDNIIGFHSDDGNYYCVAPCVSGGSSAKPVGAYYEAHVYPDCNFTELFRERAPTIMQYKREVFTRCNYNLSLLLSLVQVDEFVCDKITPEALATGCYSSLTVDWFAFPYAWKSYLAIGSADRIVRFNYNQDYSNPSCRIHSKVNSSIGISYAGAYSYITNCNYGATNKDDVVKPGGRASQQCITGALNSPTTGQLWAYNFGGVPYRVSRLTYTDHLSDPLDMVYVITVKYEPGAETVCPKQIRPDYSTNITHLLGSCISYDIYGITGTGVFQLCNATGIRQQKFVYDKFDNIIGFHSDDGNYYCVAPCVSVGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 140 - protein, 584 aa, synthetic construct
ATMGILPSPGMPALLSLVSLLSVLLMGCVAETGTEAHVYPDCNFTDLFRENAPTIMQYKRQVFTRCNYNLTLLLSLVQVDEFVCDKITPEALATGCYSSLTVDWFAFPYAWKSYLAIGSADRIVRFNYNQDYSNPSCRIHSKVNSSVGISYSGLYSYITNCNYGGFNKDDVVKPGGRASQPCVTGALNSPTNGQVWSFNFGGVPYRTSRLTYTDHLKNPLDMVYVITVKYEPGAETVCPKQVRPDYSTNITGLLGSCISYDIYGITGTGVFQLCNATGIPQQKFVYDKFDNIIGFHSDDGNYYCVAPCVSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 141 - protein, 884 aa, synthetic construct
ATMGILPSPGMPALLSLVSLLSVLLMGCVAETGTECELDVLFKNDAPIIANYSRRVFTNCNYNLTKLLSLVEVDEFICHKTTPEALATGCYSSLTVDWFALPFSMKSTLAIGSAEAISMFNYKQDYSNPTCRIHAAVTGNVSTALNFTANANYAYISRCQGTDGKPILLQPGQMTNVACRSGVLALPSDADYFGYSFQGRTYYVGRKSYIPKTSEGDIQMVYVITPNYNKGPDTVCPLKDVSAAATSLDSLLGQCIDYDIHGVTGRGVFQVCNSTGIASQIFVYDGFGNIIGFHSKNGTYYCMAPCVSVPVSVIYDKSSGGSECALDVLFKGNAPIIANYSRRVFTNCNYNLTKLLSLVQVDEFVCHKTTPEALATGCYSSLTVDWFALPLSMKSTLAIGSAEAISMFNYNQDYSNPTCRIHATVTANVSSVMNFTADNNYAYISRCQGTDGKPILLQPGQMTNIACRSGVLGMQNDADYFGYSFQGRTYYIGRKSYKPKISDDVRSGDIQMVYVITPNYNKGPDTVCPLKDVSAASSSLDSLLGQCIDYDIHGVTGRGVFQVCNSTGIASQIFVYDGFGNIIGFHSKNGTYYCMAPCVSVPVSVIYDKSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 142 - protein, 593 aa, synthetic construct
ATMGILPSPGMPALLSLVSLLSVLLMGCVAETGTECALDVLFKGNAPIIANYSRRVFTNCNYNLTKLLSLVQVDEFVCHKTTPEALATGCYSSLTVDWFALPFSMKSTLAIGSAEAISMFNYNQDYSNPTCRIHATVTANVSTAMNFTAGANYAYISRCQGIDGKPILLQPGQTTNIACRSGVLGMKKDVDYFGYSFQGRIYYVGRKSYLPKTSEGDIQMVYVITPNYNKGPDTVCPLKDVSTASTSLDSLLGQCIDYDIHGVTGRGVFQVCNSTGIASQIFVYDGFGNIIGFHSKNGTYYCMAPCVSVPVSVIYDKNSGGGGSAEAAAKASSAEAAAKEAAAKEAAAKEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVKAMKGPFPNVKFVPTGGVDLDNVCEWFDAGVLAVGVGDALVEGDPDEVREKAKEFVEKIRGCTEGSLEGRSLEVLFQGPGWSHPQFEKGSGHHHHHHHH

SEQ ID NO: 143 - protein, 544 aa, synthetic construct
ECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYGGSEAAATGTFIEQPKSKECDFTPMLVGVPPQVYNFKRLVFTNCNYNLTKLLSLFMVNEFSCNGISPDAIARGCYSSLTVDYFAYPLSMRSYIQPGSAGDISLYNYKQSFANPTCRVLATAPANLTLTKPSAYGYFQKCSRVSGEHNSVETPLYINPGEYSICRSFSPYGFSEDGEVFRRQLTQYEGGGILVGVGAKLAMTDKLEMGFIISVQYGTDTNSVCPMLDLGNSSTITHYLGKCVDYSLYGVTGRGVFQNCTAVGIRQQRFVYDSFDNLVGYYSDDGN

SEQ ID NO: 144 - protein, 283 aa, synthetic construct
ASATGTFIEQPNATECDFSPMLTGVAPQVYNFKRLVFSNCNYNLTKLLSLFAVDEFSCNGISPDAIARGCYSTLTVDYFAYPLSMKSYIRPGSAGNIPLYNYKQSFANPTCRVMASVPANVTITKPDAYGYISKCSRLTGANQDVETPLYINPGEYSICRDFSPGGFSEDGQVFKRTLTQFEGGGLLIGVGTRVPMTDNLQMSFIIFVQYGTGTDSVCPMLDLGDSLTITNRLGKCVDYSLYGVTGRGVFQNCSAVGVKQQRFVYDSFDNLVGYYSDDGNYYC

SEQ ID NO: 145 - protein, 589 aa, synthetic construct
EAQPRGSFIEQAQGTECDFSPLLKDEPPQVYNFSRLVFTNCNYNLTKLLALFQVSQFSCHQVSPSALASGCYSSLTVDYFAYPTYMSSYLQQGSTGEISQFNYKQDFSNPTCRILATVPANLSASGLLPKPSNYVWLSECYQNSFTGKNFQYVKAGQYTPCLGLAANGFEKSYQTHRDPVSKLAVTGVVTPMTSALQMAFIISVQYGTDSNSVCPMQAVRNDTSVDDKLGLCIDYSLYGITGRGVFQNCTAVGIRQQRFVYDSFDNLVGYHADNGNYYCVRPCVSVPVSVVFDRSTNTHATLFGSGGSEASPRGMFVEQSTSEECDFTKMLTGTPPQVYNFNRMVFTNCNYNLTKLLNLFQVSEFSCYQVSPSSLATGCYSSLTVDSFAFPLELSSYMQTGSAGPIRQFNYNQDFSKPTCRVLASVPSNYTALSKPSNYVHLTECYTLGYSAKQYHNTQPGQYTPCLQLANQGFSRSGQSNSVELPSGEFKTATGYIIPMTDQLQMAFVISVQYGTDTNSVCPMQALRNDTSIQDKLDVCVEYSLHGVTGRGVFHNCTSVGLRNQRFVYDTFDNLVGYHSDNGNYYCVR

SEQ ID NO: 146 - protein, 280 aa, synthetic construct
EASPRGEFIEQATTQECDFTPMLTGTPPPIYNFKRLVFTNCNYNLTKLLSLFQVSEFSCHQVSPSSLATGCYSSLTVDYFAYSTDMSSYLQPGSAGEIVQFNYKQDFSNPTCRVLATVPQNLTTITKPSNYAYLTECYKTSAYGKNYLYNAPGGYTPCLSLASRGFSTKYQSHSDGELTTTGYIYPVTGNLQMAFIISVQYGTDTNSVCPMQALRNDTSIEDKLDVCVEYSLHGITGRGVFHNCTSVGLRNQRFVYDTFDNLVGYHSYNGNYYCVRPCVS

SEQ ID NO: 147 - protein, 565 aa, synthetic construct
EAHVYPDCNFTDLFRENAPTIMQYKRQVFTRCNYNLTLLLSLVQVDEFVCDKITPEALATGCYSSLTVDWFAFPYAWKSYLAIGSADRIVRFNYNQDYSNPSCRIHSKVNSSVGISYSGLYSYITNCNYGGFNKDDVVKPGGRASQPCVTGALNSPTNGQVWSFNFGGVPYRTSRLTYTDHLKNPLDMVYVITVKYEPGAETVCPKQVRPDYSTNITGLLGSCISYDIYGITGTGVFQLCNATGIPQQKFVYDKFDNIIGFHSDDGNYYCVAPCVSGGSSAKPVGAYYEAHVYPDCNFTELFRERAPTIMQYKREVFTRCNYNLSLLLSLVQVDEFVCDKITPEALATGCYSSLTVDWFAFPYAWKSYLAIGSADRIVRFNYNQDYSNPSCRIHSKVNSSIGISYAGAYSYITNCNYGATNKDDVVKPGGRASQQCITGALNSPTTGQLWAYNFGGVPYRVSRLTYTDHLSDPLDMVYVITVKYEPGAETVCPKQIRPDYSTNITHLLGSCISYDIYGITGTGVFQLCNATGIRQQKFVYDKFDNIIGFHSDDGNYYCVAPCVSV

SEQ ID NO: 148 - protein, 276 aa, synthetic construct
EAHVYPDCNFTDLFRENAPTIMQYKRQVFTRCNYNLTLLLSLVQVDEFVCDKITPEALATGCYSSLTVDWFAFPYAWKSYLAIGSADRIVRFNYNQDYSNPSCRIHSKVNSSVGISYSGLYSYITNCNYGGFNKDDVVKPGGRASQPCVTGALNSPTNGQVWSFNFGGVPYRTSRLTYTDHLKNPLDMVYVITVKYEPGAETVCPKQVRPDYSTNITGLLGSCISYDIYGITGTGVFQLCNATGIPQQKFVYDKFDNIIGFHSDDGNYYCVAPCVS

SEQ ID NO: 149 - protein, 576 aa, synthetic construct
ECELDVLFKNDAPIIANYSRRVFTNCNYNLTKLLSLVEVDEFICHKTTPEALATGCYSSLTVDWFALPFSMKSTLAIGSAEAISMFNYKQDYSNPTCRIHAAVTGNVSTALNFTANANYAYISRCQGTDGKPILLQPGQMTNVACRSGVLALPSDADYFGYSFQGRTYYVGRKSYIPKTSEGDIQMVYVITPNYNKGPDTVCPLKDVSAAATSLDSLLGQCIDYDIHGVTGRGVFQVCNSTGIASQIFVYDGFGNIIGFHSKNGTYYCMAPCVSVPVSVIYDKSSGGSECALDVLFKGNAPIIANYSRRVFTNCNYNLTKLLSLVQVDEFVCHKTTPEALATGCYSSLTVDWFALPLSMKSTLAIGSAEAISMFNYNQDYSNPTCRIHATVTANVSSVMNFTADNNYAYISRCQGTDGKPILLQPGQMTNIACRSGVLGMQNDADYFGYSFQGRTYYIGRKSYKPKISDDVRSGDIQMVYVITPNYNKGPDTVCPLKDVSAASSSLDSLLGQCIDYDIHGVTGRGVFQVCNSTGIASQIFVYDGFGNIIGFHSKNGTYYCMAPCVSVPVSVIYDKS

SEQ ID NO: 150 - protein, 285 aa, synthetic construct
ECALDVLFKGNAPIIANYSRRVFTNCNYNLTKLLSLVQVDEFVCHKTTPEALATGCYSSLTVDWFALPFSMKSTLAIGSAEAISMFNYNQDYSNPTCRIHATVTANVSTAMNFTAGANYAYISRCQGIDGKPILLQPGQTTNIACRSGVLGMKKDVDYFGYSFQGRIYYVGRKSYLPKTSEGDIQMVYVITPNYNKGPDTVCPLKDVSTASTSLDSLLGQCIDYDIHGVTGRGVFQVCNSTGIASQIFVYDGFGNIIGFHSKNGTYYCMAPCVSVPVSVIYDKNS

SEQ ID NO: 151-199 - skipped (49 entries)

SEQ ID NO: 200 - protein, 6 aa, synthetic construct
HHHHHH

SEQ ID NO: 201 - protein, 10 aa, synthetic construct
EQKLISEEDL

SEQ ID NO: 202 - protein, 8 aa, synthetic construct
DYKDDDDK

SEQ ID NO: 203 - protein, 9 aa, synthetic construct
YPYDVPDYA

SEQ ID NO: 204 - protein, 6 aa, synthetic construct
DTYRYI

SEQ ID NO: 205 - protein, 13 aa, synthetic construct
GAPVPYPDPLEPR

SEQ ID NO: 206 - protein, 26 aa, synthetic construct
KRRWKKNFIAVSAANRFKKISSSGAL

SEQ ID NO: 207 - protein, 19 aa, synthetic construct
TKENPRSNQEESYDDNESC

SEQ ID NO: 208 - protein, 15 aa, synthetic construct
KETAAAKFERQHMDS

SEQ ID NO: 209 - protein, 38 aa, synthetic construct
MDEKTTGWRGGHVVEGLAGELEQLRARLEHHPQGQREP

SEQ ID NO: 210 - protein, 9 aa, synthetic construct
AWRHPQFGG

SEQ ID NO: 211 - protein, 12 aa, synthetic construct
PDRVRAVSHWSS

SEQ ID NO: 212 - protein, 15 aa, synthetic construct
TDKDMTITFTNKKDA

SEQ ID NO: 213 - protein, 6 aa, synthetic construct
CCPGCC

SEQ ID NO: 214 - protein, 10 aa, synthetic construct
EVHTNQDPLD

SEQ ID NO: 215 - protein, 14 aa, synthetic construct
GKPIPNPLLGLDST

SEQ ID NO: 216 - protein, 11 aa, synthetic construct
YTDIEMNRLGK

SEQ ID NO: 217 - protein, 8 aa, synthetic construct
HHHHHHHH

SEQ ID NO: 218 - protein, 10 aa, synthetic construct
HHHHHHHHHH

SEQ ID NO: 219 - protein, 14 aa, synthetic construct
HHHHHHHHHHHHHH

SEQ ID NO: 220 - protein, 4 aa, synthetic construct
RRAR

SEQ ID NO: 221 - protein, 4 aa, synthetic construct
GSAS

SEQ ID NO: 222 - protein, 5 aa, synthetic construct
PRRAR

SEQ ID NO: 223 - protein, 4 aa, synthetic construct
SGAG

SEQ ID NO: 224 - protein, 7 aa, synthetic construct
GGSGGGS

SEQ ID NO: 225 - protein, 4 aa, synthetic construct
WSYT

SEQ ID NO: 226 - protein, 4 aa, synthetic construct
VSNG

SEQ ID NO: 227 - protein, 4 aa, synthetic construct
WFVT

SEQ ID NO: 228 - protein, 12 aa, synthetic construct
GSRSLEVLFQGP

SEQ ID NO: 229 - protein, 5 aa, unidentified
RRKRR

SEQ ID NO: 230 - protein, 5 aa, unidentified
GGSGS

SEQ ID NO: 231 - protein, 5 aa, unidentified
RRSRR

SEQ ID NO: 232 - protein, 5 aa, synthetic construct
GGSGG

SEQ ID NO: 233 - protein, 25 aa, synthetic construct
TNCIAPVNGYFIKNNTRIVDEWSYT

SEQ ID NO: 234 - protein, 19 aa, synthetic construct
TNCIAPVNGYFIKNNWSYT

SEQ ID NO: 235 - protein, 17 aa, Middle East respiratory syndrome-related coronavirus
YFIKTNNTRIVDEWSYT

SEQ ID NO: 236 - protein, 17 aa, unidentified
YFIKTNNTRIVDEWSYT

SEQ ID NO: 237 - protein, 15 aa, unidentified
YFVKNDSDSDSWSYT

SEQ ID NO: 238 - protein, 19 aa, unidentified
YFITNQTTTYSVDTEWYYT

SEQ ID NO: 239 - protein, 19 aa, unidentified
YFITNQTTTYSVDTEWYYT

SEQ ID NO: 240 - protein, 19 aa, unidentified
YFVLNSTTTRAADQEWYYT

SEQ ID NO: 241 - protein, 22 aa, unidentified
YFVLNQTISTARSSEDQQWYYT

SEQ ID NO: 242 - protein, 21 aa, unidentified
YFVLNQTTSTVADSDQQWYYT

SEQ ID NO: 243 - protein, 21 aa, unidentified
YFVLNQTISTVANSDQQWYYT